(19) United States
(12) Patent Application Publication
(10) Pub. No.: US 2018/0143198 A1
(43) Pub. Date: May 24, 2018

(54) METHOD FOR DETECTING DIFFERENTIALLY METHYLATED CPG ISLANDS ASSOCIATED WITH ABNORMAL STATE OF HUMAN BODY

(71) Applicants: PEKING UNIVERSITY, Beijing (CN); BEIJING SHIJITAN HOSPITAL, CAPITAL MEDICAL UNIVERSITY, Beijing (CN)

(72) Inventors: Lu WEN, Beijing (CN); Jirun PENG, Beijing (CN); Yanyi HUANG, Beijing (CN); Fuchou TANG, Beijing (CN); Xiaomeng LIU, Beijing (CN); Jingyi LI, Beijing (CN); Huahu GUO, Beijing (CN)

(73) Assignees: PEKING UNIVERSITY, Beijing (CN); BEIJING SHIJITAN HOSPITAL, CAPITAL MEDICAL UNIVERSITY, Beijing (CN)

(21) Appl. No.: 15/540,010

(22) PCT Filed: Dec. 26, 2014
(86) PCT No.: PCT/CN2014/095120

§ 371 Date: Jun. 26, 2017

(51) Int. Cl. G01N 33/574; C12Q 1/686; C40B 40/06

(52) U.S. Cl. CPC G01N 33/574; C12Q 1/686; C12Q 1/6806; C12Q 2600/154; C40B 40/06

(57) ABSTRACT

Disclosed is a method for detecting differentially methylated CpG islands associated with an abnormal state of a human body, characterized by detecting very minute amounts of methylated CpG short tandem nucleic acid sequences in highly fragmented DNA samples with genome scale, identifying differentially methylated CpG islands associated with abnormal state of human body and determining the corresponding abnormal state of human body. Sequencing libraries are constructed by using CpG short tandem sequences as primers to perform three steps of PCR reactions on DNAs which are conversed by nodifiers, and detections of very minute amounts of methylated CpG short tandem nucleic acid sequences are implemented with high throughput sequencing technology. A group of genome sequences and methylation patterns of differentially methylated CpG islands which are associated with hepatocellular carcinoma are also disclosed; they may be used for distinguishing between hepatacellular carcinoma and non-cancerous state.

### BACKGROUND OF THE INVENTION

### 1. Field of the Invention

The present invention relates to the field of biomedicine. Especially, the present invention is a method and a system to determine a human abnormal state such as cancer via DNA methylation information.

### 2. Description of the Related Art

DNA methylation is the modification of the cytosine (C) to the 5′-methylated-cytosine (5mC) by adding a methyl group to the C5 position of the cytosine, mainly occuring at the CpG site (CpG indicates dinucleotide of which the guanine (G) base immediately follows the cytosine base along the DNA strand). Most of the CpG sites were diffused distributed in the human genome and highly methylated. However, in some region of the genome, CpG dinucleotides show the expected or even higher frequency and these regions are referred to as CpG islands (about 30,000 in the human genome), withmost CpG islands being demethylated in the normal physical state. CpG islands enrich in gene promoters, with more than 60% of promoters contain a CpG island. Demethylation of the CpG islands promises the activity of gene transcription. And methylation of the promoter CpG islands leads to silencing of gene expression by recruiting methylation binding proteins. This mechanism participates in many physical biological processes, including X-chromosome inactivation and genomic imprinting (Jones and Baylin, Nat Rev Genet, 2002).

Aberrant methylation of CpG islands is common in human cancers. It not only occurs in nearly all common human cancerss, such as hepatocellular carcinoma, colon cancer, lung cancer, gastric cancer, breast cancer, prostatic cancer et al., but also occurs in the early stage of caner or even precancerous lesion, and relates to a great many genes (for reviews see Baylin and Jones, Nat Rev Cancer, 2011; Feinberg and Tycko, Nat Rev Cancer, 2004). In recent years, a lot of genomic studies show that thousands of CpG islands could be aberrantly hypermethylated in various types of tumors, and it has been estimated that on average hundreds of CpG islands can be aberrantly hypermethylated at the early stage of a cancer (Ammerpohl et al., Int J Cancer 2012; Costello et al., Nat Genet 2000; Hinoue et al., Genome Res 2012; Keshet et al., Nat Genet 2006). Aberrant hypermethylation of some CpG islands is closely related to prognosis and drug resistance of cancer. In addition, aberrant CGI hypermethylation is related to other human abnormal states such as systemic lupus erythematosus (Robertson. Nat Rev Genet, 2005). The human body fluid (including blood, urine, saliva and excretion, ect) contains the cell-free DNA. Detecting cell-free DNA in the human body fluid is a noninvasive way to obtain information about the human state, minimizing the hurt to the body that could occur when sampling the tissue. (Schwarzenbach et al., Nat Rev Cancer, 2011). Studies have shown that tumor DNA can be release to blood at the early cancer stage. Therefore, detecting the cell-free tumor DNA (ctDNA) shows great promises for detecting, screening and monitoring cancers in a non-invasive manner. Comparing with the mutation of tumor DNA, aberrant CpG island hypermethyaltion occurs more frequently and thus have more advantages in cancer screening (Heyn and Esteller, Nat Rev Genet, 2012; Schwarzenbach et al., 2011).

However, since ctDNA is scare in amount (at the level of pilgrims) and makes up only a minority of the total ccfDNA (0.1%-1%) and is highly fragmented (70-200 base pairs), dection of ctDNA is a technical challenge. (Schwarzenbach et al., Nat Rev Cancer, 201). Current reported techniques for detecting CpG island methylation in ctDNA, including restriction enzyme digestion, restriction enzyme digestion and PCR, methylation-specific PCR (Herman, et al., PNAS, 1999), pyrosequencing (Korshunova, et al., Genome Research), Methylight (Eads et al., Nucleic Acids Res, 2000), MethylBeaming (Li et al., Nat Biotechnol, 2009), have relatively high sensitivity and are suitable for detecting highly-fragmented DNAs. However, they are able to detect single or a small number of sites. On the other side, genome-wide DNA methylation methods (Laird, Nat Rev Genet, 2010), such as the Infinium methylation array (Bibikova et al., Genome Research, 2006) and reduced representation bisulfite sequencing (RRBS) (Meissner et al., Nature, 2008), have been successfully used for cell and tissue samples. Their applications to ctDNA, however, are hampered by technical limitations. For example, the Infinium methylation array requires more than 1 microgram DNA (Bibikova et al., Genome Research, 2006). RRBS is able to detect DNA of pilgrims, but includes a size selection step that is not suitable for severely fragmented ccfDNA (Gu et al., Nature Protocol, 2011). Direct detection of the ccfDNA by whole genome bisulfite sequencing has been reported, which is able to detect the more wide-spreading hypomethylation changes. However, as the depth of sequencing is relatively low (1× coverage per CpG island), it is hard to analysis CpG island hypermethylation (Chan, et al., PNAS, 2013).

Therefore, techniques for high-efficient detection of the human abnormal states related methylated CpG islands from extremely small amount of ccfDNA remain limited.

### SUMMARY OF THE INVENTION

Therefore, it is a primary objective of the present invention to provide a detachable movable device and an electronic device thereof to achieve the effect of reducing the tear and wear of a touch display panel of a general electronic device.

In a first aspect, the present invention provides a method for detecting differentially methylated genomic sites relating to human abnormal states, comprising the following steps:

(1) Obtaining human DNA samples.

The DNA sample can come from human cells, tissues, blood, body fluid, urine, excrement or their combination. Preferably, the DNA sample is the cell-free DNA of human plasma or urine. Cell-free DNA in peripheral blood mainly comes from blood cells; comparing with serum, there is less DNA released from blood cells in plasma, which could intensively reduce the noise comeing from the blood cell DNA. A large amount of ccfDNA could be flushed out by urine, so we could detect ccfDNA from urine. Comparing with plasma, urine analysis is a more non-invasive means for detection of ccfDNA.

(2) Genome-scale detection of methylation of the short CpG tandem sequences of the referred DNA samples

Genome-scale detection refers to detecting more than 100 CpG islands simultaneously. Comparing with the existing method of detecting CpG islands which can only detect one single or a small number of sites, our method can detect thousands of CpG islands. Thus, the present invention has a significant improvement over the prior art. The key to the improvement of present invention lies in the setting of short CpG tandem as the target. Short CpG tandem refers to the short DNA sequence (7-9 base pairs) which contains three or more than three CpG nucleotide pairs. These short CpG tandems have three characteristics: a) There is a large number of copies in the human genome. So they could be the targets for genome-scale detection. b) These short CpG tandem sequences are highly enriched in the CpG islands, so they contain important methylation information that can be used to determine the abnormal state of the human body. c) These short CpG tandem sequences have a relatively high melting temperature and can therefore be efficiently amplified. Taking CGCGCGG as an example (FIG. 1), out of all 23,726 CGCGCGG sequences used in this study, 20,525 (86.5%) are located within the 9,373 (34.2% of all 27,435) CGIs in the human genome. Its melting temperature is 43 degrees Celsius, which is close to the conventional PCR reaction annealing temperature. Therefore, CGCGCGG is a good target site for genome-scale detection of methylation level. For there is a large amount of CGCGCGG in human genome and it is enriched in the CpG islands, CGCGCGG is one of the most preferred short CpG tandem for genome-scale detection of methylation level. In addition to CGCGCGG there are other good choices for target sites, such as CGGCGGCGG (87.6% of the 11,276 CGGCGGCGG are located within CpG islands), CGCGGCGG (83.6% of the 12,322 CGCGGCGG are located within CpG islands), CGGCGCGG (80.8% of the 11,354 CGGCGCGG are located within CpG islands), CGCGGCGC (90.3% of the 9,947 CGCGGCGC are located within CpG islands), CGGCGCGC (86.6% of the 7,885 CGGCGCGC are located within CpG islands), CGCGCGC (60.7% of the 33,818 CGCGCGC are located within CpG islands), CGCGCGA (76.8% of the 5,923 CGCGCGA are located within CpG islands) and CGCGCGT (57% of the 10,553 CGCGCGT are located within CpG islands). Targeting several DNA short tandem simultaneously could further improve the detection range and the detection sensitivity. The guanines outside of CpG in the DNA short tandems improve the melting temperature and amplification efficiency. Professional staff in this field know that the specificity of hybridization and amplification of nucleic acid sequences is mainly dependent on its 3′end sequence, while the effect of 5′-end CpG nucleotide pair is gradually reduced. For short CpG tandem sequences, the first 3 nucleotides at 3′-end are the most important for hybridization, followed by 4 to 6 nucleotides, and then 7 to 9 nucleotides. There are eight preferred sequences for the 3′-end of primer, CGCGG, CGCGA, CGCGT, CGCGC, CGGCGG, CGGCGA, CGGCGT and GGCGC, in which CGCGG and CGGCGG are prefered, and CGCGG is the most prefered. Atypical short CpG tandem sequence such as GGCGCGG, which has less CpG nucleotides (2 CpG) than the typical one, could also be used for genome-scale detection of methylated CpG islands with lower detection efficiency. Longer short CpG tandem sequence (more than 9 base pairs) and more CpG nucleotides will further increase the melting temperature, however, with less targeting sites. As described above, CpG nucleotide at 5′-end of the primer has less effect on the primer specificity compared with nucleotide at 3′-end.

(3) Comparing the DNA methylation level of short CpG tandems described above in DNA samples mentioned above with those in DNA samples of normal individuals, we could identify the CpG islands with aberrant hypermethylation level in abnormal human body and,

(4) using the differential methylated CpG islands, we could diagnose the corresponding human abnormalities.

On one hand, the present invention can be applied to clinical research, large-scale analysis and identification of differential methylation CpG islands associated with abnormalities of the human body; on the other hand, it can be applied to clinical molecular testing, by identifying the differential methylated CpG island to predict if an individual has an abnormal state.

The existing DNA methylation assay, such as the Infinium methylation chip and RRBS, can be used to analyze the differential methylated CpG islands associated with abnormal human status in tissue or cell DNA samples, but the results can not be directly converted into clinical application of free DNA in clinical molecular testing. The advantage of the method of the present invention is that it is possible to directly identify a large amount of differential methylated CpG islands associated with the abnormal state of the human body in the cell free DNA sample, and thus the results can be directly applied in clinical testing.

Tumor is one of the important applications of the present invention. According to one example of the present invention, methylation level of 132 CpG islands were identified to be significantly differentiated between hepatocellular patients and non-tumor individuals by analysis of the cell free DNA of hepatocellular patients and non-tumor individuals. The sequences of 132 CpG are as below (1˜132). Aberrantly methylated CpG islands suggest that these individuals suffer from hepatocellular carcinoma. 132 aberrantly methylated CpG islands can be divided into two groups: the first is type I markers (68, no. 1˜68), the second is type II markers (64, no. 69˜132). The type I markers are differential methylated between patients and normal people in both tissue and plasma, however, type II markers are only differential methylated in plasma.

The correlation between some of the type I markers and hepatocellular carcinoma has been reported (Shen, et al., 2012, Hepatology; Ammerpohl, et al., 2012, Int J Cancer; Song, et al, 2013, PLoS One). However, the existing method cannot tell which of them also showed differential methylation between hepatocellular carcinoma patients and normal individuals in cell-free DNA samples. The type I CpG islands markers disclosed by the present invention correlates the cell-free DNA methylation detection and whether the individual is suffering from hepatocellular carcinoma directly.

The relationship between differential methylated CpG islands (type II markers) in plasma and hepatocellular carcinoma was not previously revealed, since the difference in methylation level of type II markers between hepatocellular carcinoma patients and non-cancerous individual lie only in plasma instead of tissues. This is because that Type II markers are hypermethylated in normal liver tissues, but hypomethylated in white blood cells as well as in cell-free DNA from plasma of healthy individuals. These hypermethylated loci appear to be contained in liver cells under normal circumstances but are released into the blood when malignance occurs together with the necrosis and apoptosis of tumor cells, thus these loci in the cell-free plasma sample are detected as hypermethylated ones. Besides HCC, it could be possible to detect hypermethylated type II markers in cell-free DNA under other circumstances of liver damage. Therefore, the result that hypermethylation of the type II differentially methylated CpG islands in cell-free DNA from plasma disclosed in the present invention may imply that the individual in the testing is likely to suffer from HCC or other aberrant condition of liver damage.

A group of CpG islands (no. 133˜187) with low methylation level in noncancerous individuals are included in this invention. This group of CpG islands (belong to tissue differential methylation CpG island), although are not differentially methylated between plasmas from hepatocellular carcinoma patients and noncancerous individuals, they are aberrantly hypermethylated in tumor tissue with certain frequency. It has a low methylation background (uMePM<0.1 in non-cancerous individuals) and high methylation detection efficiency (mean of MePM in FMG>75), making it widely used to detect early neoplastic diseases.

Secondly, this invention provides a method for detecting the level of methylation of the short CpG tandem nucleic acid sequence on a genomic scale, which is Methylated CpG Tandems Amplification and Sequencing (MCTA-Seq). As shown in FIG. 2, it includes the following steps:

Step one, treating a DNA sample with a modifying agent to form the modified DNA wherein cytosine bases instead of 5′-methyl-cytosine bases of the DNA sample are modified to uracil bases.

The agent for treating the DNA sample modifies cytosine bases but not 5′-methyl-cytosine bases followed by the formation of single-stranded DNA. The modifying agent can be selected from bisulfite, acetate or citrate. Preferably, the agent is bisulfite. Optionally, bisulfite treatment of the DNA sample can be achieved by using commercial kits such as MethylCode Bisulfite Conversion Kit (Invitrogen) ` EZ DNA methylation-Gold Kit (ZYMO) or EpiTect Bisulfite Kit (Qiagen).

Step two, providing Primer A and DNA polymerase to the modified DNA to allow for at least one round of linear amplification to form the semi-amplicon capable of anchoring Adapter Primer C at one end.

The primer A is composed of two portions: a 3′ end and a 5′ end. The 3′ end is used for binding and amplifying the converted DNA fragments, which is characteristic for a range of random sequences capable of binding the converted DNA fragments. Preferably, the 3′ end contains only C, A and T except the CpG dinucleotide. Since the majority of C are converted to U after treatment of modifying agent, this design may improve the binding efficiency. Preferably, the first 7 nucleotides at 3′ end contains at least 1 nucleotide pair. On one hand, due to the conversion of CpG in non-methylated CpG island to UpG by the modifying agent, this design allows preliminary enrichment of the methylated CpG islands. On the other hand, CpG may increase the melting temperature of primers and improve the amplifying efficiency. More preferably, the second nucleotide at the 3′ terminal is C. On one hand this may increase the melting temperature of primers and improve the amplifying efficiency. On the other hand since the 3′ end of the primers contains only C, A and T, except CpG, the C as the second nucleotide at the 3′ terminal may limit the formation of dimers. Preferably, the portion between the 3′ and 5′ end of the primer A contains a unique molecular identifier. This sequence add a tag to each amplicon before the exponential amplification in step four, allowing for the identifying and reducing the product of PCR over-amplification. Preferably, the sequence of the unique molecular identifier is HHHHH (H=A, T or C), identifying 243 different molecules.

The 5′ end of primer A is used to anchor Adapter Primer C; it allows Adapter Primer C to bind to its reverse complementary sequence for PCR amplification. The word “anchor” describes the function of the 5′ end to join Adapter Primer C to the amplicon via PCR.

The DNA polymerase can be any suitable polymerase, such as Taq polymerase, ExTaq polymerase, LATaq polymerase, AmpliTaq polymerase, Amplitaq Gold polymerase, Titanium Taq polymerase, KlenTaq polymerase, Platinum Taq polymerase, Accuprime Taq polymerase, Pyrobest DNA polymerase, Pfu polymerase, Pfu polymerase turbo, Phusion polymerase, Pwo polymerase, Vent polymerase, Vent Exo-polymerase, Sequenase TM polymerase, 9° Nm DNA polymerase, Therminator DNA polymerase, Expand DNA polymerase, rTth DNA polymerase, DyNazyme™ EXT polymerase, DNA polymerase I, T7 DNA polymerase, T4 DNA polymerase, Bst DNA polymerase, phi-29 DNA polymerase, and Klenow fragment.

Preferably, the DNA polymerase is capable of strand displacement. Since one single DNA template may bind several primer A, especially in CpG island regions with high CpG density, the DNA polymerase capable of strand displacement may enable the primer at 5′ end to extend, resulting in linear amplification even with one-time amplification. It can be any suitable polymerase with the strand displacement activity, including but not restricted to DNA polymerase I (Klenow) large fragment (New England Biolabs (NEB) Cat. No.: M0210S), Klenow fragment (exo-) (NEB Cat. No.: M0212S), Bst DNA polymerase large fragment (NEB Cat. No.: M0275S), Vent(exo-) (NEB Cat. No.: M0257S), Deep Vent (exo-) (NEB Cat. No.: M0259S), M-MulV reverse transcriptase (NEB Cat. No.: M0253S), 9.Nm DNA polymerase (NEB Cat. No.: M0260S) and Phi-29 DNA polymerase (NEB Cat. No.: M0269S). Preferably, the DNA polymerase is deficient of exonuclease activity. In one preferred embodiment, the DNA polymerase is Klenow fragment (exo-).

Linear amplification refers to that the amount of amplified products increase in a linear instead of an exponential relationship to the amplification times. As mentioned above, the DNA polymerase capable of strand displacement may enable linear amplification even with one-time amplification. 2-30 rounds of linear amplification are alternative. Many DNA polymerase with activity of chain replacement such as Klenow fragment are inactivated during DNA denaturation and need to be re-added.

Step three, amplifying the semi-amplicon using Primer B and DNA polymerase to form the full-amplicon enriched with methylated CpG islands and capable of anchoring Adapter Primer C and D separately at both ends;

Primer B is composed of a 3′ end and a 5′ end. The 3′ end allows for selective amplification of the methylated CpG tandem sequences, which is the stated Short CpG tandem sequences. Technical personnel in this field knows that the CpG or CpG tandem sequence located at the 3′end of the primer is easy to form a primer dimer. In order to avoid the formation of primer dimers, the first nucleic acid at the 3′end of the short CpG tandem nucleic acid sequence primer is designed as A or T. Preferably, the 3′and 5′ end portions contain a random sequence of 2 to 10 nucleotides in length. Preferably, the 3′ portion of Primer B contains only G, A and T. Random sequence can help increase the melting temperature and it only needs to contain G, A, T, for most of C has been converted to U after treated with modifying agent and in the complementary strand, most G have been converted into A. Take CGCGCGG as an example, its melting temperature increase from 43° C. to 53° C. (AAACGCGCGG or TTTCGCGCGG) or 66° C. (GGGCGCGCGG) when adding 4 D (D=G/A/T) at 5′ end of primer, thus it is able to effectively amplify at conventional annealing temperatures. The 5′ end of Primer B is used to anchor Primer D. It allows Adapter Primer D to bind to its reverse complementary sequence for PCR amplification.

The polymerase can be any suitable polymerase mentioned above. Preferably, the polymerase is hot-start. Technical personnel in this field can understand that hot-start DNA polymerase increases the specificity of PCR reaction. Especially, it prevents the formation of dimers in primers with the short CpG tandems before amplification.

Step four, amplifying the full-amplicon exponentially using Adapter Primer C, Adapter Primer D and DNA polymerase to form the final-amplicon via PCR.

The word “Adapter Primer” herein refers to that the function of the primer is similar to the “adapter” used in conventional methods for constructing the high-throughput sequencing library (such as Illumnia's TruSeq DNA Sample Prep Kit and Applied Biosystems (ABI)'s The SOLiD™ Fragment Library Construction Kit), which allows for binding of the DNA fragments to the flow cell for subsequent amplification and sequencing. Different from the usage of ligation reaction to add “adapter” in conventional methods for establishing high-throughput sequencing library, the present invention adds “adapters” to each end of the amplicons by means of PCR reaction via the anchor sequences at the 5′ end of Primers A and B. Technical personnel in this field can understand that either Primer C or Primer D can contain “barcode” sequences, which facilitate to simultaneously sequence multiple libraries in one flow cell. The Primer C and D correspond to the adapter sequences of a given high-throughput sequencing platform, which includes, but not limited to, the Illumina's Genome Analyzer IIx, HiSeq and MiSeq platforms, ABI's SoLiD, 5500 W Series Genetic Analyzer, Ion Torrent PGM platforms, Roche454's GS Junior and GS FLX+ platforms.

Step five, separating and purifying the final-amplicon to form the library for high-throughput sequencing, then sequencing the library and analyzing the data.

The approach to separate and purify the final amplicons can be any suitable method, including but not restricted to magnetic beads-based, column-based and gel electrophoresis-based purification. Preferably, the purification method is able to achieve size-selection for the amplicons. Preferably, the amplicons are separated using 3%-4% agarose gel electrophoresis and the fragments between 160 and 250 bp are excised and then purified.

The high-throughput sequencing platform for analysis includes, but not limited to, the Illumina's Genome Analyzer IIx, HiSeq and MiSeq platforms, ABI's SoLiD, 5500 W Series Genetic Analyzer, Ion Torrent PGM platforms, Roche454's GS Junior and GS FLX+ platforms.

The method for data analysis is not limited and can be any suitable software for data analysis and sequence alignment, which includes, but not limited to Bismark, BSMAP, Bowtie, SOAP and R packages.

Thirdly, the present invention provides a kit for detecting methylated CpG islands using high-throughput sequencing. The kit comprises a set of primers including the Primer A, Primer B, Adapter Primer C, Adapter Primer D, and the DNA polymerases, as well as instructions for the kit.

Fourthly, the present invention provides a method for detecting hepatocellular carcinoma including the following steps: 1) The DNA sample can derive from human cells, tissues, blood, body fluid, urine, saliva, excrement or their combination. Preferably, the DNA sample is the cell-free DNA of human plasma or urine. 2) Nucleic acid sequence no. 1˜68 is the type I markers hypermethylated in the plasma of hepatocellular carcinoma and nucleic acid no. 69˜132 is the type II markers hypermethylated in the plasma of hepatocellular carcinoma. If aberrant methylation of type I marker or type II marker or both of them is detected in the plasma of an individual, it indicates that the individual may have hepatocellular carcinoma. The methods for detecting methylation level include, but are not limited to MCTA-Seq, methylation-specific PCR, restriction enzyme digestion and PCR, pyrosequencing, Methylight and MethylBeaming et.al.

### BRIEF DESCRIPTION OF THE DRAWINGS

FIG. 1. The number of various CpG tandems in human genome and the proportion in CpG islands were shown.

FIG. 2A and FIG. 2B. Schematic of the MCTA-Seq for detecting methylated CpG islands based on the high-throughput sequencing. The underscore part of primer A indicates the unique molecular recognition tag. \textcircled{1}, \textcircled{2} and \textcircled{3} indicate different barcode sequences. Note that different molecules have different amplification sites.

FIG. 3. Gel results of MCTA-Seq. The MCTA-Seq results of the genomic DNA extracted from WBCs (lane1) and the DNA sonicated to an average size of 200 bp (lane 2) were shown. M:marker, 20 bp ladder, TAKARA. bp: base pair. NTC: no template control.

FIG. 4. Fragment Analyzer result of the sequencing library constructed by MCTA-Seq.

FIG. 5. Correlation between short CpG tandem and MCTA-Seq sequencing results. X axis indicates CpG island with different number of CGCGCGG tandems, Y axis indicate the methylation detection values of human fully methylated genome (MePM: methylated alleles per million mapped reads, refers to average methylated alleles per million mapped reads). The sequence logos of the starting 3^{rd}˜13^{th} nucleotide of read2 are shown (black box indicates the CGCGCGG sequence, the 5^{th}˜11^{th} nucleotide). Bits: degree of enrichment of the sequence.

FIG. 6. The throughput and reproducibility of MCTA-Seq. The MCTA-seq result of two technical duplicates (X axis and Y axis) of fully methylated human genome standard sample is shown in the figure. The total number of CGIs detected in FMGs (average MePM>8 in two replicates) and Pearson correlation coefficient (r) between two replicates are shown.

FIG. 7. The number of CpG islands detected in different kind of samples. FMG: fully methylated human genomic DNA; HePG2: human liver cancer cell line; Hela: Human cervical cancer cell line; WBC: human white blood cells. The error bars represent means±s.d

FIG. 8. MCTA-seq result of CDKN2A (CYCLIN-DEPENDENT KINASE INHIBITOR 2A) gene region. The genomic region of the CDKN2A gene contains 4 CpG islands, including one promoter CGI (p), two intragenic CGIs (i1 and i2) and CGCGCGG tandems showed with triangles. The intragenic CGI i2 contains two CGCGCGG tandems (triangles above and below the rectangle indicate the CGCGCGG tandems positioned at the forward and the reverse strands, respectively). The scale is MePM, indicating all MCTA-seq reads of HePG2, Hela and WBC.

FIG. 9. Reproducibility of MCTA-Seq and differential methylation detection. Comparison of methylation profiles between technical duplicates of WBCs (a) and HePG2 cells (b) shows high reproducibility of MCTA-Seq. (c) Comparison of methylation profiles between WBCs and HePG2 cells shows differential methylation between two cell types, mainly the hypermethylation sites in HePG2.

FIG. 10. Heatmap showing the sensitivity of MCTA-Seq. The figure below shows different amounts of FMG diluted into the WBC. Each experiment has two replicates. Right of the figure shows the promoter or intragenic CGI (CDKN2A(i2)) In the heat map, black color indicates low and white indicates high DNA methylation values [Log 2(MePM)].

FIG. 11. MCTA-seq result of VIM (VIMENTIN) gene region. The grey box indicates the promoter CGI of the VIM (no. 2). The triangles above and below indicate two CGCGCGG sequences positioned at the forward and the reverse strand, respectively. M(%): the frequency of methylation, GE: the haploid genomic equivalent. Rep 1: duplicate 1; Rep2: duplicate 2; Reads: sequencing reads; UMIs: unique molecular identifier. The arrow indicates the shortest amplicon (30 bp).

FIG. 12. The MePM scores of the promoter CGI of VIM were plotted against the varying percentage of FMG to WBC; a linear fit was observed. The error bars represent means±s.d.

FIG. 13. MCTA-seq result of SEPT9 (SEPTIN9) gene region. The grey box indicates the promoter CGI of the SEPT9 gene. The triangles above and below indicate two CGCGCGG sequences positioned at the forward and the reverse strand, respectively. M (%): frequency of methylation; GE: genomic equivalent; rep1: duplicate 1; rep2: duplicate 2; Reads: sequencing reads; UMIs: unique molecular identifier.

FIG. 14. The MePM scores of the promoter CGI of SEPT9 were plotted against the varying percentage of FMG to WBC; a linear fit was observed. The error bars represent means±s.d.

FIG. 15. Unique molecular identifiers reduce amplification bias. Scatterplots showing the replicates of the 7.5 pg experiments using read counts (MePM, a) and UMI-adjusted counts (uMePM, b). uMePM: unique methylated alleles per million mapped reads.

FIG. 16. Principal component analysis of HCC tissues.

FIG. 17. Differentially methylated CGIs in tissue samples. Volcano plot for differentially methylated CGIs between the HCCs and the adjacent noncancerous liver tissues. The x axis shows the fold changes of the average methylation value between the HCCs (n=27) and the adjacent tissues (n=27), and the y axis shows the q-value as the FDR analogue of the P value (−Log 10(q-value)) for a two-tailed t-test of differences between two groups. A total of 1,605 CGIs (tdmCGls) were differentially hypermethylated in HCC tissues (q<0.05, HCC vs adjacent tissues>2).

FIG. 18. Number of methylated CpG islands. Boxplot (left) show the number of CpG islands under different threshold in HCC plasma (P(HCC), n=17), cirrhosis plasma (P(Ci), n=11) and healthy individual plasma (P(N), n=9). And the boxplot (right) show the number of CpG islands under different threshold in HCC tissues (T(HCC), n=27), adjacent tissues (T(Aj), n=27) and normal liver tissues (T(NL), 3). Two-tailed MWW.

FIG. 19. Differential methylated CpG islands in plasma. Volcano plot showing CGIs in plasma that are differentially methylated between the HCC patients with small tumors (n=13, the largest tumor diameter<5 cm) and cancer-free individuals (n=20, including cirrhosis patients and healthy controls). The x axis shows the fold changes of the average methylation between the HCC patients and the cancer-free individuals, and the y axis shows the q-value as the FDR analogue of the P value (−Log 10(q-value)) for a two-tailed t-test of differences between two groups. A total of 635 differentially methylated plasma CGIs are identified (HCC patients vs cancer-free individuals>2, q<0.05).

FIG. 20. Venn plot view comparing plasma differentiated CGIs and tissue differentiated CGIs. Type I markers (376) are CpG islands that show differential methylated level between cancer patients and cancer-free individuals both in plasma and tissues. Type II markers (259) are CpG islands that show differentially methylated level only in plasma.

FIG. 21. Classification of type II markers. The clustering shows that 102 CpG islands, called type IIa markers, show high methylation level both in adjacent tumor tissues and normal liver tissues but low methylation level in WBC. The rest of the type II markers are type IIb markers. White color indicates high methylation level, black color indicates low methylation level. Due to color limitation only part of the CpG islands are shown.

FIG. 22. Differential methylaion level of type IIa markers. Results of RRBS show that 94 IIa markers could be detected by RRBS and they show hypermethylation in normal tissues (T(NL)) and hypomethylation in WBC. Two-tailed MWW.

FIG. 23. Results of CpG island in SHANK2 (i2). Take the second CpG islands in SHANK2 gene (SHANK2 (i2), no. 79), a typical type IIa marker, as an example. (a) Results of RRBS, T (NL): normal liver tissues, two-tailed t-test, the error bars represent means±s.d. (b) Results of MCTA-Seq, T(HCC): tumor tissue from hepatocellular patients; T(Aj): tumor adjacent tissues. (c) Results of MCTA-Seq in plasma samples, P(HCC): plasma from hepatocellular patients; P(Ci): plasma from cirrhosis patients; P(N): plasma from healthy individuals.

FIG. 24. Example of high-performance type I markers. Boxplots and roc curve show four representative type I markers out of 68 type I markers (AUC>0.9) including CpG island located in the promoter of VIM (no. 2), CpG island located in the promoter of RIMS (no. 64), CpG island located in the promoter of TBX2 (no. 25), CpG island located in the intragenic region of KCNQ4 (no. 38).

FIG. 25. Example of high-performance type II markers. Boxplots and roc curve show four representative type II markers out of 64 type II markers (AUC>0.9) including CpG island located in the promoter of APAK2 (no. 130), CpG island located in the SH3PXD2A (no. 70), CpG island in the IGF2 (no. 75) and CpG island located in the promoter of APBB2 (no. 116).

FIG. 26. Roc curve of CpG island in SEPT9 promoter and boxplot showing the methylation value in plasma and tissue samples.

FIG. 27. Cancer detection by combination of 132 high-performance CpG islands. Boxplot showing the number of positive CpG islands with MePM exceeding the 90% percentile of the normal people. Roc curve shows the specificity and sensitivity of the detection method.

FIG. 28. Multiple sites detection of hepatocellular carcinoma. There are 3 indicators: 1) the number of loci passing the 90% specificity threshold (upper panel), 2) the uppermost number of standard deviations (SDs) above the 90% specificity threshold (refer to Top M-score, middle panel), and 3) the total number of SDs above the 90% specificity threshold (refer to Total M-score, bottom panel). Those indicators were detected in 68 type I markers (left) and 64 type II markers (right). A threshold was set (dashed lines for each indicators which could distinguish hepatocellular carcinoma patients and cancer-free individuals. “Positive” was defined as methylation level exceeding threshold. To get high specificity, 5 or 6 of the 6 indicators are positive. To get high sensitivity, 2 or 3 of the 6 indicators are positive, in which as least one type I is positive. P(HCC): plasma of hepatocellular carcinoma patients, case 1˜17 are training group, sorted according to tumor size: 1(P50), 2(P29), 3(P41), 4(P3), 5(P37), 6(P57), 7(P62), 8(P77), 9(P10), 10(P39), 11(P60), 12(P36), 13(P44), 14(P59), 15(P74), 16(P58), 17(P51); case 18˜27 are testing group, sorted according to tumor size: 18(P92), 19(P119), 20(P109), 21(P101), 22(P114), 23(P85), 24(P115), 25(P80), 26(P81), 27(P113). P(ci): plasma of cirrhosis patients, cases 28˜38 are training group, cases 39˜44 are testing group. P(N): plasma of healthy individuals, cases 45˜53 are training group, 54˜72 are testing group.

FIG. 29˜FIG. 39 MCTA-Seq results of 132 high performance type I and type II CpG island markers in 72 studied plasma cases. Ranking of study objects is the same as in FIG. 28.

FIG. 40 Total M score has a linear relationship with the tumor size. Total M score of type I (left) and type II (right) markers have linear relationships with maximum diameter of tumor (formula and correlation were shown).

FIG. 41 Comparing the performance of ccfDNA methylation classifiers and AFP. Heatmap shows MCTA-Seq results from plasma of hepatocellular carcinoma patients (n=27) and cirrhosis patients (n=17). The heatmap above shows M score of 68 type I markers and 64 type II markers. The heatmap below shows 3 indicators of type I and type II markers.

FIG. 42. The genomic region of the CDKN2A could be detected by different short CpG tandems. p: CpG island in promoter of CDKN2A; i1 and i2: CpG island in CDKN2A; triangle: CGCGCGG short tandem sequence; arrow: CGGCGGCGG short tandem sequence.

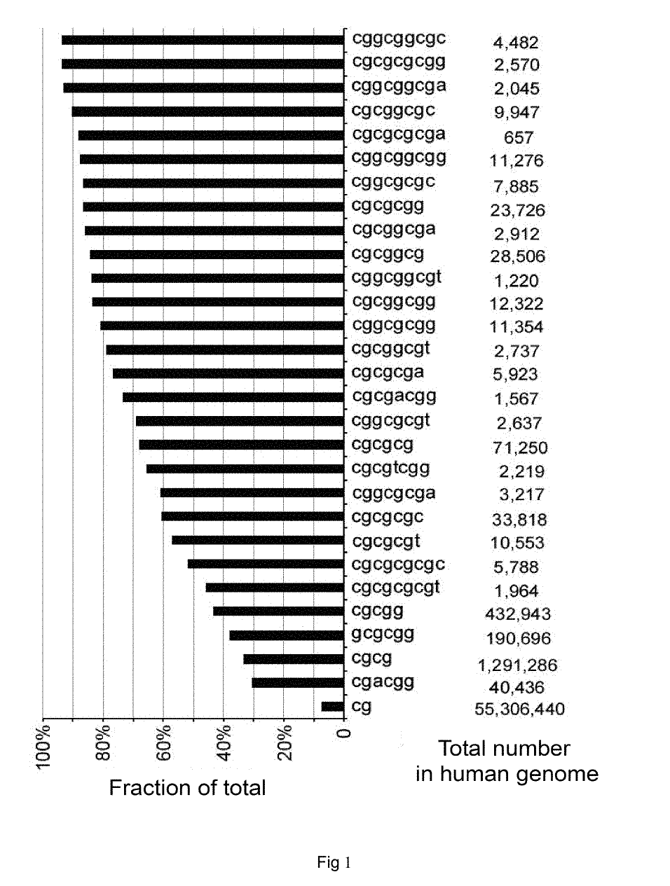

### DETAILED DESCRIPTION OF THE PREFERRED EMBODIMENTS

In order that the present invention may be more clearly understood, preferred forms will described with reference to the following examples and drawings. It will be appreciated by persons skilled in the art that numerous variations and/or modification may be made to the invention as shown in the specific embodiments without deportioning from the spirit or scope of the invention as broadly described.

### EXAMPLE 1

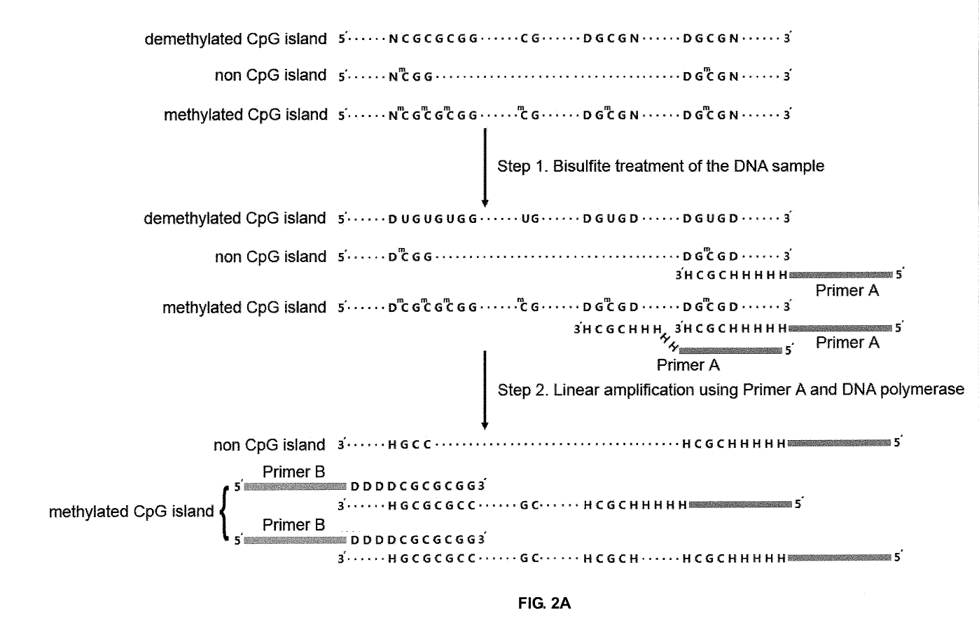

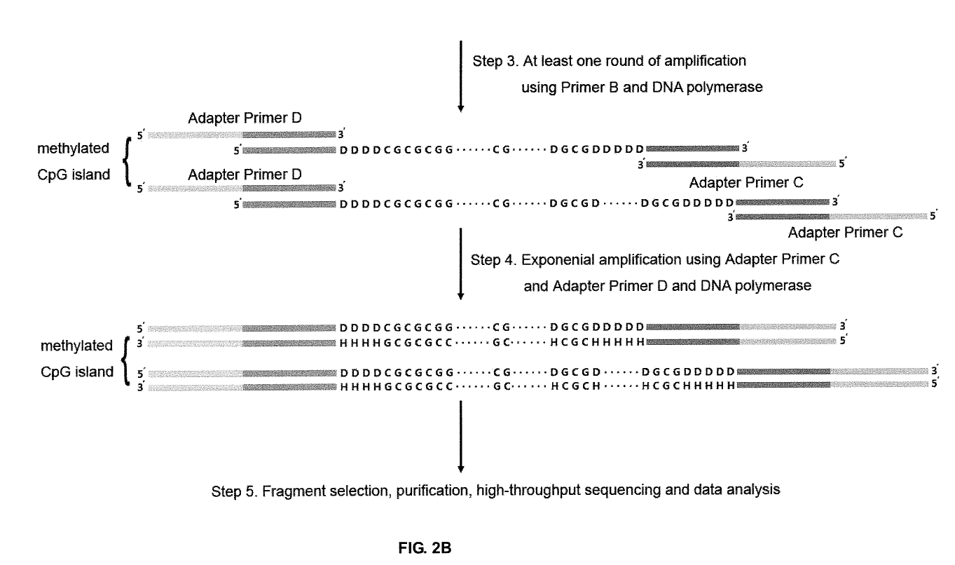

### The Methylated CpG Tandems Amplification and Sequencing (MCTA-Seq) According to the Present Invention Operates in the Following Manners (FIG. 2)

Bisulfite conversion of the DNA sample

The bisulfite conversion is performed by using the MethylCode™ Bisulfite Conversion Kit (Invitrogen) according to the protocol provided by the manufacturer. Detailed steps are as follows:

1.1 Preparing the CT conversion reagent: Add 900 μl of sterile distilled water, 50 μl of resuspension buffer, and 300 μl of dilution buffer directly to one tube of CT conversion reagent; mix by shaking or intermittent brief vortexing for 10 minutes for dissolvation; keep protected from light at room temperature;

1.2 Add 20 μl of the DNA sample ranging from 500 pg to 500 ng to a PCR tube;

1.3 Add 130 μl of CT conversion reagent to the DNA sample, and mix by flicking the tube or pipetting up and down;

1.4 Place the tube in a thermal cycler and run the following program: 98° C. for 10 minutes, 64° C. for 2.5 hours and 4° C. storage for no more than 20 hours;

1.5 Place a spin column in a collection tube and add 600 μl of binding buffer to the column.

1.6 Add the sample from step 1.4 to the binding buffer in the column; close the cap and mix by inverting several times.

1.7 Centrifuge at full speed (≥10,000×g) for 30 seconds, then discard the flow-through;

1.8 Add 100 μl of wash buffer prepared with ethanol to the column, and centrifuge at full speed for 30 seconds; discard the flow-through;

1.9 Add 200 μl of desulphonation buffer to the column and let the column stand at room temperature for 15-20 minutes;

1.10 Centrifuge at full speed for 30 seconds; discard the flow-through;

1.11 Add 100 μl of wash buffer with ethanol to the column and centrifuge at full speed for 30 seconds; discard the flow-through;

1.12 Repeat the wash in step 1.11 one more time, then transfer the spin column to a new, clean 1.5-ml microcentrifuge tube;

1.13 Add 10 μl of elution buffer directly to the column matrix; centrifuge at full speed for 30 seconds to elute the DNA.

2. Lineal amplification by means of Primer A and DNA polymerase.

2.1 Prepare the following reaction mixture in a PCR tube for the DNA sample obtained from step 1.13.

| Contents | Volume |
|---|---|
| The DNA sample (from step 1.13) | 10.5 μl |
| and water | |
| NEBuffer 2 | 1.5 μl |
| dNTP (2.5 mM) | 1.5 μl |
| Primer A (5 uM)* | 1 μl |
| Klenow (exo-)** | 0.5 μl |
| Total | 15 μl |

*:Primer A: An equimolar mixture of the following four primers. The total concentration is 5 μM:
5′-TTTCCCTACACGACGCTCTTCCGATCTHHHHHHHHCGCH-3′;
5′-TTTCCCTACACGACGCTCTTCCGATCTHHHHHHHCGHCH-3′
5′-TTTCCCTACACGACGCTCTTCCGATCTHHHHHHCGHHCH-3′,
5′-TTTCCCTACACGACGCTCTTCCGATCTHHHHHCGHHHCH-3′;
(H = A/T/C), the 3′ portion of Primer A randem sequence is underlined by a wave line, the 5′ portion of anchor sequence is underlined by a straight line, and the unique molecular identifier is underlined by a double line.
**:add at step 2.3

2.2 Place the tube in a thermal cycler and run the following program: 95° C. for 2 minutes and 4° C. for pause;

2.3 Centrifuge at 13000 rpm for a minute in 4° C.;

2.4 Add 0.5 μl of Klenow Fragment (exo-) (NEB catalog: M0212S), mix and briefly centrifuge;

2.5 Centrifuge at 13000 rpm for a minute in 4° C.;

2.6 Place the tube in a thermal cycler and run the following program: 4° C. for 50 seconds, 10° C. for a minute, 20° C. for 4 minutes, 30° C. for 4 minutes, 37° C. for 4 minutes;

2.7 Run the following program to inactivate the Klenow Fragment: 75° C. for 20 minutes and 4° C. for pause;

2.8 Centrifuge at 13000 rpm for a minute in 4° C.;

3. Amplification by means of Primer B and DNA polymerase.

3.1 Prepare the following reaction mixture in a new PCR tube:

| Contents | Volume |
|---|---|
| Ex Taq Buffer | 0.5 μl |
| Primer B (10 uM)* | 0.5 μl |
| HS Ex Taq (Takara catalog: DRR006B) | 0.3 μl |
| Water | 3.7 μl |
| Total | 5 μl |

*: Primer B: 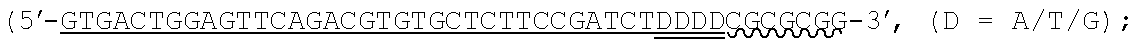
the 3′ portion of short CpG tandem sequence is underlined by a wave line, the 5′ portion of anchor sequence is underlined by a straight line and the random sequence is underlined by a double line.

3.2 Add the mixture above to the products (the semi-amplicons) from step 2.8, vortexing.

3.3 Centrifuge at 13000 rpm for a minute in 4° C.;

3.4 Place the tube in a thermal cycler and run the following program: 95° C. for 3 minutes, 50° C. for 2 minutes, 72° C. for 1 minute, 4° C. for pause;

3.5 Centrifuge at 13000 rpm for a minute in 4° C.;

4. Exponential amplification using Adapter Primer C, Adapter Primer D and DNA polymerase.

4.1 Prepare the following reaction mixture in a new PCR tube:

| Contents | Volume |
|---|---|
| Ex Taq Buffer | 3 μl |
| Adapter Primer C (100 uM)* | 0.6 μl |
| Adapter Primer D (100 uM)** | 0.6 μl |
| dNTP (2.5 mM) | 3 μl |
| HS Ex Taq (Takara catalog: DRR006B) | 0.3 μl |
| Water | 22.5 μl |
| Total | 30 μl |

*:Adapter Primer C:
5′-AATGATACGGCGACCACCGAGATCTACACTCTTTCCCTACACGACGCTCTTCCGATCT-3′; the underlined oligonucleotide sequence is the same as the 5′ portion of Primer A;
**:Adapter Primer D:
5′-CAAGCAGAAGACGGCATACGAGATCTGATCGTGACTGGAGTTCAGACGTGTGCT-3′, the underlined (by a straight line) oligonucleotide sequence is the same as the 5′ portion of Primer B and the barcode sequence (corresponding to the Illumina index 9) is underlined by a double-line;

4.2 Add the mixture to the product (full-amplicons) from step 3.5, vortexing;

4.3 Centrifuge at 13000 rpm for a minute in 4° C.;

4.4 Place the tube in a thermal cycler and run the following program: 95° C. for 3 minutes;

4.5 Run the following program: 17 cycles of 95° C. for 30 seconds, 65° C. for 30 seconds, 72° C. for 1 minute, and then 4° C. for storage.

5. Size-selection, purification, high throughput sequencing and data analysis.

5.1 Prepare 4% agarose gel, add 1×SYBR Safe (Invitrogen);

5.2 Run the products (final-amplicons) from step 4.5 in the agarose gel by electrophosis;

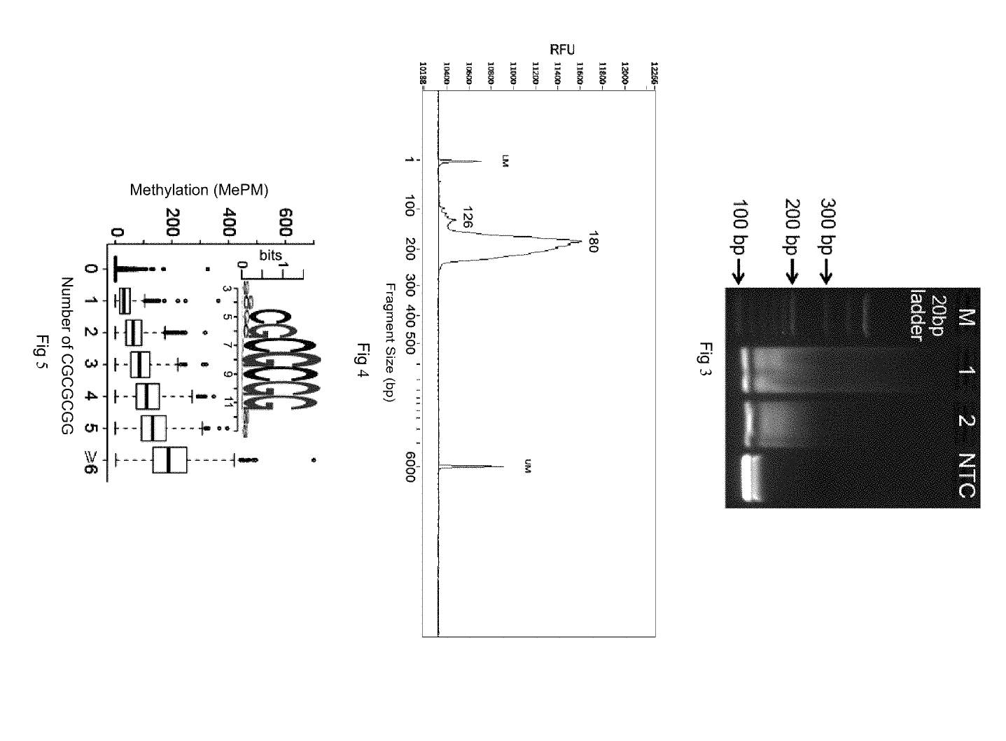

5.3 Imagine the gel (FIG. 3, sonicated DNA sample and MCTA-Seq DNA samples without sonication have the same amplification efficiency indicated that MCTA-Seq is highly efficient for amplification of heavily fragmented DNA samples.);

5.4 Excise the gel for DNA fragments ranging from 180 to 250 bp;

5.5 Purify the DNA from the gel by using the QIAquick Gel Extraction Kit (Qiagen) according to the manufacturer's protocol, obtaining the library for high throughput sequencing;

5.6 Analyze the size distribution of the library by using the Fragment Analyzer (Agilent) (FIG. 4, results show that size of DNA fragments in sequencing library range from 180-250 bp) and quantify the concentration of the library by using QPCR;

5.7 Sequence the library on an Illumina HiSeq2000/2500 sequencer at pair-end 100 bp reads to obtain about 10,000,000 raw paired reads;

5.8 Data analysis: firstly discard whole or any subsets of adapter sequences and low quality reads, then map the reads to the human genome reference (hg19), and then perform subsequent bioinformatics analysis.

### EXAMPLE 2

### The Throughput, Replicability, Sensitivity and Unique Molecular Identifiers of MCTA-Seq

We validated the MCTA-Seq method by applying it to the fully methylated human genomic DNA (FMG, Chemicon/Millipore S7821, CpGenome Universal Methylated DNA), genomic DNA extracted from human white blood cells (WBCs) and two cancer cell lines (HepG2 and HeLa cells). For these and subsequent samples, the MCTA-Seq libraries were sequenced using the Illumina HiSeq2000/2500 system, obtaining on average of 7.5 million pair-end raw reads per library. Lambda DNA was spiked into the samples for assessing the conversion rate (average 98.7%, 97.53˜99.31%, table 1).

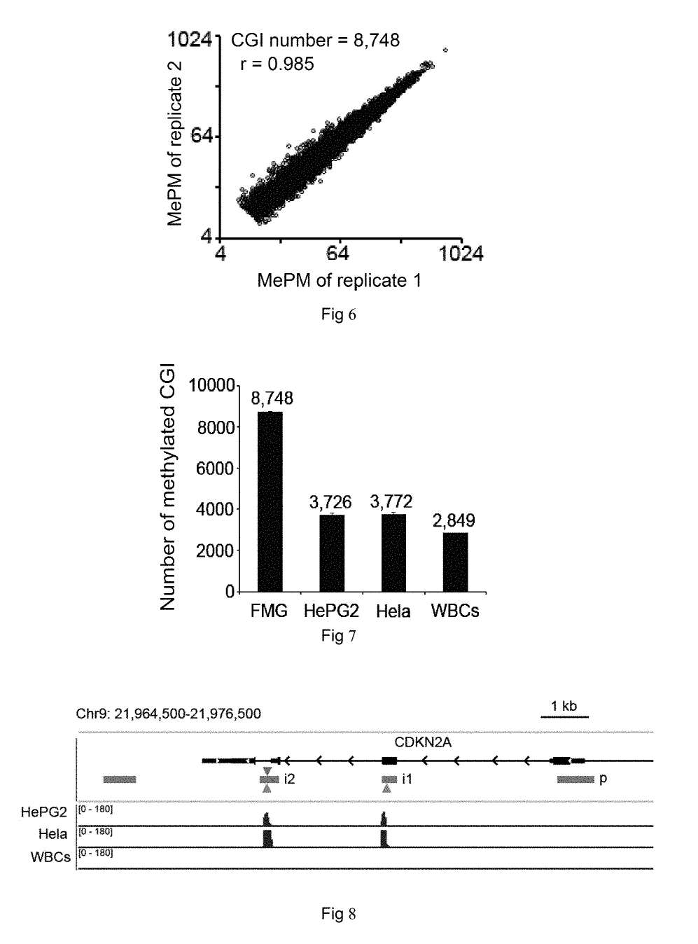

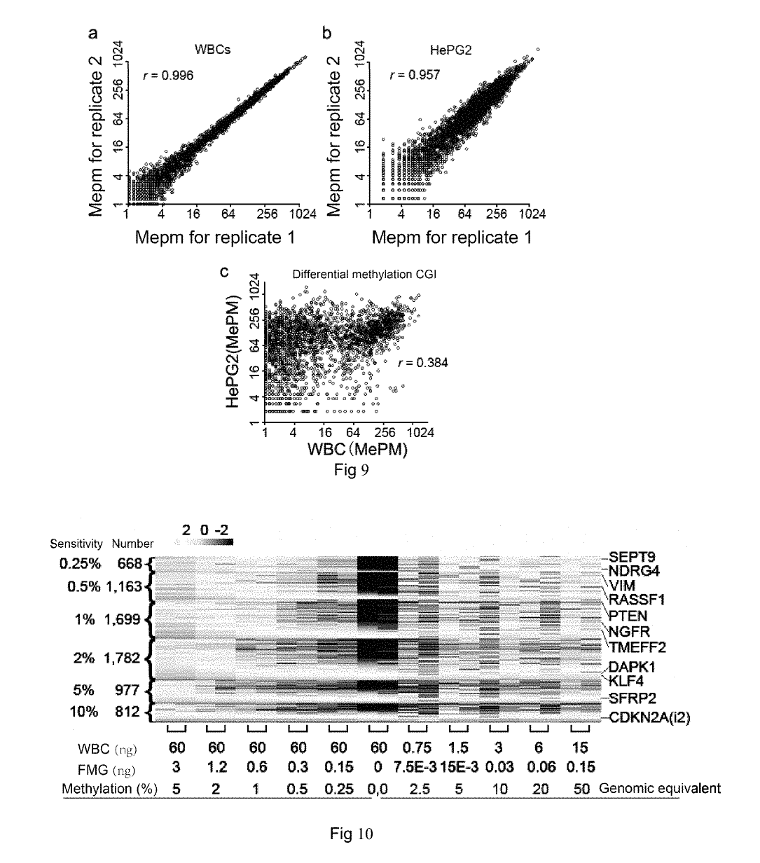

The results demonstrate that the aligned reads predominantly started from genomic CGCGCGG sequences (FIG. 5). The data of FMG show that, out of all 9,393 CGIs containing one or more CGCGCGG sequences, 8,748 (93.3%) were efficiently detected (average methylated alleles per million mapped reads (MePM)>8, FIG. 6). The detection efficiency of a CGI is positively correlated with the number of CGCGCGG sequences within the CGI (FIG. 5). MCTA-Seq detected on average 2,849, 3,726 and 3,773 methylated CGIs in WBCs, HepG2 and HeLa cells, respectively (FIG. 7). The methylation profiles were highly reproducible between technical replicates with average correlation of 0.974 (FIG. 6 and FIG. 9). In addition, the differentially methylated CGIs were clearly revealed when comparing the results of the cancer cell lines with those of WBCs (FIG. 8 and FIG. 9). FIG. 8 shows the result of CDKN2A by MCTA-Seq: 1) Almost all of the reads amplified initiate from three CGCGCGG short tandems in CDKN2A indicating the high specificity of MCTA-Seq amplification; 2) HePG2 and Hela cell lines have strong signal in two of the three CGCGCGG tandems while WBC has no signals which indicates that the CpG island is differential methylated and was shown by MCTA-Seq.

To analyze the sensitivity of MCTA-Seq, we spiked FMG into WBC gDNA in serial ratios of 1:10, 1:20, 1:50, 1:100, 1:200 and 1:400. MCTA-Seq can detect a methylated allele at a frequency as low as 0.25% (FIG. 10). In addition, the sensitivity should depend on the low detection limit (LoD) of the method, and could be further increased with the increase in sequencing depth. To determine the LoD of MCTA-Seq, we performed another dilution experiment in which the absolute amounts of FMG were serially decreased to 150, 60, 30, 15 and 7.5 pg while maintained the FMG:WBC=1:100. The result demonstrated that MCTA-Seq can detect as little as 7.5 pg (equivalent of ˜2.5 haploid genome) of methylated DNA. 84% (3,005 of 3,579) of the CGIs that were not detected in WBCs were detectable in at least one 7.5 pg replicate; 47% (1,687 of 3,579) of these were detectable in both 7.5 pg replicates (FIG. 10). The detection ratios substantially increased when the amount of methylated DNA rose to 15 pg, which is consistent with high sampling stochasticity in the 7.5 pg groups.

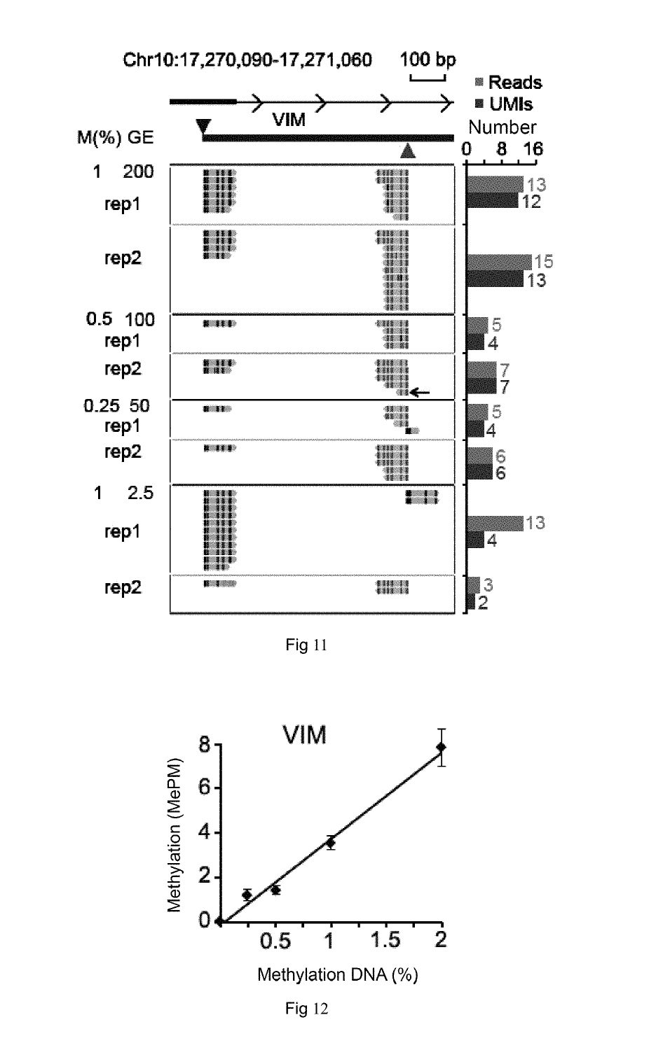

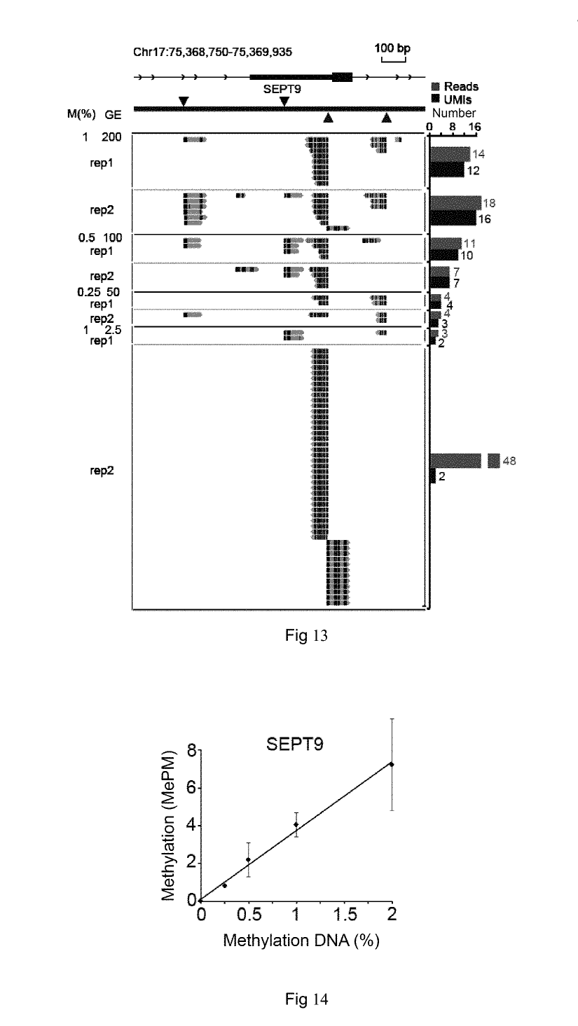

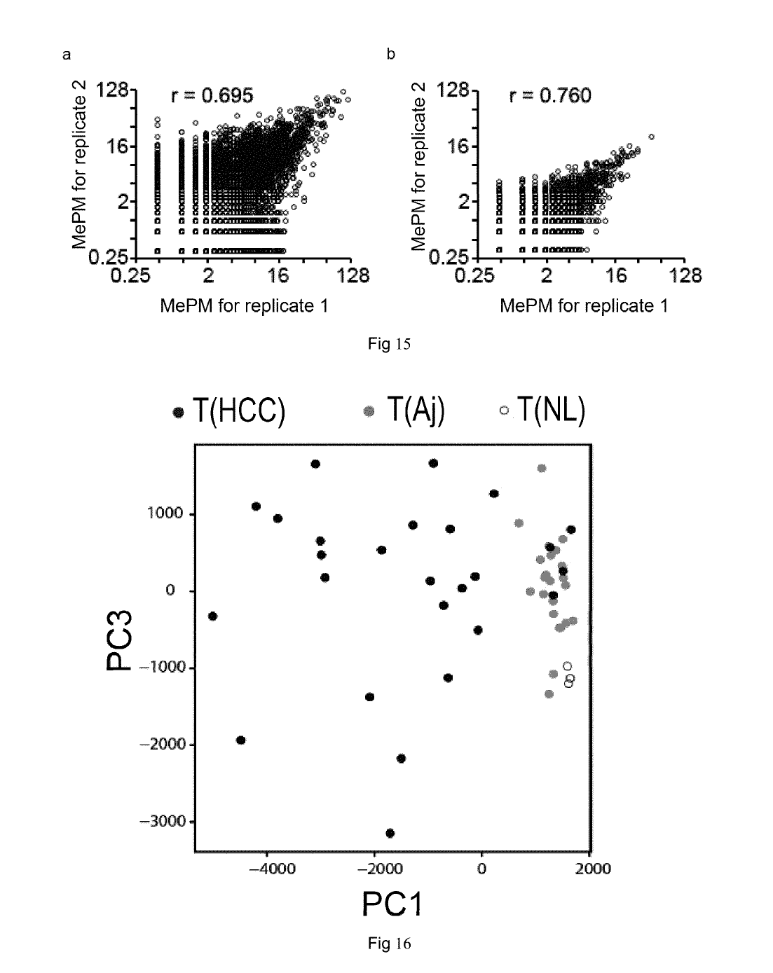

Taking CpG islands of promoter of VIM (VIMENTIN, no. 2) and SEPT9 (SEPTIN9) as an example, the amplicons predominantly initiated from two and four CGCGCGG sequences located within the CGI, respectively; the shortest amplicon was only 30 bp (FIG. 11 and FIG. 13), and the methylation value was linearly correlated with the lowest dilution range (FIG. 12 and FIG. 14). And the data shows that the increase in reproducibility was evident in the 7.5 pg experiments after normalization with UMIs, which were PCR over-amplified to the most extent (FIG. 15, FIG. 11 and FIG. 13).

### EXAMPLE 3

### MCTA-Seq of HCC Tissue Samples

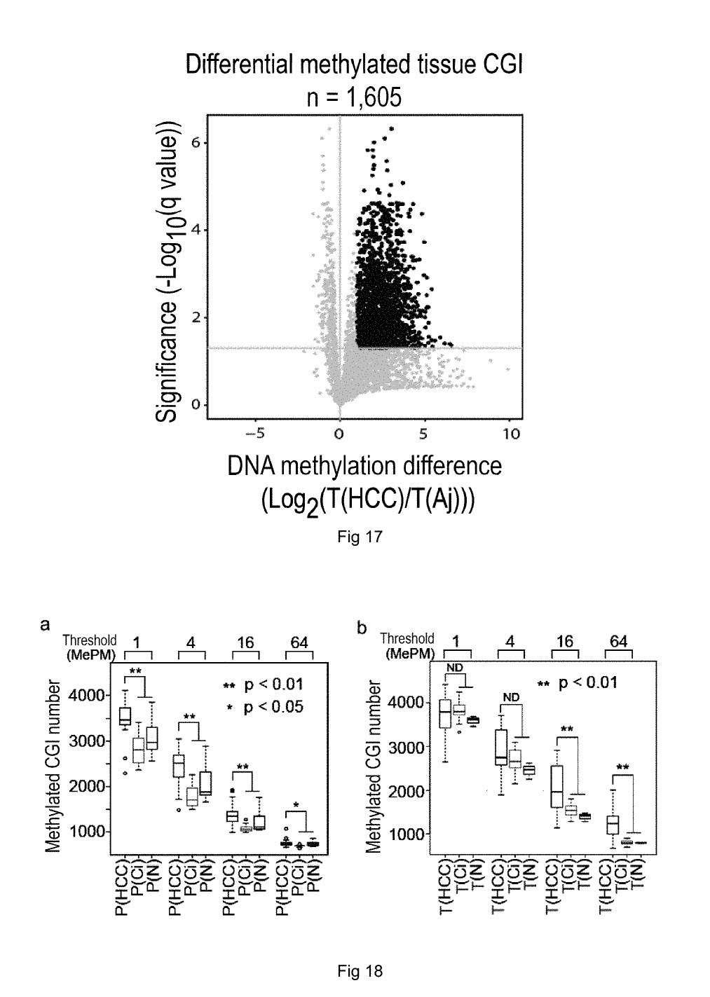

We performed MCTA-Seq in 27 pairs of HCCs and matched adjacent noncancerous liver samples and three normal liver samples obtained from patients with hepatic hemangioma during surgery (Table 2). Sample collection and further analysis were approved by patients themselves with signatures and the research was approved by ethics committee of Capital Medical University Affiliated Beijing Shi Ji Tan hospital. The principal component analysis (PCA) of tissue samples demonstrate that MCTA-Seq can successfully distinguish most cancerous tissues (23 of 27) from noncancerous tissues (FIG. 16). A total of 1,605 hypermethylated CGIs in HCC tissues were identified (referred to as tissue dmCGIs or tdmCGls, two-tailed t-test, FDR<0.05, average methylation fold changes>2, FIG. 17).

### EXAMPLE 4

### MCTA-Seq of HCC Plasma Samples

We performed MCTA-Seq in hepatocellular carcinoma patients' plasma (17 training group, 10 testing group, clinical information in Table 3), cirrhosis patients' plasma (11 training group, 6 testing group) and healthy individuals' plasma (9 training group, 19 testing group). Sample collection and further analysis were approved by patients themselves with signatures and the research was approved by ethics committee of Capital Medical University Affiliated Beijing Shi Ji Tan hospital. To prepare plasma, 5 ml peripheral blood was collected using EDTA anticoagulant tubes and the plasma samples were prepared within 6 h by centrifuging the blood tube at 1350×g for 12 min at room temperature, and transferring the plasma to a 15-ml tube, and re-centrifuging at 1,350×g for 12 min, and transferring to 1.5- or 2-ml tubes, and re-centrifuging at >10,000×g for 5 min and transferring to a new tube. The prepared plasma samples (about 2 ml) were then stored at −80° C. immediately. The plasma cell-free DNAs were extracted using the QIAamp DNA Blood Midi Kit (Qiagen) according to the manufacturer's protocol.

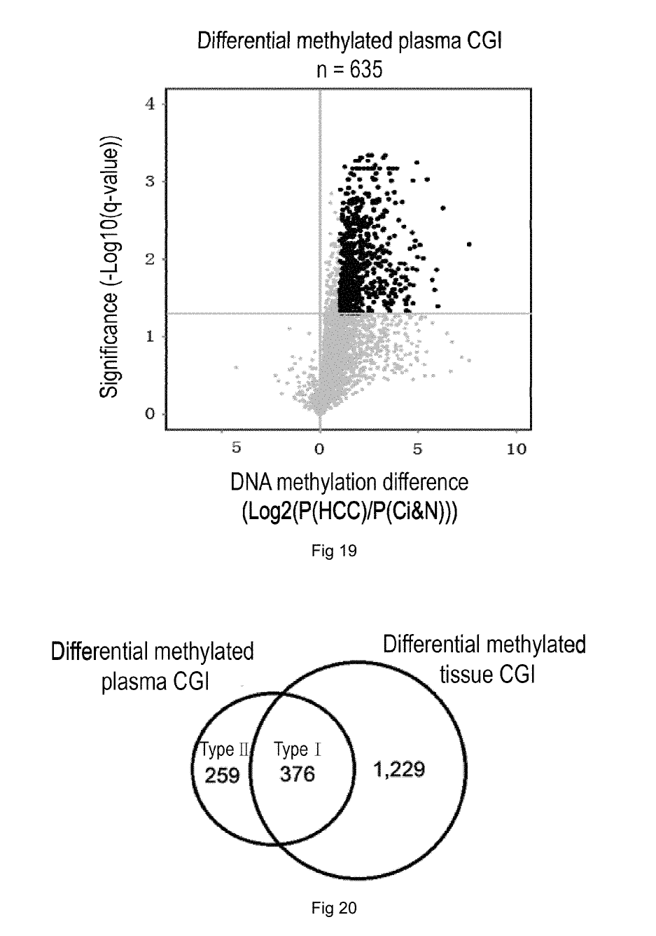

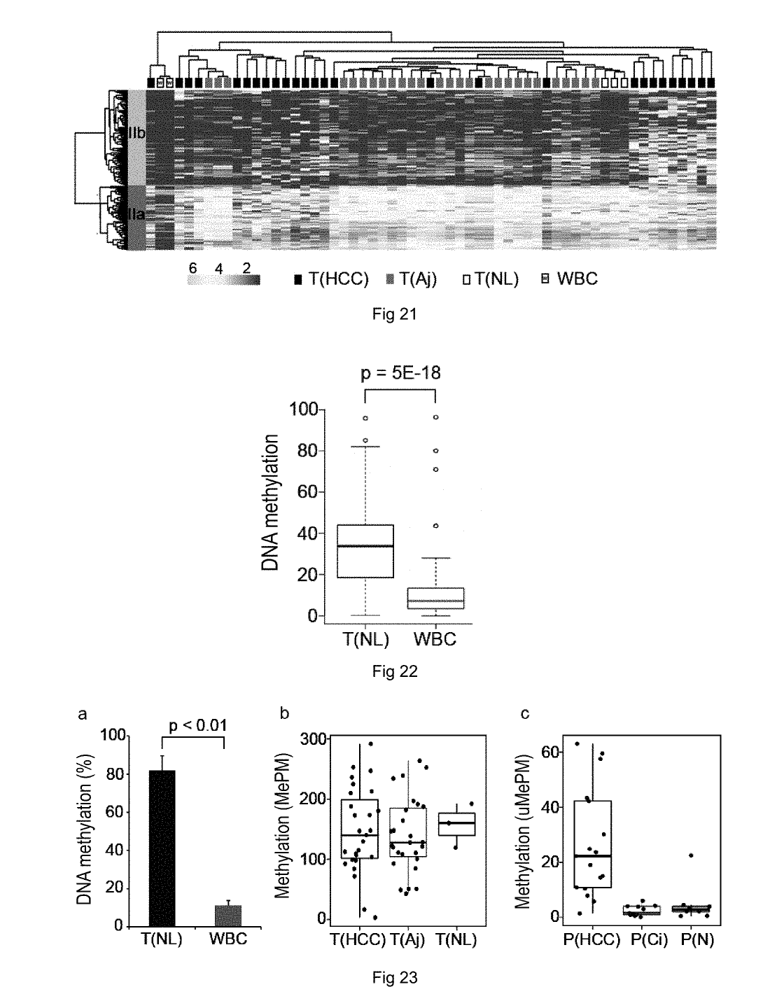

The overall number of methylated CGIs in HCC patients was significantly higher than that in cancer-free individuals (MePM>1, HCC vs non-cancerous individuals: median 3,465 vs 2,837, P<0.01, two-tailed MWW test, FIG. 18). The difference between plasma hepatocellular carcinoma patients and non-cancerous individuals gradually decreased with the increase of MePM threshold while the difference between hepatocellular carcinoma and adjacent tissues increased with the increase of MePM threshold (FIG. 18). This suggest that tumor-related methylation changes in cell free DNA predominantly occurs in low methylation range, significantly lower than the changes in tumor tissues. We found 635 aberrantly hypermethylated CpG islands in plasma of hepatocellular carcinoma patients (referred to plasma dmCGIs or pdmCGls, FDR<0.05, average methylation fold changes>2, two-tailed t-test, FIG. 19). Among them, 376 CpG islands are hypermethylated in tumor tissues, which were referred to type I markers and the rest of 259 CpG islands were referred to type II markers (FIG. 20). Clustering heatmap shows that type II markers could be divided into two groups. One group is type IIa markers (n=102) which are hypermethylated in tumor tissues, tumor adjacent tissues and normal liver tissues while hypomethylated in WBC showing tissue specific characteristics (FIG. 21, Table 4). We performed RRBS which confirmed that they were significantly more methylated in the normal liver tissues than the WBCs (median 32.8% vs 8%, P=5E-18, two tailed MWW test, FIG. 22, Table 4). We found that the type IIa markers are likely to be intragenic CGIs (53%, 54 of 102 markers), which is in agreement with previous reports that tissue-specifically methylated CGIs are often intragenic. CpG islands in SHANK2 is a typical intragenic type IIa CpG islands (no. 79, FIG. 23).

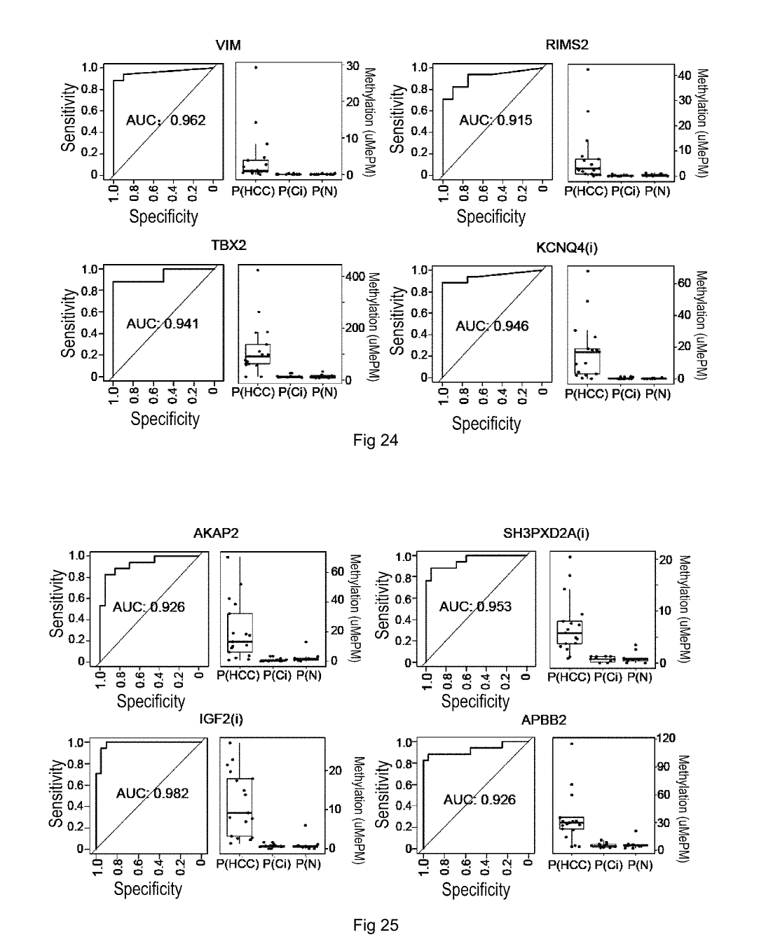

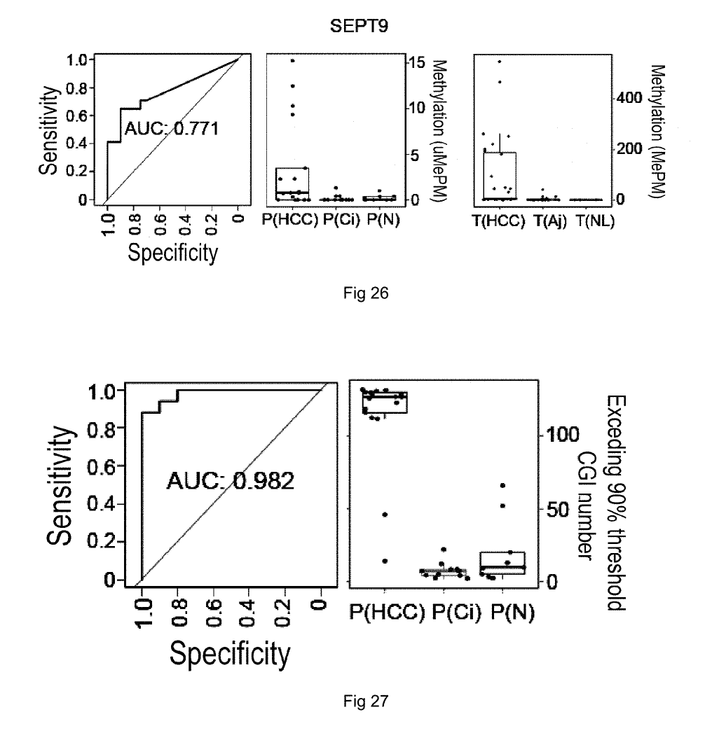

Then, we applied the receiver operating characteristic (ROC) curve analysis to find markers with best diagnostic performance. Out of 635 differentially methylated CGIs, a total of 132 top HCC-detecting markers were identified using an area under the curve (AUC) cutoff as >0.9. Among them 68 markers are type I markers, 64 markers are type II markers and most of the type II markers are type IIa markers (n=56). Detailed information and data of these two types of markers are provided in FIG. 24, FIG. 25 and Table 4.

In addition, SEPT9, for example, which showed good ability to distinguish between other carcinoma patients (colon cancer) and normal people had poor AUC (SEPT9 0.771) in hepatocellular carcinoma, suggesting that different tumors have different methylation patterns. It is possible to distinguish different types of tumors by methylation patterns of genome-scale detection of differential methylated CpG islands.

### EXAMPLE 5

### MCTA-Seq Diagnoses of Hepatocellular Carcinoma Patients by Detecting ccfDNA Methylation

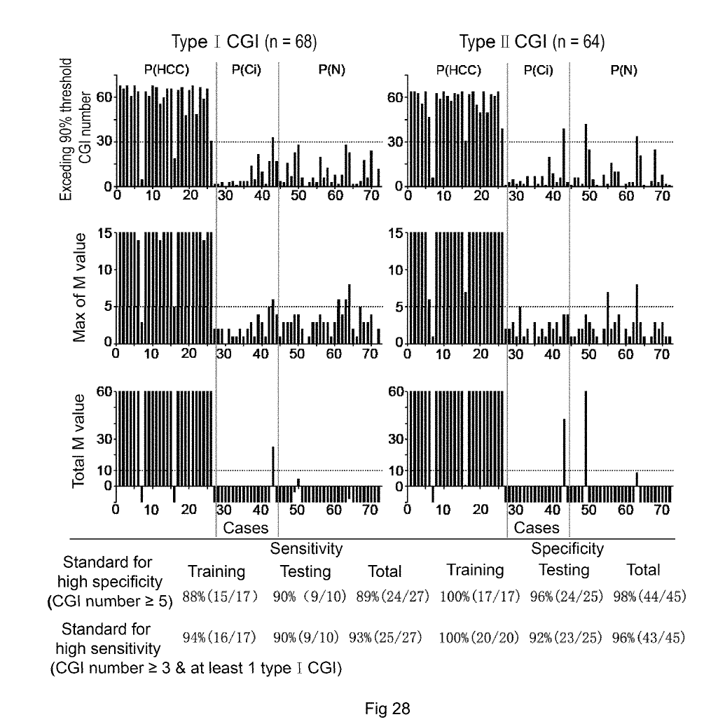

For each plasma differential methylated CpG island with high AUC value, a threshold corresponding to 90% specificity was set (i.e., for 90% of tumor-free individuals, the methylation level of the CpG island in cell free DNA was below the threshold) to analyze the number of CpG islands exceeding the threshold in 132 high-performance CpG islands. The results show that DNA from 132 high-performance CpG islands could be detected simultaneously in most of the the hepatocellular carcinoma patients while in most of the non-cancerous individuals. The correlation between the 132 CpG islands was weak. Counting the number of CpG islands exceeding the threshold corresponding to 90% specificity increases the specificity from 90% to 100% with AUC value 0.982 (95% CI: 0.952-1, FIG. 28).

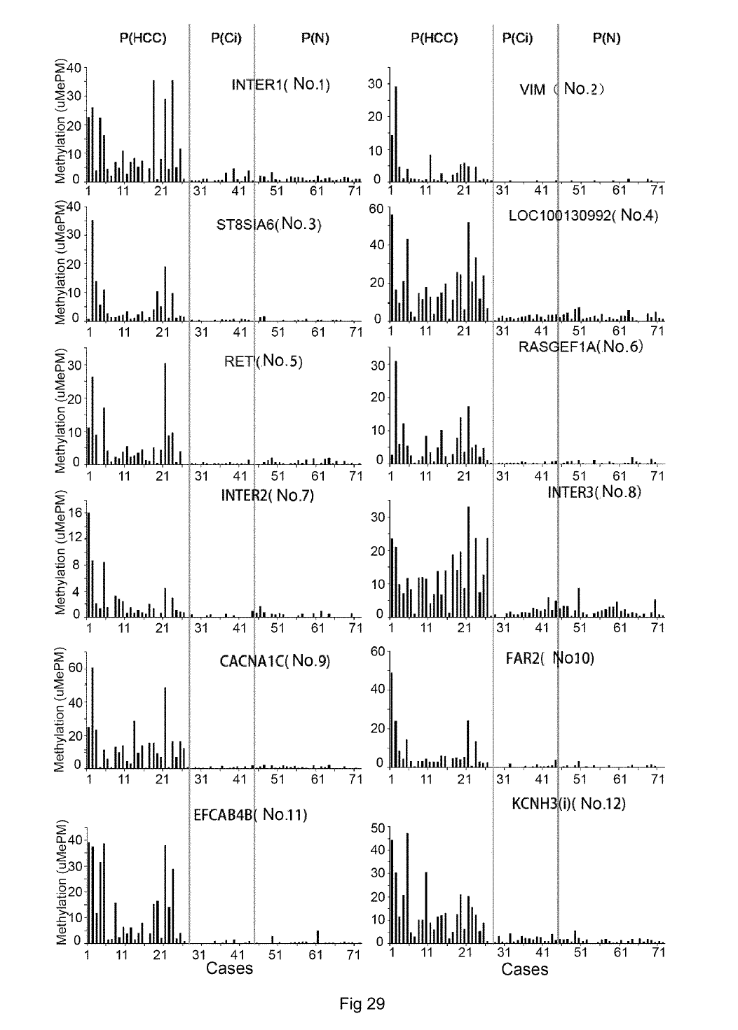

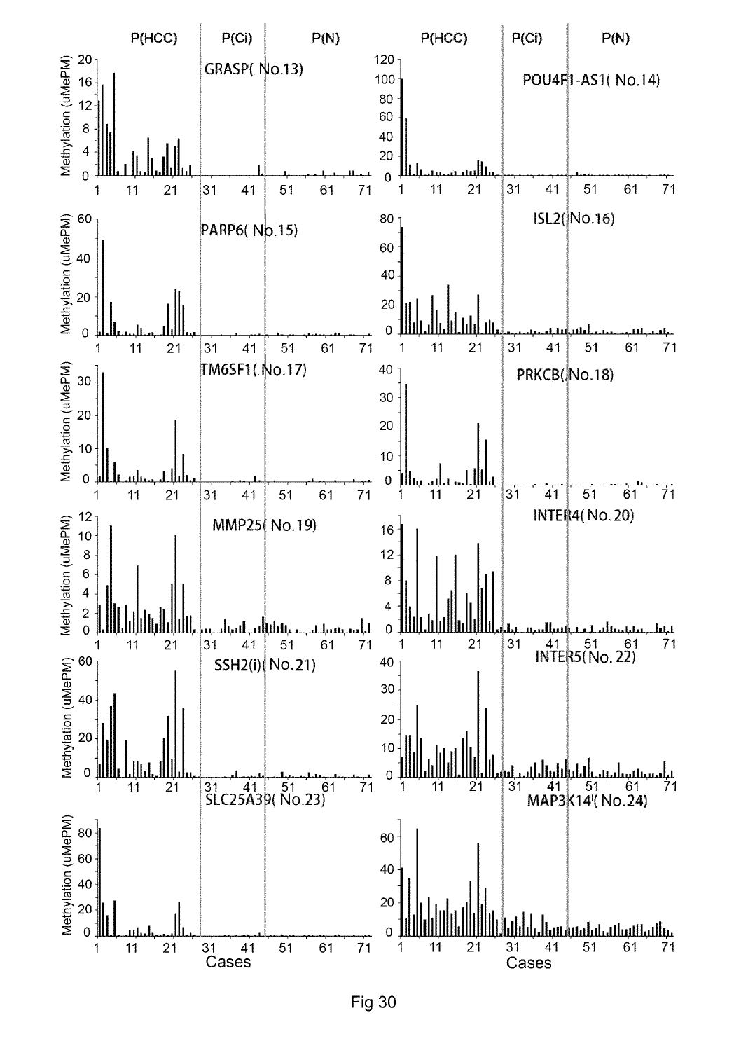

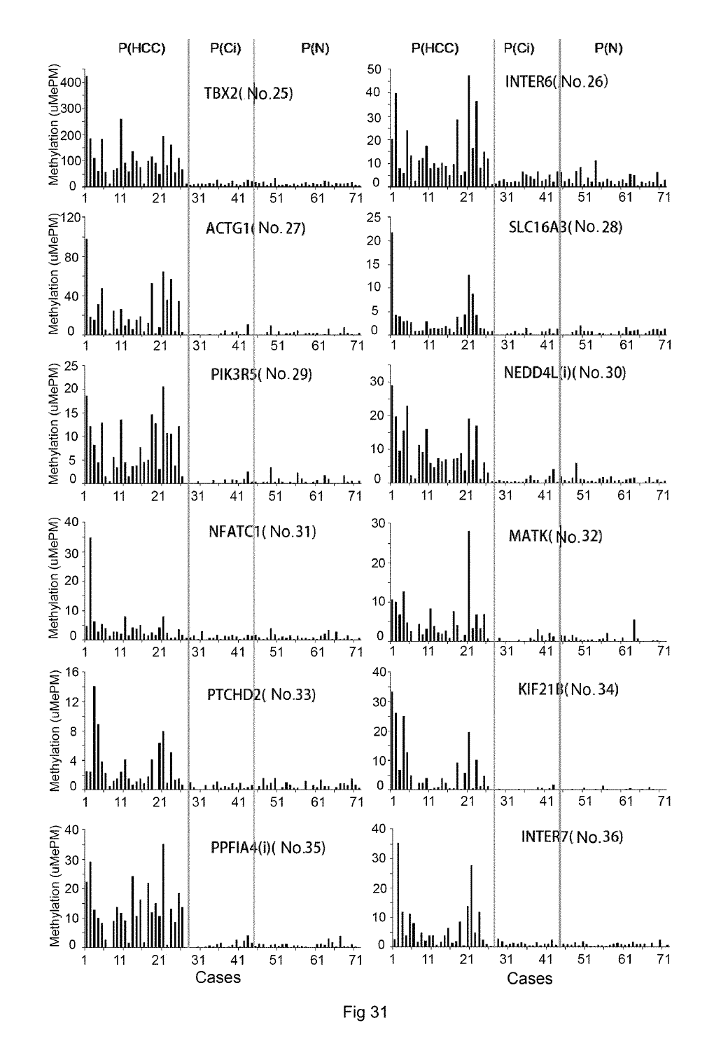

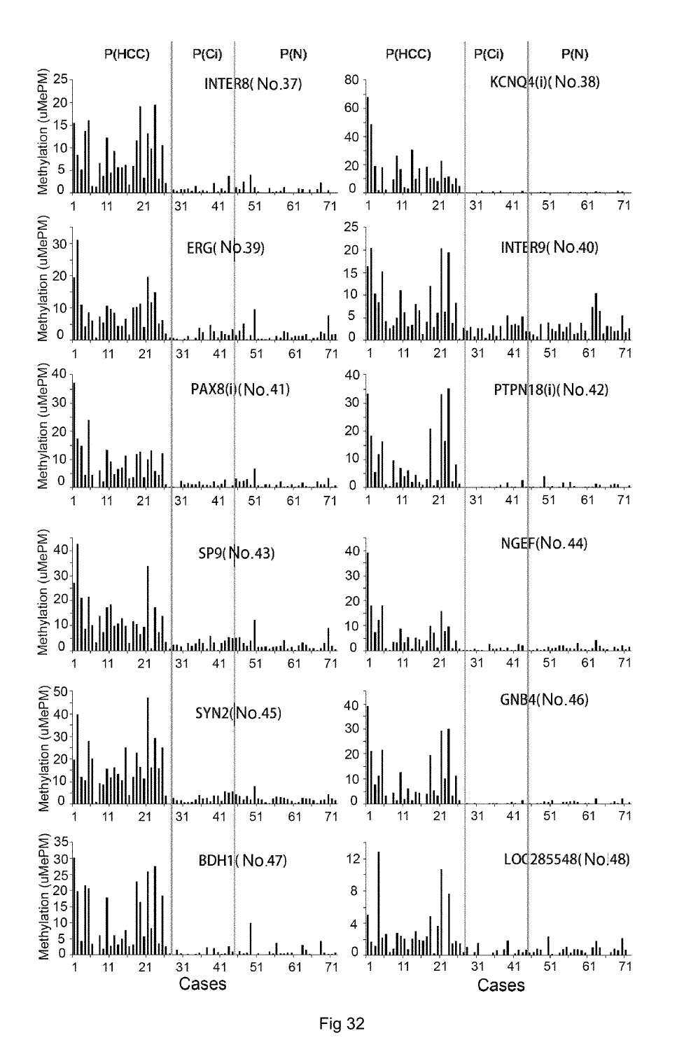

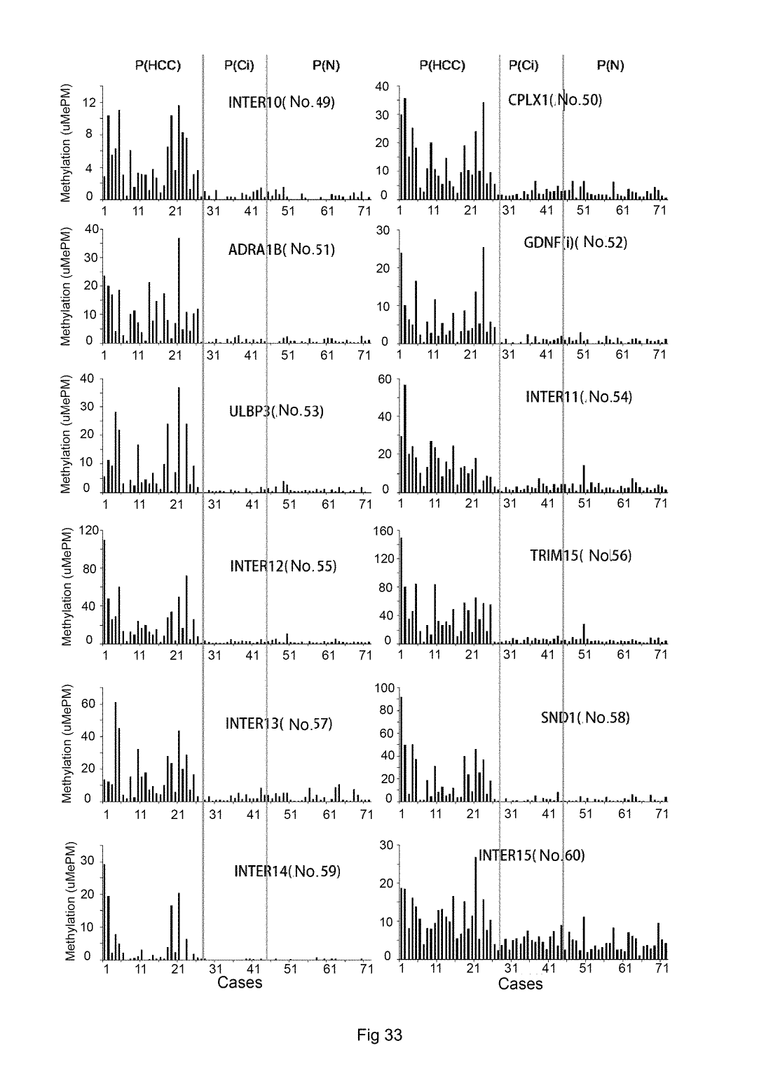

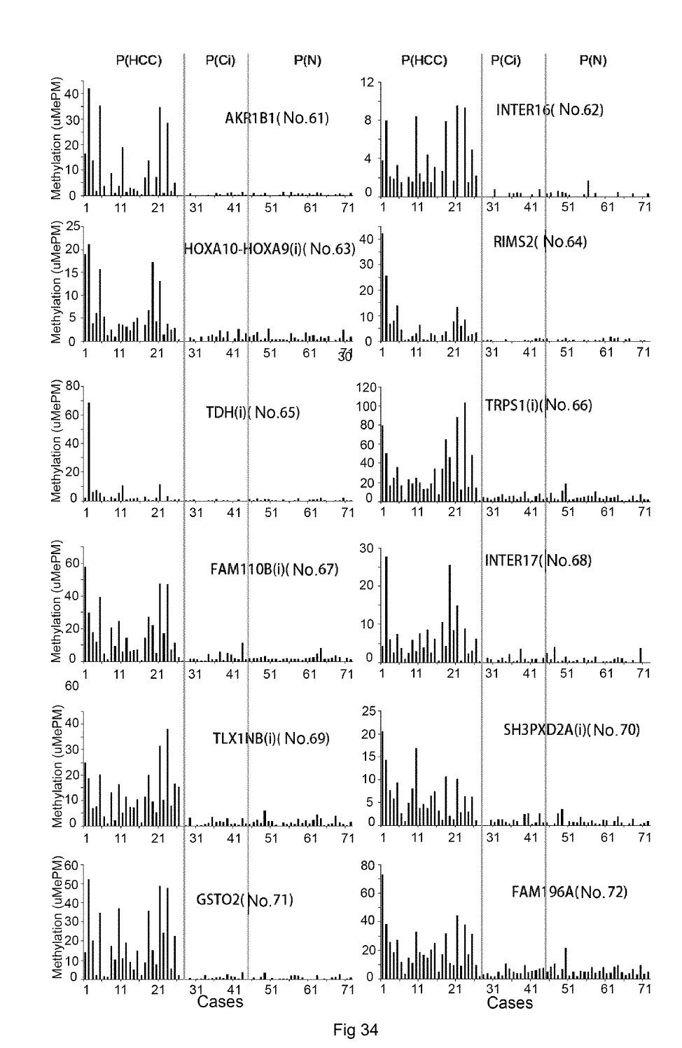

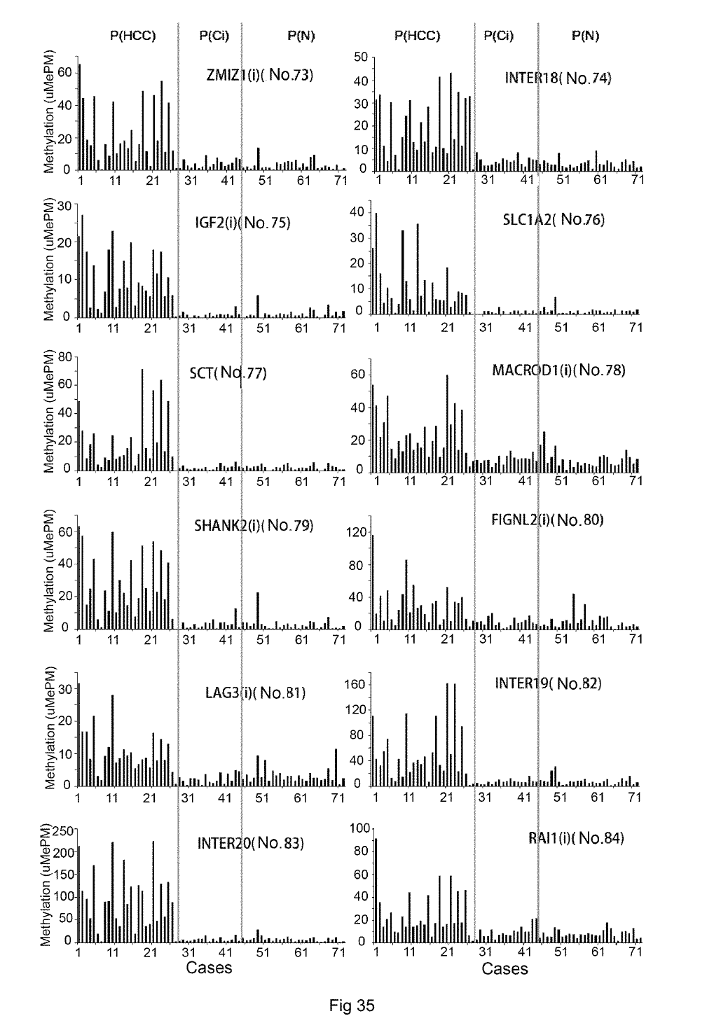

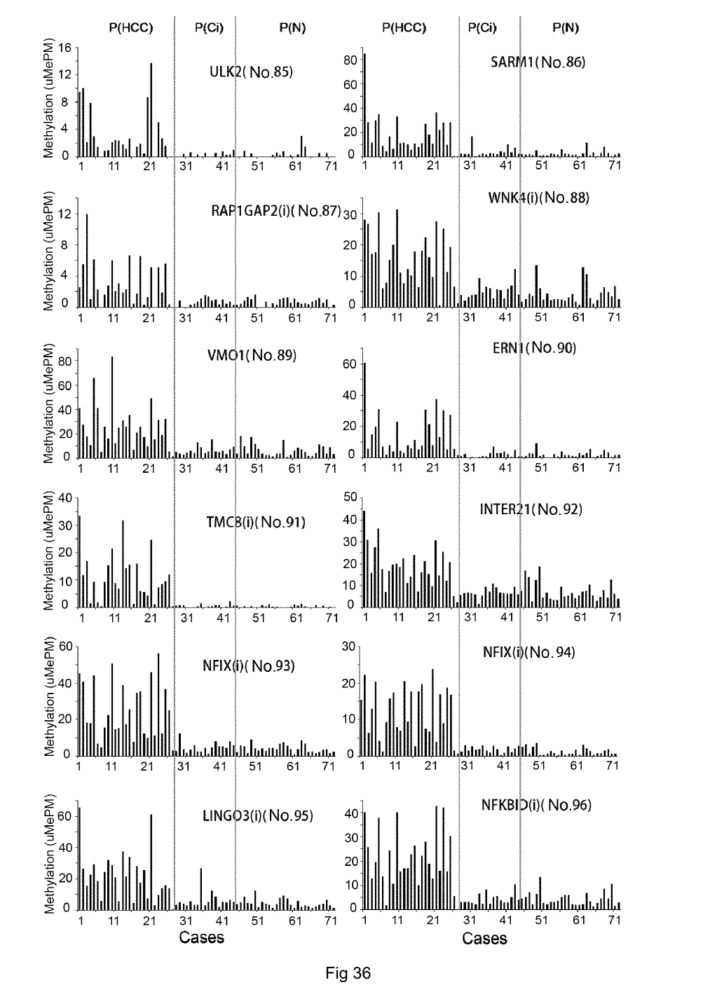

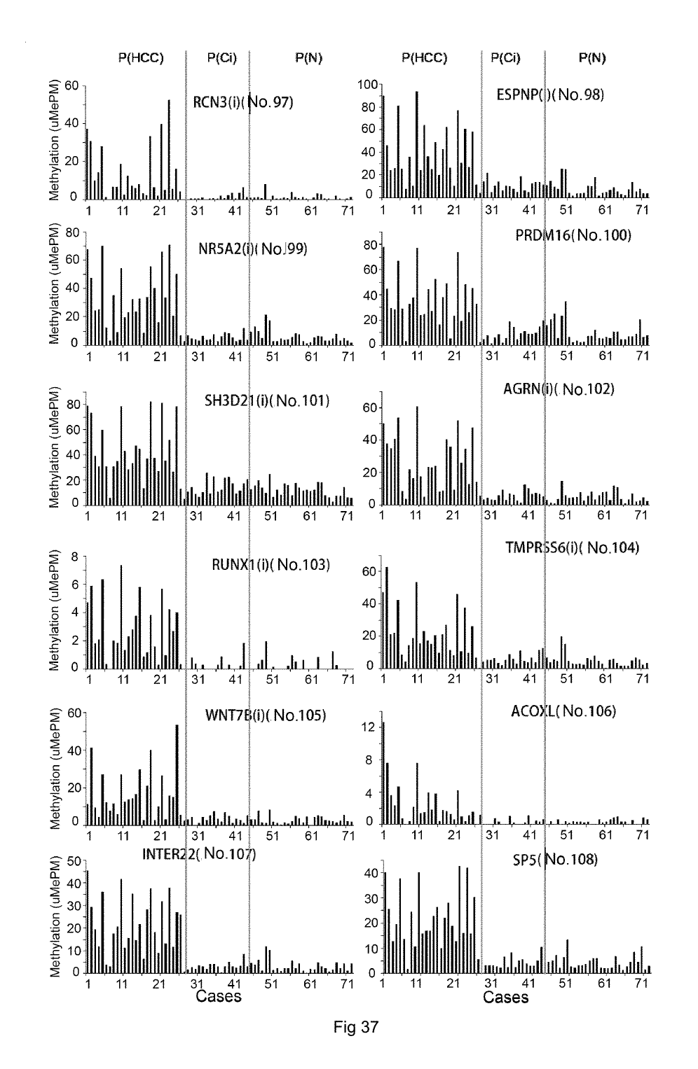

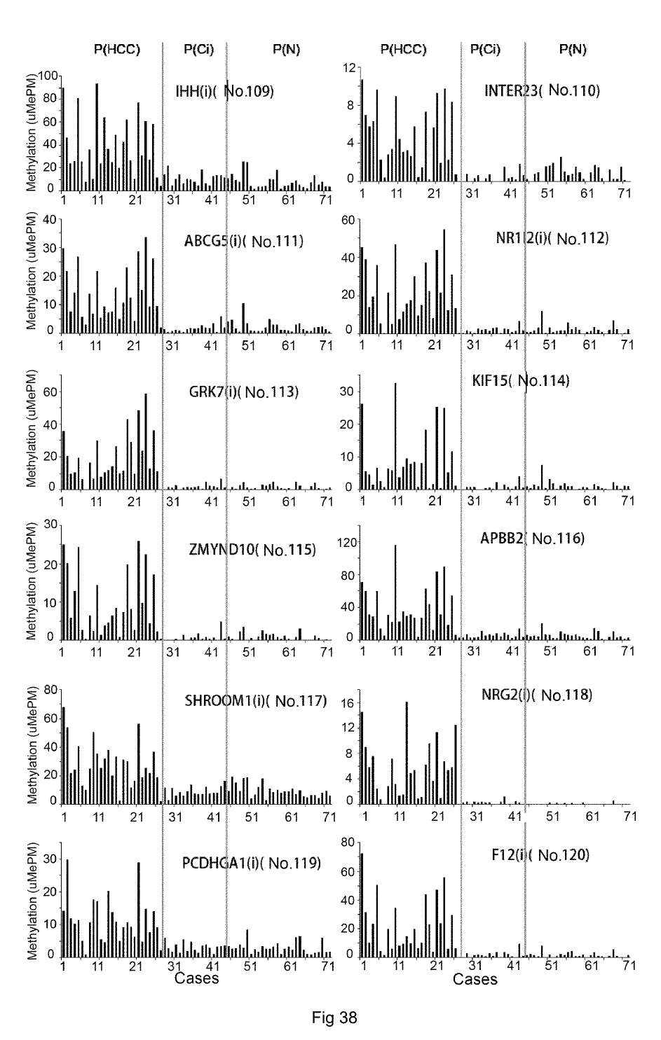

We next sought to add quantitative information and established the mutilple sites detection mode for HCC (FIG. 29). The number of standard deviations (SDs) above the 90% specificity threshold was calculated, named as the M-score (FIG. 6a). We measured two types of M-scores, the top M-score and the total M-score. The top M-score provides information of the uppermost hypermethylated locus and is expected to maximize the sensitivity of the assay because it uses the extreme marker to make a positive call. The total M-score represents an overall hypermethylation evaluation. In total, there were three types of parameters: the number of positive loci, the top M-score and the total M-score. For each parameter, we typically set a threshold just above the uppermost value in the cancer-free subjects to define a positive call (the number of positive loci≥30, the top M-score≥5, and the total M-score≥10). We independently calculated these parameters for the type I and type II markers, resulting in a total of 6 values in the final diagnostic panel. Further, we established two types of criterions with the high specificity criterion requiring 5 or 6 positive calls and the high sensitivity criterion requiring 3 or 4 positive calls with at least 1 from the type I markers.

The performance of the diagnostic panel in the training group was examined. The high specificity criterion had a sensitivity of 88% (15/17) and a specificity of 100% (1/20), and the high-sensitivity criterion had a sensitivity of 94% (16/17) and a specificity of 95% (1/20). To test our results, we analyzed a new set of 35 plasma samples, including those from 10 HCC patients, 6 cirrhosis patients and 19 healthy subjects, and performed the experiments and analysis in a double-blind manner (Table 2,3). Both criterions identified 9 out of 10 HCC patients (90% sensitivity), whereas 1 cirrhosis patient showed positive detection in the high-specificity criterion (96% specificity) and additional 1 healthy subject showed positive detection in the high-sensitivity criterion (92% specificity). The overall sensitivity of the training and testing groups was 93% in the high sensitivity criterion and 89% in the high-specificity criterion, and the overall specificity was 96% in the high sensitivity criterion and 98% in the high specificity criterion. FIG. 29˜FIG. 39 show the MCTA-Seq results of 132 type I and type II markers among the 72 individuals plasma samples.

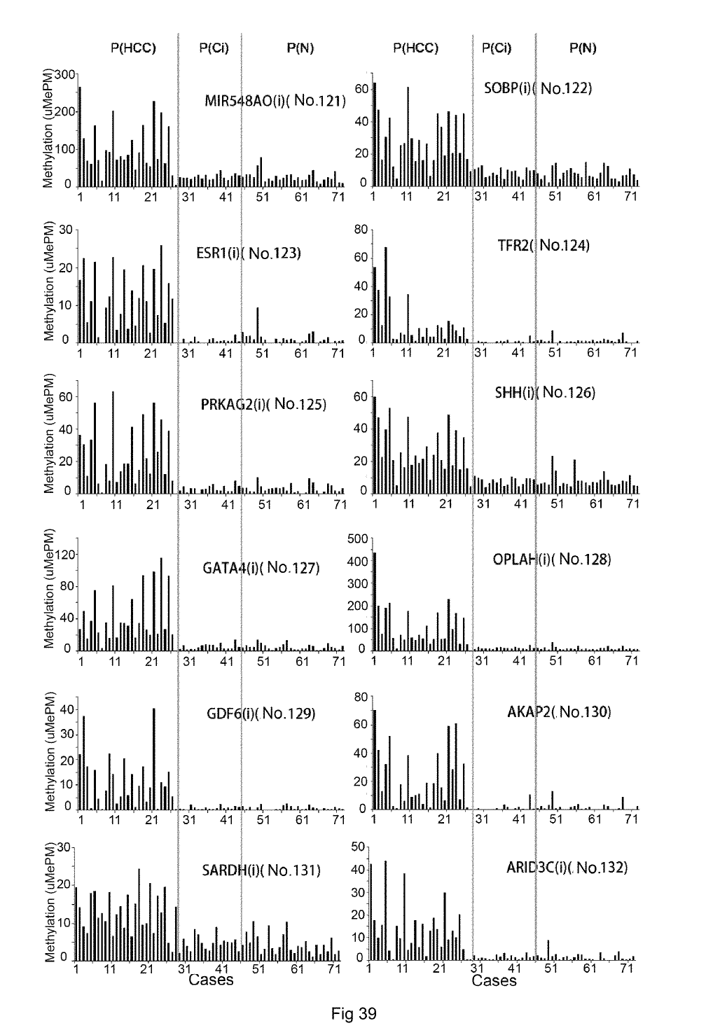

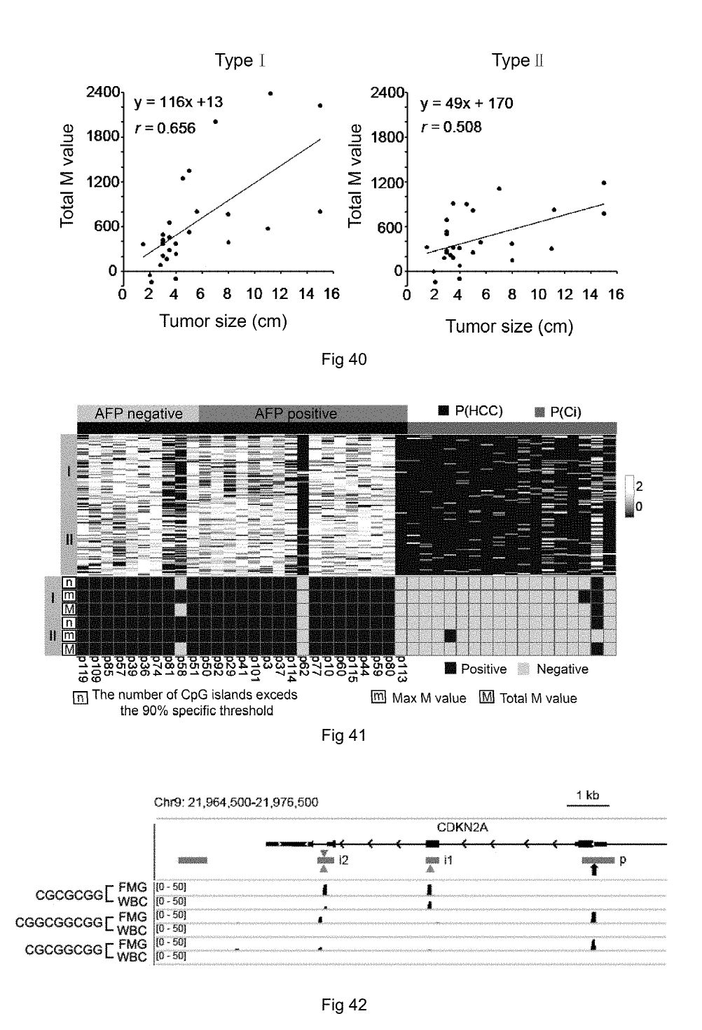

We further associated the MCTA-Seq results with the tumor size. The total M-scores of both CGI types were positively correlated with the tumor size (type I: r=0.656; type II: r=0.508, FIG. 40). Then, we compared the MCTA-Seq results with those of the AFP serum assay. In our study, 37% (10 of 27) of the HCC patients exhibited false-negative results in the AFP assay (<20 ng/mL, FIG. 41, Table 3). Importantly, 90% (9 of 10) of these AFP-negative patients were positively detected via MCTA-Seq using the high-specificity criterion, and all were positively detected using the high sensitivity criterion (FIG. 42).

### EXAMPLE 6

### Target Other Short CpG Tandem Sequences Except for CGCGCGG in Methylation Detection by MCTA-Seq

Taking CDKN2A as an example, it is possible to further increase the detection range by simultaneously targeting several types of CpG tandems (FIG. 32). CDKN2A promoter CpG island does not contain CGCGCGG short tandem sequence, so CDKN2A could not be detected using CGCGCGG as the primer of MCTA-Seq. But it contains a CGGCGGCGG site. Using CGGCGGCGG or CGCGGCGG as the primer, CDKN2A promoter CpG island can be detected with high efficiency.

TABLE 1
Sequencing information

| | | | Raw reads | Uniquely | |
|---|---|---|---|---|---|
| | | | (pair end, | mapping reads | Bisulfite |
| | | Sample | millions | (millions | transformation (%, |
| Name | Groups | number | Mean ± SD) | Mean ± SD) | Mean ± SD) |
| | FMG, WBCs, HePG2, | 30 | 7.9 ± 2.8 | 3.2 ± 1.2 | 98.86 ± 0.19 |
| | Hela and dilution | | | | |
| | experiments | | | | |
| T(HCC) | Tissue | 27 | 6.3 ± 2.3 | 2.1 ± 1.0 | 98.80 ± 0.18 |
| T(Aj) | Tissue | 27 | 6.7 ± 3.0 | 2.2 ± 0.8 | 98.63 ± 0.47 |
| T(NL) | Tissue | 3 | 5.3 ± 0.8 | 2.8 ± 0.3 | 98.90 ± 0.16 |
| P(HCC)-tr | Plasma, training | 17 | 8.4 ± 2.4 | 3.0 ± 0.7 | 98.70 ± 0.33 |
| | group | | | | |
| P(HCC)-te | Plasma, testing | 10 | 9.6 ± 3.1 | 3.8 ± 1.1 | 98.53 ± 0.42 |
| | group | | | | |
| P(Ci)-tr | Plasma, training | 11 | 7.1 ± 0.8 | 2.6 ± 0.3 | 98.34 ± 0.24 |
| | group | | | | |
| P(Ci)-te | Plasma, testing | 5 | 9.4 ± 1.4 | 3.5 ± 0.7 | 98.79 ± 0.30 |
| | group | | | | |
| P(N)-tr | Plasma, training | 9 | 7.4 ± 3.1 | 2.9 ± 1.0 | 98.65 ± 0.54 |
| | group | | | | |
| P(N)-te | Plasma, testing | 20 | 8.9 ± 2.1 | 3.1 ± 0.8 | 98.68 ± 0.40 |
| | group | | | | |

T(HCC): HCC cancer tissue; T(Aj): adjacent tissues of HCC; T(NL): normal liver tissue; P(HCC): plasma from HCC patients; P(Ci): plasma from cirrhosis patients; P(N): plasma from healthy individuals; tr: training group; te: testing group.

TABLE 2
Clinicle information of HCC patients and healthy individuals.

| | | | Sex | | | | |
|---|---|---|---|---|---|---|---|
| | | Age | Male | Female | | | |
| | | (years, | (samples/ | (samples/ | Tumor size (cm) | | |
| | Groups | Mean ± SD) | percentage) | percentage) | >5 | 3~5 | ≤3 |
| T(HCC) | Tissue | 59 ± 9 | 22(81%) | 5(19%) | 10(37%) | 6(22%) | 11(41%) |
| T(Aj) | Tissue | 59 ± 9 | 22(81%) | 5(19%) | 10(37%) | 6(22%) | 11(41%) |
| T(NL) | Tissue | 55 ± 8 | 1(33%) | 2(67%) | | | |
| P(HCC)-tr | Plasma, | 61 ± 10 | 15(88%) | 2(12%) | 4(24%) | 7(41%) | 6(35%) |
| | training group | | | | | | |
| P(HCC)-te | Plasma, testing | 63 ± 11 | 8(80%) | 2(20%) | 4(40%) | 3(30%) | 3(30%) |
| | group | | | | | | |
| P(Ci)-tr | Plasma, | 56 ± 11 | 8(73%) | 3(27%) | | | |
| | training group | | | | | | |
| P(Ci)-te | Plasma, testing | 50 ± 8 | 4(75%) | 1(25%) | | | |
| | group | | | | | | |
| P(N)-tr | Plasma, | 62 ± 15 | 5(56%) | 4(44%) | | | |
| | training group | | | | | | |
| P(N)-te | Plasma, testing | 55 ± 17 | 16(80%) | 4(20%) | | | |
| | group | | | | | | |

TABLE 3
Clinical and pathological data of HCC patients.

| | | | | HBV | Nodle | Tumor | Vascular | TNM | AFP |
|---|---|---|---|---|---|---|---|---|---|
| Number | Group | Age | Sex | infection | types | size | invasion | stages | (ng/mL) |
| p50 | training | 68 | male | Yes | Satellites | 15 | yes | T3aN0M0 | >1210 |
| p92 | testing | 77 | male | No | Satellites | 15 | yes | T3bN0M0 | >1210 |
| p29 | training | 56 | male | No | Unique | 11.2 | yes | T4N0M0 | >1210 |
| p119 | testing | 50 | male | Yes | Unique | 11 | yes | T3bN0M0 | 4.41 |
| p41 | training | 57 | male | No | Satellites | 8 | No | T3aN0M0 | 59 |
| p109 | testing | 74 | male | No | Unique | 8 | No | T1N0M0 | 2.61 |
| p101 | testing | 76 | female | Yes | Unique | 7 | yes | T2N0M0 | >1210 |
| p3 | training | 42 | male | Yes | Satellites | 5.6 | No | T3aN0M0 | 78.5 |
| p37 | training | 64 | male | Yes | Unique | 5 | No | T1N0M0 | 675.9 |
| p114 | testing | 39 | male | Yes | Satellites | 5 | yes | T4N0M0 | >1210 |
| p85 | testing | 63 | male | Yes | Unique | 4.5 | yes | T2N0M0 | 16.2 |
| p57 | training | 76 | female | No | Unique | 4 | yes | T2N0M0 | 2.29 |
| P62 | training | 51 | male | Yes | Unique | 4 | yes | T4N0M0 | 20.23 |
| P77 | training | 61 | male | Yes | Unique | 4 | yes | T4N0M0 | >1210 |
| p10 | training | 56 | male | Yes | Satellites | 3.5 | No | T2N0M0 | 768.5 |
| p39 | training | 72 | male | Yes | Satellites | 3.5 | No | T2N0M0 | 3.65 |
| p60 | training | 70 | male | Yes | Unique | 3.5 | yes | T3bN0M0 | 446.5 |
| p115 | testing | 60 | male | Yes | Unique | 3.3 | No | T1N0M0 | 27.4 |
| p36 | training | 44 | male | Yes | Unique | 3 | No | T1N0M0 | 5.74 |
| p44 | training | 63 | male | Yes | Satellites | 3 | yes | T4N0M1 | >1210 |
| p59 | training | 73 | male | No | Unique | 3 | No | T1N0M0 | >1210 |
| P74 | training | 68 | female | No | Unique | 3 | yes | T2N0M0 | 12.6 |
| p80 | testing | 70 | male | Yes | Unique | 3 | No | T1N0M0 | 36.8 |
| p81 | testing | 61 | female | No | Unique | 2.8 | No | T1N0M0 | 8.23 |
| p113 | testing | 63 | male | No | Unique | 2.1 | yes | T4N0M0 | 126.1 |
| p58 | training | 60 | male | No | Unique | 2 | No | T1N0M0 | 3.64 |
| p51 | training | 51 | male | Yes | Unique | 1.5 | No | T1N0M0 | 5.12 |

TABLE 4
MCTA-Seq data of type I and type II plasma differentially methylated CpG islands.

| | | | | | | | | | | NL | WBC |
|---|---|---|---|---|---|---|---|---|---|---|---|
| Serial | | | | T(HCC) | T(Aj) | T(NL) | T(WBC) | P(HCC) | P(Con) | RRBS | RRBS |
| number | Gene name | Type | AUC | MePM | MePM | MePM | MePM | uMePM | uMePM | (%) | (%) |
| 1 | INTER1 | I | 0.94 | 95(106) | 32(17) | 34(13) | 1(1) | 10(11) | 1(1) | 41(16) | 31(7) |
| 2 | VIM | I | 0.96 | 52(47) | 4(6) | 6(3) | 0(0) | 3(6) | 0(0) | 3(1) | 1(0) |
| 3 | ST8SIA6 | I | 0.96 | 88(111) | 5(6) | 2(0) | 0(0) | 5(8) | 0(0) | 4(1) | 1(0) |
| 4 | LOC100130992 | I | 0.92 | 113(93) | 33(15) | 22(7) | 1(0) | 17(14) | 2(2) | 4(2) | 1(0) |
| 5 | RET(i) | I | 0.93 | 56(72) | 8(5) | 11(3) | 0(0) | 6(8) | 1(1) | 4(1) | 1(0) |
| 6 | RASGEF1A(i) | I | 0.96 | 75(83) | 11(9) | 9(5) | 1(0) | 6(7) | 0(0) | 5(3) | 1(0) |
| 7 | INTER2 | I | 0.91 | 21(33) | 3(2) | 1(1) | 0(0) | 2(3) | 0(0) | 1(2) | 3(2) |
| 8 | INTER3 | I | 0.92 | 43(36) | 16(7) | 16(8) | 1(1) | 12(8) | 2(2) | 21(13) | 10(3) |
| 9 | CACNA1C | I | 0.92 | 104(102) | 18(9) | 30(17) | 0(0) | 14(14) | 1(1) | 10(6) | 1(1) |
| 10 | FAR2 | I | 0.93 | 46(48) | 4(2) | 3(1) | 0(0) | 7(10) | 1(1) | 7(1) | 2(1) |
| 11 | EFCAB4B | I | 0.95 | 124(112) | 13(16) | 14(3) | 0(0) | 12(14) | 0(1) | 30(1) | 1(1) |
| 12 | KCNH3(i) | I | 0.96 | 100(65) | 43(17) | 24(6) | 1(0) | 14(12) | 2(1) | 29(4) | 9(8) |
| 13 | GRASP | I | 0.92 | 36(35) | 8(12) | 1(0) | 0(0) | 4(5) | 0(0) | 8(2) | 1(0) |
| 14 | POU4F1-AS1 | I | 0.9 | 135(115) | 11(7) | 4(1) | 1(0) | 10(21) | 1(1) | 8(1) | 3(1) |
| 15 | PARP6 | I | 0.91 | 75(94) | 5(6) | 7(5) | 0(0) | 7(11) | 0(0) | 9(3) | 0(0) |
| 16 | ISL2(i) | I | 0.93 | 66(55) | 20(11) | 23(7) | 1(0) | 14(15) | 2(2) | 13(2) | 3(0) |
| 17 | TM6SF1 | I | 0.92 | 23(25) | 2(1) | 0(0) | 0(0) | 4(7) | 0(0) | 8(6) | 2(0) |
| 18 | PRKCB | I | 0.94 | 72(81) | 5(5) | 2(1) | 0(0) | 4(8) | 0(0) | 3(1) | 0(0) |
| 19 | MMP25 | I | 0.91 | 32(53) | 3(3) | 2(1) | 0(0) | 3(3) | 1(0) | 4(2) | 2(1) |
| 20 | INTER4 | I | 0.97 | 46(35) | 22(13) | 6(5) | 1(0) | 5(5) | 1(0) | 48(13) | 3(0) |
| 21 | SSH2(i) | I | 0.93 | 162(187) | 23(33) | 25(11) | 0(0) | 13(15) | 1(1) | 15(9) | 0(0) |
| 22 | INTER5 | I | 0.9 | 61(70) | 14(7) | 13(2) | 2(0) | 10(8) | 3(2) | 19(12) | 6(1) |
| 23 | SLC25A39 | I | 0.92 | 109(143) | 14(11) | 28(3) | 0(1) | 9(17) | 0(1) | 11(1) | 1(0) |
| 24 | MAP3K14(i) | I | 0.93 | 67(94) | 18(10) | 23(1) | 9(6) | 21(14) | 6(3) | 1(1) | 1(0) |
| 25 | TBX2 | I | 0.94 | 577(487) | 181(50) | 117(22) | 7(1) | 107(86) | 14(6) | 19(5) | 6(1) |
| 26 | INTER6 | I | 0.93 | 61(57) | 30(17) | 29(6) | 1(0) | 14(11) | 4(2) | 43(28) | ND |
| 27 | ACTG1 | I | 0.96 | 141(144) | 64(45) | 110(33) | 1(1) | 22(24) | 2(3) | 6(1) | 1(0) |
| 28 | SLC16A3 | I | 0.91 | 42(55) | 9(6) | 5(3) | 1(0) | 3(4) | 1(1) | 12(0) | 5(1) |
| 29 | PIK3R5 | I | 0.98 | 41(36) | 19(9) | 12(5) | 0(0) | 7(6) | 1(1) | 10(2) | 1(0) |
| 30 | NEDD4L(i) | I | 0.96 | 82(68) | 23(22) | 26(4) | 0(0) | 9(7) | 1(1) | 24(13) | 3(3) |
| 31 | NFATC1 | I | 0.91 | 33(43) | 10(3) | 4(1) | 3(2) | 4(6) | 1(1) | 23(1) | 20(10) |
| 32 | MATK | I | 0.94 | 53(53) | 7(5) | 6(6) | 0(0) | 5(6) | 1(1) | 5(1) | 2(0) |
| 33 | PTCHD2 | I | 0.9 | 36(52) | 5(4) | 3(1) | 1(0) | 3(3) | 1(0) | 3(1) | 2(0) |
| 34 | KIF21B | I | 0.95 | 60(76) | 6(5) | 8(2) | 0(0) | 6(9) | 0(0) | 10(5) | 1(0) |
| 35 | PPFIA4(i) | I | 0.95 | 53(40) | 22(10) | 15(5) | 0(0) | 12(9) | 1(1) | 35(2) | 3(1) |
| 36 | INTER7 | I | 0.91 | 61(103) | 5(6) | 2(1) | 1(0) | 6(8) | 1(1) | 5(1) | 2(2) |
| 37 | INTER8 | I | 0.98 | 94(77) | 47(23) | 87(32) | 0(0) | 8(6) | 1(1) | 18(9) | 3(1) |
| 38 | KCNQ4(i) | I | 0.95 | 60(46) | 27(13) | 17(5) | 0(0) | 14(15) | 0(0) | 35(10) | 14(13) |
| 39 | ERG | I | 0.91 | 57(40) | 19(7) | 16(2) | 1(1) | 9(7) | 2(2) | 7(2) | 5(1) |
| 40 | INTER9 | I | 0.91 | 72(48) | 28(15) | 16(3) | 0(1) | 8(6) | 3(2) | 9(1) | 7(2) |
| 41 | PAX8(i) | I | 0.9 | 147(99) | 32(15) | 10(4) | 1(0) | 9(8) | 1(1) | 27(4) | 10(2) |
| 42 | PTPN18(i) | I | 0.97 | 119(104) | 26(31) | 9(5) | 0(0) | 9(11) | 1(1) | 12(3) | 2(0) |
| 43 | SP9 | I | 0.94 | 145(141) | 32(11) | 35(11) | 3(0) | 13(10) | 3(2) | 9(2) | 3(0) |
| 44 | NGEF | I | 0.93 | 62(49) | 26(22) | 7(1) | 1(0) | 7(8) | 1(1) | 33(7) | 10(1) |
| 45 | SYN2 | I | 0.94 | 75(56) | 31(13) | 32(9) | 2(0) | 16(11) | 3(2) | 7(0) | 6(2) |
| 46 | GNB4 | I | 0.91 | 61(44) | 17(10) | 15(3) | 0(0) | 9(10) | 0(1) | 11(15) | 1(1) |
| 47 | BDH1 | I | 0.92 | 95(69) | 43(25) | 92(33) | 0(0) | 10(10) | 1(2) | 26(6) | 1(0) |
| 48 | LOC285548 | I | 0.93 | 29(42) | 5(4) | 2(1) | 0(0) | 3(3) | 1(1) | 6(2) | 3(1) |
| 49 | INTER10 | I | 0.96 | 52(36) | 7(6) | 7(3) | 0(0) | 4(3) | 0(0) | 19(13) | 7(8) |
| 50 | CPLX1(i) | I | 0.92 | 113(69) | 41(34) | 62(18) | 2(0) | 13(10) | 3(2) | 57(2) | 33(8) |
| 51 | ADRA1B(i) | I | 0.93 | 63(63) | 21(9) | 21(9) | 0(0) | 10(9) | 1(1) | 43(26) | 5(0) |
| 52 | GDNF(i) | I | 0.92 | 47(38) | 16(12) | 16(7) | 1(1) | 7(6) | 1(1) | 26(1) | 5(1) |
| 53 | ULBP3 | I | 0.92 | 51(55) | 17(16) | 18(10) | 0(0) | 9(10) | 1(1) | 1(1) | 1(0) |
| 54 | INTER11 | I | 0.95 | 65(65) | 19(9) | 14(6) | 3(0) | 15(11) | 3(2) | 12(1) | 6(1) |
| 55 | INTER12 | I | 0.94 | 215(127) | 67(33) | 24(17) | 1(1) | 24(25) | 2(2) | 18(1) | 3(1) |
| 56 | TRIM15(i) | I | 0.93 | 234(176) | 116(47) | 95(13) | 4(1) | 40(32) | 6(4) | 43(4) | 12(1) |
| 57 | INTER13 | I | 0.91 | 209(163) | 77(43) | 51(31) | 2(1) | 16(15) | 3(3) | 17(4) | 9(2) |
| 58 | SND1 | I | 0.95 | 249(175) | 76(50) | 55(14) | 0(1) | 20(21) | 2(2) | 55(2) | 2(0) |
| 59 | INTER14 | I | 0.92 | 38(58) | 1(1) | 1(1) | 0(0) | 5(7) | 0(0) | 6(0) | 2(1) |
| 60 | INTER15 | I | 0.92 | 75(55) | 23(10) | 25(5) | 4(2) | 11(5) | 5(2) | 12(3) | 7(4) |
| 61 | AKR1B1 | I | 0.94 | 136(125) | 12(10) | 17(2) | 0(1) | 9(12) | 0(0) | 5(1) | 0(0) |
| 62 | INTER16 | I | 0.91 | 28(36) | 7(4) | 11(1) | 0(0) | 3(3) | 0(0) | 9(0) | 1(0) |
| 63 | HOXA10-HOXA9(i) | I | 0.9 | 61(66) | 7(4) | 3(0) | 0(0) | 6(6) | 1(1) | 9(0) | 2(1) |
| 64 | RIMS2 | I | 0.91 | 146(159) | 7(4) | 3(1) | 0(0) | 6(9) | 0(0) | 7(3) | 3(0) |
| 65 | TDH(i) | I | 0.91 | 39(53) | 4(3) | 2(1) | 0(0) | 5(13) | 1(1) | 8(1) | 2(1) |
| 66 | TRPS1(i) | I | 0.92 | 314(230) | 84(40) | 133(23) | 4(1) | 31(26) | 5(3) | 21(29) | 2(3) |
| 67 | FAM110B(i) | I | 0.92 | 171(134) | 32(21) | 29(8) | 1(0) | 17(16) | 2(2) | 96(0) | 92(1) |
| 68 | INTER17 | I | 0.91 | 33(46) | 6(3) | 6(3) | 0(1) | 6(7) | 1(1) | 5(2) | 3(1) |
| 69 | TLX1NB(i) | IIa | 0.92 | 76(39) | 48(28) | 57(4) | 0(0) | 12(9) | 2(1) | 34(2) | 4(2) |
| 70 | SH3PXD2A(i) | IIa | 0.95 | 40(20) | 33(18) | 27(7) | 0(0) | 6(5) | 1(1) | 68(7) | 1(0) |
| 71 | GSTO2 | IIa | 0.98 | 118(93) | 78(45) | 92(35) | 0(0) | 17(16) | 1(1) | 28(2) | 0(0) |
| 72 | FAM196A | IIa | 0.92 | 136(106) | 81(26) | 69(13) | 2(1) | 21(15) | 6(3) | 10(2) | 5(2) |
| 73 | ZMIZ1(i) | IIa | 0.91 | 151(84) | 129(59) | 97(41) | 0(0) | 22(18) | 4(3) | 33(6) | 3(0) |
| 74 | INTER18 | IIa | 0.91 | 106(87) | 111(54) | 49(11) | 2(2) | 19(13) | 4(2) | 51(2) | 10(0) |
| 75 | IGF2(i) | IIa | 0.98 | 40(29) | 45(21) | 72(16) | 0(0) | 10(7) | 1(1) | 49(14) | 4(3) |
| 76 | SLC1A2 | IIb | 0.92 | 41(39) | 23(8) | 16(7) | 1(0) | 11(11) | 1(1) | 11(2) | 3(1) |
| 77 | SCT | IIa | 0.98 | 47(35) | 43(18) | 31(5) | 1(1) | 21(19) | 2(2) | 36(14) | 11(5) |
| 78 | MACROD1(i) | IIa | 0.91 | 156(127) | 79(35) | 48(19) | 7(0) | 24(15) | 8(4) | 34(3) | 11(1) |
| 79 | SHANK2(i) | IIa | 0.94 | 147(71) | 140(63) | 158(37) | 1(0) | 26(19) | 3(4) | 82(7) | 11(3) |
| 80 | FIGNL2(i) | IIa | 0.91 | 99(83) | 72(30) | 60(12) | 4(1) | 31(25) | 10(8) | 32(6) | 4(0) |
| 81 | LAG3(i) | IIa | 0.92 | 65(36) | 59(22) | 53(9) | 2(1) | 11(7) | 3(2) | 55(33) | 8(6) |
| 82 | INTER19 | IIa | 0.93 | 284(245) | 176(80) | 183(65) | 2(0) | 52(45) | 8(5) | 37(52) | ND |
| 83 | INTER20 | IIa | 0.94 | 315(197) | 245(73) | 179(37) | 2(1) | 93(65) | 8(5) | 0(0) | 0(0) |
| 84 | RAI1(i) | IIa | 0.92 | 127(91) | 99(55) | 157(39) | 4(0) | 26(20) | 9(4) | 54(1) | 28(1) |
| 85 | ULK2 | IIb | 0.92 | 39(80) | 7(8) | 3(3) | 0(0) | 3(4) | 0(1) | 9(4) | 8(0) |
| 86 | SARM1 | IIa | 0.96 | 139(100) | 86(40) | 68(17) | 1(0) | 18(17) | 3(3) | 14(3) | 1(0) |
| 87 | RAP1GAP2(i) | IIb | 0.9 | 11(19) | 8(4) | 2(0) | 0(0) | 3(3) | 1(0) | 6(1) | 2(0) |
| 88 | WNK4(i) | IIa | 0.96 | 70(47) | 65(30) | 57(9) | 7(4) | 15(9) | 5(3) | ND | 3(4) |
| 89 | VMO1 | IIa | 0.91 | 80(54) | 110(39) | 118(31) | 7(2) | 26(18) | 6(4) | 87(19) | ND |
| 90 | ERN1 | IIa | 0.93 | 153(188) | 71(46) | 115(21) | 2(0) | 14(14) | 2(2) | 2(0) | 1(0) |
| 91 | TMC8(i) | IIb | 0.97 | 17(22) | 22(8) | 16(6) | 0(0) | 11(9) | 0(0) | 21(3) | 1(0) |
| 92 | INTER21 | IIa | 0.91 | 120(91) | 101(33) | 106(13) | 13(2) | 18(10) | 7(4) | 35(11) | 8(1) |
| 93 | NFIX(i) | IIa | 0.97 | 68(38) | 72(31) | 99(21) | 3(1) | 24(16) | 4(2) | 55(10) | 25(11) |
| 94 | NFIX(i) | IIa | 0.94 | 49(54) | 62(37) | 49(30) | 0(0) | 12(7) | 2(1) | 48(7) | 18(1) |
| 95 | LINGO3(i) | IIa | 0.92 | 50(60) | 32(14) | 49(5) | 9(0) | 21(16) | 5(4) | 9(2) | 7(3) |
| 96 | NFKBID(i) | IIa | 0.96 | 75(54) | 43(28) | 73(21) | 0(0) | 12(11) | 1(1) | ND | ND |
| 97 | RCN3(i) | IIa | 0.91 | 127(90) | 73(50) | 55(12) | 0(0) | 13(14) | 1(2) | 38(11) | 1(1) |
| 98 | ESPNP(i) | IIa | 0.94 | 283(172) | 159(105) | 151(50) | 2(0) | 34(32) | 6(4) | 30(1) | 15(3) |
| 99 | NR5A2(i) | IIa | 0.91 | 165(94) | 140(74) | 292(88) | 7(1) | 32(21) | 6(4) | 38(4) | 11(0) |
| 100 | PRDM16 | IIa | 0.91 | 150(80) | 152(35) | 93(19) | 6(1) | 36(21) | 10(7) | 18(1) | 13(2) |
| 101 | SH3D21(i) | IIa | 0.92 | 132(88) | 71(21) | 54(17) | 17(2) | 42(23) | 13(6) | 42(6) | 44(8) |
| 102 | AGRN(i) | IIa | 0.93 | 149(132) | 92(41) | 30(8) | 1(1) | 26(17) | 5(3) | 42(5) | 18(3) |
| 103 | RUNX1(i) | IIb | 0.92 | 11(10) | 11(4) | 14(6) | 0(0) | 3(2) | 0(0) | 47(13) | 0(0) |
| 104 | TMPRSS6(i) | IIa | 0.92 | 185(101) | 173(62) | 171(32) | 8(3) | 22(16) | 6(4) | 50(4) | 11(3) |
| 105 | WNT7B(i) | IIa | 0.93 | 36(47) | 51(30) | 9(7) | 4(1) | 16(13) | 3(2) | 13(2) | 13(0) |
| 106 | ACOXL | IIb | 0.93 | 20(12) | 11(5) | 2(2) | 0(0) | 3(3) | 0(0) | 7(0) | 1(0) |
| 107 | INTER22 | IIa | 0.94 | 105(58) | 99(56) | 97(18) | 2(1) | 21(13) | 3(2) | 63(10) | 13(4) |
| 108 | SP5 | IIa | 0.93 | 113(53) | 82(36) | 98(12) | 4(1) | 21(12) | 5(3) | 14(4) | 5(2) |
| 109 | IHH(i) | IIa | 0.91 | 234(110) | 184(64) | 236(65) | 7(3) | 39(26) | 9(6) | 31(3) | 5(1) |
| 110 | INTER23 | IIb | 0.94 | 44(57) | 21(12) | 17(15) | 1(0) | 4(3) | 1(1) | 9(8) | 1(0) |
| 111 | ABCG5(i) | IIa | 0.96 | 113(63) | 98(39) | 119(22) | 1(0) | 14(9) | 2(2) | 29(12) | 3(3) |
| 112 | NR1I2(i) | IIa | 0.94 | 192(96) | 145(102) | 125(33) | 0(0) | 21(15) | 2(2) | 64(51) | 0(0) |
| 113 | GRK7(i) | IIa | 0.94 | 95(60) | 71(28) | 59(16) | 0(0) | 19(15) | 2(2) | ND | ND |
| 114 | KIF15 | IIb | 0.92 | 17(28) | 44(31) | 94(28) | 0(0) | 8(9) | 1(1) | 13(1) | 1(0) |
| 115 | ZMYND10 | IIa | 0.94 | 82(48) | 42(23) | 31(10) | 0(0) | 9(8) | 1(1) | 26(19) | 2(0) |
| 116 | APBB2 | IIa | 0.93 | 163(99) | 113(72) | 123(12) | 1(1) | 37(28) | 6(4) | 43(13) | 0(0) |
| 117 | SHROOM1(i) | IIa | 0.9 | 126(53) | 109(36) | 196(31) | 10(1) | 28(16) | 9(4) | 68(3) | 17(0) |
| 118 | NRG2(i) | IIb | 0.96 | 17(23) | 7(5) | 2(1) | 0(0) | 5(4) | 0(0) | 4(4) | 1(0) |
| 119 | PCDHGA1(i) | IIa | 0.91 | 53(35) | 34(11) | 31(10) | 0(1) | 11(7) | 3(2) | 42(9) | 13(3) |
| 120 | F12(i) | IIa | 0.96 | 165(111) | 114(56) | 122(21) | 0(0) | 21(19) | 2(2) | 41(22) | 1(1) |
| 121 | MIR548AO(i) | IIa | 0.93 | 543(256) | 324(100) | 343(61) | 15(1) | 101(65) | 27(12) | 44(2) | 12(5) |
| 122 | SOBP(i) | IIa | 0.9 | 127(64) | 105(33) | 76(5) | 4(2) | 28(16) | 8(3) | 51(8) | 17(0) |
| 123 | ESR1(i) | IIa | 0.91 | 69(42) | 55(32) | 97(25) | 1(1) | 11(8) | 1(2) | 47(1) | 2(0) |
| 124 | TFR2(i) | IIa | 0.96 | 82(48) | 64(29) | 40(13) | 1(0) | 14(17) | 2(2) | 34(9) | 1(1) |
| 125 | PRKAG2(i) | IIa | 0.94 | 160(145) | 102(51) | 75(3) | 1(0) | 24(18) | 3(2) | 35(39) | 2(3) |
| 126 | SHH(i) | IIa | 0.91 | 261(122) | 175(66) | 137(33) | 4(2) | 27(15) | 8(4) | 39(2) | 9(2) |
| 127 | GATA4(i) | IIa | 0.96 | 124(109) | 165(60) | 234(43) | 2(1) | 40(31) | 6(3) | 33(8) | 11(4) |
| 128 | OPLAH(i) | IIa | 0.97 | 919(691) | 479(202) | 404 (145) | 9(2) | 105(93) | 13(6) | 27(3) | 6(2) |
| 129 | GDF6(i) | IIb | 0.94 | 29(45) | 21(12) | 29(16) | 0(0) | 11(10) | 1(1) | 25(1) | 7(1) |
| 130 | AKAP2 | IIa | 0.93 | 125(87) | 65(48) | 82(4) | 0(0) | 22(21) | 2(3) | 56(15) | 1(1) |
| 131 | SARDH(i) | IIa | 0.92 | 163(119) | 144(76) | 227(44) | 7(1) | 13(5) | 5(3) | 34(8) | 6(2) |
| 132 | ARID3C(i) | IIa | 0.92 | 63(58) | 44(24) | 56(12) | 0(0) | 14(12) | 2(2) | 46(26) | 5(6) |

Note:
MePM and uMePM values: Mean (SD). T(HCC): HCC cancer tissue; T(Aj): adjacent tissues of HCC; T(NL): normal liver tissue; P(HCC): plasma from HCC patients; P(Con): plasma from cancer-free individuals including cirrhosis patients and healthy individuals.

The above description of exemplary embodiments of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications to thereby enable others skilled in the art to best utilize the invention in various embodiments and with various modifications as are suited to the particular use contemplated.

1.-20. (canceled)

21. A method for genome-scale detection of differentially methylated CpG island relating to human abnormal states comprises: 1) Obtaining human DNA samples; 2) Detecting the methylation level of short CpG tandem sequences of the human DNA samples in a genome-scale manner; 3) Comparing the methylation level of the short CpG tandem sequences between the human DNA samples and DNA samples from normal population to identify the differentially methylated CpG islands relating to human abnormal states;
wherein the human DNA samples are derived from human cells, tissues, blood, body fluid, urine, excrement or their combination;
wherein the genome-scale detection refers to simultaneously detecting more than 100 CpG islands.

22. The method according to claim 21, wherein the referred DNA samples are derived from plasma or urine cell-free DNA.

23. The method according to claim 21, wherein the differentiatial methylated CpG islands are used to determine the human abnormal state.

24. The method according to claim 21, wherein the short CpG tandem sequence is a nucleitide sequence of 7 nucleitides in length containing 3 CpG dinucleotides.

25. The method according to claim 21, wherein the short CpG tandem sequence is a nucleitide sequence of 8 nucleitides in length containing 3 or more CpG dinucleotides.

26. The method according to claim 21, wherein the short CpG tandem sequence is a nucleitide sequence of 9 nucleitides in length containing 3 or more CpG dinucleotides.

27. The method according to claim 21, wherein the short CpG tandem sequence is one the following sequences or a combination of the following sequences: CGCGCGG, CGCGCGA, CGCGCGT, CGCGCGC, CGGCGCGG, CGGCGCGA, CGGCGCGT, CGGCGCGC, CGCGGCGG, CGCGGCGA, CGCGGCGT, CGCGGCGC, CGGCGGCGG, CGGCGGCGA, CGGCGGCGT, CGGCGGCGC.

28. The method according to claim 27, wherein at least one sequence of the short CpG tandem sequences is CGCGCGG.

29. The method according to claim 21, wherein the human abnormal state is cancer.

30. The method according to claim 21, wherein the human DNA samples are derived from human plasma or urine cell-free DNA; wherein the differentially methylated CpG islands are nucleic acid sequences no. 1˜68; aberrent hypermethylation of those sequences indicates hepatocellular carcinoma.

31. The method according to claim 21, wherein the human DNA samples are derived from human plasma or urine cell-free DNA; wherein the differentially methylated CpG islands are nucleic acid sequences no. 69˜132; aberrent hypermethylation of those sequences indicates hepatocellular carcinoma or abnormal liver tissue injury.

32. The method according to claim 21, wherein detecting the methylation level of short CpG tandem sequences includes the following steps:
the human DNA samples are derived from human cells, tissues, blood, body fluid, urine, excrement or their combination;
step 1, treating a DNA sample with a modifying agent to form a modified DNA wherein cytosine bases but not 5′-methyl-cytosine bases of the DNA sample are modified to uracil bases;
step 2, providing Primer A and DNA polymerase to the modified DNA to allow at least one round of linear amplification to form a semi-amplicon capable of anchoring Adapter Primer C at one end, wherein Primer A is composed of a 3′ portion and a 5′ portion, wherein the 3′ portion contains 4 or more nucleotides capable of binding to the modified DNA and allowing amplification, wherein the 5′ portion allows Adapter Primer C to bind to its reverse complementary sequence for PCR amplification;
step 3, amplifying the semi-amplicon by using Primer B and DNA polymerase to form a full-amplicon enriched with methylated CpG islands and capable of anchoring Adapter Primer C at one end and Adapter Primer D at the other end, wherein Primer B is composed of a 3′ portion and a 5′ portion, wherein the 3′ portion contains the short CpG tandem allowing amplification and enrichment of the methylated CpG islands, wherein the 5′ portion allows Adapter Primer D to bind to its reverse complementary sequence for PCR amplification;
step 4, amplifying the full-amplicon by using Adapter Primer C, Adapter Primer D and DNA polymerase to form a final-amplicon via PCR exponential amplification;
step 5, separating and purifying the final-amplicon to form a library for high-throughput sequencing, sequencing the library and analyzing the data.

33. The method according to claim 32, wherein the modifying agent is bisulfite.

34. The method according to claim 32, wherein DNA polymerase in Step2 is capable of strand displacement.

35. A kit for detecting the methylation level of short CpG tandem sequences comprising the Primer A, Primer B, Adapter Primer C, Adapter Primer D and DNA polymerase of claim 32, as well as an instruction for the kit.

36. A method for detection of hepatocellular carcinoma comprising the following steps: 1) Obtaining human DNA samples; 2) Determining whether one of the nucleic acid sequences no. 1˜68 or no. 69˜132, or a combination of the sequences, is methylated; methylation of those nucleic acid sequences indicates the hepatocellular carcinoma; the human DNA samples are derive from human cells, tissues, blood, body fluid, urine, excrement or their combination.


SEQUENCE LISTING (142 sequences)

SEQ ID NO: 1 - DNA, 1062 bp, Homo sapiens
cgggagtggagtccggccgcggacggcgtcccttccagggccggccgccagagggcgagcgcgccttcccgggcggggcggggacgggacggggacggggaggggacgggacggggacggggaggggacggggacacggacacgggaggcttccgctcgcccgcgggtggcgtctctgctcccactagagcgggccaggcctctgggttccgtttctcaagcacacagggagggtggccgcgggccaggggcggccgcgagactccacgtccacgtcccgggcagtccctccggaggttgggccgcgcggcccggggagcggagcgctgcgcttctctgcgagcaccccggggcctttaggcgcccccgtttcctcgtcagtggggggcgcggtgccgcttctccgcgggctaaactgctgaggaaggggcgagttcctgcggcccctgcctgtcccggccacgccccacctccgccccgccgcgttgcgcgttcccgcggggccccgggcacccctggggccccggggatctctgacgcccccttccctccaacccccgctgcgatcccacaggtggggtgaacagcgcccctgctgtcccacggggcgtcttcgcgggtgatgcccacactctcagcctgaagagcaggacctgtccgcgggtggggcccacccccaggcttccggtgcccgcgcggcctccagggcgcctcccccgggcgcatcctccgggcctggcagggagcgcagcctgggagtcaggggcgactggagcccacctgctcagcgcccaccgcggcggccctgccctttcccccgggagcagcagggacagccttggagcagcagctcccgcagggggaggaatggcgcagtggcccgaggctctggagctggccccctgctcgcccggggctacctcggggaagcgcagaattcgcacctttcttctccagctccacgcacccctcatggaaccttagcgaggcccagggcaggggatgctgccaggccgctgccaggctctgacgcggttgcggatgcctcgcccggtctctccg

SEQ ID NO: 2 - DNA, 2187 bp, Homo sapiens
cgcgcgcggagcccgctgagacttgaatcaatctggtctaacggtttcccctaaaccgctaggagccctcaatcggcgggacagcagggcgcggtgagtcaccgccggtgactaagcgaccccacccctctccctcgggctttcctctgccaccgccgtctcgcaactcccgccgtccgaagctggactgagcccgttaggtccctcgacagaacctcccctccccccaacatctctccgccaaggcaagtcgatggacagaggcgcgggccggagcagcccccctttccaagcgggcggcgcgcgaggctgcggcgaggcctgagccctgcgttcctgcgctgtgcgcgcccccaccccgcgttccaatctcaggcgctctttgtttctttctccgcgacttcagatctgagggattccttactctttcctcttcccgctcctttgcccgcgggtctccccgcctgaccgcagccccgagaccgccgcgcacctcctcccacgcccctttggcgtggtgccaccggacccctctggttcagtcccaggcggacccccccctcaccgcgcgaccccgcctttttcagcaccccagggtgagcccagctcagactatcatccggaaagcccccaaaagtcccagcccagcgctgaagtaacgggaccatgcccagtcccaggccccggagcaggaaggctcgagggcgcccccaccccacccgcccaccctccccgcttctcgctaggtccctattggctggcgcgctccgcggctgggatggcagtgggaggggaccctctttcctaacggggttataaaaacagcgccctcggcggggtccagtcctctgccactctcgctccgaggtccccgcgccagagacgcagccgcgctcccaccacccacacccaccgcgccctcgttcgcctcttctccgggagccagtccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaagtaaggctgcgcccatgcaagtagctgggcctcgggagggggctggagggagaggggaacgcccccccggcccccgcgagagctgccacgcccttggggatgtggccggggggaggcctgccagggagacagcggagagcggggctgtggctgtggtggcgcagccccgcccagaacccagaccttgcagttcgcatttcctcctctgtccccacacattgcccaaggacgctccgtttcaagttacagatttcttaaaactaccactttgtgtgcagttgaaggcccttgggcacaatgagagccagtcctccaaactttcagaaagtttcctgccccttctggcaggctgccaatcaccgggcgggagaaggaaggaggggaaggcggtggagggagcgagacaaagggatggtccctcgggggcggggatggcggggctgtcctgtaggtctgtgcggccaccgtgattgcccctctgcgcggtgcccgaagtcccgctgaaacctgccgagggcagcaggtctgaaagctgcaggcgctagttgcgcggaggtggcgcagctgctctggaggcgcagagcgaatacgtggtgtttgggtgtggccgccccgcccctggcggtttcctcg

SEQ ID NO: 3 - DNA, 1314 bp, Homo sapiens
cgcgccctcttcaaacacacgcacactcttacactcactcgcgccgtcccagcctgcctactcccaacgagtccaagccggtgcagccgcgccccatgcccggctcgcagctcccgagccccagtccgcccgcgctgggcgccgctgtacctgttagtggcgcgcggtaccgcggtcgccgggctccggagcgtcctcagcgctgcgggggtgccgtgggtggcctccctgctttcctccaccagaatcctgcacagccggggagaaaacttaagttgctacaccccgccgggagctgttggctgaaaggctgggagtcgcgctccttgcctggcagggagatctcgcactccccggctcccaggccaggtccccagccccagagttggaagagccttagggcgggaaggaagagacagcaaggaccagaatggggagcatgagatcctgatgcggaacccgacggcaggtcccgctccacagctgcctcgcccacagcgatgcgcgctagagcccccgcagccctgaccggtccctctgctccccagccgccgtccaccggtcggagggtcgcctccctgcgaccccctcccccactccactctcaggccgtcgccgcccgtgggctgctccccgccgcggccaaaagtctccgcgcccgaggggaagcgatgggcggcttgggggtccgggggcgccaggcggggcgcgcggcgcggggcgcggccggcgggtgggtgggttacctggcgcggccgggcgcgtctgccgggcaccagagcaggcgcagcagcagcagcagcagcaggctggcgagcagggcgagcagtgcgccccccggccgcatcctgagccacaggcgccgccgccaccgctgctgccggggcgaagcacagcccgggcggccccgactcgcggctcccgccgccgccgccaccgccgccgtggccgcagccccgcgcacccacagcgttcacaggcggcagcgaggggcgcccgcagcccacccggcagagtctccgcggcgggcggagaagaagccgcgttcgggctcgccccgggctcccggccccggcccgcggagcgccgcgatcggctggcagatgacgattcgccgagctcgccgcctgtcctcccggaggcgctcggagctgctgcgggctccggtcgccgctgccactgctgctgccgccgccgccgcgcggggtcacctgaaggttggggcgcggaagctcaactccgtgctgattgggctccaagttttctgcgccctcgcctcgtcccgagtgcccgcgaatcccccggacgcccacg

SEQ ID NO: 4 - DNA, 2032 bp, Homo sapiens
cgcttctgcggagaacccgagcctcctcctgggcgggcgacgctcggctgatgggaccgggccgcgcaagcctcggatgcagccgggtggggggcaccaggcgctaggcgaaggagcgcaggaccaaaggagatgcttggctcctaagataacacctttaaaaagaagaagaagaaagaaagaaaaacaaacaaacaaactcgatacttagcctcacgatccttgccaccccctccctgctccgccacccacacacaaaataaagtgagccgggcgggggagtgcgcgccctgaaagggaacaagaagcaaggcgcctgtgacacaaaatggagaaagaggaaccgggagtggaaaggaagaaggaaaaccgagtaaaggaggttattcataagaacaggaaaggggccgaggggtagagaacacccgaaggaaagcgcaggaggccctcgtgagagggcaagggggaaaagatcgacgaagtaagagcgaaaagaggaaaggaaagaaagactttctagatgcggagtctccattttcagcagcgccatattcgcttttcctctcgccgcttttggctgggttttgtgtgcggcggtggacaggcgccagcacatttctgcagcttcagcagctctgctcagacacctggggagggccgcagggcccgggcgggggacgcgcgctcttggcccaggcccgggcgcggccccctccccggcctggcggttcctgcgcagcgccagccccgcgctcctctaggctgcaaagggggcctccgactcgggcccggcccggaggggacacctcactcggggccccgcggcagcccccaggggacgcgggccctctagtttcgcccacggtgtggctgggcatgcgtgccaaggggatgcgtaaccgagaaccgaaaaagacgaacttaaccttcggggaggaggagagggcgcacgctgcgggtaccggggcttgggggcgctcctggaggtgacggggtcacctccacctgcgcggggccactctctgggagccggcgatcgatgcccgccgcggcgcctggcccaggcccccaggcctctcgcggctcccggaaaaacgtccgaggtcggggctcggccgggagccccaagcgggtcccctggcctcgggacgccagtccgggaggcggccggaactccggttttcgcggttctcagcgatattaggcgcggccagtgtctgaaagctcctcggggttacgtcctggggcgactggaggcggctcacgactcagaaagtcggggcttctcgccccggatccccaccgctggcgtctgtcgaatccccacctcggtcctcgggtagaggcgcaagcgcgccccaagaccattctcttcgcgcccacccccgttattcttcactttcacttgacatcgttcttcactcaaggttaggtcctcgctagtgccagagaagcccctgggtccccagtgccctcagccccgcagcgtagggagcagccggcccgggagataaggaggccacagccatggaggccccaggcccggagtccccgcgccgtcgggttttgagccgcgcggctggccaggaccgccggctccttctctgtcccggtcctccgctcgcagccccgcagaggtgacggtacctggaggctggaagcgacggtttcctcgggccggtggagggaaacatgtcagggtcacagcgaaggaaaccgagacccacaggggtccccgggcgtccgggacgcgtcgctggttttgctcctagaaggaaaggccttcccgcttttgttccaccggctcaccaggtcctcccttcctggccaactctaacctcagctttgtgcgtgtcacccggccccgggataatcggatcctcccagacccgggaatccatcagaagcgctacccgtacttggcgccctgaaagttccccaagcgcagcggccgcgccctcgagtgcatccactccgcgagccacgggcggcgcctgcccacccgcggctcggctttgcgtgttgtaagccg

SEQ ID NO: 5 - DNA, 1019 bp, Homo sapiens
cgagtgaggacgcagctgcagcaggacgtaagcacagtcatcgctgcaaactgcaaactcgtaagcacagtcatcgctgcaaactgcaaactcgtgctccgagcgctgccctcccctgtggagcggaggaggggaggcctggggccgcggcggtgtgcgccccgctctgaccgcagagcccccttcccgaggaaagcggctggcccggtcccggctggtgatcacgcggggcccctgtctgcttggtgcgcaggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggtgcttgtccgcgcgtgctgtggtctacccagtgtctgtctccggccacagttcgtttctcggtcggtttagtgtccgtgtagccacccaaccgtgtggccgaccattcgcgctttcatttgtccttcgcctccgtctgcgccgtctgtcctagggggaggggaagggggagtcctgccagcacccagctgggccttgcctcgggaggcaaggaccaggacgaggcccgagggctcgcgtctggggcatacttgtgccgctgcaggcgggcgcggcgcgctgcccgggcggggagcatctgccgggagggcactccctcccaccagcagttagcccccaacgggagggcccttgagtgaccacgagcagagccggggattggagaaggacgggaaggcggatcacctccggcgccgcccgccccgcccttctccggctcgcgctggtggagcgcgaccgccacctgctgggcctcggccttcctgcagccggcccacccagcaggggccgtgggagagtgggcg

SEQ ID NO: 6 - DNA, 400 bp, Homo sapiens
cgacagcgctgaaaggggtgtgaccccgaggggaggcgacgcgctcccgcgccctagtccacaggcatgcacgaccccacccagcacagggcctagggcatggggactggccctcttggctgaaacgactccgaccctctcggaagatgcccgcgcggcctctgcccccggggagaggggactgtgcccgatgctcaggcgccgcgcccccatcctccaatcccccagaacgcaccacaggccaagcccctcctgcgccagccaagactccgcgccagcccacaccagccccactcagaccgctggaccccgcccccggcccccgcaccacccagcccgcaactctgcagctttcttcgcccacgccgcacagcctccaaaccagcggattccgcccacg

SEQ ID NO: 7 - DNA, 609 bp, Homo sapiens
cgcggagagggcgcttagtgcgtgagctaccgtcgttccccgcgactcagccgagcggcatccgcacaccctgctagcctaggaagcgcatcgagggagcccgctcaagctgaagcgctgcacgagctgtgtcttccttctctccgccgcgaatgcgggcaggatgcgagcaaaagacccggcaaagagggcgccgagggggcgcagccggggcgctgggcggacccagaggggacatggagaggaacgcggaaggtacgcggtagagacggggaggtggcgcgaggcgggacatggtgagcgacccgaggggcgcgcggactgggctcgcggacggggctcgcggacgtagagcagtcgaggcgcggagggggagcgcgcccccgtgtggccaggcggggaggcgccacggccggggccgtgggggcggtgggaagcgctccgcgtccgggctctcagcgttccctggcctgccccggctcgctaggctttgatggctgcttccgctccccataaaggcctgtttcatggccgacgccggcatgggctttacgtctgccctcgggctccctgcgggctgcagagggcagcggtgaaatcccctgtccg

SEQ ID NO: 8 - DNA, 724 bp, Homo sapiens
cgtacagtgaatgcgtctgcgcacgtacagtcctcgtgcaccctttttctctctctggacagaatttcttggggatccttcgccccccacccccacccccggccgtatttctccctaggctcttattaaaaccaggtgcacggaaggcggcccattctggacagaaaagctttcgtttgacccaaagctcccccttccggcgtgttttctagagggaattcgtgagctgagccgaggagcgagcaggcggcttctcctgaccggggagacgcgggaggtttgcaaacagaccttggcctgggctgcgcagggacaaagtgttcaagggcagcaggggaggctgagggagtacacggctgggaacaagggacactgccgcccccagccacgcaggagcggtgtctgcaccactgaaagtcccccggggcccacagctctatccaggcggaagggaagcagccaggccgccgcggatctgaacttgaatcccgggtggaccggacgccccggccggcgctcctgacgcgcggaggagctggcgagaacctgcagatctcagggctcgcccgcggtccggcgctcggttcaggacgtccgggaaccccgcagcgcttaccctctgcagcaggcgtcgctggcgcctccaagggcaggggcgggtccggcgggtacaagtcgcgattgacgcgacggcccaggcaggcactgtgggcttcaccgcccg

SEQ ID NO: 9 - DNA, 923 bp, Homo sapiens
cgacgcctgcgacatgaccatagaggagatggagagcgcggccgacaacatcctcagcgggggcgccccacagagccccaatggcgccctcttaccctttgtgaactgcagggacgcggggcaggaccgagccgggggcgaagaggacgcgggctgtgtgcgcgcgcggggtcgaccgagtgaggaggagctccaggacagcagggtctacgtcagcagcctgtagtgggcgctgccagatgcgggcttttttttatttgtttcaatgttcctaatgggttcgtttcagaagtgcctcactgttctcgtgacctggagttaaccggaacagcgtcttcattcatttctgttgggaccagacgcggagcctgggtgcgcgagccgccctccgggaggaaggcgcccggctgcgtctgcagaggcggggagaggaggcggcgagggtcccggggcgcgaggaaggcgcctgccctctcccagctcgcaggccccgggcccggccgcgcctccgcggggagagcaccccggcttcccgcgcgccctcaccaaaaggaccctacagcaaacgggtgtctttcgactctgcttgtagaaaccatttgcacatattctgtacgagcctcgctgtctccctagagccagggccctgcggatttggagaagggagcggggcaggacttccaggaggaccccaacccggcccggagagggaggaggaggcctccaggggcgcggagctctggggatgggcgtcgggccggcagtggtgcggctcactccgtccctgcccacctgcgacgggatcccccgaccggcacgggccacgccgagctcccggccagccgccggcccgcaggcagcgcgagggaggagctgcgccgccggctccgcccaaccaggtggtgctgagcttccgctgagcg

SEQ ID NO: 10 - DNA, 920 bp, Homo sapiens
cgatgctgtcgcccccgcgacctgtagggccttcgctggcccctcaccctcctcccgccccgggctgaggctgccgccggtttcgcgcccagtgccgaggcagcggcggagccggggaaggtcggagggagggtggtttcctcccgccccacacccagtctccgagccggatatatagagtgtcacgtttgggagccgaaagactggagccgtttccttgtggctggagcgcttcccgtagcctcggggaaggagcaggatttagaggaccactagttggaccccatcctcgtgctggaggaacaggtattccagggtccccagccctggcgctcgcagagcccacgcgctccaaaagcagagaccggactgggggtggcccaggagggagggctcggctgggttcgcagggcggggcggttcccggcgggacctcgaggagggtgggtgtgaggccgggtgggaggagagaagcatttgcggaggctctgccccctgaagctgcccccggctgcaccccaatcgtcaccggccgtttacgcgcggttggcgtcggcggagcccgccaaggtggggcgcgcgggggttgcaaagggcagaaagccgggaaggacccagcaaggttaatgtggggggccgcgccttggggactggggacccgggaagagaggcgcggggccgagtcccacccaggcctcctgggcccttggggctattcctggagagagcgcgaaaatgccggggccacgtgtgaaacggcagcggtcctaggtcccctcccggtgcctgggaccgccagggcgcccagtcctggtccccggagcaggtcctcgccagcccccagccccaccccaggctgcgcacgtccctaccctcccacctgggcctcgccggggtcgtggctgacgcgcgctggggcg

SEQ ID NO: 11 - DNA, 538 bp, Homo sapiens
cgagttccatgtgcggcagacacgcgctcacacttcgcagccggaggagcaggcaggaagcgccgccccccggccgccccggccgcccaccctcaaccttcctccacgttaccttgggcgtgtaggcttcccgtggacctgcccgcgccgcccgctcggctctcagctgtcaagcagccgcctgctccgcccgactctttccgctccgagccgcgcgggctggaccagcccctcagcggagctgaacgggaagcggcgccccctggacgccctccgggacgtgcgcgcgccaagcggtggggtccgggcctgcaggcgcgacctgaggccgccagggaggctgggtggcgagggcggggcggctgccgacttagccggcctgactggtcgggcttccggggtcccgctgctccctaggccccttgccccgattcaggctcccaaggtggggcatcagtccggccctcgctggctcactggagaggtggtgtctgggagtgcacccccgaaaacccggcagcagcccggcgtcactccg

SEQ ID NO: 12 - DNA, 649 bp, Homo sapiens
cgaggccaacgggacggggctggagctgctgggcggcccgtcgctgcgcagcgcctacatcacctccctctacttcgcactcagcagcctcaccagcgtgggcttcggcaacgtgtccgccaacacggacaccgagaagatcttctccatctgcaccatgctcatcggcggtgagcccggccgcgcgcgtcttcccggggccactcccagacttctgcccgcaggcggcgccgtcccactccccggagggcctgaggccgggggctcttccgctttaggtggccgcggaacccgaggccgggggtgggggagacttcgcccgcacagcggccttcaggccttgcctaggagcgtgcgggccgaggacagatgagcccagcgtggtgtcaggccgggccaggtcccctaggtgaccccctctcgtcaccctcccccagccctgatgcacgcggtggtgtttgggaacgtgacggccatcatccagcgcatgtacgcccgccgctttctgtaccacagccgcacgcgcgacctgcgcgactacatccgcatccaccgtatccccaagcccctcaagcagcgcatgctggagtacttccaggccacctgggcggtgaacaatggcatcgacaccaccgaggtgcggcctccg

SEQ ID NO: 13 - DNA, 1229 bp, Homo sapiens
cgcagccccgagccccgagccccgccccgggggcctcatcccgccccttcgcgtccgcggctcgttttcccccactgagcgcccagctcccgcagtttccccggccgtcgagcgccgtgggcggggctccagggcggcggcgcctcgcggggagggtcctccgtgctgggggcgaggccacccgaggcagctccccgcccgcccccaaccccgccccgctctcggagcctataaagggaggcgacccgcggcccgcccggctggcatcccccagccgccgccagccccgccgaggggagccagcgccgtctctgaggggcgtccggcgccggagccatgaccctccgccgactcaggaagctgcagcagaaggaggaggcggcggccaccccggaccccgccgcccggactcccgactcggaagtcgcgcccgccgctccggtcccgaccccgggaccccctgccgcagccgccacccctgggcccccagcggacgagctgtacgcggcgctggaggactatcaccctgccgagctgtaccgcgcgctcgccgtgtccgggggcaccctgccccgccgaaaggtgcgtcccccgcccgccttcaggatctgctcagcccctctccgactccctacagggcctgctgactccgcagtgccctctcctcggcgtccgcggagtcccccaccttcttccccggcccgctgggtgcctcgactccccgcgttccccgctgctgcgaaggccgtggccctcgcctgcacaccgcgcccaggctcggtggctcttaactccgcgccccatgcacgccccctctctccctccttgactcctcccagcaccccccttctcctacccgctccatctggctttctgccccccatgccccgcctccccgtggccaggtgtcctgggtccccaggagcccctcgcccgagggacagagacagccccaggcaagttgaaggtccgagagcccccggtgggagaagcgggccggtggctgcgccgcgtgcgttctcactctgaggaagtgcgtggggagccgctgactccggatagcacacccttccgaggggactccccgattcctgggctgggggcctgccgcctggccccacgtctgacgtacggggcgcgagggccactgctccctggacttctgtcggaaccggacgcagtgggaggggtcgcagggcgcccgcggggcaggaaggatgcgggccgcg

SEQ ID NO: 14 - DNA, 2375 bp, Homo sapiens
cgcaccacgttctttttgaggtccagtttctcggcgatggcggcgatcttctcggacgagggccggggctgcacggcgaagtaggcctcgagggagcgcttctcgggcgcggcgatggaagtccgcttgcgcttcttctcgccgccgttgaagagctcaggcttgttcattttctcgcgctgggcgccctcggcctcctcgagccacgcctgcaggatgggcttgagcgcgatcatgttgttgtgcgagagcgtgagcgactcgaacctgcagatggtgctctggctgagtgagcccacgcccgggatcttgaggttggccagcgccgagcccacgtcggcctgcgtcacgcccagcttgatgcgccgctgcttgaagcgctccgcgaacgcctcgagctcgcgcgggtccgtgtccgagtcgcagatggacgccaggcccgctgcgcccaccacggccgccgccgccgatgccgctgccacctgcccggccgccgccgccgccgctgccgctgccgcgccgtggtgcgccgccgccgccaccagcccggggtgcggcagcccggacggcatgttcatggcggccgccgccgcggggtgcgacaggtggcccaggctgtgcatatgcgggtgagggtgcgcggagccgcccaggagcccgccgcccgggccgccgccgccgccccccgggccgccaccgccgcctcccccggggccgccgcccgggccgccgccgccgcccgggccgccaccgccccccgggccgtcgtgggcgccgccgccgccggccgccgcgcccgcgccgcccgcgccggccatgagcgcgagcgacggcgaggagatgtggtccagcagatcgccgggttcgagcgcctggtggtggtggtggtggtggtggtggtgcgccagaggcaccgtggaagtggacgtgcacggcacgctgttcatcgtgtggtacgtggcgtccggcttgaaaggatggctcttgccctgggacacggcgatgtccacggccgccagcgcctcggcccgcgccagcagcgtctcgtccaggctggcgaagaggttgctctgcagctgcaggcgacacaaaccaaaccaaaaaaaccacaaaaccaaaagagcaaaacaaaacaacagaagaaacacacacacaggccggaaagcacagcatgcgaagggcaaacacaaagcaaccaaaataacaacgggtttgggggcagtggagagcgggaaagacggagagggggcacattgacgaccagggagggggcagacgagaagggatgggagcgtggagagggggacagaagtagggagaaagggggacacaagaacacattccggaaacgggcgtgggagacgaaaaagagggaaaagaagaaatggaaatgtaactcgcagctggggacccgtgtcacacacccgagcacgcacagagactgcctttctgaggcgtgaaattaacagagaaagtagagggaagtcgagagtcctggccccagcgctccctactccgatcgcaggcgcctgacgcggaggcagaggcagacaggaacacgacattaagggccgccggacaaaacgtgcctcgctgcgggactgctctacaccccctgcccgcagttccccgcaccgggacctgcacacaccagcccccgacatgcagcgtttcggagacggggaggggcgggcgcgcgggccggggccgcgggcgtggggcgcttaccggcggcgtgggcaggcaggcccgccggatggcctcggagctggagtgcagcgacgggtacttgtgctcagggagggtgggatgcatggcaaagtgaggctgcttgctgttcatggacatcatcgtggcggcttggcatgtatatccacaaacactccgaaagtccgcgggaaagtgcgtacgccggctcacccggcctcccttcggaggctgcagccgcggcggtcgcggcggctggcggcggccccgccgcgggctgctgctgctgctcctgctgctgccaggcgctccctctgaccgcgcagagcgccgcgcgccggcctcgcggtcccgcttctccgacagctctagccccgcgcgccgacgggatgcactcctctaacacctgagccccacttctcgcggccgtcccggggagctctcgcgagagctcgcggccccaccgcgctgacaggcatcagctgtctccgtctgtctgacgcgcgctctccttctcggcggccccgtgcgtctgcgcgcgcgcgcgctcgtccggccgcgaccagcgcgtgggagccgctcttatagtgaccaccagcaaggacagcgcaggtgatgcacctgtagctacccgggcatgcgcactgcccgcctcacctttcccaccg

SEQ ID NO: 15 - DNA, 723 bp, Homo sapiens
cgactcccgcaggttagcgctatctcgggcaccccgcagggagagaggacacggctgccccgcgaaaacactctctggcacccgtgcttccaggacaggccctgaggcctctcccggggaagcagttacacctgttctccggggctcccctgaagaccccaaaaacacccaacacctgcctccccaggaaagacgctctgggcccgatctatgccgcccggccgccgggcctgagtgaagcctgtccagcctaggaggggccctcagaacctcccggtccaagcctctcacttcgtccacccctgcggagacggggcgagccccgcacccttcccgtggcccggccgcaccgccctgtgcccgtggtggcggccacacaggccagcctggcgagtactcacggcgaccccgggccgcgcgcggccgcagccgacgggacgagcggcccgggacgccccgcgggggcggagcggcaggccaaggggcgggggccgggcctggcgggcgaggtcgcgcaccccacgcctcgtcgttccgtcacgggcgcgctcagccactctgcgcacgctcgcgcggtgacacgatggcacgtggcccgctcctgaccaacagcctgacggcctgctcctgggcgcgcctcagcagccgacgccagccttgagacgcctgcgcggcggtcgctagtcgggaaagggtgcggagtggggggtggcgcctgcgccg

SEQ ID NO: 16 - DNA, 3253 bp, Homo sapiens
cgacttcttccggagggcttcgctcgttccgagggtcctgcgactggaggaggcacatccctgagcagccactccctccccgcagcggcctgtcgccgagctcccgcgccctgcttgagctcgggagagatcgctgcggggcagtccggcccgggagcgagagaaagaacgaaaatgcacagctctctccgagtaaggcgaacagatggagaggaagacgaacaattaaacgaaacaaacagaataaaacagaaacgaaatcgggccccggggggctgggccacccggaggttgggctcggggcgggtgggtggcggcccctcgctaacctctggcctcaccctgtcctggcagacgctgggtcgggccggcagcccgcgttgcgcccgcacgtgcacaagcagacggagaagacgacccgcgtgcggactgtgctgaacgagaagcagctgcacactctgcggacctgctacgccgccaacccgcggcccgacgctctcatgaaggagcagctggtggagatgaccggcctgagcccgcgggtcatccgcgtctggttccagaacaagcgctgcaaggacaagaagaaatccattctcatgaagcagctgcagcagcagcagcacagcgacaagacggtgagcagccgctgggccggaggctcgagtcgggtgggggctgcgcttccgccgcggtatctgcgtgcccttttctgggcgagccctgggagatccagggagaactgggcgctccagatggtgtatgtctgtaccttcacagcaaggcttcccttggatttgaggcttcctattttgtctgggatcggggtttctccttgtcccagtggcagccccgcgttgcgggttccgggcgctgcgcggagcccaaggctgcatggcagtgtgcagcgcccgccagtcgggctggtgggttgtgcactccgtcggcagctgcagaaaggtgggagtgcaggtcttgcctttcctcaccgggcggttggcttccagcaccgaggctgacctatcgtggcaagtttgcggcccccgcagatccccagtggagaaagagggctcttccgatgcgatcgagtgtgcgcctccccgcaaagcaatgcagaccctaaatcactcaaggcctggagctccagtctcaaaggtggcagaaaaggccagacctaactcgagcacctactgccttctgcttgccccgcagagccttcagggactgactgggacgcccctggtggcgggcagtcccatccgccatgagaacgccgtgcagggcagcgcagtggaggtgcagacgtaccagccgccgtggaaggcgctcagcgagtttgccctccagagcgacctggaccaacccgccttccaacagctggtgaggccctgccctacccgccccgacctcgggactctgcgggttggggatttagccacttagcctggcagagaggggagggggtggccttgggctgaggggctgggtacagccctaggcggtgggggagggggaacagtggcgggctctgaaacctcacctcggcccattacgcgccctaaaccaggtctccctggattaaagtgctcacaagagaggtcgcaggattaaccaacccgctcccccgccctaatccccccctcgtgcgcctggggacctggcctccttctccgcagggcttgctctcagctggcggccggtccccaagggacactttccgactcggagcacgcggccctggagcaccagctcgcgtgcctcttcacctgcctcttcccggtgtttccgccgccccaggtctccttctccgagtccggctccctaggcaactcctccggcagcgacgtgacctccctgtcctcgcagctcccggacacccccaacagtatggtgccgagtcccgtggagacgtgagggggacccctccctgccagcccgcggacctcgcatgctccctgcatgagactcacccatgctcaggccattccagttccgaaagctctctcgccttcgtaattattctattgttatttatgagagagtaccgagagacacggtctggacagcccaaggcgccaggatgcaacctgctttcaccagactgcagacccctgctccgaggactcttagtttttcaaaaccagaatctgggacttaccagggttagctctgccctctcctctcctctctacgtggccgccgctctgtctctccacgccccacctgtgtccccatctcggccggcccggagctcgcccacgcggacccccgccctgccccagctcagcgctccctggcggcttcgcccgggctcctagcggggaaaaggaaggggataactcagaggaacagacactcaaactcccaaagcgcatgattgctgggaaacagtagaaaccagacttgccttgaaagtgtttaagttattcgacggaggacagagtatgtgagcctttgccgaacaaacaaacgtaagttattgttatttattgtgagaacagccagttcatagtgggacttgtattttgatcttaataaaaaataataacccggggcgacgccactcctctgtgctgttggcgcggcgggagggccggcggaggccagttcaggggtcaggctggcgtcggctgccggggctccgcgtgctgcgggcggggcgggcccggtggggattgggcgcggccgagggggtggggctgggcgcatgcgcgctgagggccctcccagaagcaggagcgccccattggcgacccctgggcacctggcggcccaggcccgcgggcaggaggcgggacccgtttgggcgggaaacctgcggcacttgtggcggttgagcggcggactccggcctagggttatgcgggcgagggcgcgccccgctcccggaaccagccttggcctggcgcctgccgagccccgagccccactctcgctcggcgccgcgggccctcccggcgcttccttccccagcggtccgcggccctgggtgaggctcgaagccccgcgcgctgcccctggccctgcagcacagcagtgaggccaaccccggccagagcgggcggccgtgacgggccgtgccccgcgctgtccccggagtggatgcgcgacgtgatcaatccgcggcatttattaattccacagtctagacagaggcgggggccgggcggcggctgcttatctgcgtttggcatcacctgcaagcgatacaccgaggggccgcaacgagcccccgctaggagcgcggctgccg

SEQ ID NO: 17 - DNA, 1061 bp, Homo sapiens
cgccgatggctactggagcggagaggttccgaggtttcaggggcattttgtcctttcccacttctgggccgtgggctcccccatagacgatcccgcgggcccttcctcctctgacaccctcggatctggttccacctttcccggaccgcagctcagccccgcgcctctccagctcttcgatgaccggttcccggccgtgccccgggccctccctcggagcgcgcgccgccaggggcacctgtccctgcgcgggggtgtcgccgcccctcggcgccgccgggcgcttgccgctgagccgtggcgcccctggcaggagcactgcaaggacgcctcccgggactgcaccgcggccctgcgcacttggggcgacggggccaggcgcgggggacggctgcgggctgggagggcgcgcggaggagacaccgctgaggggacgccgctgagggcgccacgcggggggcgcggctgaggcgcctcgaagggcaagcgccaagggggcgcgggcgccgccggaagctggggcggggcgcccagcgggatgcggtgaagggcgagcggcgcggcggctgcgatgagtgcctctgcggccaccggggtcttcgtgctgtccctctcggccatcccggtcacctatgtcttcaaccacctggcggcccagcatgagtgagtgagccggcgcggcgggggtcgcgccgaggggcggcgggagttggctcgccgcgacgggagcctcgcaacttttccgagggggctgggaccgtccgccgcgggacagaggttcgtggccgcaggggctccccgcgcctggccagactaggggggcgccccaggggtcgcacgggccgggtcttggagccgggccctgaggtgcccaggctggcgcatttcgggatgttggtgccagcaccgcgccaggtgcccggggtcacaggcgtcaatacggccatgcccctgccctggaggggcccaggggccggtggggagcggaacaaacacggacactacgaacctggccgtgtcagctcgtttgtgccttgttctttttgcatctcacgggaacg

SEQ ID NO: 18 - DNA, 1161 bp, Homo sapiens
cgcagaggccggggtggaattcgctgcgccccaccccttccacccgctccccttcgccccgtaggtctttccactctcgctcctcccctgggcacatctcctgaacgcagccccgggggccgaggacggggtggggtggggggcgaggctcgggtccgacgaccccgggctgcggtcccggcgctgcagagctgcggctgtgcacgcttagccgcgaggcccgcggtagcccgggcgccgatatgtaaagcagctggcagcgctgggcggggcctgggcgcgatgcaaatgaggagggcggggctggcccggggctccgcctccctcccccgcagctggggccagcggtgccaagcgcagctggacgagcggcagcagctgggcgagtgacagccccggctccgcgcgccgcggccgccagagccggcgcaggggaagcgcccgcggccccgggtgcagcagcggccgccgcctcccgcgcctccccggcccgcagcccgcggtcccgcggccccggggccggcacctctcgggctccggctccccgcgcgcaagatggctgacccggctgcggggccgccgccgagcgagggcgaggagagcaccgtgcgcttcgcccgcaaaggcgccctccggcagaagaacgtgcatgaggtcaagaaccacaaattcaccgcccgcttcttcaagcagcccaccttctgcagccactgcaccgacttcatctggtgagcgcgcgcgcgcagggcaccttcccgggcccccgagggcagcgccgcgccagggaccccctctccgcgccctctgcgccctccgcgccctccgcaccctgggaccccgcgtctccggactcccggctccggaccctgctgcccgggactcccggatggacagtcctgccgttgccctgtccccaccctggtcccagacgggccgccgcggggcgcctcctgccctctcctgctctcaggcgcctctagagcgcccaggggcggcgtcgcgggagcctttgctccacctgactaggagcgcgcggggtctgtgcctgccctggagggcagcgcctcgggtgctctccgacccggggttccctatctctccgcctgcttccgggcgcgaggagccctcgccccccaccccttgtttccggggggggcggcg

SEQ ID NO: 19 - DNA, 1433 bp, Homo sapiens
cgcgggatcgcagtgtttgcctttgcatctccagctccgcatcctgctcgctgtttattttgtgagcagctccggcggctgcccctgaactgctccgagactgagcagtttaactcacactcgcccacccgccctccccaggaagcctcccgcccgcttcctccagttccttccccacctgtccggcctcctagcccggtgctctcggcgcgggaggaggcggctcgcagacccccaggtctcggcttcccggatctcccccgcctactcactccccggtccccggctcccggcactggcaggcgctgtggcttccgcgcagtggctgcagcatttaaatgtccggcacttagggagaggggtgcagtcgcggagcagccgctgctcccggcatctagaggggcccaaacccccgcgtgcccaggccgagccccgcgcgcagcccacgtgggctgagggactgagtattggccctcgggcagctgcaaccgcgcacagctggagctggcggggcggagggcgggccgggaccccgcggggaaggaggcttccttcccagcctcctgctgcccggaaatccccccggctctcagacaacctgccccacggggtcctggggggaaggagaggggcgggatacagggatggggagggctggctgagccgaaggcccgggccgccctcccccaggccgcgcggcttcgagaaagcagaagcgagccctgccccggggagtcccacaggagcggggatctgggaagagaaacttgcggacgagggagttagagccgtggacccccggcccccgcccgggcgtcctgcgtcggaggagggtctctgggtcgggcaggggccacctcttggcccgccccttgtcgctggacgtccgaagattgggccatttcccttgccgcgcccttaccgcatatcggggtgcgcccggcccggcccggcccgccccacccagccctccgctcgcgcccggagaggaggggccgctggcgcagcgccccgggaccccgagaggccgccgcggcacatccagacctccgccgctcccgcgccctctcaaccatcctgggattcccgggcccacccgacccagcggcgcgaccctggccctccgggaccctccgctgactccaccgcgcacttcccgggacccccacacacatcccagccctccggccgatccctccctactcggtgccgggtgccccccgccctctccaggcccggatctcctcccccaggtccccggggcggccccagccaggcccccttcgaaccccgccggcggcccgggctggggcgcaccatgcggctgcggctccggcttctggcgctgctgcttctgctgctggcaccgcccgcgcgcgccccgaagccctcggcgcaggacgtgagcctgggcgtggtgagcgcggggtccg

SEQ ID NO: 20 - DNA, 1118 bp, Homo sapiens
cgagacacacgagaccactcctgccggggaaaacccatgagggggtgggggcggccgcgccgcagggaggggccctctgctgtcccagccaacgtcccatgacgattttgcacccccgccagcagtgccctcggctggacacacctgcgttgtgggcagccggcagcgcgtgacccactgtgcacctgcgccttgatgtaggggggagaccctgcagagagcgcttccttccgccctgtccagtgccctccctgcctcacccctcttcccgcaggcccgctcagggcgctgaagggctcctgaccgtgggtccgtccttcgctgcgtctggggggcgtcccccgcgctgcctctgacccgcccggccctgcgtttgtctgaagcccctcgttccgcatctccggaccagggtccgggggcgtggtctgctgggggcgggcccgggttgtggtttgagagccgctcggcctgtctgggcctccgcctccgggtcgccctccagccttcctgccgccgcttttccagtcttgcccaacccggtatttctgcgcttgcctcagccacccgcaggagcgcaggccgcccttcgtcctcccgtcctctgcgcccacaggaccccgggccccgcccagtccgcaggtcccgcaggtccccccgggcgtgtcttcctggccctgcgacccccgggacagcgctgagaacacgcggaaggtggggagacgcgcggcgctggccgggtccctggcgccgttctcgcggtgcagcctgtgcctgggagtccggccgtcccacctcagaaccagagcaaagcgggagatggggcggcggccgctggggagaaggcagccccgggtcggaggaggaaggaggcgggggcggagcggagtccgcggtggggctgccttcttccatcctcccctgggcggggcgcaggatgcgggggcgcccagccgagggagggaggccggggaggacactcctgtgcccgagggctccggcttcttctgctcactccctcctgccgaacttttccactcggccatttacctcctcaggcactggctgccctcatcaggccgaccaggtcctgcccgagggaacggcgtgtgtgacccacg

SEQ ID NO: 21 - DNA, 580 bp, Homo sapiens
cgcacggagacctccaggcaagcaggatgcgcgctcgccctcgcttgattgtttgctccccaggatgctggggaaagcggctgctgaaagagcaaactttctgagcgttcgcggaggcggagggcgcgcggcggactcacagctggttgatggcgttctgcgagatgaccgcgttggcggagaagtaaggaatcatgtcggggccaccgaggctgcaggtgcaagcgaggggcgccttcctcttgcgggccaacgtgctcagggtcatggggccggctcaaagtgcacaactccgcatcctggcctctgctctgctccggcacgaggcgcaggctgcgcgtgcacccggcggcggaggcgggcccgccgggtcgcgcggaggcagccgccgaagccccagggctcggcggtagagtccggactgacgggcgtcgccagcagttcgcccccacccgcgtccggggcgggacgggcgggtcctgagagcggagagggtaggcccggccggcgccccgccccagatctcgggacgcccgcgcggatcccgcagcgcgcgtcacagcctttagggcatccggcg

SEQ ID NO: 22 - DNA, 370 bp, Homo sapiens
cggggaggctgggcgtgccaagctggaccggtccggccaggctggaagcccagctgcccgctttttctcagtgcctggagaccgcccgccttttcagcaccacggtcagcggccttttcgcaggcgcgcgccccagccccgccccgggttgggctggcatccagcctcccggcccttctgcgtgtccctcggcccaccggcatcccgggctggaggtgtgggtggggcaggcgaccggggtatgagaggcggtggggtgcctcctccgcgcgtctctgcgagtatctgtctcccccatgcgtccgctgctcctcatagaggcgtcgggagagtgtgttcgcgactttctcgcacagcggctgcgcccgcg

SEQ ID NO: 23 - DNA, 479 bp, Homo sapiens
cgccttctggacggcgagggtgacgcactggccctcttcctcggtgtcctcaggtgcgtggcggcggcagtgagaatgagttcttcgagatcgccgggagaccacacaataacgacgaagttacttgccgggctacccgccgagacttgaaaacttcgcggcgacttctctcgccggccttagcatcgcccccatttttcaggttacaggacaggcctgcaactgcaaaacgctttgtaaaccacagggccccgcgcgggtgtgcgccactgggttagctgagctgcagcccgggccgggcgggctgggacgtgagcaagccgggggagacgggagagccatggggagccagatcccaccttcgcgtgaccttgggcaaatcacaacctcgcctaaggcccggaatgactctgcgcggagaggaagagcactgagtgcgggagggcatcgacgttttggtgttggttgctctcgtatcg

SEQ ID NO: 24 - DNA, 708 bp, Homo sapiens
cgcgcatgcgtgtccgtgagagcacatgggtgtgcaccgcgcaggggctgaggctgggagccgtagtcaggggcaccgggtgggctttggcggcgggtcgacctgcctttcaggtgccgggggtttgggagcaggggaacgggccctgggcggattcctgaccgcccccttcgcgcccctccccctctcactttcgctttccccgcctgccccaaccctcgcccccgggcgctcggccgctcccacctgtcacccccatgcagaccgaggctctgtcctggaggcggggagcgggctggtggatgctgcctctccgcctccagtgtgctctttggctgagggactggtgggcgctggaaatattagaggcgccggtgcctgtgatgtcgccgcctctgtgacgtccccgccgcgaccccacaggcagggagccgcatttaggtttcgggcgggcggacgggggacggcccgagtgaggcgtcctccgaaggctgcgggcccgaagccagaactccttcctctccgaagacctcgggggtggggctcgcgcttccgcagcgggcctagagcgagaggcaggcggcaggtaagcctggctgtgctggacaggacttgcacccgctctgcacgccagcggctcagggctgccgcccttttcgtggtcccagggcccttccaagaaccggctaaaccaacccaagccgcgcg

SEQ ID NO: 25 - DNA, 10206 bp, Homo sapiens
cggaggcggcacagctggagcccggattgtggcacgccgtcaccgtgctgctccggggaatcccgacccgctccctgcgaaagcgtttccgaacgcgaacccagagcctgtgaacgcgccggcaagccccccactcccccaccgccgcccgtcgcaggtgggcccgtcctaggggtccttcctgcgctctaccccgtctctcaagtcactcagtcgatcgccccgttcacgctcccgtgatcccagacatccataaccacgatctcgcctccatgcacatccaacgcacgacggtgcacagacgtgcacctgacttctgcggaccaggtgtctcaagcgtacagcggccacccgcggaaccgcggcccggggccagtgagtcgtctgcagctccccgggttaggggattccccagaactccgggaaagtcacccgaagtccatccgggatgcggccttggttctcggccgcgtgatctcgcccttaggtgcagaacgacgcccttccagggcccacagctgccaggctgggccttgccctcgcatcccccgggaagaccagggacggggccacacaggccgtggctgcggagacgcttccccgggccaccccgcgaccaggagggagtcgagccgcccgctctccccgcgtcccccgccccatcccagccagttggccccaccctgcgcgggataattgggacgggagggaggcgacgggagggcggcggctccagagagactgcgcgcctgtcagcggcaatttgtttaagtggacgggacgggccgggccgctgcgggctggggtcaccgaggccgcggcccccaccccaaccagacctgggaccgcgggggagcccggtccggccgctaaaccgggctggctggcgccagggctccgggaggtgcggtccggcggggaagccgtgatgggaagcgactctgtccagggagtgtccttcaccaccacactcctcacgtccaggcagtgatcgacggcctggcggcaccctcacagcgggcccatagcacggggccacacacgtcccctgagcttagcctgggcacattcgtctgccaccgagggcttaagccagtctgcagcccgcgccccgtcactcggacgcaagtccgtcgtccgctctgccacgcggccgcacagcccgagcttcctgctgcccactgcccgcggggtcacgcaaccccggccctgcacacagaggaggaagcggcggggaccccctgccacctggaggctcaacccggggaaggtcccagcagtgccccttaaccctgcgagaggtgacggggccggaggacggaagggcagccacgccaggctcaggaccgcggccccacccggcccaggccgtccccgctcccgccccatttcactgttgcgggaagcgcggggcgcacagaccgcgcgccaccagcccaagcccgcccgacgccctggccggagaccgcctgcgagagggaggggcggccgagcgcgcgtcccccgcctgtctgcgggacctggccgatgcgaagtgacaggggccggagctttgttgtggagcctcggccgcctggcgccagccgcccccgcccgcgccctccccctccagccccgggcggcccgcgaggcgccccccggggcctcttaaaggaacacgcacactcggcccccggggctccgccaggggagggggttcctgggaagggggagggggccggcctaggtgggggaggggcccagcggaggcctcacggcgccgctccagggcctcccaggcctccccagcgccagctttcggtgagcctcgggcattcctagctggagagtttggctgggggtcgctacagactcttcagagcggctgcagcaggacctcagggtgatcgcgggaccgcggggcggggcgagtggtgggatcctggcccggacagagccccggaaagccagagggaaggtcggtgccactgtaggtggggtcgccgtccccagatgcagccgctgcactgtcgccgccaggtttctcggtcaaataacccaggcctgagtgtttcaaactcttcaggcctcggacccctggaccagctatggatggactaatgaaatggactaatgtctaaaacgcacacatttagatgtacacatactcttacaaggacaaaagtatgtgtctacacacacacacacacacacacacacacacacgcgcgcgcgcgcgcgcgcgactttgcgaatgtattcacggattcctaggccggtacctgggttcccggctaaggaaagctgccctggttattatctggtcacctcaccggctgcgcaaacgtgtccacaacgggtccctccccgagaggccacatctcgcctaaggtggagccagcaggtatttgcctgtggaaaactgcagtggatcctgccccgtctgcgtagactgcgcagctcggagtcaaagattcgttctggccagagaggaggtactaagagaggtgcgacctagccaagaacgaggtcgtggagaaaaccctgtgggggcggcgctcacatatgcggtgatgaggagttgtgagggccctggggtcggccccgaaaagtagggataacggtcgcagaggccatactgcttccgccggtcccgaatccgcactgccaggagagatccggccttcaaacgctgctcccaacgttcaacaacgaagtgccctgcgaccggtgtgcgcagtgggcgggcgcctgcatgggtcaatgttggatgtacgcgcccaccacgcggttgtacgggtgggaatgtcacccgtggctagtgcatgctctgtcttttccttcagaaaacgaccttcaggaatcagcctgagtgttcgcgcccgagcccgattggaagcaggtgcgtggtcgcttcactctccccgtgcacaccttgagttatagctctcgctgcgcacagagggcaccacacggggcccgacacacacacacacacacacacacacacacacacacacacacacaccatcacttcatgggggggaaactgaggctgccggaagacagaaaacgaagactgatcacagaagagggggcttcaatcacaggtgctcgcagacaccccaactccctcacgcgttactcagcactccttaacttaggagacgtgcgcttccgagagatgagaagaaactaaaaaagtggttggcagggcgaagagaggaagccggtatttcggaagaaggagactttcgtctccggtaccggatagggcgaaaattcgttgggtcgcgggtggagaatccggggtccgcccgcgcggtgccgccctgtgctcagcgaaaggtgcgctcaccttcctggccccgaggatccgctcctcccggccgctggaaagctcagggcggggcccgggcctgcggagaactccaggttccttccgagtcttccggtgaaggcgggcgcgggtagggggtccccgcgtttccccagcgcaaggccgcgtcccgctcccgcccgcgccggccccagcagggctcggccatggctccactcaccagctcccgccacctgggatccgctcccggacctcggccgcccgagcctcccgactcgcccgcccaccggcctcgctttccagtgcctgctgtctctttccggggcgcagggacccccaggcggcgccacgacccggagctgggtcggaggccaggctggggctcgggccagagcggccgggcctctgggcgcgggagaaggagggcgccccctctccgctcggggtcgtgtcaatgctttgcacttggggccggcgtgcggctgcgggtccttccccaaggccccgggacccgggctcccctcgcctcaggccctttcggcgggtcaggtcggccctccgcgctctccagtccggcgcaggcagccgcaggcgcgggcgggcggtgggggccggggcaggaggaggggtctcggggcccggcggcccgtcattggttaatattttattctgttgacatgttttcttactgctgaggcttccgacaccttctcccaggccccccctcccggccggagcttggcctgagctgtcaaaaccccgcccccggagacccacaattggtccaaaaagcgtaaaatcagcaatcaaggggggcctggctcgttagcgcaggggatccgagctgggcaggacatgtgagatagtcacagttttccagagatcacgacaagatctaaccagtcgcgcgtggtccccggcgccggagcgggccagctcagcccggcccagcccggccccgcgcagagcccccgccgcccccgcgcacagagccgggtgccccttgcggtgcgccggacgggaagccccgaggagcagctgctgcgcccgccacccgggtcgtccgtccaccgcgcgcgccgccgcccgggccgggggtccgagccgcgcgcccccggccccggccccggcccccgggcgcctgggccggatgtcccgatgagagagccggcgctggcggccagcgccatggcttaccacccgttccacgcgccacggcccgccgacttccccatgtccgcctttctggcggcggcgcagccctccttcttcccggcactcgcgctgccgcccggcgcgctggccaagccgctgcccgacccgggcctggcgggggcggcggccgcggcggcggcggcggcagcagcggccgaggcggggctgcacgtctcggcactgggcccgcacccgcccgccgcgcatctgcgctccctcaagagcctggagcccgaggacgaggtggaggacgaccccaaggtgacgctggaggccaaggagctgtgggaccagttccacaagctaggcacggagatggtcatcaccaagtccgggaggtagggctgccggccggctggaaggcgcgcgggcgggcgggcgggctggggcacgggactgcacggatcagagcagagctggggactcccggctcccggctcccggctccaggttctggccctgacgccacgcttcgctcccacggacaaccaagttgacttttctcgtttggcaccggagcgatttttttttaaaacaaaaacgctaaaatcctccgaatgaaataaaacgaaaacatactgctaaagaatagccccaaccgttctgagtcccagcgcagaggaggccccgcggcttggccctggcggtctcctccgccccgcgctctcgctcttctgcgtccgggtccgtctccgagctcgggggaaattcagcctctctcagactctgctccgaccccgaagcccctagtgggacctgggcccagctaaccccacgctggttgctcggttcctggcagcgagccccgggtcagccgagccctccgcctcaccgggctgggagccctttctccaccgcgggcatccgagccctggacgcatccgccgccccaggccttaaagcctgaagaaagccacagccccggcccctccacccttcgactcagccaccgagaatccaggcctcgggttcaccctcttcccccgaactgcacggccaggatgtcttatcaggctctgtagcccagttcccaatacaaacaccctccagatttatttctgggagcttccgtcccaagtgggtatttcccctgaacgaatttcggggagattaaggagaagcgagaatatttctagagggcctagacttctctcctgggcatcagctttcatctcagatcagggagaaagagggtccccagatctgagcacaaggcctaaaggaggctgtttaggagagggtctgacaggcagaaatgggatctcctgggagcaacaaccacaggtggggtcgtccgggcagtgatgagagggcagagcagccgaccacaggggaaacagccaggcggcagcggtgtgcgcaacgaggagggataaataaaggaggagtggggtcctggaacctagaacagccggttccaatgggatctcctcccttccctccctcccaggcggatgttcccccccttcaaggtgcgagtcagcggcctggacaagaaggccaagtatatcctgctgatggacattgtagccgctgacgattgccgctataagttccacaactcgcgctggatggtggcgggcaaggccgaccctgagatgcccaaacgcatgtacatccacccagacagcccagccacgggggagcagtggatggctaagcctgtggccttccacaagctgaagctgaccaacaacatctctgacaagcacggcttcgtgagtgttggggcagggtggggacggtgcaggagcttgttgacccagcactgcagctgagcaggagagcagggcggcaggatctcccagggggaagcgctgggcaaacccccagagtgcccctgcccgggtcactgccctgtggtctacgtgggctgggcctgggcctgggccccagcgctctcctttgcaagcctgggaaatggtggggtcagtggctggcagctcacaatctcagttcctcaggccgtggagggtcccttgtaccgtcaatagggaggcaggtgctgccaggactgggtctgcagtgctggaccacggaggtcaccctgctcttggcctgtaggtgccgagcttggaagacctgcccatgacctcaacttgtccatcacccttctgccaagacccgcgtccctggccctcccccaagactaggggatttaaaagttaaaccaccctccccacccacccccccagcctccctaagacctttccctcctcccacaggcagatgcccaactaggaggagagggtttgggggttctctccatttatgcctgagcaaggccccagcgaacgggggtggtgtttcatagggtaaaaggaactgcccattctgaggtagggcagttggtgagcccccgaaggttggaatctcctctttgtggggttagggggtttaccagtgccagaaaggaagagagtgttaagagtggaattgtagggctctgttttgttttattttgtttgggtttcttttgtggggaggggttgttttttggttggtttttttgttttgttttttgttttgttttgtttttgctctgtctcatttctgtttccaactgggaaattttttttaatggcaagagaaaaagaagctgtgaaaagagaaaagcgagaggaaaagtagaagaaaaagaaataagggtgagaaaagagcagagacaggagaaaatggggaagaggaagaaccgatagagagagagagagagagagagagagagagagagagagagaaagtggagaggaagaggtcaggtacccaaagaatagaaaagctcgggccggggctggtggctgccggctgacccccaccctccccgcagaccatcctaaactccatgcacaagtaccagccgcgcttccacatagtgcgagccaacgacatcctgaagctgccttacagcaccttccgcacctacgtgttcccggagaccgacttcatcgccgtcactgcctaccagaatgacaaggtgcgcgcggcgggcggtgggctaagcccctgcactgacgcccctcaacacgtgcaggcccaaccgtccgttcatcgcccctcgaagccccctgcacgggatccgcgctcttgcggcccgcccccgagcaccgagcctcgcatccatacgcgcagcactcacggaagtcctcaaggcgccctctcaatcccaccgcgcgcacacacaggctcccctggggcgagcgaacagctgggccgtcgttctgatggagatgtttacttaacaattagctgagactccgggcccgcgctgacatttttacattttattcgtttatttcttggctcaggaatgagaatgtaagtgaagagagggcgcccgaggcaatctgcccaggctctaccgatgctcagaacccggggcccagttttcactctctcggggggagccagagggtgggacaggtgacacagccccccaaagcactcctggctactgctctgggtcagtctaggccccagaccccttgggaggcgcttagagaattaccttccaatcaaagggctttttctgcttgcggcttctccacccttgttctagtggggcagagcatcacccctctaggcctgtgcccttgtgcgccactgtcgagtgaaagagagggtacgagtgtccgtgccggtcgttgtggctttgtgaaccaaggtgtacgctgggctgtgcgtgccccgtttggggtgtgttggatacctgtggaggcatgtgaattcgtttggttccgtaggcgtttgttatgtctgtgcacacttgcgtgtgtgcgagcccgtgagtgtgatttggctttgtgtgtacctgcagccttgcgtgtgaactgcagtctctgttcgcgcctgctcgggtgcggagtcgtttaacttgcgcaagcccgcgggcgggtgtgcatgcttgggctggttccgacactgtggcactgctttaggcctgtgtggggctgtctaggcctgtctagggataattcctcgaatgaagcttttggaggacagaccccgtcgctttccgcgaggccctcccaccggcctcagctcggggcgtcccatcccaggcgggtgggtaccaagtggacgctccccgggtcttccctctgcggccagcacccgctgacctcggggtgccaccgaccacgctggaagagccacggcctgacttagcgccgcccccttggtccccgcagatcacacagctgaagatcgacaacaacccgtttgccaagggcttccgggacaccgggaacggccggcgggagaaaaggtgagaggccgaggacagcagccctgtggcgggtgcccgcgggagcagcgagcaggagggatggagggatgggctccccgctgacctggttcatccgctcatccccaggaagcagctgacgctgccgtctctacgcttgtacgaggagcactgcaaacccgagcgcgatggcgcggagtcagacgcctcgtcgtgcgaccctccccccgcgcgggaaccacccacctccccgggcgcagcgcccagtccgctgcgcctgcaccgggcccgaggtgagggtcggaccggaggagggacagggaggtggcggcggggggtcctcaggtcgctgggctggtcttttgctgagccacccgctaacctgaaaggccaggaaggaaacgtcggcgagtgtctgggatggggtttccgtcccgggactcccctacgagggcggtccccggtagccagaagatccggccggactccgagcctggccccttgggcgccgtgtaattctatagctgtagggtttcctctccagggctgggttccttccactgtaaatctggggtcttggattaggtgacctgcagctgccctttgcccgactccagcagctcctccgactcggcctccccgagagcagcccttcccgagtgtccctgatcctgctcgtcggcctccgcccaggcccctgtaatccgcgcgccctctccccgcagctgaggagaagtcgtgcgccgcggacagcgacccggagcctgagcggttgagcgaggagcgtgcgggggcgccgctaggccgcagcccggctccagacagcgccagccccactcgcttgaccgaacccgagcgcgcccgggagcggcgtagtcccgagaggggcaaggagccggccgagagcggcggggacggcccgttcggcctgaggagcctggagaaggagcgcgccgaagctcggaggaaggacgaggggcgcaaggaggcggccgagggcaaggagcagggcctggcgccgctggtggtgcagacagacagtgcgtcccccctgggcgccggacacctgcccggcctggccttttccagccacttgcacgggcagcagttctttgggccgctgggagccggccagccgctcttcctgcaccctggacagttcaccatgggccctggcgccttctccgccatgggcatgggtcacctactggcctcggtggcaggcggcggcaacggcggaggtggcgggcctgggaccgccgcggggctggacgcaggcgggctgggtcccgcggccagcgcagcaagcaccgccgcgcccttcccgttccacctctcccagcacatgctggcatctcaggtaaggcctgtgaccccgcggcagcgccagcgagggagaaggcccagggagctggcggcgaggccagcg

SEQ ID NO: 26 - DNA, 609 bp, Homo sapiens
cgcctcgccaggtggcaaggccttcgctcctggtccctgagcgccgctcgcttccctcccgcgtgggcagggggcagccgcgacgccggcccagcgcctccgagccgggaaccgcacagcacccggcgatggccccgagccccgcgcgcccgaagccacggcgccgctcgctgggggcctcgggacggcagggacggctgggggtggcgcggaccgcgtccccgaggcgcgtagcgcgcgatcgaaccacgcgccccgcccgctgcggtcctctaacagcccctcgcgccgccccacgtggggccgcacgggctggcggccccctcacccgattggccgccgcacagccaatcagagagcggaggcgatagcggccgcgccgattggctgcggcgggccggcccgggggagggggcgggaccccttttgttctctccgcacctccgggttcgcgcgactgtgagcgcccccgccccggagcaggggtccgcgctgggggtccggagggcgggggcgcggcccgacgccccacccccagcttgttcactgccgccagggcccagcgcctgaagcgcgggtccttcgctgggacgagcgctttcatctccg

SEQ ID NO: 27 - DNA, 4191 bp, Homo sapiens
cgcctccggacaccggaaccgctcattgccaatggtgatgacctggccatcgggcagctcgtagctcttctccagagaagaggaggatgcggcggtggccatctcctgctcgaagtccagggcgacgtagcacagcttctccttgatgtcgcgcacgatttcccgctcggccgtggtggtgaagctgtagcctcgctcagtgaggatcttcatgaggtagtcggtcaggtcccggccagccaggtccagacgcaggatggcgtgggggagggcgtagccctcgtagatgggcaccgtgtgggtgaccccgtctccagagtccatgacaatgccagtggtgcgcccagaggcgtagagggacagcacggcctggatggccacgtacatggccggggtgttgaaggtctcaaacataatctgagaagggacaaggggcggcttagtcagggacagagacccacggccaccccatgctcacacgccacaacatgctgcatgccagtgtgatgtgtggagaaaagaaaagaacgcaggcagaaaccaaatgagaaacctggaggcttcagggaggaaatgccgggagaggaacagagcctggaacagcgaaagaaacacttaaatgtcagaaatcaagccgggcagaaaatgactggggaaaggacgggaggagcacgggcgtcggccgagcctcacctgagtcatcttctctctgttggccttggggttcaggggggcctcggtcagcagcactgggtgctcctccggggccacgcgcagctcgttgtagaaggtgtggtgccagatcttctccatgtcgtcccagttggtgacgatgccatgctcaatggggtacttcagggtcaggatgccacgcttgctctgggcctcgtcgcccacgtaggagtccttctggcccatgcccaccatgacgccctgcaggggacgacccgtcagcctcgccggcgacaccgaacccaccccgcaacgcagaacccaggagccccgcggcgccatccactcacctggtgtctggggcgcccgacgatggaaggaaacacggctcggggagcgtcgtccccagcaaaaccagctttgcacatgccggagccattgtcaatgaccagcgcggcgatctcttcttccattgcgacctgcccggaaaaggatggactcaggcgggcgcgtctgtaacacggtcccctccccacagccacctaatgccctcccgcggggaagcctcggccctgccccaaccccagcggccgtggcctccaagatcgcaaccgcctggaaccgaaggccgggccttttacgtaacgtccacggctcggaagtctgcactgcggccgggccccgccctggacccccggcgcccccccagccccgtccgcctgacccggcccaccccgccttttgttcccggggaaggcgcgacgaggcctcggcagctggaagcggggccagccggggtcggggggcgcaggcctgcgacgtccagctcaggccccggggcggggcgctcaccggcagagaaacgcgacggcggagcggcggaagaacagagtgcgagagctggcagcggcgactgagaccgaccgcggcctcccccgccgttatttaagcggaagcggcgcgcccgcgcggcccggcggcgcgcgcggccccagaacatgtccatatatggcgatctttccgaaagccgggcacccattggcccgccgggcagggacacgtggggccccggcccgccccgcccccagcccgcaccgcctcccaacggccccgcccgcgccacccccgcgcgctcgcgaccctgcgcgggccggcgggcgggtctgagttggggcgccctccgagggtcgccgggagggccgaagggctgacgggcctggcccctccccgggactgccgcgccgtggggagggccctgctgcgccccgaaactgcctgacccggggcggggccgcgccggagctggggtgggtccccgagtccccggccacgctgcggggctttgctccctgggacgtcccttgcaatctttcccctcgggctccacgaggatctcgggcgccagtgccgggctgaggaaggggcgcggcgggggtccgcgacgctctgcgtccctgggcgcgggggagctctcaggagccctcacctcgccccggtcaccataggaaacagcccgcccgtccccaagcgcagccaccaccatgcaggtccctccacggggcgccctgccccggatctccgttcgtttgaagaccgaggcccctttctacccgacccgacacatccctgcccctcaaatcacaggcctgattgtccccaggcgcgcgctccgggttggggctaccccgcccccttatgtgctgagaaggtggtgtggagtgaggcctttgtttcattctggtcacctgtagacattatagggaaactgaggtagacaatgggagccctgacttttttgaaggtcggctccccagcttcgggcggtcggggggataccctcttctgtttacgatgctagttaagagcagcgatcagggctggcgtgggtttagccacaggcttgtccagggcgctcactcctcgccaggagcggcgccggtgctttggttacaccccgagaggcgcccggcactacggtttccttgtgcgctgctggaattttcagggttggagcgccggccgcgtaggggtggcagtgaggccgcgcagtgaggggacggactcgcccgaggtcgcagagcgttcagattgactcgacaggctggggggcggggccggggtccccacgcggagtccggggccctgcaacgttagctcgcgaccccgtgtttgggagtgacaacagcgagtcagggcaaggcggccggcccagctcacctgcgggctgcagtcagcccggacgccgagacccgtgcctaaggcccccgtgccccaggcctcccaggggtgctgagcgcccccaggcccagaatctccgggggcccgggcaaggctgtcaggtatgttccctcccgtggcgaagccggggctgggcctccaacagacccacccggactcgcgtccctgggcagaaccacacagcctgctcgacccccgggtgaggacttccacttggggggtcccaagatgcgacgtcacacaccgagggacacgaggcgggtggggaatagccacaagcccctccatcctgcgttcaccccacactacgttctgctccgggccggccggggaccctgcccctccgggtctctggtctcctccgaagcttctttgtctaggaagtgcggggaggtgccggccccgggcgcgcgtccccgtcccccacctttcttccccacgttgcggacccgaggccgccagggtcgtgggggcgggggcgaggctggcgcctcgagacccccgggttccccgccacccgcagggcctgagccgcggggaggggcggggaggggacgccgccgcgggccgggggtggagagcgacttcctcctccgcggccgcgtcacgcggcaggggtcaaggtgtggcccaggtacgcggcggggggcggtggcgcggccatatttggcgacttcggcgccgccggagccccgcccgcctccgccgggaagcccggcccggggcagcgccatccccgcccgccgccgggcctgagtcacggcgggcggggggcggaagtaacggacggggggcgcccgcccggcgctcctggccttatttagtcaaggaggccgccgattggcgcgcgggacagaggggcggcggccgggcagcctcggggcgtaggagtggcccggcgcctccgaggggtcggggcgggagccgggcgtggggggcctcggagcctggggagtcccgggaccgacggcggcggggatgcgggtctgcctgtccctgggatgcgggcgggcgggcagggcgaggcctggagcccgcggggcgcagggggctggaccgcgcgctgtgatttttctgtttagcagcggggcctcgcccaaggtcgcgcggggcctgtcctggatatgctctgggaagccagtggcgcagggcagccgccacccttctggaaccagtgccggagagggccgggcagctgggcctggggaggggaccgcatggatggagctgggggtagctgggcggcctccggagccgggagagtcggcagctgcacttccg

SEQ ID NO: 28 - DNA, 3855 bp, Homo sapiens
cgaggtccagcgggtgggcagccctcgcctccccgaaggtgggagccgccccggcctccgcgtcgggttctgggccgcgagaggggcggggcaggggcgcagggctggagtcggtggctttgtagggccccgcagggcgcggagccacctggggaaacggaagttcagggagggcgaggccgtgccccacatccaggcagcgcaggccgcgggccgctcccctagccaggaagagccgccgtccgcaccgggaccggagaggaagcggaggtctgaagggggacaggtcggacgtggcctcagagagggaggatgtgggccccggagctgagccaggcggccggaaccccacgggacccgcgcgcacctccccaccaaaggggccccggcgttggggggcgaggggtcgctgccctggaggacgttgggacctgccgcgctgcgctccgaggcggggaaacggagggacggcggggcggaggggcgaagcgagcctggactccgggggaggcgaggccgggaagacccagtcccgaggagcagagggggcgggccagctccgggccgggggcgccgacggggccggttccggttgggggggtccctgggcccggcggggttgcgggacgcgccgtcggggagctgcggcccgggagcccgtccggaggcggcgcggggtccgggcggggcgcgcaggggcggcgccaggcggtgacgtgcgcgggggacgtgccgcgccagcgacccgagcacttaaagtcgcccccggcgcggagcggaccggcagaggcgggcagaggcggcgagaggcggcgagaggcgggctgaggcggcccagcggcggcaggtaggcgcggccgcgatgctcgcgcgcgggccggggcccaggagctcccccgtgccgggcaagggggcgacctcggggtccgcgctctcacccccaccccgcccactttgtccatcaggtccgggggcgcccggggcatggactagccttctcgaccccgagcgggccccaccgagggctcccgagagccagccgcgcccctccgcgccctcgcatacctggctgcccagtctcgtctcccccggtccggcccggcgcccacccgtcgggctgccctcaggggctgggctcgggccaggagatggctgcgccggcggccaagcccggtccgggtataaatagctcggtccgcggtggcggggggcgcgggctatatttggtggggctgctgcagccctccctgtatgggtggcctccccgagggagtggggagcacttgcgctcgggccgtggcgctttcccctgttcgcaacaccctgaacactccccgacccacacccgcccttgggcaatgactaacagcccgagaccgtgcagggaagtgggggaccctctccagagccaggctgggctccctgggttcgccctacctccacccctggcccccagctcgtgggaaaaccaggcgcagcctttggtagcctcccacttcctaacccacgcctcagtttccctatacgaaaagtgggttggtcaggagcctccctgggtgttgggccagtcaagagacaggagccgcagagtggaagggaagcccttagctctggccggggcctccaggggtggctggagtcggtgagggacgatggggggagccgaggggcggctgtcctcctctctgtcccagaggcatgggctgaaagggtccctctcctggtgatggccagagtcaggcctgctgggcgggaaatggcagttggcccaccccaaggcccaggggcttgtccatgctgagcacaagggtgggaggtcttgtgtctgttggggccggggagtccaaggtcccagcttcactgcacaggcctggaagtcaggtgggtgctggcctgaccctgctgaaggagggtctattccgccagcgtggtgtggaggcggctgggctcgactccctcatggagggaaggaactgaggaggtgctaggaggccgtgggggggtgtctgcccccagagaggctttgagtgcatgggggctctgccgtggcacgtgcagcgtctgtcccccagggccccatggtgtggacagagtctggggtgggcacctggaggcctggtctcaaggcaggcttggtaacgaccccatgtgaccttgggcagggcccgccatccaggccacccagaaagccatgtggcctgcacgatcaggccagaccccactgactggagcggggcactttactttggaggggcagctaccagagggctctatggatcccagcccagccgtcaggccggacgggtcccccctactggcctgcagtgacccttatgccaggctcggctggctcagcggtgggtcctggggtgacctggccacgtgggacagaagggcctgtgccgctccctccagtgaggtgccaaatgtgggccacactctgggccccagcgggggccttccgggcagtggcgcagagccattggaggtgcccgggccctggggcaggggcggggcgggagcagacagagctcagggcgtcctcctagcgtcacctgcccgagtcaggcccatctgtgaaatgggccacgccgggcagccgcacgcaacggaaccgaaccgccgggctcctgctccgagacccgcagttcacgagtcgacgcccccgagtcccgagccgcgcccactggtcccacctctgcagcaagtggagtcggccccgccctgtggcttcgaggccccgacgcctggcgcggctcccgggctccgctgaccccggtggacccgcgcgccggcaattacggtagcgtccagtagggcgcctactgcgggcaatacgggggctcctcccatggcgcggtcacgtcccccacgtcccgcggctggcggggcggggccgggcggagcatccttccctccccctcccccgggtgggccccgcccagcctcgaccacccgcgccgacccccgcccgcagagccgggaagccggaacccgcaggcgccctccagccgtccctccggcccgggcggggattgcgccgaggaggggcctctcctggcgagggtggagttttgggttccccccttcgccccgcacctcccgccttcctgcctcctcccggtcttcaccttcccccccgggtccccacgacgcccgctgggcggcggtggggtagggggcgcctcctgacccgactccccttccagccttggccttcaggcactggctttagggtggaggggcggccttgcggggtccaggaggcctcggaggtgggggacggcggctctggaccagctccggcccccaccacctcccggccccagggcggtgacagccttgcggagaaggggtcttcaaagctctgagaccccgcagtgccagctcaggattgaagcctgcggcggcacatgcagccttcctttcccaaaggggacttgcgcccagccccacttggggcaccagtgggggctcctggccctggccctgggtgggagtggccaatccgcaaatgaggcctgtgtgtctgccccgccggcccctgccgtcgtgatgtaagcggacacagagcggcagggcacgggctgcgcgaggggtctgcgggggtcagaggtggggcgcagggcccaggcctggggcagccccttgcgcagcgtccaccccctgcaagctcggccccgacacctttgcaggcgcctggggcggggctgccgggagttgctcactgctccttccttgtttgcacaacaggggctgcgggggaagaagggaactgagggaggcccctcccccggtgcagcgccctcccgccctcggcacccctcggcagccaggcgctggccacagaggccg

SEQ ID NO: 29 - DNA, 903 bp, Homo sapiens
cgacggagggcgccttacgcgggcacttccatgactcccaggttctgacggcacaagagccctgatgcccacgctcacggacagttccccgctggagcgggcaggggacctgaccctacgacacagggcgtcctgtgtcagaggcaggaggctgaggacacagctgcgcagagccgcccagcagaggttacccggataacgataagtggtccccgcgacgccccttaagtctctggggttatctggagcgcaggtttgcagacccgctgcccccgcggtgggccaggtccacggggccggaagatagcaggatctgtccagccctcgggctgcggcggctgctcgcggagcccactctccccgacggtccccagcttgctccagctctgcctggagctcccatgcgtgccctccgcatccgccctggccgcccagtcacacacctgcagtccccgccgggcgccgagcccctctccagcggcgactccctgcgattcgggtgaccgtcgggaccaccgcgccccagctggagccgcgcggcgagccgcagcatctgcccagcctgggctcctccggcgggcggggcggagcctgtcagggggcggggcctaatggggcggggctgaccgcggagccggcctctccagccacctggcggcccggcagcgtcagccaggtgcgcgcccggccccacatcctctgggtcacgcccccgaggccgcaaggcgtctgagaagcagccccggctctgacccgccagccctcggaagaagcagaccctggccggtccatggcagtggcccgaggggcaccagcgcggagcaccagcgacccccaaggagtaaggggaggttaggcccccgcgtcggaagcgcggctttggccccgcaggagggctgggcg

SEQ ID NO: 30 - DNA, 220 bp, Homo sapiens
cgattcgagagaggcaggcatccgcggcagcagcttccaacccagacaggcttaagccgcgcgagggtgcccgggtgggtggctgcgcagggccctacctgggcgggagcggctgcagagccctgtccacgcggtgcctccccagcacggcacctcccactccaggctgttggttacctggccgggagctggcggagaccaggatttctcctcgccgccg

SEQ ID NO: 31 - DNA, 7467 bp, Homo sapiens
cgcggcaccgcgtgtgccgagcgcccctgaggatggaaagccccacgcggggcaggtggcacccaccctccgaagacgggacgggatggagcgttgagcttcggggcagctccggcccggcccgcgctggagacgcccgcatctgccaggatggcgtctcatagccctggtgctcacacatgacgccaggaagccccagcaacagtgaccgcccaggctctagaaaatattggacggggtggatgaacacccaagtgcgctccaggagaagggatttggcaccccaaggggcttttaaaacggtaagcttctaggggtgtctttgcccccaataatccatagaaacaacagtcatctaaaaatagtcttgttttctgtcctaagctccttttaactttgttagtcatcaccaatcctaaaataaaacccgtgtaacgtctcccctagtagcggctataaacaaacctacgaggaggcaggaggaagaggaaaggggcgcagggcgctcggggagcagagccgggggcccgcggtggccgcagaggccgggccggggcgcagaggccgggcgagctggccgcgctctgggccgccgcctccggaactccctgcgcctggcgcgcggccaccgtggtcccggcaacggcattaaacagagggaaacagacccgggattccgtcacccgggcggggggataaggacggctttgagagcagacaggaaaagggagcttttctgcatggggtgaaaaaattatttattgaaggaggaggaggcggcagcggaggaaggggaggggcgggaggaggaggaagagccggccgcccccgccccggccccggctcctcaggagccaagggcagcctcgccaggtcggtcccgggctcgaggaccgcggctggggtcgaggggctcagtctcccacgtgaccggctgggcgcgccccgccagacccggcctcgggattccctcctcccggcgagtctccgcccgccccgtcctggaggtggggagaaggagggcggggcgggggggacggaaactctccccgccaaatcctggccccaggcctggggacactcgcggcgggaagatttggaggggaggggagggggaggggcgtgggggcgcggcctcgctggagtccccctgaccccccgacccccgcccaccggcctgggcgtcctcccgcggcccctcctcccctcccggcgcccggtgctctggggcgcgtgccacgcctggctcggcgccgtaggggcccccgcaggtagagacccctggaaatggcctcgacgccgcaggagcgaggcggccaccaccccgctaatccgggcacgtctctccaggccgaggcctgcggtggaaaagccggggttccatttgtgctgagtcggggcggccgaatggagccaggcctcgggacgcgggacggacgggctctggccgcgcaccttcgcgggctctgcagcgcccgaccgcctcccccggcagggaggaggcgcttgtggggggcacccacggggcacagtgatccctgggggtctgcggacctcctgggccccgcagcagacacgagtttagcctttgggtttagtttaaatcacataagggtgtcgtgcaatcgatttatggtttctacacaccagacactttaacctccaaccccccccatccaaagccaacaagaaaatgcggtgccgtgttggcagctgagctgcgcccgaagagacgcagggagacgtaagagaggaaagtgtgagtggccggggggcctccccccgtcagaagtcgcgcagtcgcgcccataaaacgccccctccgggcggctagggcaggtgagcgcgtccccgggcctccccacgccggcccctgccacaggccgtctaggtcgagcagatatttacagaataaaaatgacaataactcgacgtcccgggacggccacgcaatctgttagtaatttagcgggatgggaatttcctttctagggcctgccagtgaagcgcttttccaaatttccacagcgggggaagcctgcgattttacataatgacttcagcatgccgggctttctcgacacccctccccggcccccggcccccgccccccgccccttttccagcagggccgggctccctccggacacccgcgtggactcaggcgtcccgtctggcccgttcgcccccgtttcccccgccagccccagcgcccccctgcccggcccccggattccccgttcccgcccctacgcccccatcccctccccgtgcgcccctccccgtgcgcccccctccccgtgcgccccccctccccgtgcgcccccctccccgtgcgccccccctccccgggcgcccccctccccgggcgccccccctccccgtgcgcccccccctccccgtgcgccccccctccccgtgcgcgccccgcctcttgcgcccctgcccccaggcgagcggctgccgcggcgcggggaggggcgggcgctcggcgactcgtccccggggccccgcgcgggcccgggcagcaggggcgtgatgtcacggcagggagggggcgcgggagccgccgggccggcggggaggcgggggaggtgttttccagctttaaaaaggcaggaggcagagcgcggccctgcgtcagagcgagactcagaggctccgaactcgccggcggagtcgccgcgccagatcccagcagcagggcgcgggcaccggggcgcgggcagggctcggagccaccgcgcaggtcctagggccgcggccgggccccgccacgcgcgcacacgcccctcgatgactttcctccggggcgcgcggcgctgagcccggggcgagggctgtcttcccggagacccgaccccggcagcgcggggcggccgcttctcctgtgcctccgcccgccgctccactccccgccgccgccgcgcggatgccaagcaccagctttccagtcccttccaagtttccacttggccctgcggctgcggtcttcgggagaggagaaactttggggcccgcgccgcgcgccggcggcaccatgaagtcagcggaggaaggtaagccccgcgcggcctccgcccccggacccctgcgccccccacggcccgggccgcgccccccgaccccgcccccgtcgcccgcacggaggggtccgggccagagaacgaggtcggccgggtctgtgcgcccagggaggggcgcggcgcggaccgggaacggcgtcccccagctcccagcagcccggttccgagcgcgccttctgccaataggtgtctcctctgccccagcgctgagggcgcgaggcgaggccgccgcgggtcccgccgaggaagcggctcgcaggccggcgatttggggacgggcgaggctgggccgtcaggaccacagagggcacgtgggagggaggaagggaggcttccttgggagtgaaggatgccccgtgtcttcctgagcgaggcagagccaccctctggcgccaagatcgcggtttgggggttctgcgccccccacccccaccccgagctcgtggcggcgtctccctcgcgccgcgcccctgaattccccagggccgagcccgtcacgccggctcctggacctgcaggaccaaaagatccaaagcgcggccgcagcacagccccgaatttaagccattttgaaatcctataggcaggagttccagccagagaagatgtttcaggcaagtttgaaaaggaaccgccaagtttcgagctgcttctttatagtgaaaaaaagatggcacagtgtgggtccctgaaacccggtgtggggggcgctgagacccccctgcggcgcatctcacattccagtcccccaccttagcccgtccccggcacacacccctggcgatgccgggtgggctcctgcaagtggctttgaaaatacccttgcagaggtgtgagcatacgagttagggattagggattgcaatgctaatggaaattggaagacgcttttccaaattccttaccagctctgaaatattttatataatttatttaaagagcgccagcacacctgccaacatatgtacttcataagggcagacgtccttccgagcgctgccgcttcctgcgcgaccctgataactgcggggcttgcttgagaaccgggctcccccagctctggaaaactcgctggtcttagcagagctgagcacagggccgtgagctccacggaggggggtccgtgtggtcggtgccctgacgcttcacgggacgggaggtagacaccacttcatcccaatgttagaaaatgttgacatgtttaaaccatgtcccgtgtcagtgttttacacaatgtgctcttgctagtagcagagtttaaaaaatttttttttgttggagtttcggttttaaatcctgggacacttacgccgcagtgttcattatcactccctttagaaaggcaggtctttccaagttcagatctctgtacctgaagagccggtggccgacctgtagtaaggagcccctttcttttacgcaaggaaaaaacacgacggggcagtggctagttttcgtgtttcctactgaagaaaataagctcgaaaaggtccctgctgcaaggcagtgcgccgggcagcgacgcccgaccgacatgggtgtattttatgagggctgcagaactgggccagccggaaagtgtgatctagtccttgatacacacaggccggcagcatggggacacgcgtggtttccacaccagccatcgctgagaccaccgggaagaactctgagtccttctgagtaggccccacgaggccattcctggcccatctgaggacctgggcgcctcccctgcgtccctcgggtttcgggaccatctgggtggaagctcattaggcgtcgtggttcctgaaggccccgggcttgcttcctcatggagaacgcagagaagtacaggcctgtaagtagccacccagtcagaagtcgctccagacacaatgccccacgggctggggccaaacaaagctcaccctgttctctgccacttaacttcacagcgttgtttcccagctcagattagaaaatggcagggcgctggcaccccgcttcatcacacccagcggggcttctgtctccgcagacatccccagcctctgggaggatgcgtccaagctaaacatttccatggagacacccagtgtctgtggaacgggcagctttgaatctggaccaatagctctgaaccatttgcaggaaggaagcccagcactggggcgggggaagggggggctcctaggagcagcaggggagccgccccaccgaccccttctctgggatggagaccagtgcagtcctaggacagcagcagtgagcctccatccctgcagggcccgcggcctggggccaccagcaggcgggacagtttagcaaaaagttttagtctaaacagatgataagggctgtcatttgtgtataattttatggtgggggtaaaaaggtggctggagttttaaaaacaatgtacaggccgggcgccgcggctcacgcctgtaatcccagcactttgggaggccgaggcaagcggatcacgaggtcaagagatcgagaccatcctggtcaacatggtgaaaccccgtctctactaaaactacaaaaattagctgagagtggtggcgggtgcctgtagtcccagctactctggaggctgaggcaggagaatcgcttgaactcggaaggcggaggttgcagtgagcagagattgcaccactgcactccagcctgggtgacagagcgagactccgtctcaaacaaacaaaaacctgtacaatggaaagagaatgaaacgttgtggcttctcaacagaaaacagaacttccacttgcccaaggtcggcaaaagcagcaaaactacaacaaaacaacgctccaacatccatcccaaagtcagaagtattattagaaactaaatcaggccacacggaacgatggcttatcactggagaaaaccagccagtgaaagggtcgcgggagaagcccggggacgaccctgggactggagggtttctcgcctctggaaaaggcagtgcccgcggggcaggccagagggagcgctccgaggagctttggggttgccagccttgacacgcgcacccctccgcccgggccggctcccctccgccctcagactcccaccatcctcctactattccacatgtcgggtgtatatggtgcggagagcccgggggaagttagaacacgcggcgggagaggcaggcccagggcggcctcagctaagcagcccggctttccggatccccgccgcgcacaggctcgggccgcctagggtgggacccgaactcgccttcccgccccgctcaaggaggtcatctttggaggtgcccctagaaggaccggcgggtttcccgcggggcgggggcgcgcagggcgggtgtttggaggctggggactcgcaggagaggccggtgggtgccgggcctcctgcagggggttgcaggccgaggggcgcgccacgaacccagcgggaatccctgcgcttcccggcaccgtcgggctgaaatcggggttcctagcgttggcggcctcgggctgggacctccccactcccggggctccgcagggcgcgcacgtggcgctcgggggtcaccgttgcagccgctttcccgaggaggcccggattgaaaacttctttaaaaaaaaaacttaaaaaaaaaattccgtcgccctgggcgcagccttaggtacctggtgccgctcgtggggtcccgacgcccctacccccgggtccggaggagcctgcgccgggccgggacccccagggcacaagaggccgggggacccctgcggtcgcgcgcgcgcgagggggcgggggcgggggggacacgagtttatttaaaaactcgtgtccggggagtttatttaaaactcggaagccggcggccgcgagccggttgtttatgtaaacccggaaacgccccggggggcgggggcggggcggggacggggggaggggcgggggtcacgttacgcggaggacgcgcgggcagcgccgggagaaccgaacccctggcggccgcgaccccggctcccgccccggccccggcccgacccgccatgacggggctggaggaccaggagttcgacttcgagttcctcttcgagtttaaccagcgcgacgagggcgccgccgcggccgccccaggtgggtcagtcccggagggcgcggggggcgcggggccaggtcggggtttgggggcgcccaggccccctccgcgtcctcgtcgctccccggggaccccaggagtccccggcgcgcccgggaccgaggggctacggcgggggacgctgcagtgcggggcgtcctgggctcggatgcggttcctccgagcgctcgcggcggggtcctgggggaagagcgcagcccggacacgggcgtggacgaagtggtcccggacagagggctggtggcgctgccagaggcg

SEQ ID NO: 32 - DNA, 1142 bp, Homo sapiens
cgaccgctagtggctgggaagtgggcggtgctggggggtgagtctcggcccctccaagatgcgcctcccatgaaatcctccagtcccacgtgtactcacacgcatacggatgcatgcacacgcacagaaacacacagggctcggacacagctgcgcgcagacaagcaaaggaccacgtccacgtggaaacaggtgtgcgcacacgtccccgcgcatccgcgcgcacaagcgctccacacctgggaacgtgcgcacctgtcccccgcccccggcacacacacagccgccccacactggaacacctgggcgccagcgccgggccactcgggcacgcagggcgctacgcagggccgcgcacacgcttttcggccacacgcgcacagccctccacgtggacgcgcgggtacacacgcccggggctcgtagccgcgcacacccgcacccaggaaaccccagccaccaccacgcggggtccgatcgcctctgagcaccctccgccccagccgcgcacccgctgcgcgcccgggacgcgcacactcgcgcacacaccggcccttcctgcgcacgtcccgggcccacccccgcctagagccctcggggtttccccccaccccggcctcggggtctctccacgtctccccgccgacgtgctcacctgctcagggggcgcccccgagccgcgccccgcgcccgcccccaggagggcctccgcgagccggctgcacaccccgaggcggtcccggctgcacaacttggagcgagttgctccgtttcctcattttgggggcgaagaagggtcggggagtgggggaaagcgggaggcgccgcggcctgggaggcccccgcgggtcctagcgccggccgcactgcggtctcctccgcgcgccgccgccgccgccggcccctccccgccccgcctccgcggcccccggcgcctcccgcgcgccccgggcggggtcccggggcgcacgcgggctccacgcctggccctgacctggccctgtcgggcgtgcaccccccacccaccctgggaagcggggttcttggagcgtggaacgggcgccgccagtgcgcgcggcggggaccgggggtgagggagagtgagctgcccgtccggggagataagcaaggcgccaagcccg

SEQ ID NO: 33 - DNA, 1673 bp, Homo sapiens
cgaggaagggacggcgccaggagcctgggcttggcagctccctccggagggcgcccggggatgcccgcgagtgtccaggaggctggtgacctgccaaggctgggcgggctggctggagaggagcgggagcgcgggcccgggagagacttgggtatcaggaaactcggaggcggggctggggggtggtgcctggcccggaccgtctgcaggggcggggaccgacccccaggggtcgctgcgccttgcgcttcccccgcccgcgggcgccagggccgggctgaaccaccgcgcggatcggcagggggagcggcggcgaagacagcgccagacagcgggtcccagagaccgcagcgcgcggcgtggcgcagggtctcgccggccactgattcaaggcgcggcggcgcggagcccgagaggcgccagggattgggcggcagcgcggctccggagcctggctcggacacccccgcgacccgtggtaggagcccggacccaaacaaaggcgggcggcgaataaaaggcggcggcggagcggagtgctcgggttgcgagctgaggactgggattcgcgcgcagcttcccgcggtctgcttgccctggagcggagggggagccccagcctcctgcggctccgagaagcttccccctgcgacttccgcgaggagacgagtctgcgcagcgtggtggccgccgccccccgaccctctgcgcactctctcccgcgccggcggctcagcctagccccgttcggccggccgaggtgagtgcacggccgcggggcgccatccgggcttggtctcggggctgggggatgcattgctaggaggagggggaagtcctccgcgccgtccgcaccccgacctgcttggcgccgcgcgcgaagttctctcgctctccagccagcccgggatgcgggcctcggagccccccgggaaagacggtcaggggtggacagaacccaagcttgccctctacgaccgggccccagctccgcaccatctccccgcgggactgcagcattcttggtcggccaaggggagttggatgttggatgtccaggaacggcaaggcttccgacctctggcccctgcttgtcccctctgagggatatctgggtccataaaagttgagcgtcagtctgaagccgcccttggatcgggctctcgcccatgctagagcggagggaggggacagagtaaggctaatgaccccctccacccactccctacctagcgtactcgtgctctcggagggacatttccccgattaacctggctgcaggtccggccggggagggcgcgtgggggcttggggcgggccgggccagcgatggatccgcgcgcgcgagggtccccaggctctgccgagaacgcgaacgagggtggaggtggagtgggcagccgggcagtggcggcgctgagggtcgggaaagattcccagcgcaaggggcgccaagtcccgggagccaaccccaccgccgggagaacttttcccaactccctggggaggccggcagcagggctgggaaggcgcctccttgggggcttctcttctcggcctctgctccacggccggcgccagcttcccgctgtcccccactcactgcgagactgcgcgcaggtctccgtcagcccactccccgggtcagcaccgcggacagcgcctcgcggccg

SEQ ID NO: 34 - DNA, 557 bp, Homo sapiens
cggcggggtcgccgctcccctgcggcagactggccagcgcgcggcgccctccatcccgtcccacgccggcccctcctccgggagtgcaggctccagcccaagcggtgctcgcgccccccgcccaaagcccacgcgagacaaagcccgaggcttctccgcgcgcccccttccccgccccgggtccctacctgacggccaccttgacgcagcagtccccctggccggccatggccctctggagctagggtctgggcgtggatcagaggcgggggtctgggggccaatgcccgaggcagcggctgcggctgcgggaggcgggggcgcgggcgcggctggcggaggctgcggcggcggcggctggaggtgacatgctcgcgggcggcaggccgaggggctcacccgcggccggccccccggggctgggcccgcgcgcctcctcgcgccatcgggcggcggcgcggagctagctgacagccggcggcgcgacccgccgctccctacggcgcggcacacgcgcacacacctcccacccggcagccacctcccgccgcagcgcg

SEQ ID NO: 35 - DNA, 668 bp, Homo sapiens
cgccgcgcgccctcctggaggaagcgcttccggccgcgggagcaccacggtcgcggcggcatgctcagcgcttccgcggagaccctcccggcgggcttccgtgtgtccaccctggggaccctgcagcccccaccggccccgccaaagaagatcatgcctgaaggtgagtaacaggcgggctgggcatggccgaggcccagccgagcgcgggcttcttcctggcaccccagggccgggccgggtggagaggggcgaggccgaggctggtgccccgcgctcctgcgctgcagctgcactaacgctccgcggggagcgtgtgtgcgcactaacccgccgctctgtgtgttctcccgcggctgccgacttctcccagctgggacggcggggtcgcagagactggagacctccacggttcggacctactcctgctgaccccccatcctcccgccccgggtctgacgggggagtgcccgtggctcggggtaagtgggccaggcccggggacgcgggcaccttgctgctggccctcggccccacgcacctgcccttggctgccggcctggccctgccgctccagtcccgcttaccagcactgcctggcttgccccttcctgcttcgtctggcctggggcgctttgcgcttggagcacgctgcgttgccgctgtcg

SEQ ID NO: 36 - DNA, 1727 bp, Homo sapiens
cgacctagggcggcggctgacgcagcccgccgccggtcagctttcaggtgagtgcctgaacccgcatcccgcccatgccttagggacccctcttcgcccagccaagcccggggcggggaggaaagtccccctcagccgcactcgggcccccaacccggccaaggggtcgccaactcgcggcactttctgcctgtttgttttcgctcagtactcacagtttagatgctaagccggctcctcttgcactcacttcctttgtgtctctctctctctttctctctctctccccctcaccaatctctttcttccctccttgtccgattctctgctgtagcttccagtggagaaaaataattattcttcaatgggttagcacggggcgcacgcacacacgcgcgcgcacacacaccccgccgaggtacccaatcaatgcacaaatcttcggggtccctttaacctctgcggatgactccgggagttaattatgaagcctatttcggcgcggcataaatatgcattaaggtaggaaataatcgatacgctgaacttctgggaactggcatttagacggactccgtctcgccgccccccactccctccatggccccacgagccacctccacagcctcgcgccttcccggggtcgcaacgcctatcacaagcttctacccgggtttcagggggcccctgtagtggcgtgggcgccgcgccggtgccgggagaccccctcgtgcgccctctgcggtcggggcagaccctcagccggagcttcttcctcccccacccctgcaggccgggcccaagctgcgacacccccagcacgggctcagcctgtgaacaaactccgagctcagtactcggaggaactggcgagagcgggacggctacctgcccgcacgcccgatttccgggcacagctagaaagaccgggacgcgattcaactcccaatccggtgaatccgcctagcgccttcgggtcctgccgcccccggccagtcccgacccgcacacatatgcaggcatttgcacacacgcgcgcacacacatgcacgcacatgcacacacacatatgcacaccctcgcgcacacagacactcccgcgcgcgctcacgcccgcacacactcagggttataacaaaggaagaggggccagcgctgccttacttttgtctctcttctctcttttgcagccccccagccccggggccggcgtgcccggcccaccccgccctccgcgccccccgacggccgcggtcagccggcattccggagcagcgcgtcggagctgcaacttcgcgcctaagcgcccgcacctcgcagcccggcgggcgggagacaaaagctgcggcgccgccgccggaggccgcgcggccaaggtgtgttcgctaattgtcaggaaatgtgtgtgtgtgcggccgggagcccgcaggggcgggggaggggacggaggaagaggagacccacccctcctacccccgccactcgccagcgccgggtacccgaaccgggattagagaataaattatctgctctcttctcattgggctgtggctgaggacgccgtcggcggcgccgggctgggagctctcccggctctttgcattcaccgcacagctccggctcgcctcgcttcgtggaggccgagagcccggggcctcggctccagacatcaggggacaccagccggctggccgcgccgccccgcccctcccctcccccgccacccg

SEQ ID NO: 37 - DNA, 765 bp, Homo sapiens
cgggcctccgcaccgctcggccatggcgagtgaaagacccggggctccggccgtcggagcacctgaccgccggggctacccgcatccgctgcatttgcggagaaatgcctcctccggcccgcccccctagcaaacagcggccccacccgcagcccatgcgggcggtgacccgatccagatgctgcccgtgccctggggcactctccaggacccccaacctcattgttctcctcggaccgcagaccccagacgttcggcaagagcccttgcccccaccccgggggcgtggttctccgcccggtccgcgctacatctttaagcccctggaggcgccagggccccccgcagccccaggtgagctagcccgagagcaagacctgcatgaggccggacagggaaagtggcctggggcgaacagaggcggggaggcggggctgagcgagcgagggggatcccccaggagcaggaggagctgggcaaggggggcggttgcggggaggggtggatggttcggaatgggcgtggtcttgccgaatgggagggtctaacgcgagtggttggcgtcccgcgcggggcccaaagtgtgacacgggtgggcttggaaccgggaccttagctactggtggggctgaagcccaccctgccattgttctgccacggtcagtggggcttgctcctccctatgggaagcacttacggccgggagcgggaatcccgtcaccaccgcggtggggaagccgcacggaaatccacggggtgcggacg

SEQ ID NO: 38 - DNA, 751 bp, Homo sapiens
cgtgatgatcgtggttttcggcttggagtacatcgtccgggtctggtccgccggatgctgctgccgctaccgaggatggcagggtcgcttccgctttgccagaaagcccttctgtgtcatcggtaatgaggcgcgccccgcccgacctgacccctgaccccaggagccagggtgacgcctttactcccaatcccgactctgaccctggagacccgcacagattcagcggaccctccccctccctccccagtcccctgccacatccccagaagtccccgcctccgggtccgtgcgcggggtaggctggctgtgatctcgccgcccccgcccctgcagacttcatcgtgttcgtggcctcggtggccgtcatcgccgcgggtacccagggcaacatcttcgccacgtccgcgctgcgcagcatgcgcttcctgcagatcctgcgcatggtgcgcatggaccgccgcggcggcacctggaagctgctgggctcagtggtctacgcgcatagcaaggtgaggcctgcaagccgcgcgcggagacccgagggcgtgtctggggcacgaccagagcgggcagggcggagagggcgaggtcagaggggctgtctccgtgctgggaaggggtgtggcctgcgggggttggagccctagcaggaggcgaggtctaagcgggtggggcgaagtggattctgaattaggtaggggcgggcctaggagtccggaccggatcagggggcggggccggagaggcg

SEQ ID NO: 39 - DNA, 1422 bp, Homo sapiens
cgcacccgactgcacgcgcctccctgggaccccgcactggcgagccgtccctcccggcgctggacgccccacctgtcccggcgccgtcggaagagccctgcagagtgagcccctagggccaccccgctgccttcctccccgtccccgcagccgctgtagccagcgtgggagccgggcaggtcgcgtggggcgctggacttaccgagaggttagcgccgccgtcgggggagggcgtgtcccgcacgtgtgtgtccgcgcgtgtccgcgcccgcgtgtgccagcgcgcgtgccttggccgtgcgcgccgagccgggtcgcactaactccctcggcgccgacgggctcagcccatggtgtgggcgccgggttcagggaggctccggctcccgccaatcgcggagcagggcggccgccccgacgcgcgacttgcgaacccggagtgcgcgccgggggcggggcagggacgcgtgggcgggaatgcacgcgcgtggccaggtgcgcgtgggtgcgagcgcgcgggagtcttcgagtgcgcgcgtgtccgtgtgcgcgtgggtgtgggtgtgctcgcgtgtccgtgcgcgcgccctcggggccaagagcgcgggccgcgcgggcaggtttggacactacgcgccctgctcgggtttaggacgcggctggcggctccgggaggaagaggaggaggaagaggaggaggcggaggaggaggaagaggaggagggaggcggaggagcagaggctgggacggccggaggagagagtccggccccagcaggaaaggaacccagcacgcgactactggaggggcgaggagcaagggaaggaccagaggggaggagagggggtcaggggagggaaagtgtgggagggggagccgcgggggtgcgcgctcctcctaactcggggtcgcttttagcgagaagtgatatttttccagcctcattgaaaaccgttgcaaacattgaaaatagtaaaagtgaaaacggttgcgacactaagacgtttataaaatgaccgcggactgccctcgatgctgatgagaactgggacgacgtcgcgcagagcgtccggagggagaggcgcggtgcccgggacctcggtcgtggggaggagccggatacagccgccgggacgcggggatgccgagggcaccgcggcctggtctgaccgacgcacgcggtgacagccgcccttgggagacgacggcgtctgcaaccagcagcctccaaagggtgcagccaggaggctcagcttgtccgcctccggggctcggggctaagccccttccagaagttttcgtttctacgcgaattccgaccgcaaaatccagcttacctgagcgccgctcctcttctctcatgtccctcgggggccgtcacgaccgtcccggagcgatctctaggaacctgtcccgcagcg

SEQ ID NO: 40 - DNA, 2535 bp, Homo sapiens
cgaaaccgccaagcgcgtccaaagcacgggttcgccaagcgccccccccgccccactccacattcccttccccgccgactcagcctccgtagctcgcggacggcccctcctcacgccagcccaggctttttttttttttttttcttctattttaaggttgtcttttaatgacacaagcgacatttggagacaaaaggacacatctcttcctgacccacctccaaccccagctgacggccgccctgagcctggcgtagacggcccggaacgttccctgcgtgggttccgtccatcccgaacccctgtccccgcgccggctccgggggtgctcggggggccgcgtggggtctgtgacgtcgcctcgaggctgcatcccggtgacccggcagcccctggcgctcgcgggaggcgggcgggcgcggaccccaggctttagggcgcgattcctgcagctggctgccggcccgaggttctggggtgtctgaggtctcgggcggggcgaggacgtttctccggctcagcccccccacctcctgccctgccgccccccacacccagctccccacggacgccaagaggcgcctcccaccccggcgaggacccgcggggaaacggggcccaggcgcggcgactgcggaggacgcgcctcggccccagcgccctggtcctcggggcgtccggctgcccttgcccgaggccggggcgggcgctcagcgccgcggaagaaacgcccgggcggggacgcacagcgaggcgggctccgcgggaagtaccgggaaaacggcgcggagcggaacagcccggagccagcccagagaaagggcggcctccccctgcagcccccggggcccagcaacgccccgccccaggccacggcacgcgaacgccgggcgggcggaggggacagggcggcgggaccagcgcggggcgggcagcgtctgggcacaggctcacgagccgctcggggcccctgcccgccgggcgctgcggccccagcctccgggccggggccaggcccacagagaacgggaagcgggcgctgcagggggaccttcggggttgcggcacggctgggaattcaacggccattcccgtcccgtcgggcccacccgcccgcccgccaaatccccgcccaccgcggatccgccccgtccaccaaggtctcgctcaccaacggcccgccccgcccaccacaggcctggcccgcccacgaaggctcgccccgcccatccacggccctgcccaccacaggcccgccccaagcctgggccccgccccagcaagggcactaatcccctaacccccgagccccgcccactgctcttgcccttctcccgcccgccccgacttaggcctctccccggcctggcctcccgtactcagccccgcccctgccccgcccccaccccaccccaaatgtgacctgcccctcccactgcccgtcccctccccgcttcacttcattaggcccccgccccgattcttgccccgcccgctcaatgtcccgcctacgaccccgcccactactagtgaccggcccgcccgttcgaggccccgcctcctccccgcccaatgctcggccccgccccgcgcgtcattgggccaggtccccgccccgcccccagccccacccccgcccctttgactggagttggctttttccagagaactttccgggctcggattggccgcgccggttgatgttccgccctttcattggcccgcggcgcccgctttcgcgagcctgcccttgggcgggggcggggccggcggcggcgcgcggcgctagattggttagcggcgcagaggggcgggcccgcgccgcgcgcagcgctcgggctggagcgcctgaagccgcgaccgccgcctcagccgcctcagccgcggtcgccgccgcttcggctgacttaggctccgccgccgcctcctcacccgcctcgccccgaccccgcccgggcctcccggcctctccccgccggcccgcgggctcgccgtccggcgcagggcaggtgagtgagcggtggcgggcggggtcggggtcgggcgcccttgcgggttacggggacccagtccccgacggaggccgagtgacggggcgaccccgggccgcggcaaacttcgcccgagccggggcgggcgggggtcccgggctgggtcgcgccgtccccgaccgccgccggcgcctgcggcggcccggggcagcgaggggactcgcgctccctcaaggtgcggtcgccaggcctggcccgcccgcctccgccccctccccaaattcggaagttgcggcgggaagccgagaacgtctcggatgtgggtcagaaaacgtctggactacttgaggcttcagtttcaaaacggcacaaatttcatctcttgattgagaagagatctggggaacaccccggagcggccgcggcggctggagggcggcggggcggggcgcggggcctcctccccctggacggccgccgggagagcgcggcctcacccctcggtgaccctgcggagagccgcgcgcgggcggtggggcgcgcg

SEQ ID NO: 41 - DNA, 1447 bp, Homo sapiens
cgctcgtggttccaggacgcggaccgtaaagaggtgccagggagctctgctcggtcccagggctccccaaccccgccgcgccggggaatggctgcgaggtctctcccctgaggaccttgaagggaccccgcgccttctgggggcagccggacgattctggcccaggtctctgaggggaatgggggccgggagggagcattcgtctgcagctctagagagaacccgagtgagaaatcttccggcgccccagggggttcccgctcccctcctagttgggccatggccctgggactggagactgcgccgcagagcccgggcccccgcagtgcgcttggctaaatagagatctcagcctcgctgcgcgcccgatccgaccctccttattataacaaatgtcccgtttgcagagcgggacatcatattcgcggagaattcattctgaaatattgatttcctctcacttcgccggcccaaatcgatagcgatgcgccctggccccataaatcgccttctctgcggccggccgggctccatccatcactgcgggccgcgctgcgcctccaccgctgtccgcactcgctgcgcgccgggacggctgcgcccaccagccctgaccctggagctccttcccagcaaagaccctgcccagccctggccgggaagggggaaaacaggcatcgcggcctgggagctcgagtgtgtttgaaggccctgcttccgggcgcaggtggggagggggttttgactccgcgagcggcttagtcctcaccagccggacgccccgcgcgcggcgcccggcctgcccggtgctgggccaggaggagtcggagagcacctgagtgcgctgccggctcccgggcccgggaccaccggctccattgccgttggcttgagagatgcggggagctcacagtccagccaggggagtcactgggaaggactgcgcgcacccgaggaagaggacttagaggtcggggacggggaaacggcaagtccagcaccggtgcagcccagggacagagcacgaagaatgtccacactaatgcgccatgcaaggctgggaatggagctcaaaggaaaccccctctaactccacagagatcccctcaccgatccatgtggtccctcgaaaccgggcaagggcggctcagctggccgggtggaaccccagtgcccgagaacagcgaggcggctccagcggagggccaggccccaggcgccccagctgggtccccacgcgggtgggtcccgcgaatcccgtgctgacgctctcgagatccaaccacccgagcgcacgcacggacgctcagcggcgcgggggctcggggatcctgaccacaccgcgttcttaccagatctgatggagttgtgaggcatcgccggggagtcgctcgcagcccgccgagggctcggggcttcctcccgtaggtccgggccgcgcctgccg

SEQ ID NO: 42 - DNA, 793 bp, Homo sapiens
cgcaccgcctcggcgggaaggaagcccgtggtttgcgcgctgagcagcctccccactcccgccctgcccagcgccgccgtttcacttcctcccggccctccctgcctgcaggagcatctctgtgcccgggtccccgggccacgccatggctgacacctacgcggtggtgcagaagcgcggggctccagcgggcgccgggagtgggacgcagacggggacggggacggggacgggggcgcgcagcgcggaggaggcgccgctctacagcaaggtgacgccgcgcgcccagcgacccggggcgcacgcggaggacgcgagggggacgctgcctggccgcggtgagtcgaggcttgctccttctcagggcatcatcctgctgtgatccgcagggcggaaccatcctgcagtggtccgtcaggccctggcggccgcggggaccccagccgctagcctggccacgccccgacgctgatgtccaggcgtccctggccacgccccggaagcgccccctggcggtcgcagtcgcggtccaggggcagggttcctggcaatacttgtcttggccgcgcccaaaggctggaggccacgtccggggtgtgtgccttctcccgcccggcgccctcggctctccccttcgggcctccggggaagcgtccccgctaggggtggggtcttgggactccctggggcttccggagctgacccgtggggggtctgctgccctcagttcctgctgaccaaagtcctgccggatctggcgcctacgaggacgtggcgggtggagctcagaccg

SEQ ID NO: 43 - DNA, 3176 bp, Homo sapiens
cgccctcgccccccccccgcaagtatcccccactactcccccccacccccgcggccctagctagctgacttgactggcacgcgccgggagcccgggctcgggcccctcggagcgtctgattggctgcggggcagctccggtctgctctgcctgcgccctcattgggcgagaggcgcagccagcggcacttcaaagcgggtgctcctcgcacttaggctgagtttagccggcgggagcctggagtccgctcggcacgagcgcggggacgcgggagccgcgcgggacccaagcagtttttccgagcagccgccaggctcagccccgctcccagcctcgctgcgcagccagagacctgctatggccacgtctatactcggggtaagtcgcaagcgcggcaacgcatttgcttgttttaaccggagtaatttttcgttatggcttctggggtctgcggctccggagaaactgttgctgctagactgcagcttcagcttctattagcactttccacatttctgggactgatttttccccaagtttttggaacccagaaagatgcctttgcaagaaaaggccttcttaatcaagtcctttttcatttggtttcaaaaaaaagctagaggtttcttttcttcccctcgtgatttataccccatcccccgcattgctttggggattttgttgcaattatgcgacaatggtgtttccagaaaacagtcttaatcgttttgcagtccatctagtcacgctaataaacatttacatttccgcggctccccaattcgcccatgactttgaaatctgtctctggccgcttatcctcggattattgttttcataatcgctgtttttgtgtgtatgtaaactttaaaaccaaacgttttctattaaaaaagaaaagtcaagtcgtagccaccctgggagagctgcaagtttcgcccgtccctggcagctgtttcggccgcctccgccctcacccggtgtttgttgtctctggtctctgtgtttaccttcgcgcttcaccccgaagggcgacttggggtcttctgggagatgagccggtttacggcgacaacccaagtccggttgtaggcagggttgcagcggggaacacggaggcatctttgatttatagcctcagcgaagtgcaactttccggcgcctggaccttcgcccgggtgatcgcccctgctccgcgcccggtgcggcgactcctccgcgggcgcgtggggaggaggcgcgaggcggggagccaggaccccccgggagcagccgcaggggacgaggctgctcacgggtgcactttgtttcttcccccacttcctcttgccctcctccttcttgctccctcccccatcccacccactctaggaagagccgcgcttcggaacgaccccgttggccatgctggcggcgacctgcaacaagatcggcaacacgagcccgctgacgacgctgccagagtcgagcgccttcgccaaaggcggctttcacccctggaagcgctcctcgtccagctgcaacctcggctccagcctctcgggcttcgcggtggccaccgggggccgtggctcgggcggcctggcgggcggctcgggcgccgccaacagcgccttctgcctggcctccacgtcgcccacgtcgtccgccttcagcagcgactacggcggcctcttctccaactcggcggctgccgcggcggcagcggccggggtgtccccgcaggaggcgggtggccagtcggccttcatttccaaggtgcacacgacggcagccgacgggctgtacccgcgcgtgggcatggcgcacccgtacgagtcctggtacaagtcgggcttccattcgacgctggcggccggcgaggtgaccaacggcgcggcgtcgtcgtggtgggacgtgcacagcagcccgggctcgtggctggaagtgcagaaccccgctggggggctccagagctcgctgcactcgggcgccccccaggcctcgctgcactcgcagctgggcacctacaaccccgacttcagctcgctcacgcactccgccttcagctccacgggcctcggctcctccgccgccgccgcctcccacctgctctccaccagccagcacctgctggcccaggacggcttcaagccggtgttgccctcctattcggactccagcgccgccgtggcagccgccgccgccagcgccatgatatcgggcgccgcggctgccgccgccggggggagctcggcacgctctgcccgccgctactcgggccgcgccacctgcgactgccccaactgccaggaggcggagcggctgggcccggccggggcgagcctgcggcgcaagggcctgcacagctgccacattccgggctgcggcaaggtgtacgggaagacgtcgcacctgaaggcgcacctgcgctggcacacgggcgagcggcccttcgtgtgcaactggctcttctgcggcaagcgcttcacgcgctcggacgagctgcagcggcatctgcggactcacacgggcgagaagcgcttcgcctgtccggtgtgcaacaagcgcttcatgcgcagcgaccacctgagcaaacacattaagacgcacaacgggggcggcgggggcaaaaagggcagcgacagtgacacggacgccagcaacctggagacgccccgttccgaatcccccgacctcatcctgcatgactccggcgtcagtgccgcccgggcggcggcagcggcggcggcggcagcggcggcggcggcggcggcggcctccgcgggaggcaaggaagcagcgtctggccccaacgactcttagaggccgggcgagaggcgcgagcacacaagcgagtagagacaccgagaacgaacgagaggttcggagggcgagcgagcgggaggcgggagggcaggggcttcagtgacgcccccagggcccgggctgggcgcgaggtggagccgctcagggctcccgggctgcggttcgcccgctgtgcgaggagctcccctctgccttccgcgcccggataagaatcgaacgcgtggtccggaaacaaaagcgaaccatcctccgacacaaacactttaaaaactgtactcccagacgtacacatacaccggagacctacaaccacaagcacgcacactcgcgcgcgcacacacataccactcgcccaaacttctcgagcg

SEQ ID NO: 44 - DNA, 2139 bp, Homo sapiens
cggcgggaatggggtggatcatccgcagggcctcggtgagcgtggtggggctgtcggccagggccggccactcctcgggcgtggccccatttcccggggtggaggacgactcactgccagagagggaggaggacaggggctggttatttttaagcaaggattcacctcggctggctagcgaggggcgtgcgcacaggcggctgctagccagccggaccttacccgggaccgtccaggggcagcggccgcagccagcgccgcccctcccggccgggcgggccccaaaagccctttctgtcaccgcaccagggcgcgaccgggtgatgcatttccacaccagcccgcccaaacctccatggttttggagctcccgggcaggcggtggaaacttggcgcaccgtgcccactctccggcgccgctccgacagcccgacgggtcccgcggccaggaagccactcggcgcccctcgccgtcactcgacccccggcccctttcggactccgatcctcccgtccccaggccacacggcgcggaaaggggatgccgagcgggacgcgcacgaccagggcgcccaggacgagggcgctggaggagactccgggcagggaccggggtcccaggggcccgggccggggctcaacacccacccgatggggtgcgggcccgacggggcccgggggtgggagtaggggcggcgggggcccgcggaggaggagtggggataggccgcgcagggggtgcccgggaccccgggcgcaagctgggaaagaggcacgcgggggcggcgcgccggggccgggacaggcgcccgtcctcacctgccgggcaggtgtcccgccgccgagtcgcgcgcgtcgctttccgaggtggaactgtcgtggtccacggcgcaggcggcgctgaaggcagcggccagcagctccatagggtcggcggcggggcgggtgcgggggccgggtcgggggccacgccggggccctgggctgggtcgggggcgactagcgggctgcggagcggggtcgcagcgcgcgcggctcgaccggagctgcagccgccgccgccaccgctgccgagcactcccgcgggcgggcgcgcgcctggaggctcccggggttgccacggcaacgagggaggcgggaggggcgcgcgcggcgggggcgaggccgggcggcggcggggcgggggcgcccgggctgggcgcgacccgggccccgcgagcgccgactgcgggctggggcctgccgcgggctcgcgccttcctcccccgcgccgccgtgccccgacccggagaggggcagagctgcggaggccctggagctgcgcctttgcctgccccgcccactcctcacggcgggggaagcctggaaggggcgggcgaggccaccctcggctccccagtgcccgcagacctccgccttgcccacgctgggcaccccctgcggtctcccgcagggtcgcagctcggcgacccgctctgaagctccgagcccggcctcggggaatgtgtccccgctggagggggtctccgtcttgtctctgctccaggcggagactggggtctcaggggctccttgcaagcccgctcctgagcctcgcggtggggagggggccggcgcggaggtctttcccgggcgagtctggtcgcaggacgagccacgctcagctacgcacgaccctggcgggtctgggatgggttcgggggcaccagaggaggagaaaccaagtgcccgaaacctctcctgggttgccctgcccggtgacatcgactcctggactctgcgccttcgcgttagctgggtcagtccgctcgcggatcagaaagagcagaaaggacagtcggaagagcgaggcctggaggtgcctacgggccgagccaagccgccctctgctcagggatcatgctgtctgctcaggtgtggcgaggatatctagcaagatgccccccgcctaccctcgcgaaggcccctggcgcgctgcccggtgtccttttccggaggccagccggctcacctgggtgggcccttgggtgaccgcggcctgcgcctgcagccgggcctgtcctttttacggccctgaggaaaccccaggggcaggggtggagtgcggagcatagtcccccgcacgctccaacccacccg

SEQ ID NO: 45 - DNA, 975 bp, Homo sapiens
cgccctccccgcatagtcacgctcaggttgctggaggcctgggtcgccccctcctgcaagccccgagagtcgccagacgcgcggctaggtgccctccgtgagcggggcggggccgcctctgcggcgcggggcgggggctatctctggccccgcccctgccccgcccgctccctctccgcgagcctcgcgccgcgcgggttgcctggcccagaccgccgctgctgtctgcggggtctggtgccggggcctgagtctctgctggctaagccgacgcctcagccgcctcagtcgcctcaatctcgccttccgccctcgctctccctccgcgccaccggaccccgtagccccgcgcgcccccagacctttaagccagatgatgaacttcctgcggcgccggctgtcggacagcagcttcatcgccaatctgcccaacggctacatgaccgacctgcagcggcccgagccccagcaagcgccgacgccgccgccccccggcccgggcgccgcctcggcctaggcggcgcccccgaccgcctcgccgggaccggagcggacgccgccggccgcctaggcgcccgcgccgtaagacgcgccgaagaccgcgcccccccccccgcgcgaacaccgtcggtgggcagcagcttattcagctcgctgtcccaagcggtaaagcagacggccgcctcggctggcctggtggacgcgcccggtgccgcgccggcagccgccaggaaggccaaggtgctgctggtggtcgacgagccgcacgccgactggtaggtgccgggcgccttcgccctcgggggtcggggtccgccggcagccgcaggccaggagaggacggtcccaggtcgccgaggaaacgtttcagaccaataaaaaggagggtccccgaggtcccgctgggaggaggtgcgggctgagcgcgcgcgcgtttgcggggtgagggagggcatccatcagcccggaaaggcg

SEQ ID NO: 46 - DNA, 858 bp, Homo sapiens
cgaatacccgttattttgggggcacgcacgggctcgtgctctgagttcctggaaggaggcctcggggagtgacgagaaaccaggggggtctgcaggacttggaccgccgaccgttcctcgctccccggggcgagcggtctggaccgcccgggaagtgcctgcgccggcggtcgtggggccagttcccgcgtggcagctgggcgcgacacaggcgcgccctcctcgtccctcccgggcagcgtcggccgcccgagcccggggagacccgccccgccccgcgccgtcacccgggccccgttccgcaggggtggctcgcggcgccccacgtccctgcgagaagcccgggatcgcttcgcggggcgcaccgacgagccgccgctcgcgagctcgccgcctacctggagggagctcaggcccgcgtcgaccgcgcgctgccggtgtccgctgggcgctcagcagcccctggagcgcggagccggcgtggagagcgcagctcacagccgagaccagagccgccggccacacccagtcccgcacctcccagcagccaactccgcggcgcgccggagccggggcggggacgtggctggaggcgcgaggcgcgaggcacgaggcgcgcgggcccggcggggacgtgccggggacgcgcagaccctcggagcgcgcgcagcccgggcggggggcgaagggagcgggcgccgcgcgcagcttctgcttcctattttctctctcgtttcctgccagaaggagaagaaaaacattcattcccgggcagtttcgttttcttgttggtgtggcgccggggcacgctgctaagtagcgcccgtctaagtggcgtggccagtttcctttccggcg

SEQ ID NO: 47 - DNA, 1523 bp, Homo sapiens
cgcggagttcgacgccgcgcgcagcaccaaacacttccccagaagggcgcagcctggaggcggccgcgcgcaggacgcacgcccaaggcgcctgggccgctagggaccgaccggagcgctcaaacccacagggtatctatcaggcgcgcgtcagcacgtaagcagagcgcgagaacgcccgagacggcggcgcagccaatcccagagcagcgctccccaacggccggccggagggcggggagagaggggggctcggccagagccacgcttcccattggttgagacagcaccgcccagccaaagcccccttgtcctcgcgcgggtgcgccgcctggactcccaccctggccagtcccgggcccaccaccactctggcatccccagcctgtccccgcggctcctcctccttccccggccctgtggcctgcctgctgtccagcattcttccccagggcgctgtggcgcggagggaggactccccaaaccccgggaacagagccttgcggaggaccccggaggaggaggaggaggagggacgcggacgggactgggagattcggtcgggaaggggacagggcctagagcccggggctagaaggcaggacacctggcttcggtctgggctcggcctttgactacgacgtgaccttgaggacggttccttcccttcgtggggcctgggtccccttcagtaagatgcgccggcaggaaacgatgagtcgtttcacctgggagctgcagggccgcgtgtttaacccagggcctcctcctgcctggcacattattccgaataataaatgccagatcatgctgctggagacgccggcagaaaagccgagagggagactccctggggtctgggcgcgatcggccatcctacccttaccccgctgcggccgcagggctcggcctgtagggcgcgagccccccttccccacccgtatcccggcttcggagctaaaggaacacgtgaggccgaaccaaaccgagtagagggcgccagggaatcccgcccgagctgcagagacgcggacccctcctcttacccaaaaccaaatgggcagggggctccgctgcagagggctcgccgaggctctcgccttcacccggcgccgcgcaggtgcccgctctcctgcggggagagccgagggggggcgctgccgcagggtctttctggaagcccctccaccagcactcctgcggcctgcggcctccgccacccggacgcttctacacgcgaagcgcgcgctggggctacgctgggcggggctcacggcggcgtggctgtcggggggccgccgagccgcctcctccgcgctgggtgcccggctggcgctcctcccgcccaaggtgccctgatcgctaggctcccggtgcggccgtctcgcccctaagccgggcgccagatcccccgccgccttctcgctccccgaccgctgggcgggggcgagggccagggagatggagccaggtgcggaaagggttgcagcctcgcgggctgaggccggagggtgaaggcgccg

SEQ ID NO: 48 - DNA, 1386 bp, Homo sapiens
cgttccgcgtcttccggaggcgcacagcaagggagtctcctggtgtcatctcgggccattacggcgcttagtcgatcattaggtctgtgggtgactgtaggcagagccgggagttgggtgcttggcgcggagggtgaactcgcgggtcttgagctgcaactagaagctgctcctagcaaggggtcgggtggagatgaaaagactgtggggaaggggaggtggttgtatgcccagagatatcccggcgtaactacggagtccaggcccagagtggcggtgtgtggcgaaggcgcagcaggcctgcgcctgtcggaggacggcagatctggacgcgaccgcgcgggcgtgcctgggtcccgcttcctgcccatcgagggcgacttccttcgggctcagctgtgagctccaggaactctgcaacctcccgacgggcgccacgctccggcttgggccggcccgggagttggcggagctgcgcggcccggccgggcggccggtgaggctttcgagaggcaccgggctcgctcagcgtccgcgcctctctaccccgccgcgccccctccgcgcccacgggcccattttttcctagtcttctgcctctcttccttcttcctccctcccctcctcctccacacctttgtcttcttcctcctccctcctcctgcttctcctccgcagtttggatcctacgggccccctacgtgccgcgcccttcggtccccggaaccgcccgggctgggggtgctgctgcgcggtgagacttccgcgggcggcgggcggagagcgggatttgactgcgaaagccaagggggcggggaaggggaagccccggccctcgggggcgcgcagtaaactgctgcacacgtgcccgcgctcccgaggggggcgcccagtgggctcgggatttcctggctgcgccaccgcgcgcctcgcccccgacgcccgccacctgtgccctgggccgccttccctgacgccagcggccggaggtcgcgcgcgccgggtgctgcccagcaacgaaaggccatgcggggttcgtccctcagatgccggtgaccttgattgaagtcacctgccttctctagcttccagtcctttaaaacgagggtttggacccggtgccttctgaacccggtgcttcactgagtgagcaagtgaagggtgtgtgtgtgtgtttggggtgggggcgggggcgaggggagggaggggcagcgttgccggctccccagagccaggtccaggcagaggtcgcgcccagcactgaccgtcggtcccaatccgcagactcccgagggcgatccaaaccgcctaagcatcccaagcgtccagcacccgcgcctgcgttcctgagtaatccagcgccctgcagcgtttttcatgcacacg

SEQ ID NO: 49 - DNA, 285 bp, Homo sapiens
cgcttttatgtcgcgccagactcctccggtgaggagcccgcagagctgctgaaaggggctttgagaggcccgggcgcgcggctgctgcgagctgcgcgccccccgccggcctgagtggcaccgcgtgcccctgccggggcgcggctcctgcactgcgggcgcgcaggcggcggcgggctctgccggcgccccttattagcatgcaccctgggtgcgcctggcctgctcccgctgcttccgggcctctctgcgcccctccgagtctctgccatccgcctgggtgcg

SEQ ID NO: 50 - DNA, 1931 bp, Homo sapiens
cgccgtggagagaacatgcgcgggggaaagccccggtcctccggctggcgaaatggacctctggaaggaatggccacacggtgtgaaatcactggtgtttattggctgtgattccatccggagagaacacacgcaggggccccgacatgcaggaggaggcgcaggcgcaggacagacggacagaggacaccacggtctaagctaagctcgcggcccggggcgccatgcgctgggaacggggtgcgcaggttctacgagaggacgccctgtctgctcagagctggctttgtaaggtgtgaaaacaggagtttttaaaagacacgacccgggagaagtcagtgagagggcacagggcgagcaggacggacagcgacgtccccgcgggccgcgtcgctggggcgcagaggggcgcggtggtctctgcccggaggggcgtcggtcgttagtattgcagtctaacgttatggcttctctaaagctatgtaaggtcatgaaggtcaatgccaagccacgccctggcccgaaacacgtggagacttgatgcatttttgatgtggacgaaagggcccgggggcgaggcggcgcctgtcaagataaaactcattaaatgcaaagacctcatttacctgagattcaacaaattgtgatgcaaattaaacatgaatggaggagaaacaggggctcggatgccgccccgcagggccaccaggtggattaggccacacacgcgccagctgcgcgcagggaaccccgcaggccccacccgaggagctgcccacggaggaggtgctgggcaggggcgcagggtctccagcgtccggtgcctcgggcctctgcggctcctgtggagggtgcagtgttcaatggccgagggcaggggtcctccccagggagaagcagcagccgcgtgggcggagaggctaggaggccgggcggggggcgaggacttgggaagagcggggtgacgggggtgggggctgggcgtcccccaaacctattgctttgtttcctttagtttagaagtgaacacggccgtggcgttcgtaagaagcaaaaccttccagagaggagaggaaaggacgcggacagagacggatggacaggggcgcgagggggccaggccggggggcggagagcggccgcgtgcggacgcaaatactcacggcggagcgatccgaaccccggagtccgcgtgcaggagggggacccaggccccgccgccctcgccccgcccgcagccccccgtctgctatggctgtgctcctgagtgcgggcccgacaggcgcccaccgccgcgaacgcgcgcacaggggtggtcgcggggctgcccttcggcggccctggcctcgggcgctgctgggtgggcggtaaacagcaagagaaaggggcgtcccaggcgatggggctccagccacccgcgggcagaggagcacggggcggactgggggggtcctgggggcgcgcgccccttgccgggtgagggaggcggcgggcgcgggcagggcgggcctggggctatggcttatatcggcgtgggggctgcgctctgctcgtccctcaggggcctccgcggaggatctgtagggggaggggcgggggctccgcggggccgctgtcccgcgcgcggctacttcttgagcatgtcctgcagcggcccgggcaggtacttgatgacggtgtccaggatgctctcgtcctcctcctccacctcgtccccgcagcccggcgggatggccttcttgggccgcgtcaagctcccctcggagttggcctccatggcggcctgggcctcggcctcgcgctcctccttcttcttgatgccgtactgcgggggaggcgggggtcagggcgggggtcccggcggctcccgggcgggccgcagcctccctgcgccagggactggcccaggaacccccgaaggcgtgggtgcg

SEQ ID NO: 51 - DNA, 923 bp, Homo sapiens
gcgctttcgtgcgcatcctcgggtgccagtgccgcggccgcggccgccgccgacgccgccgccgccgtcgcctgggcggctgcgcctacacctaccggccgtggacgcgcggcggctcgctggagcgctcgcagtcgcgcaaggactcgctggacgacagcggcagctgcctgagcggcagccagcggaccctgccctcggcctcgccgagcccgggctacctgggccgcggcgcgccaccgccagtcgagctgtgcgccttccccgagtggaaggcgcccggcgccctcctgagcctgcccgcgcctgagccccccggccgccgcggccgccacgactcgggcccgctcttcaccttcaagctcctgaccgagcccgagagccccgggaccgacggcggcgccagcaacggaggctgcgaggccgcggccgacgtggccaacgggcagccgggcttcaaaagcaacatgcccctggcgcccgggcagttttagggcccccgtgcgcagctttctttccctggggaggaaaacatcgtgggggggaggggagggcggggcggaggggggaggggagcgtccacgccgggtagacgcgcgccccaaggggaaccggggggagggccggggagaggggcagctgcttttctggcaggggcatgggtgccaggtaccacgcggaaagccgggccgagcatgctgagagcctgggggacccgacgccgccgggatttacctctctctctccctctgtgtatatataaacgagtccctctctacttgtatttaaccgtgggtgcacgtgagtgtgactgtgcggtgtgcgtgtgttccgcttgtgtgtgtgcgtggggccgccctgtgagggcgcggcgactgtgcgcccaggaggcaaccgggggcgttgtgtgtgtcgtgacttcg

SEQ ID NO: 52 - DNA, 457 bp, Homo sapiens
cgtgcggggctggctgcgggtgggggtgcgagggggtccgccggcggcccccccgcggggagggaacggttcttacagtcactgctcagcgcgaagggcgcgcggcggcggccgagggagcggtcttcggcgggcgcctcgggaggcctcttaccggcgggcagcgggaaggcggacgcggtgtggagcagcaccaggcagacagccacgacatcccataacttcatcttaaagtcccgtccggcggcggcacctgcgcgggcaggcgggaggtgggggagagaaccgcagaatgcacgttaagcctgggctccctgcgggtccctgcgcccctctccaacctcgtcaccgccctcctccggcaccgctgcaggccagcggctcctcgggcactcctgccctccttgcaagtccccagttcgcttctccctcctcagcgcgccccaggagcagctcg

SEQ ID NO: 53 - DNA, 615 bp, Homo sapiens
cgcaggaagcggacctggcgaaaaagagcagcgaatcggaactgagcgtgggggcagtccggggagatcgcgccggtccttctagaaggcttccctcctctgaaacccgctgcagtccacagccccccaggtttggcccccgccccgcttaggctccatccccgaactcaccggcccgccccgtcccggaccagtcgaatagcaggtacggaagaatcgcgaggcgcggaaggatcgcggggctggcggccgctgccattgtagaccaggagcgcccgggacacaaggcgcagtcgatgtggagaccagcgtatgaatcacttcggccctcgcatgccagctagtgattgagtgtgctcggccccgtccggtcccgtcccgccccgggatccaggggagcctacgcctgcccacgtttggagggatgctccgccctatcccgccccgtcccgcccttccccgccccaccccaggctgggacccggggagcctatgcctgctgtggctccgcttcgtcccgccccgggctcgtggcctgacagcggccccctctggccaacccttaccctccaccccaggctccgcgcgggccaggacacgattccttcttgaccg

SEQ ID NO: 54 - DNA, 2068 bp, Homo sapiens
cggttgccgcgcgtggacttacaagcccacgaagccatcgttcgtcacagcctggaaaagggagggggcatcgatctggtttatgtttggacctcgaggctccaccagcgctggcgcaggcaggaaggaggtgagcgccgcccgcgtgcacacaaggcgcgcggcccccaggcagccgcgcttccacagcgggcaggagcgcgacccccggcgcgggcagctctgcgcgaaggtgggcgacggtgggggcacaagaacagcagccgggggcgaaggaatcagccccgcagcggctcctaagcctccccgctggccgcggctgcgccgctggagacccggcgagtgacttccccggtgcggccgggtcccggcgccacacgcccttttcctttctcaggatccgcggccgtgcagagaacaggaagacgggctccaaccccacctggttcgtgtcggggggtcgttttgcggtggggggagtccgaaccgcgccacgggcccgcgagacgcccacctctcgcgtgcgtggagcccggggccgaggccaggcccgctgcgagcagcggaggcatgtgctgatttgcatgagaacgggcgggtcgcgtggctcagctggcggccccggcgcagatgacggctcggcagcgggcccccgccccctcctctgcgccgcggctccttcccctggaacccgcggatcatctgctggggctgtcccgcgccccatctccatgacaaagcgcgtgtttacctgcggccggagcaccggcgggcacgcctttgtcccgggatcagattaccgcagcagctgctgcgggcccgccccgtgcgaaccccggagctgcccctccgaactttgtgccgactgaacgcgccgggacccggggctggggggccgggtgttggggggttagaaggaggccgcgtgtcccagggaggggctgttttcagtgcgatgattgccggacgctgagatccgcgacggggagtgagcgcacctgagggcttcctggaggtggtggcggcgccaggcagcattacgaggggcaccagtgcaaggcccaaagcgcgagggcacaggcggcgcggccgtgcggcgggtcatccctgggccactgcgagggcggagggcggcgccagggccctcccggtggaccccagtcgccgtcgtgacggccccacgcagcctcgcgcccggccgccgcctcgtgcgggactcgtgcccgcattcccacctcaccgggtgcagccggcccggccccgggctgtgcgaggcgacggcgcacacctcggagccctgggggccggggtcggcgggcggggccggggcgcgccctttgtgcgctcttagcggaaaccgcgcgttcccagaggagcgcggcccgccgagaccccggatcccgacgccaggaggtgctgcaggagcccgcgcggggtgggggtctccggcccaggcccctcggtacgggccccctgcggccgccaccctccgcctccgcgcggcggctcctttccggctgcatccggggggtcgaggcgtcgcacctggaaatcgcagcctgtgactttgtaaaaaagtgaatggggaagaaaaggcgctgagaggggcgatgggcggacgggccgtgcccaccacagggaggtcgcggcacctgtcgctcctggttcccagggcggcctgtcaatcacccacgctgggctcagcttgccgggagcgggggcacgcggggggcttcaaggcccagagctcagaggctcgcaaacaataaactcagaaacgtggcgcgcacccgcagggcctgacacagcctctggagtggcccctgggcctgtctggctgcagggttggttggccgtgggcgtgtgcggctcagggtttcctccacgccctttcacgaatgtccccacgggaccccgcgtccccccaggctgctccagaggagtcccggagagcgcggccgtggaagctttcgaacgcgccgtgcttccccgccgcccccgcctccctttctgtgagcatctgccggggagaacagatgtgggcacctgcggtgaccggctgcgtgcg

SEQ ID NO: 55 - DNA, 723 bp, Homo sapiens
cgggcaactcggcgctggtgctgctggcggtgcgcgacccgcgcctgcacacgcccatgtactacttcctctgccacctggccttggtagacgcgggcttcactactagcgtggtgccgccgctgctggccaacctgcgcggaccagcgctctggctgccgcgcagccactgcacggcccagctgtgcgcatcgctggctctgggttccgccgaatgcgtcctcctggcggtgatggctctggaccgcgcggccgcagtgtgccgcccgctgcgctatgcggggctcgtctccccgcgcctatgtcgcacgctggccagcgcctcctggctaagcggcctcaccaactcggttgcgcaaaccgcgctcctggctgagcggccgctgtgcgcgccccgcctgctggaccacttcatctgtgagctgccggcgttgctcaagctggcctgcggaggcgacggagacactaccgagaaccagatgttcgccgcccgcgtggtcatcctgctgctgccgtttgccgtcatcctggcctcctacggtgccgtggcccgagctgtctgttgcatgcggttcagcggaggccggaggagggcggtgggcacgtgtgggtcccacctgacagccgtctgcctgttctacggctcggccatctacacctacctgcagcccgcgcagcgctacaaccaggcacggggcaagttcgtatcgctcttctacaccg

SEQ ID NO: 56 - DNA, 545 bp, Homo sapiens
cgcttcgacggcctcccggcggttctgggcttcccgggcttctcctccgggcgccaccgctggcaggttgacctgcagctgggcgacggcggcggctgcacggtgggggtggccggggagggggtgaggaggaagggagagatgggactcagcgccgaggacggcgtctgggccgtgatcatctcgcaccagcagtgctgggccagcacctccccgggcaccgacctgccgctgagcgagatcccgcgcggcgtgagagtcgccctggactacgaggcggggcaggtgaccctccacaacgcccagacccaggagcccatcttcaccttcactgcctctttctccggcaaagtcttccctttctttgccgtctggaaaaaaggttcctgccttacgctgaaaggctgaagtggggcgcgcgaagggcggcgaagcggagacggcggctctccgggatccagctccgcccctggccagtgtgcggcccgggggctccctgtgcccgcgtgaggcgagagaacaggggacttgagtctcgaacagcg

SEQ ID NO: 57 - DNA, 1057 bp, Homo sapiens
cgccgcgccgctcccacgggctatgaagagttctacttccctctggtggaggccacccggctccgctgtgtcaccaaatgcacgtcgggcgtggacaacgccatcgactgtcaccagggccagtgcgttctagagacgagcggtcccgcgtgtcggtaaggccccgctcaccatcagcatcagtcgagccccgcccactcattctaggatgaagcctagcctcacgcgccgccccggctctgcccccaggccctacagtggagcctcgtccccagagtcccgctccaagcccatccccgttgccctacagtggagccctgccctggagctctgctccgtcgccctaaagtgtagccccgcctccttgatggggttgagtccaatcccctggttctgggatagaccccgcccactcattctagggtggggccccgccccttcgttctagggctgaaccttgcccccttcttctggggtggagccccgcccccttgttctagggtggatccccgccccctccttttagggtgaagccctgcccacttgatctaaagtggaatcccgccccctcacctagggtagagccccgccccctcgttctagggtggagacccgtccgcttgttctacggtggattccggccgcttgtctagggtggaaccccccagcttgccctagggtggaacccccccgctgccctaggctggagccccgccccctcacccgcccccgcggggcccaggtgcacgcgtggaccccgagcccggaggtgaagagggtctgaccctgcgatctcccgcagctgctactccaccgacacgcactggttctctggcccgcgctgcgaggtggccgtccactggagggcgctggtcgggggcctgacggccggcgccgcgctgctggtgctgctgctgctggcgctgggcgtccgggcggtgcgctccggatggtggggcggccagcgccgaggccggtgagcgtgcggggggcggggccggggggcgagggcagccaaggggtcccaggcgggccggctctgtctgaccgcgcggcg

SEQ ID NO: 58 - DNA, 1695 bp, Homo sapiens
cggagccggagccggcgaagccgcggagttgaggcgcgcctgggagagccaaggcccggcgggctatgcaggtgcatgcccccccctccgcccaaggagcggcgccaccagcgcttcccggctttgtccttggaccctggcaccggctcgctccagccgcgggggaaggcgcttcatcgccaaagtgcgctccggcggccccggcccgctctgcgggccgccgccggagggagtgcgggggcgccccgaagccgcccaggccgcactcgctggatgctgcgctccctctcccggcgtcggccgctccgggccggcggcagctgattgcggtcgtgagctcgcggagcggcgagagttaagaggaggtgttcgcggcgccttcgccctccccagacacacaccccctttctcctcgcctcttctcggagggtggaagagacaaaaacggggggtcagttctccagtccgccggctgccggcacgcggggcggcgctccgtccggcccccgaggaccagcggcgtggcggctgcgctcggacccggtccgccggccggaggagcgcgcggcgcccgcggtcgcgcccgcaccgcaggccccttcagagagtccgggggcgggcgcgggtccggcggcggcggcggccgcagcccccggcggcgcgcaaccgcccgtcgccgcccgccgctgcccggcccccggcgcgccgccggctccggctttgtgcggagggagcgagttcgcggcttcggctcccggcgccctcagtggccgcgccgccagcggactgctgcagcggcgtgagcagcgggggtcgcggctgcgggaaacgctccggagcccaggaacatagtccccgctggctagcggcggcagcagcagcagcggggcccctgcgcgcggcgcccaccgtctcctcctcgcgccgggctcgcggtgttgcaggcggcagccacgcagactgctctctcatccttttgtccttcagtcagaacgtgaatgtactgctgacgcatactgttctgggagaagattagcgtgatgcagtgctcttatgtattagcgccgctccccctccgtcgcctgcctcgcggggttaacgccggcgccttccagcgccgcgccggccggcgccgcgcagccccgcgcctccggccgcccgtcttcctctttcctccttccttcctcctctcttcctctctccccctcccccctcccccctccccgggccgcttcctccccgccccctctagacgagcggagcggggccagggcgagcgggcgagcgagccggagtgagcgtcaagtgaggggccgcgcgcaagtcgcaggcgttcgcagctattttgggccggtcggagggagcagttgggtggccgctgataggctggagcggcgcggggctgcggcgggagagcccgcgccggcccccgccctccggcccgcacccggggccccctggctctgaagagcggggctcaggggcccggcggcccgcggcggggctgctgcatttggcctgcctctcgcccggcccgcgtcatcctcacccccgctgactgctgacggggcgcggactcgcgttcggagttcggaactgcgtggtggggactcgggggacggcggccggggccacgaggctcccaggaccgagagcccgggcttcgctgctgcactgccgccagcccctccttcg

SEQ ID NO: 59 - DNA, 360 bp, Homo sapiens
cgcttcggcccccagcgcagctgcagaggggcccccctcgacgcatacactcaagagcccgaccgcgcggctgaaatcgcggagctcggagccgcggctggctgagcgatcgcggttcctgggctgcgtgcgcgccccttggagctgaaaggagcgccaggatcgggggcgctgcaccgggctgggcccctcaacgctcgcagaccgggccgggctgcagctggagatggcagcaatcccgggaggtctccgggcctcttcagggtgcgtccaggaggcgggttccgtgcgacgcggcgcagcccacccccacgagaccgcttaacttcgcgggggcagcctcgggcgctcggagacgcg

SEQ ID NO: 60 - DNA, 358 bp, Homo sapiens
cgagatggcctcagcacgccctgcaccctcgtccgcgcgactgccgccgggtgcccgccccagccgctgtgtccctggtgcttcctccgccagggccacgtgccggggaagccaaagcgggagtctttcgatcttggcccagcccttagccctcggtcctttgctctagcatctagattctcgtgggtcaacaaagtcactggaaacccaacgaacttcccgacatttttcgaaaaccgtatgcgtgtgcttctgtgcgtttttcctctgatcgaggtagggccctgtagggttctgtttggacgggaggctggagtggttccttcccggctgcggggtcggaggatgcagagattcg

SEQ ID NO: 61 - DNA, 948 bp, Homo sapiens
cgaggcggcgcctggccgaggagctccgcactgcgccttgttaacagctgggtccgggcaaactcgcgccacctaattctctgccctctaccggcttgagatgcttattcctcagggagggcaagaatcgaaacgattgagaccaactatttacaagtgaggcaaccgggctacaggtgcctcgagggaaaggaggctgggtctgcgctcccacaccttgccgacggctccacgtccctgtctgcgcccgaggtccaccggtctgtgaagcccactttccccgcctccctctcgcgttgtgccgcttggggcgcccgctaggacccaaaatgcggactggtgggccgcccatcaagggttgcccgcggtagggcgggggctcccgtgagctgctcccaaaccccactcgcaggatgctgccctgacgcccagggccaacccggggagccaaggcccccggtctctgcaaggccgaccccagcccgctggggaccctgcaagcccgcgcgtggctcccagcacgccgggcgtccgcggggcgagctgctcagggtcactcggggtccctcgccaatacaggccgcgcggagagtgtgaggcgagccccgggcccgcgcccccacgagcacctaccttccaggtacccaaccccaggatgggcatcttggcgccgttgttgagcaggagacggcttgccatggctgctgcgctccccagacccccgcccagtacggtgcggccttggccgcggcgcgtacctttaaatagcccgtgaggtcggcagaaagggcgcctgcggttggcgcgccgctgcgcgaaggagccttctgattggttgcgctgggggtgccgcggcggccttccccaagggtcggcggggatgcttcttccgcctggtcctagtggcagcggattctttccgcccccaacgctcgcccgcgccccgcg

SEQ ID NO: 62 - DNA, 351 bp, Homo sapiens
cgcgagcatctccccccgcccccggctccgcccaatttcccacctcttcccggagcgcacagacgccagctgagtgggaggggcctagctgcgggcggagcacggaggaggaagggcacgaggaagaggcggagcgggccgaggcgcgcggagctgggtttgctgagtgcttccttcctcggccccacctcccgccgggcaggatgccgtttgatcattggctggtttaataaagagggcgtgtttattttgcataaatagtgggcgggcacccagggctgctttgtaactcgagaacccggtgtcccgcctgtctcccggggggtatgggcgggtctcccgccagcctcg

SEQ ID NO: 63 - DNA, 1980 bp, Homo sapiens
cgcttccaggccggatttgccttttcttcttcccgcaacgaagattcccgcccctcagcaactttgaaaaaagcatgggggatcgtaaactcgaacttcgccggttaatgggcttatttattggcgctggcggctgcttattttggatgccttacaaacatccgcgctatctgcgggcgagctactttccctccctccccctccccccgcgtgggccgcgccgcgcaggctgggcagggaccagggctctgggtcctcccggccacaggaaagagcgcacaggagggggcctgctcgctggtgtcctcgtccctagtcagggggagctgaggccagcgccgaggacgtcttgctgtggggcgctaagccggacatgaattttactgcgtccccacgcccaaatattaaaaagcaagttcacaaggtcagcctgcctgcagcttgggccaaggccggccggctgctgcgcgggctcctagttttctgatccttctcctccttgtgtctgcctgtctgcccgcctgactgcagccctctgcagccctgcttacccagggaatccttctccggcgaggctttgctgctctcggaaggggccggggagagctcctccgcggccgaggacgacgcgtgcgcctcctcgtcgccctgcgagcccccgccgctgccgcaagccagcgtggggggcggcggcgaatcgagggctcgctccttccgggccgcatcggccgagccggaggctagcgcgggcgggagatcgaaaccgcgccccgggggctgcgcggggaacgggccagccccgagttgctgcgcgccgccgccgccgctgccatagcccttggcggtgccgtaggcctgagaaaggcggaagtagccaggcactggcaccccgctggaggtgcccagggcgcagccgtcgggcggcgggccccgcgggaagggagccagttcggcggcggtggccgagactttggggcatttgtccgccgagtcgtagaggcagtaggagctctcttctttgatgttctgcgcgaaagagcacgaggtggcctgcggcgctggctggggtggttgcggcgggggcggcggctgctgctggggcggcggcggcggcccgtcaggcggctccatccggcaagaccggggcgcgtctagccacaggtctatgggcgagggcccgtagccgtgcgccccgggacctagacccccgccaccgccaccgctgcccggcgacgctgcctcattgcgcttgccgcccagcgtggggaagagcccgcagctctgcagcccgtagggcaggtcggcggcgggcggcaggtagaccccgccgtgggcgtagtaaccgccaccgccgccgccccccgcgccaccaccaccgccgcctgcctcgcctctgcccgagctgatgagcgagtcgaccaaaaaagagttcgcggcggggctctccgagcatgacattgttgtgggataatttggcgaagggagcagatagccctttctggctgacatttcttgtgcaaaacatgctgaatacgattagcaatccccccgcaccgcggcgggcgcccgcagccaatcccgagccagagtttccgcgcgaccactcccagtttggtttcgtaggcgcggggccgctctccgagggcgccctcagagcccgcgattgatataaatatgtaatctgtattgatgggccaggagacgcaccccgacaccttggcccgaaggccgggagctgtgggggctgccccaacgtggctggtggggggcctggccattgggctcgccccgcccctacccggacgtgagccccataccggggtcccttagaagggcccttgggccccgcgcagttaacaagtggggtgtttatggtgcgcgcccagtctgccttgggtgctcaccatccctgtcgcagaagctgccactagtccccggtgtactctaaccactgaagcggccgtgtcggggactcacgcg

SEQ ID NO: 64 - DNA, 3043 bp, Homo sapiens
cgcgcgtgtggaaggctgggtggggtgcgcacacacgctcacttgtgtacgtacacacacacacacacacacacacacacaggcacacacacccgagctcagtcacccacattgcttgcccagggcgcctggaagagctggcgagcccgcccagctctgttcacccggccccgccccgccccctccgggagggctctccgggcggcgcccagccccgagcagagcaaaggacggcggcggccacctccgttctccggccgctggtttctcgtctttcctcgtcctttcattgaacccatctccgtgcttcgaaaatctgactctaacccgatctcttcgcgtctccgcctcttctttctagagtgagcgccaaaaagggcctgacagacagaagcctttggccccagcaccggcccagcccgtctctagacgattcttgctcctttcaccctcacagcctccagtggtcgcttcatcttcgcaccctcccggccaaccctaactctcctcgtctctcctcgcgctgtctcgcgtcctcccctcaggatccttccgcacattctcagcgtccagcgcggtttcccacaacttcctcacgccccgctcccctcgccctgtccccgccctcgacaccacctgcgttccccactcgctccaacctccctcccccgctatccccacttgtgggcctccagctctctgcccctttcctggcccccatccctgacaccccagggacccttcctccctcctcacgttctccctccttccaggatcccgccccgacacttcggggccctcccgctacgcgcactctttctcctcaggtcctgacacctgggcgccccctccctgtcacccaccttcagctccagccctgactctcgggcgccttgccacccttacgctccccgccccgccccggtccctcgggcgcccccactcgccgcctctacctccctacctgctacacctggcacccctgcccccacccctgctcatactcttcccccgcccccgacacctcgggcgccccctcacacgctcctattctccacacttccgtccccgaaaactcgggcgccctctcccttcacgctcagtctctcctccctccccgcccggccccggacccagcctggagatcgcgctcgggaggggcggctgccgcccgcggggcgcccgcggtgcccgggcctgggcagcgaggaggtgacgccgcccccgcgggatgagcccgggaggcggaggggcggaggaggtgctggcggcggcactgagcggcggcggcgcagggcgcgcgggccttccgcgccgactccatcgacccaaggggcggcggcggtggcggcggctgagcgaccctgggccgggcgcgtgatgaggaggggccggcgccagaccccgctgcacgtcggagctcgcctggatccgggcgttggcagccgaagggccctggccccgggactctccgccgctagcccccgtcatatcttctccgctttcgcttctccactctagccgggggtggggtgggtggggttggggtctccgcgggggtttccggccccgcggcccgctcccgggtgtgcctggaggagttctccctctgtggcgcgcgggagccctgtgatgcgtcagccggcgggacggatgagttgcttctccgggaaaccgtcctcgcttcctcacgaccctctcggctcccgcctgggtgcccctcgggccggcagtactccgcctccgggcgctcgaagcgagttccccgggggcttgttcgcaggcaccccttcccctccgaggcggcgcgcgcgctcccggccctgaccgcggccggacacactcgcgccccggtccgcctgtcgccctcccgcctgctccctccagtcaccccacccttagctgtccccgccaccttactccaccaccctcccccgcctctccgcgcactccgcgtcccggcctccagttcccctttcccttgaaccgctcacttcacagcccttcgcccccgggaagaagaaacatttcccgaagcgcactcctcagccctccttccccacgcgctcgccctcccctccccctgcttttcttgggggaggggggctgtcgccttggattgaaggccattgatttgtatgtatttgtcccagcgctggaggctgccccagccgccgcgccggtgccgccgctgccagtggagttgcctccccgcttccctagggtggttcggctccaccaaacatgtcggctcctgtcgggccccggggccgcctggctcccatcccggcggcctctcagccgcctctgcagcccgagatgcctgacctcagccacctcacggaggaggagaggaaaatcatcctggccgtcatggataggcagaagaaagaagaggagaaggagcagtccgtgctcaagtaaggacctggctccatattcccgcctctctccctgccctccgccccctcgcccactgccctgcggccgcctgcgcgccccagttcgccgccctccctcccgctggcggcgcccaggccacgagggctgcggccagcgccggccgcccgggctgttttaggggtgtctgagagcaggggtgtgtgtcggggagggagggcgccaaggccggctgaggtgagggtggcgagccttaggcggtgtgattttccttggcgcctttccggatttcctcgctggtcatcttggctccggggccccagcgggactggggctgaacccaggctctgtgcgtaccctctcctttcccgccgcgctgaggcagtgactggggcacagaatccaatatggccgtgcacaggtgctccctggacggacccgggcgaaggcgcgctggcaggggatgcggacgccaccctggtcccacgcctccgcggggcggctctaccagcaacgcgggacagagcagggctgcccacagtggctgcgagcagcgggcggcggcgcgggctaggggcctaagctctgtcgcggtcgggtgggtgtgcgtccgccgccatcttccagcccctccccctcggcg

SEQ ID NO: 65 - DNA, 864 bp, Homo sapiens
cgccctgcgtttcttccccacgtcacttgccagggagccgctaaacagcaagaccgcgcgctctgcggctccagagtgcggatttcggtcgcgtgcggctctgaccgcgtcgccccatccctggcggggccacgcacggacgccatggctggcgccgcggagccgggcgatgcgcgcggactctcccggggccctgactgtccctgagtcctccctgcggggggcgtgcgcggcccgccccccgcggcgccacgcggcccctcctcggccggggattggtgcgccgggcggggcggggcggggcgggataaaggcgcggggtctggctgcgcggggtctgcgggcagctccaactctgggttcgtagtttgcgctgggtgcgcaggaaggtcagtgtgggggtcgcccgacatttcccccccgcggaggtgggagccgagccacatcttggagtggggactggccgcggagcgggttgcccagggccggccgaggtcggggcgagccctgcgcggcgctggagactctgcattcccgggcgcgcgcagggtccccggccgtggtcgcagagtcaggaggggcggctccggagcccggcgcggggagggcccaggcgcagtcggggttggcagggcgcgacactcgctcccctccacttttgaaagggcttcccacgccgagaagaggggcgggcatggccggcccggcgaaaccggtttgtacagactttgggaagccatcgcctgcggagggtgggaccccacagcttgtccacctgcccaggctgagacctcgtgtcctagtcctggatgccccacgggtttctcgtcccgggcagcggcgcacgggaggagaagactcccg

SEQ ID NO: 66 - DNA, 238 bp, Homo sapiens
cgtctgtccgcttggatgtcccaccgctgtcaggcatttaatcaccggccagtgtcccctgacccgcgcgacacatggcgcatcaaccgcatcgcagaggaagtctgccccttcctcagcccctacggaagcgcccgggctgcaaggccctgccacatggtacggacagggcacagaccgctcggccaagctgtcctgagccgctctgaggcgggtgcaccaagggatgcgacacccg

SEQ ID NO: 67 - DNA, 1247 bp, Homo sapiens
cgggcgtgttcagaaggcggatctcaccaatccggaggcagctggaagagttcgcgcgtgggaaacctcagccctttctttcttcccggcacagcgttgtgctgccgcgcgtcggcgcaggcgggggcgcaaggcccgggtcgtccccctcgcgcggtcgggaaaacagctactgagctgcggcccacgaccctcggccgcgcaggggcggggcgggggcgctgcggggtttctctgttgctagagccgtgatgtcacccgcccctcctaacccgcggttggttgttttgtgtttcagctctgcctgcagttgaacacaaagacctggaggagactcccacctccttcagggagaagaaaggaggcagcgaagcagattaaaggaacttgtggcttgtggcctttttgtgcaagggccgcctggaaggggagaaaagtgtatgaaagccaccgtggcggttaatgagctgtgagatgaggcgctgctgcggccgctgctgctgacactcgctcccaggctgcacccgccgcccttggcgcttcatgtacatgtgtctattcaggccttgcggaggcgcccagaagagcatccaacacggctgccggggaaagaccgcccaccatgcccacggagaccctacagacaggtagcatggtgaagccggtcagccccgcgggcaccttcacctctgctgtgcccctgcgcatcctgaacaaggggccagactacttccgcaggcaggccgagcccaaccccaagaggctcagcgccgtggagaggctggaggccgacaaggccaagtacgtcaagagccaggaggtgatcaacgccaagcaggagcccgtgaagcccgccgtgctggccaagcccccggtgtgcccggctgccaagcgcgcactgggcagccccacgctcaaagtgttcggcaaccacgccaagaccgagagcggcgtgcagagggagaacctgaagctggagatcctgaagaacatcatcaatagctccgagggctctagctcgggctcggggcacaagcacagctcccgcaactggccgccccaccggtcggaagccactgacctgcaccgtcactccttcgcggagtccctgaaggtctaccccacgcagggccgcaggagcccgcaggagggcggctcccacgtgggcaggagactgctggagcagtcagccgagtccttcctccacgtgtcccacagctcttcggacatccgcaaggtgaccagcg

SEQ ID NO: 68 - DNA, 2087 bp, Homo sapiens
cgaataccctcagcgttccgcacgccgggcggacgcccttccgcagcggccacgcgtgttctgggttcagcgctcctgcgcgtcccttctcgccgaacccaaggtgctttgtcccggcggccacacaaggtcttcggctttttgttctccttttttaagagactggccgcagtccccagcgattcagccggcccaaccaaaggcagtggatctactgaaatgaaaaggatcgagataatacacaggcagatgaataaggggggggatgctggaggtgcaggaagcatcctgggagcggccgccacggccaagcccgaactgcttgctgccttcgctggggaaggcgcaggaggcgagccggcgctggtggtcgctccacctggcaggaggaaatgataaccatgataaggaacgtgatgatgatgatgatgatgatgatgatgatgatgagcatgatttaaaaaaaatcagtctccctcttggtgaaccagagcatgcgtgccggagggactgcactgctggcggcggcggccgggggaaggagagcggcgcaggaggcaaaggttgaggaggcagcaccctcggatgaataataacaccgcgccgcgcaagttgcggagccgcctggaaatccagcccttcgcgtgaactcgcgtcgagcgcggattgcaggggatgaggaagaagggggaggggaggggagggtggaggagggagggggaggggtagaggggaaggcagagagggacagagagaattgggagccttttaatttttccttaccaagccctaatctcgtgcgccgtcctctttggctgaggtcgagctcctttttaaaaattattactcctttaaaggattattcaggtaaaggaagccgcatctgctgacggttccgaaaacaagaggattaggagcccgtccacagatccaggcccgcagacccccaccccctctgcaccagcacacgtcattctcactgcccccgggtacctccctctccttcgtgctcggggagagctgtgccaaggaaaatgcggatttggggaaagtgagaagtgggagagctcggaagagcgggaaagccaaggccggaggtggcgattcgcacccccacactcacacgcgcttccgtttatccgtttctatcccgggaaaagggggcgcctgcccgagcccacgcgcgccgccgcatctgcagcggtcggcgccccacgctccctgaacgccccccaggcggcaccagtgccgggcagagtcccctcggcggccgcgggcgtcaaatcgacacctgactccacagctcccttccctctcctcttcttccacctccccgggggtccaggcaccgccgtgcgtccactcccggtctccacggcttaggcagacggagtgggggactccggggacccgcgcgtcctctcctcctcggccctggcgggaggagccgggctggggtttccgcgggccgcggcgcgttttagggctgccggggactgcggcggatgcggttgacatcactgcggcggctgccgggccgctgcggcgacctgagcgccggtgcagcctcccccaccccgcctgggcgggcgtctgggcaggacagctgcggggggttgggagaggagtcggggagagggggacgatcgccccatcccccgcccccccgcagtcaaggtgaacatgaaggagagccggcgcgcgtgcccgcgcctgggaggcgcgcggccccgcccctcccggagccctccagcgcgcccgggccagatggacgcccgagattagaggcgcagaagtgcgctccacgacgggcccacccgtcccgcggacatcagacctacttcggggaggcgccttttgttgtggtggccgtggcggtgtttgtgtctttccgggagcacagggtgagcttggccttcaggaatgactcagaatgaatccgcctataagagccaaactaaagacgaccccccgccccagccccccaaccataacccaacgcctgccatgagaaaccttcggactttccaaggggtcagtgcctcagccacgaggggcagccctgacccgcgcggttcacgcacacg

SEQ ID NO: 69 - DNA, 574 bp, Homo sapiens
cgcacccgcccctcgcccctccccgctctagcttcccgactttcgtggggaaggggcaccggaacgcagggcagctgagaaggaccgggcagaggagagcgacggcccctcccggtgtcagcttcctcgccgcagtcccattaatcacagtgccgcagtccagttggggatcccgggcgcccagggccgcccgcagccccagggacaatgggattatttccggggagagaggccgcctcgggccggggctcaggcggagacgcctccttccctcaatccgtccctcggcacccaaggagcgcctgcggcggccggagacgagtggcagagcggacgcgggtggcgcgcgggtggcagggcctggccgacctaattcaattagctgggccgccgccgcctccgtgcgactctgggcccgccgggactccagctctgggcagctgctctttgtgcacgaagaccatgcatatggatgcggttcctcccccttcggcgctcaccccacccacacccacttctggcacctgcggcttgccccgccctcccactccttccccgcgagggaaggcgaccg

SEQ ID NO: 70 - DNA, 892 bp, Homo sapiens
cgcaggaaagccccgcgtctctattaacccttccatcaccgctgcaccgcgagagggaccggagaagtcttagcactgcgcagcagcggccgccgcagcccgggggtgaaggacccggctgctcaccgtgcgggggcctagccccttcacgagccacccacgcactccccatgcacccacccccacacccactagggacagcgaggtagcgggagcccgagcgagtcggtggaaggcgaacgagggttctaggaccaagggtctcaggggccatggactgaggaggggcaagtgaagcgagtacccctggcccggcaggacccgtaggctggcgtgcaaggacaacccccccctccccgcccccaagaagtcctctgcaggatcggaggggcgcgtgcacgaagcgggaaggaaggctgaagggaaaacgcccggaggctcccttacctgatttcctcttggaactgccatagtccctgaaaaagaagcaacaacagatagacatggttagggccgggcgcgagcgcggggctgcagctgcaggggcgccctcgggctggctgcggtcggtccccggtgccgctgctcatgtgatgcccacggccggctagccgcgcgctccgcagccgcacgcactgcgcgccgcgcccgcccgcccgcccgcccgcgggccccctgccagcccggcccccaggcgcgattcgcccgccccgccccgccccgccccagcccgccccgctaactggggctcaccttccctaagccttggcggggaagggcgaggcaggttctcctcacttcccaaactgggaaactgacgctccgggatggcgctcgccggaacccggcccctacccagcgctgggagcccgctctccccgccaccggagcg

SEQ ID NO: 71 - DNA, 505 bp, Homo sapiens
cggagggcgacctggagccgactgacttcacaaaggcctcctgccgcaaaccttcagcggccaccaaagccccggctgccggcggcggaccacctctgctgccgcgcgcctaccggagccgcttggccctagtgctttccagcggatttcccctcaggtgcggagccgggtgccggggtcccacagccaaccactaccggttcctctttcgtcagccaccggcgccggcaggacccgcgaatcccgatctccaggagcctgtaaggaggccgcccattggctcagccgcactgctgggcaggtacttccaaagctttgaggattggctgatgctctgggcgccggggctagttggcgggtaggatcacgtgcgaggggcaggccccgtctaggccccgcctccttgctgctgctgccgccgccaatcctggtccggttgcccgagttcccggaggtctctcgcgggacctctctcaccgccaccggtgggtccgttcggcctgcg

SEQ ID NO: 72 - DNA, 1662 bp, Homo sapiens
cgaccccgggctgcccgcgggctccctcctccctcctgccctcgcccgccagctcgcagtctcttaggtctgcagcgttggttggtttcccacgcctgtcccggtctgactcgggctgccaggcaccgggaagccccgcggcgccgaccccaacccgcagccccgggaaggtgccaaccccagcgagtggaggagacgagcatttcacgtgggcatctgccttcactgctgctcgcgcctctcctcaacggccggcgtcagggcccctctgttccaactcatcccccccccgaagttaatcagccgtacttgtactttccagggcgctttagtttggaaaatacttaaaagcctcgaaaccttccggggagacggccgcccccagatctcccagttttgcctggctagggctgtcgggcgctcacgaagctgtggcgtctgtctggaacccacatcgcagaggcgcagccccgctcctccctgacgtcgccaaagttgccagacgcccctcagcgacccggtcgcgccagatgcagttgcataaccttgccccccacctgcgaccgcaccgggtccgctcccggctgcaccggggtgtcccggccccgggagccgccgagtttccggccaagccggtttggggccgagtgtgtgcgcccgaatcggggtcggcgcccgtcccagcggtgctctgcccgcggccaagtacgacgggcgcgggggcgcgggaggagggcacgcggggctccgcttaccttccgctgcttacagataagtcgaggtcgatgcagcgcctggctcgggcatgtctcgctgcggaccctggcaagcctggggcggcacggagcactccggacagggcatccgcggccagactcgccgcgctcacgcggaggaagcctcggctccggggacttggcccgctgcgcgcctgctgcccgccccctccaccgctgccggcccggccctgctggcccggccggcccccgcgccgcagctcgcgtcgctcgcgtccctccggccccgcttagcgacgcgcgcggggaggcagcggctgggccagctccggggacgcccgcgcgctcgcttgctcgcgacgcggcggagccagccacagccacccggcttcgcgctcctccgatgtcctcatttactgcaattgtcttcggcgagatctcagagccagggccaggaggcgggggcggggcagaggagggagcaggagggaagtcctttccgctgctccaggtcccagctactccgcggagccgggcggccagaggcgagggcgccccaagccgcgcgcctgccgcctgtgcgcccggctggcaccgaggcgggcggagccccgggccccgcggggcggaggtggggcgggggcggcgcggggccgggccgggccgggccgcgtgcgtggggtgggggatagggagggagtggaggagccccctcccgccgccgccagagctggaggaggaggaggaagaggaggaggaggtggaaggagctgccgccggctgccgccggctgccgcctgcgcgcggggctccgccacagcgcgtcctccgggtttctgggctgcaggcggcgctgcatggcccgggaacttgttgagtctaccctggagccgcctccggagcagctggcgggcgggggactccg

SEQ ID NO: 73 - DNA, 1578 bp, Homo sapiens
cgaagtcggggagcgggagcgtacgtgcgcgtgttcaccctgtcctgggcccctcgctcggggaatcccgggggtgccagggcacccagggtcagggaagtaagatgccgggccgcctaccttctccaccccgtagcccgttccgaaaatcctttaaaccgagatttaatctggatgcctagccccgccctcccctcccctcccctcccctccccccggccgaggccccctccagttcagctccctcctccaccccctccccgccgtggaccctcattagcatgcccacttgggaggattcgctggggggcgggagacacccgaagtcatccaccgccagcgccttcccggcggcctcctcgggcgacagcgctccgggagccccactcgcacaagtgttgcttccaattaattgcctgggcgggggaaggaaaggggcctcgtcgccgcccccgcccggaggctggagtgctgctcgtcgggtcgtgcgttcgctcggcagcggcgtgcaccagcaccacccctgcgtgcaagtttgaaatgtgagctgcctccgattcatactcgctcgcgctccctcgcagcgaagtggctgggctgacggtctgcgcgcgcgagtgagtgcgggcggcgggctggggggcggggtgcggacggcgaggctcgcggggcggggagggcgcgcgcgagccggggctccctggagacgccaaggcaggtctgctcgcctttcatcttcctcccgcgctcctcctcctcctccctcccctccccccgccgcggctggcctggcctcccccggagcccccagccccacgcgggcacacgcagggtgggtggtcacgcccgcagggtccgcgagcgcggcgcagagcgcgggccgtgggaagtttctccggcgcgccccgtgcgcccccgccccccgcgcgcccccggcccatccccgtccctggagagctgtcgcttgcgcccgggcgcgcggctggctgcctcctcggcggcggcggcggcggccccattagcggagcctccgcctatgattggcttcgcccgggaagctggagacgggcgatgaataattgatgtgtgcggtgcggtagccggacggcggcggcggtggcgggcagcagcgagcgggagcgcgggagccggagcgccctcggagcgggcagcgcggagcgagcgggcgccggacccgcccgggcccccgcgccgccgccgccgccggggcgcggagcggggatgcaggcggcgcccgctgcctgcgcgcagcctttgttcggcgctggctgaatcctacccggagtcgctcgccgcggccgccgccggccgggccccaagcccccgagggcgccagggcgggatcgcgaccggtgcaacttctaggtaagtgcggggtcggaggggtcggcgcctgggcgcccacgggccgggccctgggctccctccgggcgcgggggtgagtgtgcgtgcctgtgtgtgacagaggagggccggtgcattgtgggtagcgccgtgtgctgcatggtgtcagccccaggcttggccgcgagagtggcaccggcgtgcaccgggccg

SEQ ID NO: 74 - DNA, 449 bp, Homo sapiens
cgcctttgttgcgcaggtgaccttcccggccgggccagtgtcccggctgctctgagggacagtcctggctccgaaagcgggcgccagcgctgagccagggcccgctcgagatcgcatcgtctctgctttcagtcgttgtccgcgcggtgctggggcctcccgggacccgggagccgcggcggattaggccgcccgccccaacctgggctttgatcttatctgagacttgtgagtccaaaagggcttagcaaccgcagccatggcagccccaacgacgtgaacatccgcacctctgagcctccccctgagaagtaccttcgaggtgaggcctgcgcagccccaggaagagggtgtgggcgcaaacctgaggtggggagcaaggcccgccggctacacggttcctgccatcctcgctgcgccctttctcccatgctcgcggcctccatccg

SEQ ID NO: 75 - DNA, 537 bp, Homo sapiens
cgggcagcggaaggaaggcgctgccggggattcaccttggctggggcgcgcgcggagcactggcgccctccccgggaacccgagcgcgggcggcgcgggcaggtagcgcggagcgccctggcgcgcacggccggctgaaattctgcttttcgttctttggggtttttcttccttttgcttttatttttttttttctttgccttctccacacggagagcgcgtttgtggcggcagcccgagcttagcgggcgtccgggtgtaaggaagaaacccaagggccgcgagggagcgaccaggacgctgcttcccgggcgctcccgggactcccagatacctgctcggtccccgcgtgggaggtccgcaggctccccctcgccactgcgccccactcgcctccgcgctgcgagcctaggcgaagccgcggccgagccgccaccgggggcaggctagtccccggcttggcttgggggcccggagcacccccggtggccacctccggcctgccccggagctttgcgcgcgccgttcgggccttcg

SEQ ID NO: 76 - DNA, 1507 bp, Homo sapiens
cggctccgccaccacctccgaggcccgccgccgccgcctctctatccgcatcccggataggggcgccaccaccccgcgcgtgaccccgctttcccgcgggtacagataaaaatcccctcacccttccgtagatgccatggtctggggaacgccccctcctcttcagcactatccggcagctgtgggcgagggagaaagcggacgccggggtgagcgcgaagtgcggccgggagcggtatttaagaggagcctctgcccgcccttccacccgcctccggggtaagccctttagcgcctcaacgggcgcaggaggctcctgcgggcgctaatccgcgtcccggctctccacggcgcgcgacccgcgctcccctccgcccgcggggatggcgcttggcggggagctccgggggctccgagggtggcttccccgagagagcgatgcgcccagggctgcaggagggcgcacgccggcgatgcgcccctgcagccgctgccacctgtgctttgctgcggggctcgcgggcgcggcgagtggcgggagcagagagtggtggcagaggacggtgagcgtgcgtgcgcgtgtgcgggtgtgtgcgcgcctggggaggcggtggaggccgctgcgctctggctcggcgccggccagggagggattgcaaggtttagccccgccggagctggggatttgcaggcgatccctctctattttcgtcgagagctgacatcacccgcgccgccgcctcgggcaactcctttaaccgccgccccccgcccccatctccagtcgcggtccctcctctgccatccctccccccagcctctcgccataaattagccaaataagaaaagaggccccgccgctcgtccgggaaggcttgggcagccggtggcaggagcccaggatctaaggagtagctattgtttcccctgaagcccgcgtggccccaggtcaccccgtgcggggtgaagcgcaggcgaccgcgcggagacccgggatgcccctccgtctccgcctccctggagcagcccctggcgctccctcctcactccccggagcacccgcttcagctcacactcacccccaaactcacaggcaggcacacccaccccatcctcccacgtctgggggtcccaggtccagcggagtcgagcggctgaaacacgcgggtggtgcagcctaaacgcggcccagctgcgctccctcccccgccgaagctcgaataaatctctggccttcaattattcagcgagattaatattaatgcagctccctgggggcggcgggcggggtgcaggggaggccgggattttttttttttttttttaggccgagagaggtcggcctcgtgggtgccggctgagaaggcgcggagagttggccgaggggtgagtggcgggggacggttctccacccactcggcgctgctcgccgcctgcttccgtgtcccgggggcggcgcacagagacagatgggcttgtgcaagggagagttcgagccttccggacg

SEQ ID NO: 77 - DNA, 1309 bp, Homo sapiens
cgggcagagcagacccgcgctgagcctggtccaggccatgctgttctctgcgtcctgctcgctgccccccgccccaaaacagagttagtcctggagctggggggtcgcgcgttgctgctggggcaccacgccaggaccccccaccccacccggcctcacctgcgcttccccaccaggccctgtagcagccgctggagccgcgcgccctcccgcaggcggctgagctcgctggtgaacgtcccgtctgagtgtcgccgggccctgcgggcggtcgggggtcggggtcagggcgcactgggggcgggtggggcccggggggcgggcgggcctggcgggcggctcacctggggggcgcggggcgcgcggcggagcccccgaggagcagcagcagcagcaggaggggccggggggccatggcggggtcggggcgcaggagctgagcgctgcggccacggccccgggcccccgcaaggcccctttatggcggcgcccggaaaggggacggcccggccgcgcccagcgatcccgggaaggtcagggccgcccccccagctgtcgccccggcccggcctcccgccccctccgccgcgctggcgcccctcggccggctgggggagggggacgtcccggcctggggggcgaacggagcgcgggcctgggacccgcagagggaaccgcaccgcgaagcctcggagcctgcgggggcggaggggccggagggcttgggggagggtttggggggggactggggggccccggggaaggggctgcggccggagttccggaggcccctggagcaccttgctggggcgagttcgtctccagcttcagcgtggcgtgactgttggggggccggtgttgccattttatctgctttgaagtgcgcgattcaggatcattcggtgcatccatccccaccatccgttctcagactttcccgtcctcccaaaatccgttcacagacgctcccgtcctcccaaaatccgttcacagacgctcccgtcctcccaaaatccgttcacagacgctcccgtcctcccaaaatcgttcacagacgctcccgtcctcccaaaatcgttcacagacgctcccgtcctcccaaaatcgttcacagacgctcccgtcctcccaaaatccgttctcagactcttccgtcctccaaaaatccattctcagactcttccgtcctcccaaaatccgttctcagactctcccgtcctctcaaaatctgttcacagacgctcccgtcctcccaaaatccgttcacagacgctcccgtcctcccaaaatccgttcacagacgctcccg

SEQ ID NO: 78 - DNA, 2608 bp, Homo sapiens
cgggggcggggcgcggagggaaagaaggggtggccaggcccaggccaggctgcaggctttggcgacctctcagagctgggagcggggtcccgaaggcgggtctgagggcagagcagagtcagaggtgtctatgggcgtccccgacctcccaggctgccccgtggtttcccgccctgcttgggggtgagcgggggttagtgggcgggttagtctagggccggggccgccgcacggggcgaggccctgcttaccagtcccggtcagggtgggctgcgggagcctcaggctggaaggcagaggacagtgagagcccgcccccagggtggaccggggcaggggcgggccgtggggcggcgcggggcacgtacccacggggaagtagtgggggagccggctccggtagatgtcctcgtccttctcgaggaacacgcagatgatcagccggtccacctgcgccaggggctgctcagcctgggccgagctgccacgcctggccccaggatcctgtgactgcctgccctggtgcaggctttcacctgcctggccctgcgcccttcacggaggctcacggctgtgtgccatcacccccatctcgccaccgccaggcgcccttgcgcaggagaaacagggaaggaaggggcgggcctggccctgcgctctgcaccctgactccctgggcgatgatgggggctggtcactctggccgggatgcggaccccaccctcgcccgggcccaggactcaccttgtccttgtgctgctccagccactctcgcagcgtggccagcacgatctcggcggccgcctcacaggggtagcctgaggcgggtggggcgggagtgagtcctaggctctggccccgcccactcccctgtccgccccggcccctcccggcgtctgggtccaccgcccactcctggtccttgcctttcttccagactcaccaaacacgccggtggagatgcaggggaacgcctgggcggggaggggtgagagggggttggaacattgtcactgcgccccgcctcccgccctcccccgcgggccgtccctcaccaccgagcggagccggtgctccagcagcaggtccagactgctcaggtagcagctgcggagctcggcagcctgactggcgctgggctccccgtaggcgatgggccccactgtgtggatgacgtctacggggggcgacggggtcagaccggcgggggtctacgcggtcctcagctccctcgcttccccctgccggcctgggcagaagggggcaggaaacacctttccccccaagcgctgagcctcctccgcgaaggcccccacccctgtgggccccctcgagcccgaatccgcacgcgcacagagccccgcgccccctccccacatctgtatgggcccggctgagacccgagcgcgagtgtgggcgcgaaggcgttgccccccagctccatcctcagcctccacgcacggtctcccccatgtgcttcctggggtgtgcggggtgggggccgggtcttggcccttaaggttaagaagggccccctgaggagttccccagcactcctgtgggggccgcacccctgatgtcccagtgacacacgggcgccctgtctgcgccccacagctgggggcgcgtcggggactcacacttggccgggagccgatagccgccggtgatcttggccttgccagtcttacagctctgcagggtccggcactcgtcggtaagcagggggccggcggcccgatgaatgcagccgtccactgcgggaagggcgggcgcgactgagctggcctggcaaggctgcggccccggggtgagcaccctcagtcccgagaagcccctcggcgatgcgaggacccatgcggagccgcgccccaaccccgtggcctccggccgcggccgctgcccccacccccgcccggaatgtttcacatctggggaaaccgaggcgcagagcgggacggccctgggcctggcgggaagtcgcgcaaggaggccggcagggggcgcacccggcccaccctgtcccttccaggccgcgccgccagggcccaggcagggcggctccgggaggcgggggccgccgggacaggaaccgccgcccgagccggggtgacggggaagcttcctccccgacgcccctccctagccctgtcccgggcgcgctgcagggcccgcgaccagtcgctgcctccgcccgggcctcagcgtcggtgcgatcccaaggcccacctccccaggcggcgccccatcacggggcggcggcaaaccctaggacccattttgcggatgaggaaaccgaggccaccttgttacagggtgatcagatctgtagctggagtctgggtcttccagccgcgcggggaggtgagcccgacgcccgggcccgagtctcctggcgcgcaaaccccagctggttaatgaaccgcctcgttagcgcatccagcttaattacggccccgcatgggcttgtttatccgagggcggctgcagggcctcgcgagcgaccctggggacccgggcgcagataaacaaccggggcgcagcgggaggcatggaggaaacgctccagcagcccaacctcgccagggcaccgcgcaggagaggcgcg

SEQ ID NO: 79 - DNA, 289 bp, Homo sapiens
cgcacgagccggcagcaggcaccagagacgcagggacctgcgcgtcgcatccagcgccccctccctctcccgtcccggcgccggcggtgtgcggcaggcaggagcccccacaatcatctgacgagcccacagtgcgcccggcggctgcctgggcgtcatcccgcgcgagcatccgcaggctgggctgggagagggcggggagggggagcggctggggctctctctgcatccccccctccagcgtccccccggtcccttgcagccttggaaggtggccggtggccggtcg

SEQ ID NO: 80 - DNA, 1959 bp, Homo sapiens
cgcggaggcggcggggacggagggactgcggctcccgcggcctcccccgcgcgccgtcagtgtccggagccgtacattttgtcccactccacgaacgagtccagttccttggcagaggccctagggcccaccttggccagcgccgcctccaggtccttgtaggagagggggcgctgcagccccgggaggcccgccccggccgccgcctgctggcacagctgccccagctcgcccccagagaagccctgcgtgccctgcaccagcgccgccagttcccgctcactgagcgcgcagccctgctgggccagcgcccgctgcaggatctgcccgcgggccgggctgtcgggcagcgccacgtagaagcggagagagaagcgccggcgggtcgcctcgtccagagccgcgggccgcgaggtggtgcccacaaccagcacgccgtcagcccccgcgccgcagcccccgtccaggcaggccaggagcggcacctgcagcgcgccccctgccgccgcgccgtcgtcccgggcggggagcagcgcctctagctcgctgatgaggagtacggagggtgggcggcagcgcgcggccgcgaaggcggcctggaggaggcgcgcgccctcggcggcgccgggcgcagccagggtcgcgccgcgcaggcgcaacagcgtggcgcccagctgcgtggcgaggcagcggcccagcagcgctttgcccgcgccccgcggcccaaagagcaggacggtccgcggcgggcgcaggctgcccgggtaggcgggcggcctgagcaggggccacaccagctcctcctccagcgccgccttgagcgcgccctggcccgccacatccgcccactgcaccgggggcccgcagtccaccatcttgctcgtcaccagctccagggccccagggtccacgcctttgggagtctcccccgacggcacggcgaaccccccacgaggagccggggcccgctccgggaacttttcaaagggctccagttgcgggccgtagacgggggagcccaggaccttgagggggacgccgccaccgtacttgcccgacgcctcctccgcggctcctggcggcttggcccggaacccgttgccccgacattcgccgttgtccgcggcggggtaggaggctccgtcagccacgggggccttggcgggctcgtacgcgtacttgcggtagcggccctcgggcccctcgtcggcggccttgcgcttcagcgacagcccggattcggcacccggcgcggccgtggggaagccataggcggtgggcggtgccggcgcgggcaggggcgtgggcgcgggcaggcccggggtcaggtagggggccgggggtgggcctgggggcggcgggagcgcgccatagccgggctgcgctgcgtagccccctgcgggatagttgtacagcggcgctgagggcccgtaccccggaggcggtgggggctgcaggagcgcggccgggggcggcgggggcagcgcggcgcccgtctgcgcgcagtaacccggcgccaggtaccccccgccgtagccggccgcgtactcgggcgccgccgatgggcccccgcacgcattgccggcgtagaggggttcagggaggttcccggctaaaactggggagccccccagggccccggaaccgccgccaccgcccgatttggttcctgggaggccttcgtggagtgaggccaaggggtagggtggctccggccctggccagggctcgggatcccctttggcgccgttgaggaaggaggcgtcgctgtacccgcccagggccggacgctcgtagggagaatccaagaccccagagtacttctctgcatagcgctttaggaggttggaggcagtgagggctgagatgtcgtcgtgtgcccaagcgtagtggcagcgttggcgacccccagggggcaactccaacttgtgggccggcgacggggtggtggaggagacg

SEQ ID NO: 81 - DNA, 329 bp, Homo sapiens
cgcccagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgagtatgtggggcgggacg

SEQ ID NO: 82 - DNA, 454 bp, Homo sapiens
cggaggccggtaaatatgtaaacgtgggtttttcggtttccgtttcgcatgaatacaaaataaactgcggccctgcccacgctggaaagttcttccttaaagttaataaaatctgtcaacttccataaaggccgagagaaagggggtgcacaggtgcagacgttcgggctgaggctcgagaagtcgaggcagctaataaaaagccctgggcaccgcgcgcctccttccccatcaattcccatcaagcgttattagagtcatctgccgtctccgaggtgcgtcccccgcgctcgccccgcagaagcgacaatgacatttggctgcgctgcaggtgcggctgtgtgtggccgcgcggccggagtggggccgcctttgtggcgctcgggtcctttgatctgggccacaaagggccgagaagctgcagcccccggctttctgcggacgactccgcccg

SEQ ID NO: 83 - DNA, 227 bp, Homo sapiens
cgttccgctaactccggctcttcttcgggctcctcggcagcgctccgggccagccggcccgtgccccaggcttgcagcgcccggcagcctcgtccttttgtggtctctgcacgggatccaaggtgccgcgcggaggaggcgggctgctcgcagtgccggggtcagaggcgccgccaccggcggcctctgcgcgcgcggggaggaaagggttaagctgcccgagcccg

SEQ ID NO: 84 - DNA, 2895 bp, Homo sapiens
cgtgggcggtgagcggccgggagaggggcgggccagctgagcgctcaatcacagccccggggctcggcgcccgccagctgcactcagaacccccctcaccgtggccctgccctgtggcgcctaattacgtgcccccagggctctggagcggctgctgggactgcagcgaagaggggcccgcgccacctccgccaggctccacgcgccggccttgagtgcgctcccctcggccgccgggaaacctagaggcgcggtttggccccaaggtccccggtcccgctcttctttgtctcggcctagttctcggctgtctccgggccgcgacccacctctcctggcggtccttgccgcatgcttctgtgctgcgccagaatcatcgagtctcagttctcgctgccttcccggccagtaggcgtctcccccagctggggagggtcccccaaaccctgtgcttctaggaccgcggggccggcaagcggcaggatctccaagctgcgtctctgatgcggctaggggaggcgcgcgatgaaggcccccctcttctccgccttctctctacgccccaccgctactacacgtcgcgctgcgccgcgaccccggcgctgcctgttggcgccttaagcggctcttctgccgcggtgcgatccccgacgccaggaggactaacccccacccgccaggcctcagaccccacgcagatccagccacacacgcaaccgcctgcagatacccaccacagccctttaagttaacccaaagccccgtacaggcgcactgcccctacatgcgcagccccacggccccacacacccaccagcccactcgcaaacaggcccacatggccacacaactgccctctggacactcagccccacgcaggcacaatcccatatacacacaaccgcacacaggcgcaatcccacccacgcagtacctcacggagtcacacaacccccctataaacaatcgcacagacacagccccacgcagcgccacatcattcagtcaaactgagacccagaaacacattcactagcttgtatccagacccccccccccaagccccgtgacattcactcgtaggacagaccagaaatatacagccacattgatacacacacggacctaaacgcacagccaagacctcacccgcaggctcgtaagtcactggaaaaatattaaagcgttccctccctcccctcctctatctttgacatacacacgcctgcgcgcgcgcgcactcttacactcacaccccccgtccctaagcgcgtcggagcctcgcagcgggtgaccccgaggccccgcggtgagagccaagggagcgtctccccggcgccccctcctcccgctcccttcccgagctcatctctcgcccccccgcccgcctcctccttccctgccccctcctcctcctcccttcccccgagtctggggcgcgggctgcgcgcagccaagccccctcatcgcgcgcccctccccgcgcgcgccccgcgccagcatcgcgcttaccgagtggatgcggccacggggccgtctctaattacctcctgacccccggcggcctgtgaataatggctactgagggcgctcgcaggggctgctaagaaggaaacgcggggcgcgagaggcgcggcgcgcgcgggagcgcgagggaaaggagcgagcctggggccgttgcaagcggctggcgagagaaaggattcgtgcgagcgctcacatttgtttctcccaaggatctcatctggccaccgcgttctggaagaggcgagagggagagcgtgcgcgagaggagagagacggcgggggaaagggagacggcgagacgcgcagcgccggcgcccgggagacccaggaggagcccgcaggtattgttggcggcggagctttgtgttttcggcgggctgctgtgggcgccagggctccccgagcccccggcccgccgccgcgcgcagacctgcccggagtccggggctgcaggctggggtcgccgcccgccgcggacacccctcccgcggcgccaggatcttggctggagttgcggatttgcatgtttgggggttttccacgctggttgagggaatcggtctcagaagaaggaatttctttctttcctttttttttttttttttttggccgcttttggccacagtttggttgtgctggggattcccccctcccctttcctccacaaagcattggtgagcggtcttgacgaatgtggtattggcggaaggcatggggctgagatggcggggctggtggccacggatggggaggcgagagatgccagagaaggaaggagttctgttcaagttcgcaatctgcgccccgaagctggtcggaccggggagggcgatccgcgcgtccgatcctgctccgggcaaccgatgggaaacttcccgagagcgcggcctccctggggactcggcgcccctgacgcctccaggcgcttcctcgggccgctgcggcccctcctcgccgcccgccagcgcttaaggtcgcgctctccccacccccgccttcctcccttcctcggcggcttccggatctggacagagctgcaggagtggcttcaaacacttcccctggagacccctccccactccccgcacgagagcctgcccagtggcccccctgccctgcgccccagtctcacgcgcggtcgagtggccaggctgacaatgaaatctcgcacctctgccggggggagtggggtggggaggaggtccctcctggggctgctgaccctgggcctcgggcagggggagggggctcctgggggaggggcttgttttggttagagacgtcgacctgggatggcgccggggcagggagggggctggggggtgggcgcggcgaaggcggcaggcttgggttcgcggccgggggtcg

SEQ ID NO: 85 - DNA, 1202 bp, Homo sapiens
cgcccaaccctcgagtctgcacggtgcagtaccgtctattttgggtccagggtaacctgggtgcaaacgctcctgcgggcggaccgcggggtgggccccgggcacacaggaggatcagcagcagccggtcggtaggcggagcgctgccagaggcttgggcctcacaacaaccctcacaggaccaaaacaaacggcgagacgggctgcgcgcacaatagcatacccgggcggctagccgagtcgggggtctcggctcgcggctgcgccaagtttcagaaccacctagccccgtgcctgccgcccggggcccggcctcgccccggccggcgctcacctggcggtgccgcccccggaagaccacggcgaaggccccgtgtcccacgagatccctcttgctgtactcgaagtcacccaccacctccatggccgcgcccccggggcacacagcggacgggcgggcggcgcagtgcggcgcaggtatcagcaccgcggctccgcgggcccggagcgcgccagcgtgcggcgggtctggggcagccgcagccccgggcccgggcggactctcatgccgagagaccggagcggaaactggggaagctgccgcgcggagccagggtcagcgaggcccggcccggcccctgccgctcatggcccggcctgccgccggccgctggctgtctggcgaagcggcctcggcgctgccgggccaggggtgccgtcaccgtcactgtgcgcgcccagagccgcacacgcgctctgaggcagtgaggggcccggtactctccatccccagctcgtcgggcctcctcggccctccccgctcggggcgctgctgcgacggcgacggccgccgccctagaacccgcaccgccgcggccccgcgcttccccgcctcgcggcggcgcccaacgccctcgcgcgcgctacccgctcccgcgcgactggctgccctccgcgacgtccgcgcgccttgaaggccgcagcgccagccggctccctccgccggggaaaggagtgcggccagggcttggcgcccgcagctgggcagatcctcggccgctcctgaggcggggacgaagggcgcgacccgctgcggccacagcgggttacgggctgcgggctccgttctgggcctggggagcagattcgagccgaccacgccccgggctccaggccgctgggaggcggacttcagagaagcg

SEQ ID NO: 86 - DNA, 1198 bp, Homo sapiens
cggtgttgctcccacgtaccgatgtcacgtggggcggggaggcggggcggagaacggagagcgtcctctcattctccacccccttcctccaagtccagcgcagggggaggtggctgcgactgactgaggggctagggaaggctggccaggggcgtgggcgtggctggggacggcttgggggtggggctgcgcaggaggctggaggagccccgcgggacccagaggcggggcgtcggcccggggaccactgctcctccggggcgtggctgcaggaggctggaggagccccgcgggacccagaggcggggcgtcggccggggaccgctgctcctccggggcgtggctgctgccgagcatctcccagctcagccgagcccgtgcccaggccacgctttgttccagccgccgcctcctctaccctacggcgtccggagccatccctcgcctgctcgctctctcctttcgcccactccctgcatctgggcctgcatcacctttgccaaccgctcccccgatcctgccgacactcctcccccaaacttctgaccggcacccttgcctggtacccttctctccattcctccccctccatcttctttccccgacccctctcgggtccctcttttcccaaaacccgggtctctccgcgtggccccgcctccaggccggggatgtcccccgcggccccgcgcccatggtcctgacgctgcttctctccgcctacaagctgtgtcgcttcttcgccatgtcgggcccacggcggggcgccgagcggctggcggtgcctgggccagatgggggcggtggcacgggcccatggtgggctgcgggtggcccgggcccgcgaaagtgtcgccgggggcaggcaccgaggtgcaggacgccctggagcgcgcgctgccggagctgcagcaggccttgtccgcgctgaagcaggcgggcggcgcgcgggccgtgggcgccggcctggccgaggtcttccaactggtggaggaggcctggctgctgcggccgtgggccgcgaggtagcccagggtctgtgcgacgccatccgcctcgatggcggcctcgacctgctgttgcggctgctgcaggcgccggagttggagacgcgtgtgcaggccgcgcgcctgctggagcagatcctggtggctgagaaccggtgagcgcgccaggccggggttgggagagcgccgcg

SEQ ID NO: 87 - DNA, 1837 bp, Homo sapiens
cgattgtgagcgaggccccgcgccgatggtagggaccaggccacagccctttccccaggagccggcgcgaccttccgaccttccgaccttccgcccttcctttccgaaggagatgaggctcgcgcgcggctggcaggggcgggagggaccacgcgacgagtgcgcagcggccaggcccgggtgggcgcggggagggcggagtggggggcgcccggcccgccaggaggaagcggggtcgccgtgaagttggttattgaacccatgtggagtcaaactcccagggccgggagagccagtgttcgcggggagccgaccccagcccactctgcatcccagctcggcgatccttgcgccgactctgcggggaggcgggggcagaattccgcctcgttcctcccatccgcaagctcccacccggggacccgggcccgggagctgcgagccggggcgggcgccgggctcaggtgccccctgtaagcgctgggcggccgggaggggacggggaccgcgcgcgggcagtttcgtttggacggaggaggacgcgcgtttcctcttggtgcctgttttctctccctctgttttaaaagcagtgacagcatcggggaagtatccggggccgggccggggagggcggcagccgcctctgcctggggagggagctcggggccgccggcgggagtgcgggcggggaccagaaggagcgggcgaggggaggcagggccggggccggggagggcgcctgggagccgggcaggggggtcccgggagcgctgtccgcctcctcctgcgcttcgcccgctccctgtgccccgcgtcctccaccatccgggcggccgaggccggggcgcgggaggcggggccggggcgggtcccggcgcgggcggcgcgtctgagcgggagccgggagccgccggcccgggccgcgccccgagcgagcctcggaaatgcccgcgccgggggagcggacggcggctgccccgcgggccccgaggagacgctgaaggtcgccgggagggaggggagagcgcgcggtcacctgacccccggggcgcacctaggcggggcggggcccatgcgaggcggaggccgggggccggggcgcgggctcaggggccgggtgcggagcgcgcggagccggggctgcggggacgcgggcgggcgaggtcgggcaccgcgggcggcctcgccatgtcccagcccgcggggaggaggcattgccgaaaggcggggatccgcgccgccgtggtgctcatcggactcctgcacaaatcccggaggcagaataaagagaaaaggtaaacccctgcagcgccggccgcttccctccgctctcctgctgcatgccagttttctttggggtggacttttccgatgggtgtttggttccgtgttggttagaaaaaagcctcctctccaggtaccgtcgtgcgaaccgagttcggagcttcgccccggaccctggctggtgcgaggagcctgggcaggaccttggcggaatcggagggccgctgggagggcgcatcggggtggatccctgacggagggaccccaagtccgtcgcccccagaccctttgcagaccgcgaccccgttccccaagcctgcagccgcggatgccattgatccagcccaggcggctccatgcgccggtgttgccatttgcttcctgcaacttggctgagccctcgaggtgcctgcctggccgagatccgcttgctctgcgctcggtgccggcaggcgaggagcaagtttggaggttgttcgctccccacccaccctctcgccccaccgccatagggtgactttgcctcttcgcctttctggcgtttgttctttcg

SEQ ID NO: 88 - DNA, 222 bp, Homo sapiens
cggctcacccacgcgtcaccctcaggttcaccatccaggacctcctggcccacgccttcttccgcgaggagcgcggtgtgcacgtggaactagcggaggaggacgacggcgagaagccgggcctcaagctctggctgcgcatggaggacgcgcggcgcggggggcgcccacgggacaaccaggccatcgagttcctgttccagctgggccgggacgcggccg

SEQ ID NO: 89 - DNA, 935 bp, Homo sapiens
cgggagagagcggcggcggcggcgccgttcaactgcggcagcagaataagcgcgcgtcgttcagcgcagtgtcatcgccgaggcctctaggtccctggatcttggtctgcaggccgcacgcgcccttggggcaatggtcactccagtctccaaagtctccccagctcagcccaggcccctgcagttcctcgccgtctgaacagcggaagcgcacgttgttcgctgctgtgttgtcaccgagggtcgtgggtgcctccacgcgaagcgagaaagccactaggtaggcgccgccgcgacaccacagcggctcactccattcgccccagctgcaaggcaaagggagatgcgttgcctctgcgggccccccattcaccaagcccttgagactttccaagccaagtgctgtgtatatgacctggtgggctgggtgctccccgacctccgccgaggagcggaggaggggtcttccatccacccttcgcctccccggtctatactgccccgtagctctggcggagccagcctgcctgttgcgacggagtatgtaatagagggtaagggacataccatcaccccaagggatcctcgacccctgcgccccaccttccagactgggactctaccacgtgcgtattgcctaggacgttcccgcgcgcgcagtgcagcctgatcccattcagtgcagtgtcgtcgccaggaatgccttggggaggctccacctgggtatcgaggagaggggcaagggcgagtcacggaattatcatccttggctctatggtcccacgcatctccgccctctccaggacctacgcctgagccccaaccttgagcgagaacccgctggcgaagaatccatcgggacacatctcaggccaggcccagtcgccccagggacccccgctggtcacttcgatgaccgccgtgtagccgttccg

SEQ ID NO: 90 - DNA, 1079 bp, Homo sapiens
cgcaggtgggcggcctcctcaggccggggccttctgggacccccaagtgccgtgcaggacgccacgaagttgcgcccgagcccgcggggtccggggagccgctgcccgtgacagccgcgctggcttgggaggctcttcgggcggccgacgcctcggtggggccgcgactcccgtcagggaagctctcccggcccggtgccgcctcctacgcccggcccgagcctcccacggctgggctcacggggcatctggccgccgccgtcgcgctcccagctcggactccagccccggcgccgcgacgctgccccggccccgacccggccgtcgcgagctgtcctccaccccacgctccccggtcactcaccccgaggccgggcagcagcagcgtcagcagcagcagcagccgccgggccggcatggcgaggactcggccctggctccgggggcggtacggacagaggacggggcgggggcgccgcgacgacagcgaggcggtgaccgagcctcagcggacgcagaactgactaggcagcggcggcacccccattcccggccctataagcgctgggtgccgcggcttcttccggtcggggcggagttgcgggcggggacggggcggggccaatctaggggcgtgttcggggcggagatgggggcgggccgagggcgtgccccgggcggggatgtgggaggggctggctcggggcggggccgaaggcggtgctggcatctggctggcttcgctcatgaggcgtcggcggctgtaaaggaccaggcctcggcatccagcgtctgggggcgctgggggatcgaggcgcgaagcttgtctgtggctgggaaagaccggcaatgccccaccaggccgtcccattaaccctgccgagggacctgccgcgtcagacttcatcggggaaaagggcgggacacgggcggtcccgggtgcctagaagcctccttttcgtctttccgccgtggtggaatcatccgcaaggtttaccgaggcgacctccggagcgaaggtttcagcagctaccggaggcttccagcaaacacggcgcagctgccccgcgcggcagtcg

SEQ ID NO: 91 - DNA, 885 bp, Homo sapiens
cgacgccggcgcagaggggacggaagggcccgcccccagcccagcgtgcacagaggccatagccaaggccttaaggctcatccaaccggggactcatatcccccccaccggcagcccggcgccccagcctctacccgtgcccgccgagatgctgctgccgcggtcggtgtcatcggagcgggcccctggggtgccggagccggaggagctgtgggaggcagagatggagcggctgcgcggctctgggacgcccgtgcgcgggctgccctatgccatgatggacaagcgcctcatctggtgggtgccacgcgggcgccagacggtgcgtggggggggtgctgcgaggctgccccgtcggatggtgtgggagcaccccgtctgcttggcccgggtgggcagggcgggtgactcaggggcgcccctgtcaccacccccgtccaggcagctgcgggagcccgcgggggtgcagaccttgcgctggcagcggtggcagcgccggcggcagacggtggaaaggcgcctgcgggaggcagcgcagcggctggcccggggccttgggctctgggagggggcgctctacgagatcgggggtaggacccgcgcgacccgcacctccccttgccctctctggagccccactgcccagatcggaaaaaggagagtggcatcatcccacctgccttcacccgggtcccccgaccaatcggcccctcccccctcccaccccttccgcccgcaggcaccgcaaacctcgggcccacgcagcacccccaggtgactgtcagcggtacctccggactcgggcgggtgggagcctggcgggcaccccacgccgtccggtgggcccgcccttggactctcagcgcggccccagccccggccccg

SEQ ID NO: 92 - DNA, 1485 bp, Homo sapiens
cggcgggaactcgcgcggatccaccggctcggcacatcctgcctacaagtagggcggcgtgtgctaggagccgggccccgggccgagtcgtccgtgccgcagcggagagctccgaccggcgcggccccaccatccgctgaccttcgcccggtccggcctggagcccggccgcggcctccctaggcctctctaggccgggcgccggttcagcgaggaccggaagcgcccgccgtccgcccttccgcctccaggccgcgggcgccaggccgggggcgggctggggacgccctgggagtgccccgggggccgccttcttccccgcaagcccggagcgggcgtcgctcccgcccccgcctcgtcttcagttcccccgtcgcttccgccgccgtgggtagaacagacccgagctgggagccaggcctgcggcccctcgaacactagccacatctggcccgccgggcctagcacggagagaggccctggggcttggcggccggggccgggagaggtcgcgcggccaaggacacccgagggcctgggctgcctccccgcccttccccgcccctcccctcggccctggaatcaatagtggccgctgacttttcacgacaattgcagctgtctaattcatcagcgaataattaaatcagaggccgccgggcctgcctggagtcggagacgaggggtcgctcctctcaaatcctgtcaaattggggagccagtgggcagcgcggagagcggacaggcctcgccctgcgccgaggctgcgagagccgcctggacccggcgccattccgagtgtagggggaagcaagcccccaggtgtggggagcaaaaggaaggaggagggcacaggcgggggccccgccgaggctggactccctcgtcagcagggccctcctggccggtgcgtggtccgggtgcccggcgtggggcagcggccgtggggcccacagacaccatcctcacgcagacttcgcgaatgtcgtggaactccatcctcgggaggcgctttgcctttgaggaagatggagaggagtcgggagaagcgcctagaaaccgcattgatttagacatcaatcctggccggctccctccgcctgccgagctgcggggccgcgccgccccgtccccgagaagcggacctagggtacgccgggctccgccgagcgggcaaagggagcggcctgcgccgccaccccgcgcccgcggcctcgacgccgcagccgccacgccaacaccgattcctcgtggccaacaggtttctcgcagccctccgagtcggcagcgccgcccgcgtccctcttccagccgggccggcgggacgccctcagagcttgtgctctctcgaggccgccgagcccactgagcccggccggggcgctggttcgcgtgggccgcggggaacaggtcccgcttctccaacgaccacggccccggcggctgcgaaacggccacctccgacccttgagtccgccagagtcg

SEQ ID NO: 93 - DNA, 3382 bp, Homo sapiens
cgacatcccgccttggcctgccgggcgggcggccggccctgccccatggcccgctcaggggccagatccgggctcagctgggtccggacggcggccctcgcgcacgtggttactcccaaggcgtgcgcaagcactcgcacaccccctggccctgcggccccgaaggggacctaggcgcgctcatgcacgtccctgtctgacaccccagcccggcaactcggacgtgcgcacgcacataagcacgctctcgtatagttgctcccacctgggtccgcagcatgcccttgcccggcgcccccagtcgggatctcaggcaccgaaagcttatatgcgcacctaggcctgacatcccaacgacactggctggcgcacttacacctataacatgcccctatcgggcaccacatgggggtccctaacacaagcattcactcccctgctttccctccgatggagctttcgagcacaggtactcgagccctgcgccccaacggggagctccaggtgggtccgtgagcccgctctggtgcccacacacatggctccaggcccacgcgggtacacacctccccagcctgcacacgcgtgtttcacatgagtcacacacctgccaggccgctcagggcatctggcttcaaagcggcattcaggttgttgcgggagccggcagggaagggaggcctaattccacctcccccatccctctcggaacacccgcctccccatatgtccctggggcacaggtgggacatcccagaactgccgctgcccagtggcgcccctgcatgtgctatatttatatttgaaatttgtacctttatatatactttttatatgtaagaaccgctcccccctcttcccgctccacgtttgtacttggattttaccacccccactaggcttcgacccaaacctgcaaaaaccatttcgccaggcagggatgtagggggaggcgggagcagccctccccctctccactgcggacggacatcccacgttcttaaagaccggggaccccccctcccccaagggccggactgcagctccgaatcccgcagccccagaaacacaatcgcgggggtgggtgctggcggcgagacgttgctttttggtgtgtgtttgctgttaccgcttaaattaaccctgagtgacggcttgcaattcgccacaaagaggccatttaatgccccccaccaccccacctcgggagaaatttccacttgcccccgagtccagctcgggccgcacgctacccgccctccaggccttcgctccaggtgcgcccgggcggtggaggcgagttccctgcgcgcatcatggacttcaggagtgagggtgggtgggttttggaggggtgggagcagaggcaggggggtgtgccgggactccccagacgctccactgcccaatcatagctctttgatatcccagcccggctggagaaagttagatggtactccagcgtctcctccgggtccccggaagagagaagctgggctgcaccccatgttggcacgaacactccctccccagtgcctctgtgcattaggtcgcgtgcctgtacgtaccctacctgagaccgcccctccgccaagtgcgcccctcgacaggtgcccgtcgcccacaggctcctttcattgtagcctccctggaggttctcaaggcgcctggcatttcctggacccgctgcaactgactcaagactggggggagggggctcatggaggtagaggtggtggggaggtctccccagggcccaggcaggaggagggaaggtcgtgtgctttccatgtcgcgggcaaaatgggtgctgacggtcactgtcacttgcatcgcacactgaggggtgggagaggacagcacatgcctggaccgtgcaggaggcccagaaccggggcgcgaaggggccgcgggacacgagcgggccgtgcgcaaagcctggcgcgcgcgtgcgcagcgacgaaaaggcgggaagcggcgcttcgaggccccggatgtcagacgcgacctgtgtggcatgtaccaggctggctccgacgacggaaccgccatgggtgctgagtggagagtggggaccgcggaccccgggggcaggtgaggggaaactgggagcataggtctggctttcgaccgtttgtgccgccagagtcttctcaatgatccctcccaggcaccgtaggccctctagtgggtctgggtggcgtcctcccggcccctcgctgctcaagcgcggctccctcttgccggggcgcggcctgcccctcacagtgacccccccgacccaccccccaaaaagtgaccccgcgctggtgggcatggaggactgtcccagttcaccatttgctgcgtcaaaggccgccgccaaaaggggctccgatgtcggcatttttgccttaaggctagtttgtcccccaggcgcgcgggggttgggggttcagggccgcctgtgcctcagtttctctcctcctgcgcccatcctgacatccgacggaggataatgcgcgttggagggctttggccgtgaatgcgatgttgatgattgctgtgattctttgggtgcttgggagagtttggggtttaactgcccccaaatccgaggacagcttgggggcgtcccttcggagaggatctctccgcgtcgcatagtgagcgtcgtcatctctagactaccccggtttccgaggagcctcaccttcaatccccgagccttctcttttgggggagtaaaggcggggaaatttaccaggggagatttttgtttccagggttggctgggtttgggggattttgtcctccagcgagcgcggggaagaaatgtgactaagtggcagaaaatgcgctttccctttctctctcttctgccttcgggccgcggggagttgcgcagactctaaaaaaaaaacctttaaaaacccaaaacctcccctccaagtccctttcgatctacgccaccttccctctcagggctgatttcttgaatttggggcgtcgaggcctccccacccgttgctaccagcccttttgcagcacttcctgggcaggccaggggagaggacggaaaagtgaaaaaagaacgtgtgcgcccagcggacccgacttaacctgttggtggaggaggaagctggcgtcgggctgaagtcccccgagccacccctggagccagagcagcccccgagcattcagagaaagccgcccgtccccagtcccctcggaagccggtcataaacccgacttctcatcacccgaattacttaaatgagctttgatggtgttaggtccttttccacgtgcggatgactcagcccgctgcagctgcgcccggggaggtgaccctggggcagcggctgaccctgctgcgccctgtaccctggacccagacgcg

SEQ ID NO: 94 - DNA, 246 bp, Homo sapiens
cgggagtctcccggggcgtggggagtaaggccccgggggtggggggccgggtgggccgggcgtgacgcgcggtcaaagtgcaatgatttttcagttcggttggctaaacagggtcagagctgagagcgaagcagaaggggctccctgtccggcccacgtgccctttccctcgacgacagtcgagggctcgggctctgtgggactgtgggagctagggtctgcggggcgcctgcccgggcgaggtcg

SEQ ID NO: 95 - DNA, 533 bp, Homo sapiens
cgggtgacgtcacctcctcggctctgcgccccgccgtccacaggcgcgaagccggagctggggttggcgaccgagaaccttcctgcctcccctccccagcaaggggcacgtcccaccaggcccggactgcccgtctgcccctgcagggcccctcgctctggtggcgggctcggtgagctctgctgcccggtctcgggagtctgggggagcgcggcgtcgacccctgtggagggaggactcgtgtttcaaggacagagcctcattaccatgcacgaccagccgcagctgaggccgcagaggcctttggcaggggggccgggccggcggcagggacggcgttggcggtggtggacgggagtgggggtctgtcctgaacaggtgggggtggtccctgtgctggcccccgccatgcgatcgcaccccaggactctcctcctcttcctaagccccagcatgtgcctggtgccgcgcgggtgctggaggcacagcggcttccacgcagacgcccgcagcaccacagcctgtgcacggcg

SEQ ID NO: 96 - DNA, 422 bp, Homo sapiens
cgagggcccggcaagccgctgagtccctacgggacagcacggggcctccggagagctgtggctccctggcctcacctgcaccccgccagcgtcgggctgctgctgctggcggcgcctctgctcttccagaagcttcttcacggtttgcgttgggcagtggctgcgctcacccctgctcactgtgcgggagaggagagggggaagtcatcaagggtccttaagtcacctaaatgccgcgggggtggcgcgcggggagtgggtttgggcaccctcctcgccctcccgcgcggaaaaactggcacatagatgccgcctgactcctccaaagccattagaagatttggggtggggcatccgcctccgccgtttccttccttttgctccgcactttccctctgacccaactttccaccaccgtcgcg

SEQ ID NO: 97 - DNA, 592 bp, Homo sapiens
cgacgccgcgctccccttcaccaccccagctcggcccagcgagaggtgcagcacgtgcaggccgagggcggggccggcgcggagagggtggccctgcatcgtagtaggtccctcccccgcagtgctcccagcaggcccgctcgttctggagaagggcgtgactcgacctgggggtggaccctaggctgaggggtagggtcctccggtgggcttggtcccgaaattgacccgcgcccctccccagttagcccgcggaccgtggactgtattttcagccccggatccgaagtgtcccagggggcgtggcccgtgcgaggggcggggcctgggggcgggaccagagcctgacgtgtgcccgccccggcttctaggcggatcgtggaccgcatggaccgcgcgggggacggcgacggctgggtgtcgctggccgagcttcgcgcgtggatcgcgcacacgcagcagcggcacatacgggactcggtgagcgcggcctgggacacgtacgacacggaccgcgacgggcgtgtgggttgggaggagctgcgcaacgccacctatggccactacgcgcccggtacgcggcgagcccccg

SEQ ID NO: 98 - DNA, 2019 bp, Homo sapiens
cggtgggcgaggaggagcggcgctgcccgcagccagggccagcgccctcgaggggcagaggcgggaccggcggcggcgaactcgcggcctcgggcaggggcggcggcggcggcggcggcggcagctgctgctccctggacgccttggggtccgcggcgcagccgttaggcacgtggttcccagggagttccgcctgcggggatgaaggtgggggctcacctcccagcttagggagaggctgaggggtctgggtcacatctggccgggggcgcgtgcagagccgcggtcaggtgtggcagagcagttggggcccacgtagcatccgcgacggctgcgccgcctgcgggggagcggaggggccttcgagcgagccgcgggcggcagggccgaggcgcgggcagccggcgggcgcgggctggcgggcacgcacctcctcgcggtgcgccatgcccgggcgtgcgaccagcgtctcgccggggcagcggcccagctgccggtagtagtcccccgtgctgggctgcttgctgaaagcgcggggcttgcggctggagtcctgcctccgtagcccgtcgtggccgtcgcaggagctcggctgcgggaagacagtgaccggtggggctcgggcaagtgcccggtaggcgccccccacgcctccccacccagctcttcacctcctgggaactcgcggccagcggccgtcggggggtgcagcagccgacttcctagaccccctgttctcacggtgggcagcgggcgaggttcatgggggcctcggtggaagggcaggctccgcccgccttacagggaggggttctgggcaccggccaaggggcacagggtcccccactgaggccagaaggggcgggcccaggggcgggccggcccagccccgacgccagggggagctagagaaggggcacctcccagcttagccttcactaggccctcggccgcactccgctctcggctgtcagaaggactgcgggtccccagggctccgcggagccctgtctttcgggggtcccgggccggagggagccccctccagagcctgtgctctccgaggctccggcttgccccggaccccgcttgtccgtctaggggctgctccaacctgccacggtgctggtggtcctgctgtgcacctggcggcgggggcggcaggacccgacacctgctttacgtgatacttcctctcaggttacagacgcccgcgcacggccagcctatgggctccgacggcctgacatcacccggggcccgccaatcccaggccgaaccccccccagccgtcgcggatgccacgggggcgccaactactctgccacacctggccgcggctctgcacccgccccgggccagatgtgaccccgccccctgcgcctctccctaagctgggagctgagcccccaccttcatccccgcccgagaggagagagggctgaccgtgggcagagggggcctctcatatttggctgccggctccgggtcgcgtccccaccgtttccctcctgcatctggaaaccatcgccatccacgaaagcgacaccgacacccgcgctcaagcctcggatttcaggggccgtaaggcggggtcgggtgacagcgcggcttcccgccccgtcgcagctgcccccaactaggcccagctcagtgagggagagtgaggcggccgggccaaagactgagtgaccgggtgggggctgtcccctgccccactctccagcccatgcgtccctgcggtggcctcagacccttcaccccgcccgacctggctcacgttgcaggaaacgcgacaccgcaggattcgttttctgggccagcccgccggctccgcgccccctgcagcccggaggctccgacgccacgaccctgctcccacctgcggtcaggcacccgcgcgggaggcgccgcggcgacacaaagagccctttgtagagcttcccggcccgggccctggcgtctgcggcccagcacgcacacagccgggagggacgcacacagccgggagggggctcg

SEQ ID NO: 99 - DNA, 459 bp, Homo sapiens
cgcgcggggaaagatgtgtctgccacaccttttgcgagcccaggaaacttgaaccgctcctgaaaatcctgatattttttcccgctgcagcccgcacgggtcaagcgcgcgggggctgaccaacactttccaggtcaggtcgagttgattgctcccagctcacgctccgaaatgcggtgctgaaccacgggaaggagggggtggggaggaaggcaggcagttgcatcctcgcgagtggggagtgcgaggcctcctcagctccagccagctgggtgaatggggcgcgcacggccccgtaacgccgagcgtggataaaatatgctaggactgcgcttctccggctcatcaaggctgcgggagcctccccgagccatcatccttcgtaattgcaaccgtcaatcaacactttaagacaaagtcagaaccgggaagggcgagccctggagacctgtctcgtcg

SEQ ID NO: 100 - DNA, 4037 bp, Homo sapiens
cggccagtgagcgcttcctccatccggggctcgggcggcgagctggctgcaagactgggcaccctggagccgtgggagccttgggagccgaatctgggttcgaatctgagcagggaagcggtgccgaccgcggaggatccaaggcagaaggcccagcgcaccgccgccgccagctcctgcccaacccggctccgctgggcttttcctctggggtggcgccggccccgcgcccctccccatccggggcgcctctgctaggacaccggggtctcggggtttctcagggctgctgcgtttcacctcctttaacgcggaggcgcggagttgcacgtgtgggtctcagtggagccgccacaggtcttattacacaacaaagggcagggagggcaaggccaggagcctcgcggggcggcgcagagaccccgcatcgcgggcgcgacccctgcctcgggctcaggctgactctgcggcagctccaagtcccgttgccgggcaggacttcggtgctccggaagggtgcggggcagcaacctggccagggcagcgcctcggtttccttccaattcattctcgattcccagatactcagatgagaaatccgcactcaggccgtcaggctcctgggcccgggccaccctgcctgcgctcagcccggcccctccgccgcagcgcctcggagtttgggggctacaggggaagaggaagcgtctggtcggctcccgtccgagctgctccacctgcgccgctggcctttccgcccgctacgcacctgggccccgagaggccagaggggagtgtccccagtggcccgggtccccgaaggtggcccagctcgggcgctcgcgcagccgggtactcaccttgggggcgcggcggcaggagagcgcgggcggagaggtgcgggcgccctgggacgaccagccgaggacgcctggtgtttggacggctgcccccgcgctgtccccccgtgcgaggcgtgttggcctccgtggcctcgcgagggaccgagggcggcgggtccgagtgccgcccgccggcctgggcagtggcggagcgcggagctggcggcgcctccgctgttgtttttgagccgcgaagcccgaagcagcagccgttgggtgggggcgaaggggaggaggcgggggccgggaggcgcgaggggggcggtggggcccggagggggcttcggaggagccgggttctggcgcgcgagccccgacgccccctccagcgatcgcagggcggggacgggggccgaggggagaatgcaggagaaaagcgggccggggcgacggggaacgcaaagcttttaaattagtaatcaaacgcagtaaaacggagcaaaaccagcccggactcgcgtccgaactatcggcgcgtgtgccccgagatccaaggaagtcgtgtggaaaaccgcagccgccgccccatcttcggccccggctggggcgggagcagcagcgcgacgcgccgcgacgcaggggcagggtggggggatagtcacctcgcctccggggccacctgggtccttttaaagtgtccggggtgcgggccccgggcgggagcgccgaacacggaatgttttcttaaagggccagttccgagctgcgctgaaaagggggggggggagaggggggagagataagtgagacgaagaccgagaaggccgaggagagccccccaatcccgcgccgaggcggcggcggcggcggcggcggcgcgacgatgaggatgatgaatacattgcaaagtttttttggccccggcgaggggtgtcagattgagtgctctgtgcgcatgtgcgaaggtgtccaaactgacaatgctggggagatgaagatagtgtgtggctgcttctggactcaaggaggaggagagagattccgcgagccgacaccatgcgatccaaggcgagggcgaggaagctagccaaaagtaagtctcccgcgctcggccgcgccgcgccgccggggcccgggccgccgggccggggcgcccgggccaggggtgcgcgtcggggcgcggccggcgcgcctgcggctccgggcccccggctcggccgcgcggcccggggggctgctccgcctcccgcgctccggggcgaccgggctcggcgcggaggctcggggcgcccgggccgcgcgctccccgaaggcgccggccccctccccgcggaaccccctccccccccaccagtgtcaggcgctcgggcccgggaacccgaggcgcgcggtgggggccggggaggggggcggagggggagggccgggggtggaggggagcgaaaagtgaaagttagcgaaaagttgacgaaaggggagagcaactttttcctccccgctcgctctctccctctctctctccgagtggctctcagtcctggtcaaatcatattccgggcttttgaaatagtgggcgctagagcaccgcctttggcgtccgccagccgggcgcagaggagggagaccccgagccggggaggggcggaggagggggcgcgggccggcgcccacccggctccgcgggcgcaggggcaggggtggcgacggcgggacagccgcagccacttggggagcaaaatggagacttttgcccgggctgggaactgtggtcgtgaagtttatccccggctgtgccccgcgtggtggagggcgctcctctgggacgccggcagcctgcgcgggccgccggggcgacagtgcctccgacaggagggacccacccggccacctcccgacccgccgctgccgccctcgggcccgcgtggacccctctgtacccgcttgcctggcacagccgggcccggcggggacagagctgccccggccaggctgcccggtcctcggtcttactttctctttcgctccgtctcggccgagccccgggctccgcgcgccccgcctgccgccagccggtccttgccgcgggccgggtgctccagccacccgcgccctcgcccgccacagcgcctccgagcagcccgcgcgcaccccgcagccccggtcgccggcgccatccgcggtgaggcgggcgggcaggtgaggacaggcggagccgcggcccggccagcgcggctcccgcagccccgccgaagccccggctgcagcgaagcggccagcgccgcgcggagcggaatcggcgccgcctgctcccgccgccccctcctctgcaggccgcccgcccgcccacccgcagaagaggcgcccgggccgctcccctcgaggtccccgcccgacccacgccgttccgtccgcgcctccggggccgggtccctccaggccggcctcgcagctcccggggccgagcccccaaacgtgagccgggtgcgcgcgggactcgcggcggttttccgaagctccgctccctcggctcggggcccgcccggctcgtcccaacagctgccccgcggccgtaggccccgcgctctgtgcaccagggaaaagcagctcccgagtttgtgccggacgctcctgcgccccgccatcggccgttcccggcccctgggtgaggcgcccccttcccgccgcgctccgctgccgggtgccctcggcctcggcctcccgtgacctctgacccctcttggtgcgaacaagaccgggcgtttcgccgccgacgcgaaggggctgtctgtgcgcggcgttgcgggccctccgcgcgtggggtgtgcgtgtgcgtgttcgggttcggttctgtgtgtgcaccgcgggcctgctcagagtcgggaccaccgggctgcgtgtgggccgccaggtcaggccctcgcccaggatgggccctaagctggaacgagaaaggggaaaccggccgcgctgacgccggggccttcgctgcaagcgagaggcggactcggcgcacccgcgcggccctggaaacttccgagaggccccagctctccgccggctccacctctgccaggtggggaaggtggcccgggaccgggtccgctcaggaccagaagtggtgcctggcagccatctggccaggaggccggagagccgggcccagcgggagcgccgagcgctggcttccttttcccg

SEQ ID NO: 101 - DNA, 2902 bp, Homo sapiens
cgcctcggcccgcaggaggaagctgaccgacatctgggaggagctgaagagcgagaaggagcagcgccggcggctggaggtgaggcgcgggtcccggcgggagggggctgacggcgagtggccccctgacaaagtctccacctcactcccgaccaggtccaggtgatgcaggggacccagaagtcccagaccccgcgcgtcatccacacgcagacgcagacctactgagggtgggcctgggaagggaccgcggcctgacctggctggggccacccacgtccttgcacacgactttgggaaacgcgagaaagtaaactctgcctagcacggcgccacgccggtctggtcgctgggggcggggcctgcgcgggggcagggcctgggcctccgcattcctgacggtccctcccaggcacatctggccaacatgtggctcccattaccgttcccaaggcctgcgcgcggctatttttatccaccggatggtgaggggcgggggagggtgtctccttcggactccagccgcgcactggagaaatggcgggcaggaggggcccaggcggggtcgggctcttgacgcccggcctagattccggaatccagctgtgcgccggggaggagccgggcgggccttctcgccagctcggacccctcccagctggcctccctgtccgctcagagggactcctgccctctcgcttgaacgcacctcggcgcagacgtgaagtccaccgtccagcccaagcacgcatgcttacaccggggctagcagcaccttagcctcgaggtcagggtcctgaggcctggtgtgtgtgtcattgcagtgtatgaacccagggcactgtggggacagggcgacgtgtgtccaggcgtttgccggtaaggaatgcgttttgaataaggaatgcgcgcgcgcttgctctgtgtgtgaggctgggtgcaggcctgtgggcttctgcttttctgctggctgtgaagtgaatgcgggcaggtgtgctgtatcgtcgggttggtgggtgggggtggtgcagctagggtaagggagtcaagctgggcctcagtcctggggcccctgcgtggcaggagcctgggggtcgctgggggcaggagtaacagagggagagactgagtcactgagaatgcagctttctttcatttggtcacttcacctcttcatcgaccccagagagggagtggggaccctgcatgctgccccctccccgcccccggggtcttctggcaggactggggaagggagcctctcagggggtggcggtccacgcccagtagcacctgggagctgtgggggccgaggcagtccgaaggtgtggggtagctctgagctcatgtgcaggtccggtaccccgagcgccttgcagcgggagccggggcccatcagtaatagtgcatgcggcccagggtgcccgtggccgtggggctgggccccagcgtgcctgtgcccagcaggtccggctgcccggtgcgccagatctcccgcaggatccgttcgcgctcctccagccgctgcgcccgctccagctcctccgccgacgtgaagacgttgacgttgaggggcccgtccggctctgcggacagctcggggcccggcagcgtgtcgtcggggcccagccgagtcaccgtgtcctcctcgtcctcgtcgtcgtcctcgggctccagggtgctgctgcgggggtcccggcgctgagccgggccccggggccgcgggcggggcgcccacgagatgctgatgacgagcaggcagagggtgagcagcaggccgaagcagacgcccagcacgaagtagaggccgaagctctcggggttggctgcggggcacagggcgggggtcacggagaggcccggacgggtgggggttgtcccacttccgaaagaccccaggttctaggtgtgggggcgaggcctgtaacccctactcgaagggcattagggaaccgccagcgggcaagagggcggggagcggggcggaggggacggacaaggcgctggggagggaaggggatggtggtcagggaagggttcaggcccccagtcttccggcgcccgccctcaccgcggatgtgcgcgtaggcagccaggctgttgctgagcaactccatgtcccttcgcggggcatccatgctgctctggggggcagctcccctaccagcctgcgaggtcagcaaagtcagatccttctcccagcctggagcccaccccgaccccttccgcgggcagctcctctcccgggagccgcgggcccaggttccaccctggagagagcgcttccctaacttcgtggcggcccggggccctgtcggggggaacccctctcccgggcgcggacgcccctccagtcctgggagcagcgccggggaccgcgggaggccccgccccctggtgaggtccccagctgaggcccggttccgccgtcccggggcgggatggaggatagggacgctgagcctccacgccccaggcgggctccgggaagggaagctgcctctctcctcgcccggccgccccgcggggacccaccggcccggggccaggcccactcaccgtcagcgccccgcttcccggctgcccggccccagcgcgtcccgcggccgcatacactgggtccggccgcccccgcccgctcgggtcccgccgcccctccggccccgctgggctcccaggccgcggcagcacgggcgggacacggaccgagggaccagccggcggaaagtttcctccgaggaaagaggagggacggggcggggcggggcgggcggggaggcggggaggaaaagggctgggagaggaaggggagaggagcgggcagccgggaggagggagagcccggcccgcgggccgtccgtcccccacaggaaaccgccggggaggccgcggcagggacccg

SEQ ID NO: 102 - DNA, 2272 bp, Homo sapiens
cgcggtggcactggtggctgccggcagctgtcgtctgcagagtgggtgagaccgagtgtggcagatggcttggtccgcggtgctggaggggactggcggagcagaagcgccctggcgccgggtccttgctttcctggagctggggcttggggaatggggtcggtcattgcagggacagaggagagggcagggtaatgggtgcgcaggggtcccttccaaaggctgcgggtggccaggggtgtgcgggggcgctgaggtgcggtcgccgagagattctggcctccagggtggtcgggcctggcatggactctaggggatggtgcacacaccggaccggacgggcccctcccttacccccggatcccccggctgggcagcggccagggagaggggcgacctacgggggcgggtgtggggacgccggactacgcgtcaggagtcccagatgggcccagacccccattccccgccccgggccgcagtgacgtcagggcccggacgttcccggaacggcctcttgggggcgttccagccccacggacccgcagggagtccccgccgcaatttgcatggggctcatttgcatgaccccgccccgcgcgggagtcgggggcgccggcgactggaggagccactgtcaatccccaggggaaagtgggcgaggcgatttctgggccgctcacctcactccaccctccagctcgcgccgcacctggggccctcccccacctacgccccgccagggcggggccgcgggcgcagacactcgcgggcacacgcacgacgacgcgcacacgcggtcgcacgcggccccccgagccccctgcggcgactccgattcacccccgcgggtgcggggcgcggacccgcccggcccagctcctgctccccaggcgcttcctcctcccgcgacctgccccggccgcacccgcctccgctccagcacgcgacgctgcccggcgcgacccctgctcccccggcccagccccaccgctgccctcgactcaatcccagttgaacgcccgcagcctcagtcccacccccggcccagccccagcgcccccagtcccacccccggccccagcttcagcctcagcgcccccaggcccagccccagtcccacccccagtcccaacaccttcaggcccaccctcggccccagcctcagccaccccagccccagtcccggcccagccccagccccagcgctcccggccccgggcccagccccagccccagccccagccgagcacccccggccccgcctgcccggcgttcccttttgtgcggcgctctcccgtccgcatccgcccgatcctctggaactccccgcggacgccggggtccctggaggcggcttcctttgtctctgctcccgccctccctccgcggcgtcttcgcccctcactcacctccccagccccgcggggacgaggggagctggagggagcccgggacccggcgcggcctccgcaggcggcgctttcttctcgctcccgacgcggccgcccctcctgcctgcccgctcctatcgccgcttccgcctctgccggggacggggacagcagaccctcggcgcccggcccccgcgcacctgccgcgcgcaccgcctctccggcctgcgcggctccgggggctccgggaagaccgagcgctggcggccagcccggggaggctgtgggcagcaggggtggctgcgggctcccagtccctcctccgcctacctcggagcccacatttgggatttctactccgggagaattctgccctcctcccacgatgagcctggaggactggagccgggacctggcacccccagggctgtgactgggtgaactctgagggcacagctgctgtccgccgccccaggggtcccaggatggggcgagcagccctgaagaggcccaagggcaccccgtgaggagcccccacgctctgagagtggggcgcagagccggagccccgggccatgcctccgctgccgctggcgcgggacacccggcagccgcctggcgcctccctgctggtgcgaggcttcatggtgccctgcaacgcctgcctgatcctgctggccaccgccacgctcggcttcgcggtgctgctgttcctcaacaactgtaggtggccgcgggcggggcgtcgcactggccaagccccaaccccgaggcccctgcacatagaggctggaggccgcggcgggtccgcggggctcccggcagcttcccggggatgggatgggtcactctgcggacccctcggtccctggaggccg

SEQ ID NO: 103 - DNA, 299 bp, Homo sapiens
cgcgcatccgcccggggacttgttggtggatccatccctcatggcggagcagcaaggggatcctttagaaaaagcaatgggcgaagtaactgaaagagcgacgcagaaagcaacagccagaaacggcggggacgcgagcggcccagacaggaagggaggcggtggcgcagctctggtgcgcagcgcgccgcagcgacggaacttctgcaaaagctgcctgcccgcgcgttatcagcggcgcgcaggcctgtggttttctcgctctcgcaaccctgctttaactgccggtttatttttcg

SEQ ID NO: 104 - DNA, 275 bp, Homo sapiens
cggcgcagatccgcacggtctccctccgcctcccgccgcaagtccccgctgctcaccgccctcgcgcaaggcgccccagcccgtaatccagcagtgcaggccgggctcgaagaagtgggagcgcgcgggcaggcagacggggcgcacggcggccgagcgcaccaccgggtggtcgagctgcagcagcgccacgtcgtagtcatggctgtcctcttcgtggtacgggtgcaggagcaggcggctcaccttgaaggacacctctccaggccagcgcg

SEQ ID NO: 105 - DNA, 2000 bp, Homo sapiens
cggtccccacccgcatgcgccctgctgccccgcccccctcaggcgatgcctccgcagccggggctgcccgccgggcctggagcgctctctgtgcccggaccttgtaattatgtcccacagcccgggaaaccgcgggcctcgcagcatctggctgcggttgcgaacttttgtttcaagatgcgcgactatttatagaaggaaaactggtaggatagtaatttcctaggagcaagaggagaaattggctgcgtctttattaaatgggggtgggggcgaggcgatgtattaggactgcaaccgcatggggggagggggtggcgcagggagcgggaaggcagcggctgccccctctggccgcgtcaggcgaccccggacgtggcccgcgtgtaaaggagggggagggggaagggaagagaaggggagggagagcaggctctgaggtttaatcagtagcatccagcttaaattcccccaaacggcggttccataaatgaaacttctttttaagtcagaggccagagcatatgcgtcccattaggaattcggcgaagccaccactgcgcgccccctgggctcactgggctggactcggccctgcccggagcttctcagagccccgtgggctctgccttgccgggcacacacacgcgcgcccacgctccgcgctctcccttggccgagacaactgggcgaacgcggccgccagcacgcaatgcgcgtcctcctcgcccccaggtgaggactcggcaggggcagggatcaggctacccagcgcggcaggaaggcgcgagaagccgcagcgacttcggggtacagtccgggcgctgtccggtgaagcgcggcgtgcacgtgcatgccggtgtttgcacgagggagcgggtgtgtgagtgcggctctcccaagtgtggcatggtattgcgcgccgcgtgggttaatatggggaggggtcccccgctggtccaggactctgctgggacgcgcagggaatctcggcgccgctcgcgtgcccgcgggccctcgcgctgggcgtgcacagcctgctctactcccgcagtcgagccgctctcaggccatcctttctttgaccccagagccaggacttctcactgccgtagctgcccgccaagtgctccacctcggcagcttaggctggcgaggcgacctaaagtcccccggctcccgcgcgcgacggtcacccggtgccccgcccaggggcctcgctgccgcgttccctgcccgcccggttccagcgctggaggcccggggggagcccacccgcccaccccgcacgccgcccgcggcactcacaagtagctgccgctggacaggaggagaaagatctgcgggcaatagacggagacgagcgcgctgcgcggcgacagtagaagcatggtgggccggcgtgcctggcggggctgaacaggctccgcgggccgggcgcctgggccagctgaggccggggtccccgcggagctccccgatgcagggcggcggcgctgggcgctaggggccgggtctcgtgggcccgggcgcagttgtcgcccgtgccccgccgctagggccgcgcatggcgctgggtctggggcagcggcgggagctggggctggagtgcggggcggggacccggccgggggcagcgggcgcgccttggctgcggtctggacctccgtccgtctgtcggtgcgaaccggctctgtcgctcgctctggctcacttctccaagtctcccgaggcgcgcagctgccattggacgggccgcccctacatccgcctcctcccccggcgggtttttttaaaggggaggctgaccggagcgtgcggtgggagccgcctccagggcttgcccaagttgccttggctgggacacccgtacccccacctttagggagccacacagctctggcctggctccagaaggtgcacgtgggggcggctccccgggggaactgcgcggaggcttgtgctcctcttggggggctgtgctcggcaacgtatgcgcg

SEQ ID NO: 106 - DNA, 5759 bp, Homo sapiens
cgactcggtgaaggatgatgcccggagggtgatctcgacctttaacattccacacacctacctccacgcaccaatcgccggaatctccaacccgcgggccgcgtgggctttctaccctgcaccgctgcagccgcggccacgggaagaggcgcgctcccggcggcccaagctgggagccaagctctaacgggtgtggcgggaagtgtggtggcccgccagcagctgccacgacgctcgctccaccgacgcccagagctgtggccgaggccgggggctggcacccgctgggccgccactctcggggattttggtggcaaagcggaggtcccgccgaggctggcgaggtgcgcggctggctgctaggaaagagatccagacggtcgcctggtgcgctggatccctgtgccctttccctgaaacccagcctggcctgactgcaacctctcccaacttcagtgccggatcccctagacaatcagggtgggctccccgctgcgagcgcgccccacagccgggtgccgccaaaggtctcctgctgttagcggtgactcacattcccagtgatttagaaaaactgtggtgccgagtgaaagaaaaaaaaaaaagcaaacacccttagacaaaagaaaaaagctccactctttccgcgagcgggaaaaaaggtttggttcagcaagtgatgctatttcagtgaatcagacgctctggggctctgcaaggaaacgcacggactgggagaaggaagtgggagcctagagttttgctctcggcaaaacaagaactcggcttgtcctcccaggccggcggtttccgtacggcaagacaaggcgcggaaaacatttttaccatctgacgctgtagtctgtcctttagaagagaataggtgaaaagtttaataaagaaattaagcgatgtggaagtcgttcccgctgcttgcagagggtggggaggcgggcctgggcaatttcggaactaggagagaccacgtcggcaaagcctgtggtcccgaacaagggccatggatttcccatttctcagatcccggctgcccgacttcattcttgtttctttcagcacaagccatcctcccttctccctccctccacgtctcttggcagagacagaaagggacaccgctctgcccgcgtccagcgcgaaggaggtgacctaaaacccatgggcccttctcacactgcgttctctgcgctccggcccagttccgggaggacctcccgacggcgcgggagaaggctgtaagggctcttggcccccgacgcggcctgggtctcgggggacgcatgaacccgccacctctggccggcttccttttagtgccccaaatagcagcgcttagcccgttgcttcccccgactcttgtttttcaaagtgttcacagggtcgcgagagcgagcgacgcgggggcggcgaggctgcaaggctgcacaggaacagacgacaagaaatagggctttcttctgcgtggttcttttccggtccctgtctgagtcgcccgaccccgcggggagcgcggagcattgcggcgtgcggcgggcttcgggctctcagcccgcgcagcaagttgtgttgcttttccccgggtgcgacgcgggacggccgcgaagagcaaagttcgtccgcggtaggaacttcgaggcccggttaggaccgatctccggcctccgagtctacttagccgtgtccacgctcgtgccttttccctcttgcagcgaaataactgggcggggcccgacgcgcgtcctccagcgactccgggaacccaaggcctccttcacctcgctcaaaattcacccgaaaatgcgctgagactttggcatggggctgcgctgtgtcccactgacgttacagaagcgcttatgaatttaaaaactgctccaggcggccggacggcgggacaacgcctcctcacttgctcctctacggcaaggccccttccttctccctgccggctgcggtggtgtgtagggtggtccctttacagagtttccttaatccatctagcctgacctcacctgcgggatgttccatttcccgatccacctcccacacccccgctgcagttgtctagaagctggggctggggaactgaggcgcgggaggagcagacaggagggccggggttggggcgccagccacgcgttgggagagcagacccgggtgcagactcagaggactggagaggcggcatggagtttggtctcctgggaggtagagtgaggtgctgcggactccagggaggaaagggatgggtggggaggcgggaggggaggagggacggggtattttgcgctcccgcaccgcggcccgaactcagctgttctgccttcactggcctcagccagtcgagggagctcctacgaggttatctgcgagcccagcaaatcttgggggtcctagccaaatgcagtgcgcaacgttctctctcacttggaggccgccattcacctcgtttttttttttcctaaaaggatttttttttctgagtgcttttttcatttccctattttacctgctttgcacatgcctcccgccctcacccgggaggcggcgaagacgcccactgggaccgcgcctggccgttttctgggcgcgtcctgcccccacggcctctgtctcttagggcgactgggcgcggaagaaaagctggagagcccctgcggggtggcaggaggagggtgcctgagtcccgcgagaggcccgggcgaggaagatgcgcagcctgctgatccgcgtcccgcccggcgccagggaccctcagagggaggagagctcaaagacctcgccccgcgccttcgcgaggaccaacccagtccccgcgcctgccccagagcggcttagaaactcagggcacagtgagagcgcagggcgcctcccgaggcttcacaccgccggccgggccagcggaggctgtgaccggagcgccccctctgtgccgcgcacgccgcggccgccgccgggttggggtaggtgagcgggaggctagggtacacttcggggtgggggatgggcgcgcacatggccgccagcaggcagagttactccggtaaacacgccagggacggcggcgcgcgcgggaggcaacccagcggggcaacccccgcctttacctgtccgagcctgcacgcgccggcggccgcggcgccgaggggggcggagcttgccagcccgcgcgccggggcgggacctagcgggggcggggtccgcagtgattgggcgtaggagcggggccgccagccagagctgggctgcagggccgcgcaggtttcacttcgctccgcgcagccgcctggtctgcagtttgttggagctctgcgtccagcgccgctgccgctgccgccgccgccgccgccgccgccgccgccgccgccgccgccactaccaccacttgattcttgcagccaccctgcgaaccctgccacactgcgatcgcatcatcgcggtattcggttcgctgcgttcccgccgccaccgcctcggcgccctttcttggcccttgttcccccaaatgtctgactctgactctcggactgagaaacgcaaggtaaatacctccaaatcccggggcgcgggcctggcaccgaaggaagactagcgctggagcccgaacggcaccacctactgcccgcttcccgtgcacgtgcgagcggagacgcttgcatcggcgccgcttgtcgccggcgggaggtggggatccgtgtggatcgggttccctctcgggcaggttctctttaaagaaaagtcgttcacgcggaacctgcaggctcttggccgaagtgcggcttgggacgggatgctgcgcgcgggcctggtgaagggtcgtaggtccgggcaggaggagaggcagcgcgtgcctttcggcgccggcccgggagctgaggacctgctcgtagtatcgggctgtgcgcccagctggaaagtctcgccgccggctccggcgccctcagagtagcggccgcgcagtgtgagggtgtttcgtgtcttggttttttggggcgcgtgggcctgttgcggaggatttgcggaactgcggtggtgatttaactgacgagacacggcttttccttttactcattcaggccaaggtttcggggccactgctgcgttcgctttctcaagcgtacctggggcccctgcttgctctgcaggagtcctgcggcctgtgccatccgcacgattccctggcagcgagaaatctaggtcctcccctgaccacctggaccgcgcaggggactagtcgcggccggcagaacctgctcggtccctgggccgggctatcttagcggctcacgcatgcccgagcacacccgacccggttcgcggtgcaggctggcggcgccgtgccctctgggacctgttgccccagggttggcgctgctggtggagggcccgacggacgccagggcggagggtgtgaatttactcgatggtcgcggacgtgcgcgtccgcggccaggggtggctcttcggctttgactcgtgtctgcggcgcaccaggagagagaggaagttgttggaggagaatagttgccacagccagggcggtgacggccgttagggtctgcccccgacgggccaaaacaaggacaagtggcgaggactgggcctggggatggccctgcgggtccgggcgcggcgcaagtcctgctttgtctccaggtgactggctgtccaggacggcgggagctgctaaggcttgtgtccggaaggagcacagggatgggcagggtggccgggccagccggccctcaaatcctccgacgaagcggcagccgccacgaccacggccagagcagcgctcggaggcgagtttgtcaacaatcgcctcgcctttggcggcctgacccgtaggcgccgcccccaccgcggctgccgattggctgcggcgcggagcgaccgcgcgcggaccaattgggaacgcgggcacccggcgccagcggcgcggggaggtcggcggtgccggcggcggcgggcgcagagcgcgaggggaggagcgggaggaggcggaggatgttcccggcggctgcggccggggcagcgcgggccagaggcgcggcgtgcggagccctcggctgcccggcggagcgcggcggcgggctggcgggaaggcgcgggctttgcgctgcgccggggactctgaacccgagtcccgggctttgtctcctgcgctgctttcgtggtgacggtcagggggcgcccggtcggcgaagggcgcgggccggacgccgcggggcttggtccctggcccggacgctgcgctctgaagggaaggcgcggacgtgagtttcggtgtgattgccttctgaggggagggtctgtgggatgaaggcggcgcggcgcgcaccggtgtcgcctagcctgcggacctagttgtagaccttgcaggcgttcgcttcgtcgccctccgccgccccctccccatttagagatgtgcacctcacggtgtgcacctcagagaagttctgtctgattcggtgcggcgtgcggtacgggagcgggagggagggagcacgggcggagagagcgccagagccgggccgagccgcgctggagttacaaactctattgtgacgcacttactacgactgacggccgctgccagaccttccccagacttgctgccctcagcattttcggcaaacaatggggccggagcagggaacgcggccagccgcctgcaatcgctgcatctgcgcccgctcctgtgctccggcgtcctacctaaccccgggaagtcagagccg

SEQ ID NO: 107 - DNA, 1297 bp, Homo sapiens
cgctcgccgacctcagctgttggggaccgggtccctcaatctccgcgtcctgagcggtcggcggcggtggggagagggcggcgggtgcccccggtcaccgcgctcggtcgggctcagtcccggcggccgagcgtggggatccgggggcccctggggtggagttgtccggaagcctgggctccactcctgctgcctcacagccttgccgtgtcccgtctgggcgcctgcgttcccaagtgacaaaatgacctcgctcctccgaggggtctcccaacctctgtccagatcttcttccccgctaagtgccctgtcaccggaggggatgtccagtgtgaccccgcgggaatgtcggggtgcttgggcccgggggtgggacagggctgctgcagggagggcagcccccgtaagaggagggaggacggcgacaccgctgagcgcagacggcgcgcggagccggggctgggcgctcaggtgcgaggttccgtccatctttctgaacccacgcaggtgggcgcgggaagggacccggcagccgccagggcagggtcagctggcggggcagccccgcacggtgaccctccttgggtcgcggggagccgggcacgggagtgggggagcgcgtctttgttggggaggagggcgggcgggacagggtaagactggggttccaggcccggggcgcgaacccgggtgggagggcgcagggggctcccctctgtccctgtcacctccacccatcgccggccgccgctaggggttccggccccgcgccctcccccgacccttccccggcggaggtcctggggcagccaagcccaccgcccggccctcctccgcggccgtcccgaagacccggagaagtggaccacagggggcggccagtgtcaggggtcgagtcctccgcgctggccgcggttccggagatccgggctttgccgagcgccccgcgccgtggccggggccggggccaggtccgggtgcggggcctggcgctgcggccgcgagctcagcctgtaggctgggctagcctccgtcggcggcgggcgctcaggagcggggccgcggcggccggaggacaaagggcccgaggtaggagcggtccctccgggcggcagcgggcggagcgggggcgcggggctgtgcggggctgctcggggcgggcactcggcgcccggcggggaaggtgcgccccgccggacgcgggcacttcctggccctggtcccgggaaggaggtggaaggtgtccgggatcggccctggcagcgtgtagggacctgggtgctgccgctgccgtgcg

SEQ ID NO: 108 - DNA, 4027 bp, Homo sapiens
cgggtcggccaagttgtgtggtcgccgctcgcgttttagaggggacaactcgttgggggagaagggggcgtgcgagtgcaggatgtgcgagggacccattgtatttgggtcgcaagcagcccacccgtcttgagtacgatattctcattccgcaggcgcgccctgggcgtgggagaaggtgggcccggaggcatcgcatccccgacccctgatggaagtgcggagggcatccttatacagtccagattccgagtccgttgtagttaggctaacctctgacgctgcatcgggagataggtcaccccgtccgtcccggtcgcagtgcggttcccgtgaggaagacgggcagagaggccgggcgcgaactgattaggggctcccccacggccggcggcctggagagtggagactgtgtccgacgggaggtgggtgggggagaggagtaggccgagctccggcggccactccgcagtgcgctctcgcgagccggggccgcgaggcctccaacgcggttccgcacccctaatgccccagggcggtgagcaccccgcggttccccgcccgcctcttcctccttccccgcccgcgcgcccgggagaggcgatccccgcgtccgcagtgccggggcaggacgcgccctgcgccctgggccaggaggaggcaggtggccctcctcggcaggaggtgagccgccagggagggatgggggtcacccaagcccctgcaagcgcgaccctgcccagagcaaagcacgccccgcgcctggtgaggaactttttctttccaaaggtggcaggaacaagtctgtactgacttttctcccgccttcgggatcctctctcgtatgatttgtggtttgtgggaccacaacaaccaggcttcctcgaccgcgccttttttccagtagctcctggtctgccaggcttgcgagagggcgcgctggcttggcggggctccgggcgagaaatcaccagcagcgctggggctttcggccccccacagcccagcccgggcctcctgggtggcctggggagaagggggcgggacagcacagcttcttcccctcgggtgggcatgacgggtcgccttgggtgtcctgccgagctcagctttgagggtatgcgcggcgcgggtgtttttcccaggtccactctgcaaaagttgtggcctgaggaaaacttccaggtcgccagcaggaacgtcacaggagagccccggtcgctcgtcaggcgggcggccctggagcgtgcgcgccacccaacgccgcgctgggcggccgcccggccccgccccaggtcacagccctgccctcggccccaccgcggcgggcgggggacacgcggcgggccgggcgctgtcggtcccacgccccaccggggatctcgcttgggtccgctgtgtgcgcgggcgggtgcgtgtgcggcgagggtgcagggtgtgcaagtaaatacaggggagagttacactctttgccgtctcaaagtccggcgcctggaagccgaaggcagatttttccagagataacaaagacactttgtcactttgggcgctgcgtcggtgcaaatctttaattgcaagtgtgtgtgtgtgtgtgtgtgtgtatgttggggggggcgggaagagtgtctcgagcaacccaagcgcgggtctccaggcggcaaggccccctttgatcaggaaaatccaattatttgtgtatatacatttcccacatagtgattacttcattaattgtcccgcctttatctcctcccttcccatcccccctaataatcagttcttttatccagaccaacaaacacaccataggagctttgtggattcaaaggatttgctttcgcttctgaaagagccgctattctttgatgattgggtagcggcaaacttcaaagccataaatcttccctctgactggctggcggcccagcaaagtccttatcaaattcttggaggtgatcctgaagcgctcagaccgcgcgcggggcgagcgagcggggcgcggcgaggggcaagggcggggagggccccggcgctcagagcaggcgccagggaggcaggctgggcggcccttcgtcctcgccttcgggtgtccatgcctcggcggcggcgtcccgctccgcagccaggggcctgcaagccgtagccatggccgcggtggccgtcctccggaacgactcgctgcaggcctttctccaggtcagggccgagcccggagggggcgggagaaaggtggaaagggccgtgtccggatctctcctggtactccgtgcaccttgaacctcaaagggaaccccgccgatgggtgcagctggagccaggcctaggggtgcccacgccaacttcacacctaatccggtgtggcctttgctggagggacgcgtggatccgggattgttcggaggtgcccggctggccgagaacaggaccggagctcaccctgctgggccctcctgctgatttcagcctggggcgggccagggaggagcgcgaaggcagtgaggacaagagagcttaaaagatgggagggagagcggccggccggcggggttgtgggtgtttcccgactcttactgaccacggagccgggaggggcccgtcggatgcctccccctcccacttcgcgcctgcttcctctaaggctggatggcgtctcagtgcaccaagtagtttagcaagaagcgctcgcgggccccacgttcaggtagcgcaggcaccaaaagtttccctcggccggtgggtgcgtgcgtggggtgcggtggcgggggagggacggccgggacggaggaggggcggcggtataatagcgggcgtgggggcggcgggctggcccggagtccgcgcttggagctaaccttttgtctcttctccgccctccctcgccgcctatgcaggaccgcacccccagcgcctccccggacctgggcaagcactcgcccctggcattgctggccgccacctgtagccgcatcggccagccgggcgcggcggcgcccccggacttcctgcaggtgccctacgaccccgcgctgggctcaccctccaggctcttccacccgtggaccgccgacatgccggcgcactcgccaggcgcactgccgcccccgcatcccagcttggggctgacgccgcagaagacgcacctgcagccgtccttcggggctgcgcacgagcttccccttacaccccccgccgacccctcgtacccctacgagttctcgccggtcaagatgctgccctcgagcatggcggctctgcccgccagctgcgcgcccgcctacgtgccctacgcggcgcaggccgcgctgccgccaggctactccaacctgctgcctccgccgccgccaccgcccccgccgcccacctgccgccagttgtcacccaacccggcccccgacgacctcccgtggtggagcatcccgcaggcgggcgccgggccgggggcctccggggttccgggaagcggcctctccggcgcctgtgccggggccccccacgcgccccgcttccccgcctctgcggccgctgctgctgcggccgccgccgccctacaaagaggcctggtgttgggcccgtcggactttgcgcagtaccagagccagatcgccgcgctgctgcagaccaaggcccccctggcggccacggccaggaggtgccgccgctgccgctgtcccaactgccaggcggcgggcggcgcccccgaggcggagccggggaagaagaagcagcacgtgtgccacgtgccgggctgcggcaaggtgtacgggaagacgtcgcacctgaaggcgcacctgcgctggcacacgggcgagcgacccttcgtgtgcaactggctcttctgcgggaagagcttcacgcgctcggacgagctgcagcggcacctgcggactcacacgggcgagaagcgctttgcctgtcccgagtgcggcaagcgcttcatgcgcagcgaccacctcgcgaagcacgtcaagactcaccagaataagaagctcaaagtcgctgaggccggggttaagcgggaggacgcgcg

SEQ ID NO: 109 - DNA, 976 bp, Homo sapiens
cggacttgacggagcaatgcacgtgggcctttgactcgtaatacacccagtcaaagccggcctccactgccaagcgcgccagcagtccatacttattgcggtcgcggtctgatgtggtgatgtccaccgcgcggccctcataatgcagggactcctctgagtggtggccgtcctcgtcccagccctcggtcacccgcagcttcacaccgggccactggttcatcaccgagatagccagcgagttcaggcggtccttgcagcgctgggagaggaatgtgcgcgaaatcaaccagagcgaaatcagccggaggagccggccgaggtgcaggtgtaggcgcagcctcgcccagaactctcccgccacacccaagggagctggaagcccgggagggtgctgtccctctcccttaggaacagagggccctgaagtgctccctccctagcatacagccgcccccggggatctggaccgcacggcggtatcacaaatctggaggaggcatggaggcaagcgccctggcggcgccttcactggtaggtggaggtcccctctctcctgtgctgtcttcagctccgggtatccatccctccacagacaggactacacctttcagcgcccggtccctctgtgcctccaaggcaggagggccagccagcggccgggagggaggagtggcagcagagccgactgcctgcggccggcctgcgaggcgtgcaaggcccgaggtgcccagtaggtgtcaccgcagcctcagggctgaggcctggggacgacaggctctggaggcgcggcaggtgcagagcgtctcggtagcttcagggggtgtgttggaggggaatcgccggcctgcctggatccctcgagcaccaccgaggattcccgccgggaccctcttataatcttaaaccggcccacaaccccgccagagatgacagggccagggaaccagaaaccggcggattagcgccagggccg

SEQ ID NO: 110 - DNA, 539 bp, Homo sapiens
cgccagcagcccgcgcggccccgcggttccacggggcctcgtccccgcccctgccccgctgtaggccccgcctgctataggccccgccccgctgtaggccccgcctgctataggccccgctccgctgtaggccccgcctgctataggccccgccccgctgtaggccccgcctgctataggccccgcctgctgtaggccccgcctgctataggccccgctccgctgtaggccccaccccgcaggaaacctcgccccgctggtggcgcgccctggttgccgtggcaacctcctccctccgccgccgggttgccggcttcgctagcctctcagcagcgccgttgccatagcaacggcgggtgggtcccgcggggcgagctgagctagggtgagcatcctgtggggtagactatggcggctgggcgcgtgcggcctgtgcggggcctccccggctgcggtgagagcggctcgaggggtcccgtgcccggaccctgaagtgcggccaagccgtggggggctccgtgggctcgcgctgcaccgcg

SEQ ID NO: 111 - DNA, 467 bp, Homo sapiens
cggtgcaggacgcagggcgcgctctagagtgaaggagtgacgagcaaagggaaggaatgggcaagcgtaggcgaagagaggagggcagcggggggtgcaaaggtactcagtttgccctctgtgagcggggggctgcacccaccttcttctggaaggagccgggattgccgcggcggatggccagcagcgcggtgtagtgcagcgtctcgcgcacggtgaggctgctcagcagggtgtcgctctgcaggagactcgggcgtcagtgtagcctaagcccccggggcgggcgggggggccaggggtgtgggggacgcgcccacctgcaggacgtaggagaagcagtcctggaactgctcccggcgcagcgcccggccgttcacatacacctcccccaggaaggtccccgcgcgccccagcctcccggacatggcgtccagcagcgtggttttcccggagcctgcggggcg

SEQ ID NO: 112 - DNA, 239 bp, Homo sapiens
cgcaacgcccggctgaggtgccccttccggaagggcgcctgcgagatcacccggaagacccggcgacagtgccaggcctgccgcctgcgcaagtgcctggagagcggcatgaagaaggagagtgagcagtgggcgcgcgggcgggccggcgccggggtgcacggctctgagtaaggacgtgccgtgggtgtgggcatgcttgtgtggagatgcgcgccgagtgtgcgcgtgaacacacg

SEQ ID NO: 113 - DNA, 584 bp, Homo sapiens
cgcctctgatgggatgcgccctccccgcccaggaagtggcggcagaaagcgagccctgagaagccaggggcaggagcggcctccgcgcgacactgcggcgctcctgattctgcggcctggggccgagcatgcggggcgggcggagcctcgagctaagtcccctggggtcccagggccgcattcctccgaggtctgcaaaggccactgcttaaaggcgcagaggagcagctgggaacgagaacaaagcggccaggcccccctcggaggaaggaaggagagagccccaggaaacagctgatagcgctaagctcagcttgtttttttcctctgctcaacagttctcctgccacggcaaacaaaacatgtacattctgattccctcttctgtttggattgtgctgtcgactggatctggtttgtgatgagctgggggaagaggcatccgcgggcgatttctggctcggcgtgccagtgtgcttttgctgggccgcgccgggatcgcggagcttcctctccggctcctttctccccgtctgcgtcgctaatccagcctggcccggccaccccaagggaagacacggccg

SEQ ID NO: 114 - DNA, 766 bp, Homo sapiens
cgagttccaccggaattgggttcgagcgcgggtgaaaagagggacccgacagcacccaccctccaagtagaggcaaaagttgaaagtggcgggctggctgcgcttccgaagaaacacgactggcgaaggctcaccttagtctctacggtgggtccctccctgtagccgacccgttccttgaagcgggccagaaacgccggctcggctggccgcacgtacgatacctggttccgcttgctcatggtagctctgggtaaagacagaagacaggttccgcgacggattcgcgacgatacagaggcgcttagctgtgacgtcactgggccacgcccccgatgcgtaggccgggcaacatggcggcctccatgcgggcgtcaacgtccgatccaagcgccaaattcaaatttgcggccatcttgagcgggcggaattcagtcgcgcgcggtgcagtcgggaggtggaggcaccggctgcattgttttcgggatcgaggggtgagggcgctatggcacccggctgcaaaagtaagtctgggcccccggcttcgttaccctatttttgcccccaaatacagctgtgaaaggatggcagcctcggaccgcccgcaaggttcttgctaggcatgaactgcaggagctgagtgaccggcggggacgtttgggaatcccgctctgtcgctcaaagacaatacgctagccgaaaagctcatcccttccttcagtgtcctgtgcgcatgccaggagtctggcgcagttctaggcgctcg

SEQ ID NO: 115 - DNA, 737 bp, Homo sapiens
cgagtggagtgcgacaagggataaaccattttcgcgcactcttcagcgatggggcgaaagtaacggacctagtcctcgggagctgtccccgccgaccccctctgccgcgacttgacccgcggcgactgcgctgccccttggctgccccttccgctctcgtaggcgcgcggggccactactcacgcgcgcactgcaggcctttgcgcacgacgccccagatgaagtcgccacagaggtcgcaccacgtgtgcgtggcgggccccgcgggctggaagcggtggccacggccagggaccagctgccgtgtggggttgcacgcggtgccccgcgcgatgcgcagcgcgttggcacgctccagccgggtgcggcccttcccagcgcgcccagcgggtgccagctcccgcagctcaatgagctcaggctcccccgacatggcccggttgggcccgtgcttcgctggctttgggcgctagcaagcgcgggccgggcggggccacagggcgggccccgacttcagcgcctcccccaggatccagactgggcggcgggaaggagctgaggagagccgcgcaatggaaacctgggtgcagggactgtggggcccgaaggcggggctgggcgcgctctcgcagagccccccccgccttgcccttccttccctccttcgtcccctcctcacaccccaccccggacggccacaacgacggcgaccgcaaagcaccacgcggagatacccg

SEQ ID NO: 116 - DNA, 278 bp, Homo sapiens
cgctaagcgctgactcggggaagggagggcgcagcctttgtcccgcaggtctcccgcgcccgcctcgctgtccccgcgagcgagcttgggctcgacccccgttccccctgaacgcacctgcaaacaaaggagagccgttccttcgccaacttcctcacctttgcctcccggtccttccgaagtcgggcggggatgccgccccaggtgctacgacaagccccagggtgccggcaccagccagcggtgccgtcgctcaagcagccgcgcgcactgccccg

SEQ ID NO: 117 - DNA, 905 bp, Homo sapiens
cgcctgaagcgctccgactccgactggcgggcctgaaggcatcaccgagcttcaaggacccggagtccaggttcatggagccttggggttgcgtctcgggcagccatccgacctcgtgatcttcaaacttagccagcgccggatgctggaactccaagggctcgggcccagggaggccagagctggagcaggcctcacccaggcattcccgcgccggcccaccgccccgacccacacgatccagctttcctggctctgagaagcaccacttccgctgctggttggcgaggggaccccggccggcagttcctggcgcgggagcccgggagcgcgccggctccccctccccgcccgggtggctgagcgaggcggagcgcgggtgagtcgccgggggccgcgctgggacagtgggccgcagacgggcgggcaggctcatgcggagctccttgcgctggaacgacgtctcccggagcactcgccgctgcgcgccctgaagccgctggcggtaggcggccctcgaggccggcgggctgggcggctcggcagcctgcgccgcggcctccgcctcggccgccagcgcgtacagcagcggggtggcctgcctgttcagtggtcccggggtccccgggacctctgttggctgcggcccactgcgggctgcaaccgcgggccggggccgcggggatgtgcaaagggcagcgtcgggcggggcggggcccgggccgccccaaaccacacgcacgtagtcccagtctaggtaaggaaggaggtctgtccccggcgactgcgtgcgcggctcggggccgccggaggctgcggagaaagagctgtaggccgagtccgcgcgcatggacagatgccacaggtccaggctgctagtggacgaggccggggaggcgcggtcg

SEQ ID NO: 118 - DNA, 674 bp, Homo sapiens
cggccgccgggcacagtggcggcgagtccgagcgcagcgcgtcgtgcgccccacgcaggcccaggaaaggtgtgctctcggccgccagcgccccgtccgcgtcgtccgcgtcgtcgtccgacgccgaggctgagccgccgcccgagccgctgctcagcgacagcgagtccctcgccgcccgtgcgcgctgcgccgccagcccgttgaggcgcgagcggcgccagcgccggggccccgccgacgtcctgcgggacgcaccgcgcgcgcgcggccgcggcggcggcgggggcgcgcactcctgcgtggtctcgtactcgtcgtcctcggggatgcggaaggggctggcaggcaggctgcccaggctgccgccgagcgcgcaggtcccgcgccgcggtccgggccccgccgcggggtaatagtagctgtcatagctgcgctgcatgtctgcgccgggcccgggcccgggcccgggtccgggtcccgggccggggggcgccgggtgccgcagtaacggctgctgctcggccaggcggtaactgatgggcgccgccggcggcagcgacacggcgtgcgccgagttgggggacgtgatctcgaaagttggcacctgcgtggccagcgagtagtggaagtccacgggcgagaggcgcgcgggcgtggtcagggccgacacg

SEQ ID NO: 119 - DNA, 1271 bp, Homo sapiens
cggacggcacgctcaagtacatggaggtgacgctgcggcccacagactcgcagagccactgctacaggacgtgcttttcaccggcctcggacggcagtgacttcacttttctaagacccctcagcgttcagcagcccacagctctggcgctggagcctgacgccatccggtcccgctctaatacgctgcgggagcggagccaggtgaggggctcggcgccgccccgggcgacccctgggggcggcactggagaagccgcccgtcctcataagggattgaacttgcatccactcctctccggccggcttggtcgctggctgcgctccacccgattctcgggatcattggaccgtttgcgcgaaaccagagtggccgattaagggatggggctccgagcaccgggggtggtggcgactgtgggcgaggggaggtgggaccgacccccacccctacactcaaaaaaggccggggcctccttcgagcttccggtgaatttcgggcgatttccgcgggtgtcgggggtcccgggaggaggcagtcacagatccacccctgcagccagcctcctaggcgccggctccggcacgcttcgccggtctgtagatttcctcttcgatttctccccagctcccagcatctgtgacttcactgttaccctccctatccccgcatcacccaaccgcacctgtctgcgggacttaggtgtgcgcgcggggctcatgcgtgtcctccctgctggccacccccacggcccacacaagttgcacgggctcgccacgccccgccaacacgtgcgcggacgcacgcacgcactcctcgcacgtgggcttacgcgaataccagctttcactgccactcgctcgcggccagattcacaggcctgttccggtccactcgcagctcccctctgccgctccctccgccgggctcaggagtactcgtagctgattgtgcgcgcctgagggtcccagatcgcggccgcccaggaccaggcgaggactccggagcctcctctcacctctcccacctgcgccccgggctgggccgggtcgcctggggggcggcctgagcgaggcgcggggccaggagcgctggagcgactgccgctctaagtgccgggcgggcaggactctacgatccttgggccagaggtccggatggtcccgggactccgtctcaagggtcggcgacccctcaacccagaagcctcgagcaggcggacaggcagagctgcccagtggccgaggcgcggcagggctccgcgttgggcg

SEQ ID NO: 120 - DNA, 1364 bp, Homo sapiens
cggccacctggcagagcgtggtctcggagggtcgcgcggcgccgcttggcaggcacaccggctgaacgtaaggcgacaggagcgcgcagctgccgtccgcatcctcctgaaggcgcaacagagctaacccgggcggagaggagcgtgaggcggggacgccggggccccaagctctcttcccgtccccgcggggcgcccccacgcacccaggtcgtgctggtagctgacgggcgagaaggcctcgtgcaagcggtaggagcgcacggccaacgtctggcacggctcacagctgtggttacggcgttcctggccgagcaccaccgtcagatcctcgggtgcgggcctgcggggggggtaggggagaggcagcgctctcagacctggccacaagcgttctggggaagccccctgctcccgaaccccaatcccgtgttccagcttcctccccgggagctccggaggggcgtggaaagaagggtggggcgggagaaggtagggcacgggttcgggtgcagcgtggaagctgcgctgggagacggaggagccgcggcccctggggcggctctgggcgggcgggtactcgccggtcctgcaggcagtgagcggccgtcagcacccagcagggggcgatgaggctgccggcgcagaaactgtggccccagtacagcgcggcgatgtaggggtgcgccccgcgtagcgccaccagcccgccaacgacgcgggtcatcgaagacagactcttgcggagccgctgcccgcagctcagtgggccgttcctggtcagggaaggcggctgctcccgcttcgccggcaaggctgtggaggagcaggggctgaggacggagagcccgcggccggctggccggaatctagctcgcccggcgccctctcggctcctccttcccccccccacttcctaacctcccggggtctgggactgaggcggggtccgggtcgtgggctgaggcttcggcggtgccggctgcgcgggcatgagtgggacatgaagcctaggggacaccggggtcggaggcgccgcctgggttggggtctggcactgtgccaggtcgcagtactcccagctcagccggtcgcggttcagcacgaagcaccacgggcggatgtcgttgtccgggttcctgtagccacacgacggggcgccgttagagcgccgggagcccggagccctggggcggaggggtcacccagccccacgcggcgcaccggcagaaggcgtggccgcccagtccccagttccgcgcttgctcggcagtcacgttccggtaggtggcctccgaggcccacggctgacagggcgcacccgagagcgtggtcctggccaggccgcggtagctgagcccgcg

SEQ ID NO: 121 - DNA, 1010 bp, Homo sapiens
cgagccggagaagatgcgcggggcgcgtgtggctgcgggaagagcttctgcattgtgttgggtacgctgcggcgcggcaatgttcgcaagctccctggatgcacattgcctcttttctctctttctttttgtcagcggttcagcgaggaagaatgcctccaacccagcccaatccaggccagtacgcactcaccaacggggaccccctcaacggccactgctacctgtccggctacatctcgctgctgctgcgcgccgagccctaccccacgtcgcgctacggcagccagtgcatgcagcccaacaacattatgggcatcgagaacatctgcgagctggccgcgcgcctgctcttcagcgccgtcgagtgggcccgcaacatccccttcttcccggatctgcagatcaccgaccaggtgtccctgctacgcctcacctggagcgagctgttcgtgctcaacgcggcccagtgctctatgccgctgcacgtggcgccgttgctggccgccgccggcctgcatgcctcgcccatgtctgccgaccgcgtcgtggccttcatggaccacatccgcatcttccaggagcaggtggagaagctcaaggcgctacacgtcgactcagccgagtacagctgcctcaaagccatcgtgctgttcacgtcaggtgaggctgcggtcgcggggagggcaggccgcgccggcagcgagcgcaggccccgcgccgcccgggcctggccttcggagcctcccgcgcggccggtgcggggcgggccgggccctcgctggacactgagcctggcccgggtctgtgacccccgcgcggtgaccggctggccgcgccgctgccatcttgggagcgcggcggtgcgggaaccgaggcggcgcgggtgcggccggcgggcggccgggcctagtcgcccggacccctacgtggggccgcggctgccgagggtgtgtgtctgaggcgggggggcggtgctgtggatccgtctggctgcgcgcgcgcgcgcg

SEQ ID NO: 122 - DNA, 1530 bp, Homo sapiens
cgggcctccccttggcgtcccgcctcggagccctcccatggtgatgaccaaccgcggcccggtgccgctgcccatcttcatggagcagcagatcatgcagcagatccgcccgcccttcatccgcgggcctccgcaccatgcctccaaccccaacagccccctgtccaaccccatgcttcccggcatcgggcccccgcccggtggccccagaaacctgggccccacttccagccccatgcaccggcccatgctatcgccccacatccaccccccgagcacccccaccatgcccgggaaccccccaggcctgctgcccccgccgcctccgggcgccccgctgccgagtcttcccttcccgccagtgagcatgatgccaaatggcccgatgccggtgccccagatgatgaatttcgggctgccgtcgcttgccccgctggtgccgcccccgaccctgctcgtgccgtaccccgtgatcgtgcccctaccggtgcccatccccatccccatccctatccctcacgtcagcgactccaagccccccaacgggttctccagcaacggggagaacttcattccgaacgcccctggcgactccgcggcggcgggcggcaagccaagcggacactccctgtccccccgggactccaagcagggctcgtccaagtccgcggactcgccccccggctgctcgggccaggccctgagcctggcgcccacgcccgccgagcatggccggagcgaggtggtggacctgacgcggcgcgccggcagccccccgggccccccgggcgcgggcggccagctcggcttcccaggcgtgctgcagggcccgcaggacggcgtcatcgacctgaccgtgggccaccgagcccggctgcacaacgtgatccaccgcgcgctgcacgcgcacgtcaaggcggagcgcgagccgagcgccgcggagcgcaggacctgcggcggctgcagggacggccactgcagcccgcccgccgccggcgacccaggcccgggcgccccggcgggccccgaggcggccgcggcctgcaacgtcatcgtgaacggcacgcgcggcgccgccgccgagggcgctaagagcgcggagccgcctcccgagcagccgccgccgccgccgccgcccgcgccccccaagaagctgctgtcgcctgaggaaccggcggtgagcgagctagagtcggtcaaggagaataactgtgcttccaactgccacctggacggggaggcggccaaaaagctgatgggcgaggaggccctggcggggggcgacaagtcagacccgaaccttaataaccccgcggacgaggaccatgcctatgctctgcggatgctgcccaagaccggctgcgtgatccagcctgtgccaaaacccgcggagaaggctgccatggcaccgtgcatcatctcctcgcccatgctcagcgccgggcctgaggacctggagccgccgctcaaaaggaggtgcctccgaattagaaatcagaataagtaaaaggtttgtatgtccgccgggcg

SEQ ID NO: 123 - DNA, 949 bp, Homo sapiens
cggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggtacccgcgcccgcgccgcccgtcggggtggccgccgcgcccggcaggagggagggagggagggagggagaagggagagcctagggagctgcgggagccgcgggacgcgcgacccgagggtgcgcgcagggagcccggggcgcgcggcccagcccgggggttctgcgtgcagcccgcgctgcgttcagagtcaagttctctcgccgggcagctgaaaaaaacgtactctccacccacttaccgtccgtgcgagaggcagacccgaaagcccg

SEQ ID NO: 124 - DNA, 671 bp, Homo sapiens
cgcgcaccagcagcaggcggcccactggatccacgcccctggcccgcaggtcctgcaggtcttcgggccgcccgtagtgggcgtacaccagctctccctggggacacgaggacggtgaggcgcgctccccgcgtccctccttcgagacccaggaaagggctcgtgccgcgccccatcctaggagcgggcagggggtgctcaccgtgacgttgccgatggcgctgtaggggcagtagacgtcagggtcctccagcggcagctgctctccgaccttcccggcctcatcgacccagtgcagggtgttggggtgagccctggggagcggggctggtgagcgccccgagccgcgtccccctccccgcgcgccccccgcccgcgcgcgcactcacggatccgggaattgcagccccacgtagtgcgtgtcggtccacacgtggtccagcttctggcgggagagcgccgcgcgaatgtcctgagtcagagcggccatcccggccgagcctgccacccgttcccgaaggctggtttgcctaagcggggagaggtggggacttttctgggcgcccggaggagagggagggaagagggaaggatgccagagacgtggggtcgccaggggcaggggcggcgaggggttcctgagtttaggaaaagagcgctccccgcggacg

SEQ ID NO: 125 - DNA, 364 bp, Homo sapiens
cgagtaactcgtccgcagcgctcgcggggagccaggaccagggcagacgcgggaaatactgccctcgggcggccgcgctgtgctgaaaagggaacacaggacaccccacaggcagcctcggtgccacttcggggtctctctgtcccaccacggggtgcgctgtccggaccgcggcctggggacgttcttccgccactagagggcggcccaagccccgagcccgcggccccgggaggataggatgcccgtggcgtggacccgcgcagcacagcctccccgcgcggcctctccagccgaaagagccggcgccgccacgtggaggtgtaggtccggcctggcgcggacaccttcgtgagtgggcccg

SEQ ID NO: 126 - DNA, 3722 bp, Homo sapiens
cgcggtggcccccgcacccggagcgtcggcagcacctggagcggttagggctactctgccgccgccgcccccgcggtccccgccgccgctgtccccgccgcggtccgtgcgcgcgggcgccagtgcagccaggagcgcgtgcgccaggcggaagggcgcgaaggcccggtgcgcccagctgtgctcctcgatgaccgcgtagcacgaggccagcacccggttgatgagaatggtgccctgggccgtgagcggcgcgtaggcgcccgcggcctcctcgcttagggtcacgctgtgcacagcggcgggcaggagccggcggtccccgtcacgctcggccaccacgtacacgcgctggcccgggcgcacgcggctggcgaacagcgcccgaggccccagtgcgcccccggaaggcggccccgagcccgaggacgcctcgggctccccggtggccgagtcgttgtgcggcgccacaaagagcaggtgcgcggcggtgagcagcaggcgctcgcgcggctcccgcgtctcgatcacgtagaagaccttcttggcgccgtcgtcgcggtccaggaaagtgaggaagtcgctgtagagcagccggccctggtcgtccgccgccagcacgcggtccccggggctcaggtccttcaccagcttggtgccgccctgctccaggtgcaccgtggccgagcccgggaagcagcctcccgatttggccgccaccgagttctctgcgggtgaggagaagggaaagaagagaggacagggcattgagttccgagagggaggcgcgtctcggggaggagggcgcacgcttggtgcccgcgctcctaggccaggggtgcgcaaggcgcggggcggggcgattgattccaggcgggtcccgcacacacctccctcccccaaccccactgccccagggacaggattccgacacattccttaacgactctctaagtctgggctggcggtcacagaggttggctggcgtttcctgcagaggccggtgacactctttccttccgccttcctccaaccccacccccagctccgcacacacccggcctcgccgcgaatccatccagagggtgtttgctctgcacttggattcctcaggaagcctccttgagctcccgcgggcgccgggcgcaccctccttccctcccgccccggcgcggggcgcggcgcctgctcgcgcactcagcacccgggctgcgcggcggaaacccctggcccgcctgcccgcctgcccgcctgcccgcagcaccctgccgcgcctcggcgtgggcctgcggaggggcgggggcggggagtgaaaaatagctgcagtagttgtagcaactggacaaacattccagagattggccgaggtgtgaaaatccgaggcttgtgtggattttctaattaaaatccaataaagggcccagcattgtcattgtgatttctgggataaacaggagacactcttggaaggggacacttccattttctcccctccctgttctcagctcactctctccccagctcgcgggcctccccctccctatttgctcaggtgcaacccccccccaccccgtgtgcaattcacaagcactatttgcacagccaggcctctcttccaagcactcaagatgtgtatttgaattttaaatggcggggcctgttcggagcgctggaagcgccgggtctcaaggccccttgactacccgctttctcgagccctctgtcgctctctgaggctcactgtccccaggacaataattgaagttggggcggagcgcagggggccggggaggagggcggggagggccgtcttctttgcattccaatctccgggatcatgctttttgcaaatagagattgccagaaagaatcttcctgctggaattacagggggcttttaatcttctcatcggtttctctggcatgaagtgcggggctggccgttggaggccctccgtagccccccggaatgcgaccccgggcgctggcgagcccgagtcgtcgagtcgcacggcccggcttgacacgctgcgccccgcgcccccgcccgtgtgctcagaatgccaatgagcggctcccgcgccgcggctccgctctcccggcgcgatccgggggatggattagcgctgctcacttgggaggaggcctccggagacccccgctagggggaccccgcgggcagggagccggggaggaggttttgcgcagggggcgccctgggcccaggggccgccgccgagcccgcgaggtgggggcgccgcctcctgcctgcctgcagccgggagctctccgggccgcacacccgcccctgccgcccccgctccacgcctttgctttctgtggggacgagaagccactctagcctttacctacaagtgtgccttgacaccttaacagtgttttcctactaacatagtgccattaacatggaatttaggagacaagtagggaagcctggaagggggttgaggaaggcagagtttccccgaaattcaccgtgaacggctttgctaaacgtgccaatttatgcgggcggagaagagcgctgtgcacacagcctcgggccggaggtttgcgtgcgcgcgatggcgcctcctccacggctgttcctcgccggcaacagcgtgtggcgttgcaggcccagagaagacagcctcgcgccgactcgggccggcgtttcggggcctgcccgccgcccagggcgtcccggcccggctcccagtaggaacgcaggctccgggcgcgccgcctccagatgctggaggtggttgcccctgggtttactgccgcccaccaggcctctccccaaaggcagcttaggatccacgggcgcccggcggggaggagccaggacgcgggctccggggcaggatagacacccctgcgcttgggatagcgggtgggggcggttggttcttaagccctaaacgagaggaaggagctcgcccagacctggctaaccgcgcagggctgtcttccctcgcctggaagggcctagcgcccctcccccagtcacgctttcccaccccctgcagtgggggctccagaagcgggcaggaagccacaggaccccatctcggggcatggagaaccttgggctctgtggcctcaaaggtaggggtgatttcgaggggccggcacctcacagggcaggttccaccgcggaaacgcagtcatcgcccagcgaccctgctcctggccctcagcctccccccaggtttctttttctcttgaatcaagccgaggtgcgccaatggccttccttgggtcggatccggggggccagggccagcttacctgctttcaccgagcagtggatatgtgccttggactcgtagtacacccagtcgaagccggcctccaccgccaggcgggccagcatgccgtacttgctgcggtcgcggtcagacgtggtgatgtccactgcgcggccctcgtagtgcagagactcctctgagtggtggccatcttcgtcccagccctcggtcacccgcagtttcactcctggccactggttcatcaccgagatggccaaagcgttcaacttgtccttacacctctgcgaagacaaggggacccccaccgacggacacgttagcctgggcaaccgccacccctcccggcccctccatcagcctgccctgcccagtctcaaggcgcgagggccatgcggagaggtggggagacgggggttggaatggcccagacacaccgaagagccg

SEQ ID NO: 127 - DNA, 1995 bp, Homo sapiens
cggcaccgcgcggcctccggagctgcacccccaaatccccgtggcgacttcatttgaagcgtggaagaagcaaccacgcaagtggagagtgggttctgaaagctctgggatgaaccaccctctctctttctgtcgttcctcttttaggaccccggctgcggcgaggaggaaggagccagcctagcagcttctgcgcctgtggccgcgggtgtcctggaggcctctcggtgtgacgagtgggggacccgaaggctcgtgcgccacctccaggcctggacgctgccctccgtcttctgcccccaataggtgcgccggaccttcaggccctggggtgaattcagctgctcctacatcagcttccggaaccaccaaaaattcaaattgggattttccggagtaaacaagagcctagagccctttgctcaatgctggatttaatacgtatatatttttaagcgagttggttttttcccctttgatttttgatcttcgcgacagttcctcccacgcatattatcgttgttgccgtcgttttctctccccgcgtggctccttgacctgcgagggagagagaggacaccgaagccgggagctcgcagggaccatgtatcagagcttggccatggccgccaaccacgggccgccccccggtgcctacgaggcgggcggccccggcgccttcatgcacggcgcgggcgccgcgtcctcgccagtctacgtgcccacaccgcgggtgccctcctccgtgctgggcctgtcctacctccagggcggaggcgcgggctctgcgtccggaggcgcctcgggcggcagctccggtggggccgcgtctggtgcggggcccgggacccagcagggcagcccgggatggagccaggcgggagccgacggagccgcttacaccccgccgccggtgtcgccgcgcttctccttcccggggaccaccgggtccctggcggccgccgccgccgctgccgcggcccgggaagctgcggcctacagcagtggcggcggagcggcgggtgcgggcctggcgggccgcgagcagtacgggcgcgccggcttcgcgggctcctactccagcccctacccggcttacatggccgacgtgggcgcgtcctgggccgcagccgccgccgcctccgccggccccttcgacagcccggtcctgcacagcctgcccggccgggccaacccggccgcccgacaccccaatctcggtgagtaggagcgcgagggctggggcgcgtgagggccggggcaggggccgtcttgagccctgtcgagggcctcttgtttttccaccaacgccttcgttgggctggggatggtgcttcactacctcgagtttctagggaaggcagaagccagtgcggggctggcgacatcacagccccagaagaccggcttctgtggaaggggccgggcctgcccgccggggcctcttctgagatggtgtcagggtcggagtgcggcctccccgccatcccagacatcgaccgtggccgcgctgcgctgtgggtgacgcgggaggacagcgggctccctggagagccgggggcagcggcctgggatttcctcgtggaaggtgctggagattgctgagtttctgcgcccctttcctccccgcccgccctcgggcctccgcagggaactgattacaatggtttggaccgcagaccttctgggccatttggcggcccagctggaggatccctcggggtagctgatgattttcccgtcgggggtctcacaccgagaacaaaggagggatggacaaaggagacgccggggagatgcgcggaacaggagccggcactgtgcgggtgccacccggccgagcgcgtgggcgcatcatgcgggcagcgggggggggggcgcacacgcccggtcagtgtccgggaacatagggacctcaaacgcgcttgttcatgacacccgagttaaatggagactttgcagtcgcttgcacgcgtggagcctcctcttctcgcg

SEQ ID NO: 128 - DNA, 4742 bp, Homo sapiens
cgaatggggaaacgcttctgtcgcgtgccccggcagagctgcgcgcagggcagaaccaaaggtccctccccctagattggaagctccatggggtagggccacaaggccgtcaccactgcgggctggccttttgggggactttgcggggcgctgggcgggagtccatccctcctcaggtgtcaggtctttgaactcaggcgcccgacaccgcggggccgcagagccacagagagacgcctagggggtggggtggattctgccgcccgggcagctggagtgcgcggcctattccatgctgacctgcgtgggggcgggtacaggggagccggaggcagcgcacactgttcgctcctcaccagcgctgcgccgcccgcactcaaggcaggtcgtcctccacgtgccgctggctgcatcccgatcccgcttgcgtcgggcggggctcagaggttcccggtgcgaccttcgcgggtggatggggcgggacgaggaggatccagggtggggatttgagatcaggtccctttcgggttttctttttgaagcgcccctctgcctccgcccgcgcctccgccaggctcgctgcgtcagcacctcaccggctttgcacacaggctgtgccacgcggccttcacccctgtgactccccgcagctcgcgcggatgcaccgacgagtcagcttgtcctctggaagccaatgagtctccccggcgccccgtggccgaagagctaggtgtaccatgcaatccccgcccgactggtccccggcgaccgacgccccgcggccctcgcccttcccgcccccggcgctgcacgccctccacggcttggcccgcgcgctgctgttcccggcctactgggccctgcaccagctgcagggtggctgaggagggcggcgggcggcgggcggcgctgctgctgctgctgctggtggtcggcccgcccctggcgctgcccggcctgccgctctggctggtgccgcaggtttggcgccgccccttctgctaccgctcccctccgctgtgctgggcgccgcccgcgctctggcgcccgcctgccgaacccgggcgctgcttcgtcttcctcaccgccaatctgtgcctgctccccgacgggcaagcgcgcttcggcaacctaccgcacagccagcgaccggccgaggccattggcgccgcgcggcccgcgctctacagggctacggactgcagccagccgtggtccagggcgccgcgcgggacgctggtggcctcgctgcccgtgggtctggactttgtgtgcctgcaggaggtattcgatctgcgcagggctcgtcgcttggtgaatcgcctggcgcccaacctgggcccgttgctgtacgacgtgggcacgctcggcctacagcccgggcggcacctcaagctgctggacagcgggctgctgctggcctcgcgctacccgctgctgcgcgccaccttccgttgcttcccccacgcacgtggcgaggacgcgctggcctccaagggactaccgtccgcacaggtactggcctatccggtgcagcccgcagagggggcgggggttcggccgagaaggcggagaaagctggagccgtccgtggtgttgcaggcgaggctgggcagcctggacgggcgccgcatcgtgggattcctgcactgcacgcatttgcaagcactggctggtgagccctgcggatgggtcctcggaacaggtcccgcctctccatagcggctgctgatccctacattcgcctgcaccccgccccccacctcccaccctttgttcactgcccccggcaccggaattcctgagaagtgcctgtcggggctcacagatcaggaactgcggagacgggagctgactgcccaggatgcgggtccgaatctggagccaagcccaccccacccccccctcccccacgcagaagacgggcccttgcgcagcaaacagctgacgccgctgctggactgggccgagaagtttggggccgagaagtttgaggccgacagagaacaaagtggagaggtcgtggctttcagcgtgctcctgggcgacctaaacttcgataactgctggaaaggtccagcaggagaggagggcgaggaggggcggagccaggaggcccggccccgcccaccgcgcgcccgcctctctctgcagaccaccggctagagcaggagcacgagctcttccgctgtttccaggatccctgccggctgggcacgcgccaaaagcagccctgggccctgggtatcgcgcttggggggagggtacccccgccggctgggcacgcgccaagagcagccctgggccctgggtatcgtgcttagggggagggtatcggagcgggaagtggacctggggagcgccgtcggctgaggctctggctgatgccgccctcccccggatcccccagggaccgcgctgagcacctccgtgctccaccagtccatggcctcctcccccaagatgccgaggcggtgagttgcgacctggatgtaggcactgcccgcccgaagcgcgcggaggggccctggccttgatgacaccgcccccctaccagggccctggagcaggagaaagggcgccacctctacctggccggccttcccggcagaagccgccgagctaagccctggagaggtcggcgcctggactacatcacgtaccgcggagttcccgggtggctgggcctgcggcactgggacgaccctcaacctgactcccgcccccaggaggtggagcaggtgacgttcagtaccgccctggaggggctcacggaccaccgggcagtgcgcctgcagctccgagtctcagtgtcctcctaaggcaagcacagatgaggggcgcgcggctggcgcgcacagacacgactcggagcacgaactaggcgccgtagctgcgtccccagaaccgggagacttaaggcatctttattgcgggatcctcacacggcctcctgggcccggcgatactcatagacgctgccgtgctcgggaaaggccagtgcttgcgggggcgaccccggcggtggggcggggtcctccgggtccccatagccaccgccgccgggcgtgtggagacagaacacatcctgttggcgcgggggggggcggggaggcgggctcagtgcaggcgtggcccctccctgtcacctgcgcagtcccctccccaccgggcagcgtccatcacccgcccccatggaccacggaggcccggtacgcgccccctttccttccggggccaaattaaactcggcctctggtcaggtctgcaactgttccccctctgtgggatggggccgggaggggaggggaggggagggggacaggacaggccgcggcccttaccccggggtacacggtcaccgacgtcttgccgcccagattcaccgtccggccgtttttgcggatcagcaggtttaggccgcgggcgccaggctcgccccctgcggagggaggcgaggagtccagagagaccaggggcgggggtgggggcgggggcggggagggccgcgagggctggggaaccgcgcaccgtggagcccgtatggccggaaggcgcggcgctcggtcagcactgacagcagcgcctcctcacgaaagagcagctcgcgggtgacgccgtcgccgcctcggaagcggcctctgccccccgagccccccgccgcagctcgaagcggcgcaggatgaccgggtacctgcgagggcgaggacgagggcaaagactcagcgtggcccgagccccggctcccacccggccgtgcccccagcctccgcgcacgccgctcaccggctctccaggatctcagggtcggtgatgcgtgtgttggtcatgtggctgtgcacaccgctgcgcccgtgccagctgggacccgcgcccgcgccgcccgccaccgtctcgtagtagcccatgtgggcgttgcccagggtcacgttgttcatgcagccctggtgagggcacctggtcgctgcgctgggctggggcagggcgtccccacctcgccctcccgctcctactccagcctgcccacccagtccgcccccgagctgcgcccaccccgcccccgcacctgggaggcggcgcaggccccaaaggcccccaggatgacatccaccacgcgctgcgacgtgagcacgttgccgcccaccaccgccgcctcgggcgacgggtccaggatggagcctcggggaatgaccacgcgcactggcgccaggcagccctgtgcggggcgggcggctctcaggagctcttggggtgggctccgggagaaacagcaccccaccccccgccccagcacccgtggggtcagaccgtggctgggcccaggaggcgcactcagggtgacctcgggctggggccccccctcgcgcacacacccctgcgaacctggttgagtgggatgtcgcggcccaccagacagcgcaggcagtagatgagggcggacagggttacggcccgcggtgcgttgagattaccaaacacctccggcccagtgccgctgaagtcaaacacggcgctgccctgcgcgccccgagggaagggagaggctgtcagcggccgcaccgtgcccctgccgcctagaggcactctccccacggcccacctgactcaggctgatctgcacacggaggcggatgggggaaccgtcgtccatgtggtcttccgaggacacctccaggggcaggccccgggcctgccgggaggttccaaaggcacgcaacatgtctcgcacggccagctcagcg

SEQ ID NO: 129 - DNA, 1267 bp, Homo sapiens
cgggccaaggcggcgggaaaggcaccgctacctgcagccgcacgactccaccaccatgtcctcgtactgcttgtagaccacattattgcccgcgtcgatgtatagaatgctgatgggagtcaatttggtgggcacgcagcagctgggcggggtggagccggggtccatggagttcatcagcgtctggatgatggcgtggttggtgggctccaggtgcgagcgcagcgggaagtcgcatacaccctcgcagtgataggcctcgtactccaggggcgcgataatccagtcgtcccagcccagctccttgaagttcacgtgcaggggcttcttgctgcagcgtagcctggacttcttgccgtgccgcttgccatggcgactggcgaaggccgtgcgccgccgccggcggccgggcgagggcagccaaggcctggcatccggggcgcccgacggcggcggccacgacccctcggcgcccgcgcccgggcccgcagcctcggccgagcccagctgctcgcgcatctctgcgaacaggttcttgcgctgggatctggtgaataccaccagcagggcccgctcctggggaggccgcaccctccggccgaagcccagactccgcaggtccgggggcggcggttgctggggtccccgcgcgcgcgcctcggcctccccggcgtccagctcgccccatgcggcccgcagctccaagcacagctgcttccagggctggtggcgcaggccctgccacacgtcgaagacttcccagccggccggcggcgccccctgcgggtccagggtccgcgcgtccagcagtaggggcgaaaggcaagggaagagctgcacgtggagcggcccggctggtggcccccagggcgctgagggcgcctggcgaaagagccgcagctccgcgcccaccagctcttctttgtctgagagcatggacacatcaaacaaatacttctgtctccggagaggagtgtgcgagagatcgtctgcgagataaaaaataattacagtcagtttcacttaagggggagatcagcccggtgctcttcggccgccccgggaggaaaagggcggggagtgggggcaggtcggccgggcagtccagcttgcccggcccagggcctgaccaccccggctccccatctggctggtgcatggcgcggggaagggggcgcgccaggacgggccccctccttcgcgtcagctccggactctcagggcggaagtcggtggctgctggcgaggcggcggcggcggcctaccg

SEQ ID NO: 130 - DNA, 270 bp, Homo sapiens
cggtccgtgggcgctgggctactggaggggaagcgaggaggcggggaaggggcgggccccaggagcaggcgggcggggctccccgccctccagcgcgcccggaggctaccactccctgcagatgcgctggccccagcccggggctgccgctcgccttcccccggagtctcctggacccccggagtctcctggacccccggagcgggaggcagcggccgcgcggcgctggactggagcagaaccccaggactgcgcccccgggagcgggcg

SEQ ID NO: 131 - DNA, 462 bp, Homo sapiens
cgtctcatgccagcctcggcgcagctccggagagcgggaggcggaggctcagagcggtgcagccccaccgggccccagcccgttgcctccgcccccacctcaccccatccccagcagcaccacttccgctcaggcctggctgctggcaaaatctcggcacagagggaggagggggagaggaaaacgcatgattcctcctcaaaatggagtcagccgaaaaaagcgtgaatgcagagcccgaagagactcctgggggaggggagcccctgcagggccagccgagggccggcgcaatggcttatctgagggacaggcagaaggacggacccccacggtggaccccagctacgcaccgtgtcgtggtggggcgggaaggcgaaggtgtactcgtctgccagcagcctgcggtaggcgtagtcctcgtgcgcgcggctcccgtaagccccgtagtagtcgtactcg

SEQ ID NO: 132 - DNA, 569 bp, Homo sapiens
cgccggaggggcggggccgggacccaaggctgggtcctgagcgccccgagggggcggccctgccaagccgaagagcggagtagcggtgtaagcctggcgacggccctcgcgccgattgctgtctatggcggcctggagctcccctggggagctgagcgctcgagtctcgcactcgtacgggtacaggtacttcatgtacctaggggcggacagcgggcggtgagtgtatgggaggctgcacccagctggtcaagtcccacagccggacgcccgcccggggaccctcccccctcccgccccaggccgaacccgtgcccccttcccttccgctcccgcccctggcctcactgggtgcgtagagtgaaggcggccgaggtgatggtggtgggtaggctgaggccgcgcgtgacttcccgccacactttgcggttgatgacttccaccaggccgcccttggcggtcaccaggcgaaacagagcgtacaggtcgagcacctgcttcgccatgatgggcacgcggttcactggcgtccctggtggggagcgggctgccgtcaggacactgagacg

SEQ ID NO: 133 - DNA, 10 bp, Artificial Sequence
  Synthetic
aaacgcgcgg

SEQ ID NO: 134 - DNA, 10 bp, Artificial Sequence
  Synthetic
tttcgcgcgg

SEQ ID NO: 135 - DNA, 10 bp, Artificial Sequence
  Synthetic
gggcgcgcgg

SEQ ID NO: 136 - DNA, 39 bp, Artificial Sequence
  Synthetic
tttccctacacgacgctcttccgatcthhhhhhhhcgch

SEQ ID NO: 137 - DNA, 39 bp, Artificial Sequence
  Synthetic
tttccctacacgacgctcttccgatcthhhhhhhcghch

SEQ ID NO: 138 - DNA, 39 bp, Artificial Sequence
  Synthetic
tttccctacacgacgctcttccgatcthhhhhhcghhch

SEQ ID NO: 139 - DNA, 39 bp, Artificial Sequence
  Synthetic
tttccctacacgacgctcttccgatcthhhhhcghhhch

SEQ ID NO: 140 - DNA, 45 bp, Artificial Sequence
  Synthetic
gtgactggagttcagacgtgtgctcttccgatctddddcgcgcgg

SEQ ID NO: 141 - DNA, 58 bp, Artificial Sequence
  Synthetic
aatgatacggcgaccaccgagatctacactctttccctacacgacgctcttccgatct

SEQ ID NO: 142 - DNA, 54 bp, Artificial Sequence
  Synthetic
caagcagaagacggcatacgagatctgatcgtgactggagttcagacgtgtgct